(19) United States
(12) Patent Application Publication
(10) Pub. No.: US 2026/0295086 A1
(43) Pub. Date: Oct. 1, 2026

(54) EPIGENETIC TARGETS FOR ENHANCING CANCER IMMUNOTHERAPY

(71) Applicant: Duke University, Durham, NC (US)

(72) Inventors: Charles A. Gersbach, Chapel Hill, NC (US); Sean McCutcheon, Durham, NC (US)

(21) Appl. No.: 19/477,032

(22) PCT Filed: Apr. 19, 2024
(86) PCT No.: PCT/US2024/025594

§ 371 Date: Oct. 20, 2025

Related U.S. Application Data
(60) Provisional application No. 63/497,688, filed on Apr. 21, 2023.
(60) Provisional application No. 63/498,755, filed on Apr. 27, 2023.

(51) Int. Cl. A61K 48/00; A61K 38/17; A61K 45/06; A61P 35/00; A61P 37/04; C07K 14/47; C12N 9/22; C12N 15/11; C12N 15/113

(52) U.S. Cl. CPC A61K 48/005; A61K 38/1709; A61K 45/06; A61P 35/00; A61P 37/04; C07K 14/4703; C12N 9/226; C12N 15/111; C12N 15/113; C07K 2319/09; C07K 2319/71; C07K 2319/81; C12N 2310/14; C12N 2310/20; C12N 2310/531; C12N 2830/48; C12Y 201/01037

(57) ABSTRACT

Disclosed herein are compositions and methods for modulating T cells. For example, the compositions and methods may be used to increase memory T cells. The compositions and method may be used in combination with Adoptive T Cell Therapy (ACT) to enhance the ACT.

### CROSS-REFERENCE TO RELATED APPLICATIONS

This application claims priority to U.S. Provisional Patent Application No. 63/497,688, filed Apr. 21, 2023, and U.S. Provisional Patent Application No. 63/498,755, filed Apr. 27, 2023, the entire contents of each of which are hereby incorporated by reference.

### FIELD

This disclosure relates to genes to target for repression or activation to increase T cells such as memory T cells, as well as compositions and methods targeting the genes to improve adoptive T cell therapy (ACT).

### INTRODUCTION

Adoptive T cell therapy (ACT) holds tremendous potential for cancer treatment by redirecting T cells to cancer cells via expression of engineered receptors that recognize and bind to tumor-associated antigens. Receptor-antigen interactions can initiate complex transcriptional networks that drive multipotent T cell response and lead to cancer cell death. The potency and duration of T cell response are associated with defined T cell subsets, and cell products enriched in stem or memory T cells, provide superior tumor control in animal models and in the clinic. Given the association between defined T cell subsets and clinical outcomes, precise regulation or programming of T cell state may be one approach to improve the therapeutic potential of ACT.

T cell state and function are largely regulated by specific transcription factors (TFs) and epigenetic modifiers that process intrinsic and extrinsic signals into complex and tightly controlled gene expression programs. For example, TOX6-10 and NFAT11 program CD8+ T cell exhaustion in the context of chronic antigen exposure. Conversely, T cell function can be enhanced by rewiring transcriptional networks through either enforced expression or genetic deletion of specific TFs and epigenetic modifiers. Ectopic overexpression of specific TFs such as c-JUN, BATF, and RUNX3 or genetic deletion of NR4A, FLI1, members of the BAF chromatin remodeling complex, and regulators of DNA methylation can alter T cell state and improve T cell function through diverse mechanisms. There is a need to find and develop regulators of T cell state and discover T cell gene networks and their corresponding phenotypes, In order to enhance T cell phenotype and improve the efficacy of T cell therapies to help kill cancer cells and control solid tumors.

### SUMMARY

In an aspect, the disclosure relates to a composition comprising a modulator of a gene selected from ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. In some embodiments, the gene is ZNF217 or ETS1. In some embodiments, the gene is RBSN, PRDM1, GATA3, or RUNX3. In some embodiments, the modulator is an inhibitor and the gene is ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1. In some embodiments, the modulator is an activator and the gene is FOXO1, MDB2, YY1, DNMT1, IRF1, CTCF, GTF2B, or HIC1. In some embodiments, the composition further includes a modulator of BATF3. In some embodiments, the composition modulates T cells, and modulating T cells may include increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof. In some embodiments, the composition modulates gene expression within the T cell. In some embodiments, the composition increases expression of IL7RA in the T cell, or decreases expression of CCR7 in the T cell, or a combination thereof. In some embodiments, the modulator comprises a polypeptide, or a polynucleotide, or a small molecule, or siRNA, or shRNA, or a combination thereof. In some embodiments, the modulator comprises siRNA or shRNA, or a polynucleotide selected from SEQ ID NOs: 337-369 or a fragment thereof, or a polypeptide selected from SEQ ID NOs: 370-402 or a fragment thereof. In some embodiments, the modulator comprises a DNA targeting composition, the DNA targeting composition comprising: (a) a Cas9 protein and at least one guide RNA (gRNA) that targets the Cas9 protein to the gene or a regulatory element thereof; or (b) a meganuclease, or (c) a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof. In some embodiments, the second polypeptide domain comprises a meganuclease.

In a further aspect, the disclosure relates to therapy including a first composition comprising a composition as detailed herein; and a second composition comprising a modulator of BATF3.

In a further aspect, the disclosure relates to DNA targeting composition. The DNA targeting composition may include a Cas9 protein or a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity; and at least one guide RNA (gRNA) that targets the Cas9 protein to a target gene or a regulatory element thereof when the DNA targeting composition comprises a Cas9 protein, wherein the target gene is selected from ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. In some embodiments, the gene is ZNF217 or ETS1. In some embodiments, the gene is RBSN, PRDM1, GATA3, or RUNX3. In some embodiments, the gene is ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1 and the DNA targeting composition is an inhibitor of the gene. In some embodiments, the gene is FOXO1, MDB2, YY1, DNMT1, IRF1, CTCF, GTF2B, or HIC1 and the DNA targeting composition is an activator of the gene. In some embodiments, the gRNA is encoded by a polynucleotide comprising a sequence selected from SEQ ID NOs: 73-204, or comprises a sequence selected from SEQ ID NOs: 205-336. In some embodiments, the Cas protein comprises a Streptococcus pyogenes Cas9 protein, or a Staphylococcus aureus Cas9 protein, or any fragment thereof. In some embodiments, the Cas9 protein comprises the amino acid sequence of one of SEQ ID NOs: 26-29, or any fragment thereof, and/or the Cas9 protein is encoded by a polynucleotide comprising a sequence selected from SEQ ID NOs: 30-39 and/or the Cas9 protein comprises an amino acid sequence having at least 90% or greater identity to a sequence selected from SEQ ID NOs: 26-29, or any fragment thereof, and/or the Cas9 protein is encoded by a polynucleotide comprising a sequence having at least 90% or greater identity to a sequence selected from SEQ ID NOs: 30-39, or any fragment thereof, and/or the Cas9 protein comprises an amino acid sequence having one, two, three, four, five or more changes selected from amino acid substitutions, insertions, or deletions, relative to a sequence selected from SEQ ID NOs: 26-29, or any fragment thereof, and/or the Cas9 protein is encoded by a polynucleotide comprising a sequence having one, two, three, four, five or more changes selected from nucleotide substitutions, insertions, or deletions, relative to a sequence selected from SEQ ID NOs: 30-39, or any fragment thereof. In some embodiments, the fusion protein comprises more than one second polypeptide domain. In some embodiments, the second polypeptide domain comprises a polypeptide selected from VP16, VP64, p65, TET1, VPR, VPH, Rta, p300, p300 core, KRAB, MECP2, EED, ERD, Mad mSIN3 Interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3. In some embodiments, the second polypeptide domain has transcription repression activity. In some embodiments, the gene is ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1. In some embodiments, the second polypeptide domain comprises KRAB or Fokl. In some embodiments, KRAB comprises the amino acid sequence of SEQ ID NO: 45, or any fragment thereof, and/or KRAB is encoded by a polynucleotide comprising the sequence of SEQ ID NO: 46, and/or KRAB comprises an amino acid sequence having at least 90% or greater identity to SEQ ID NO: 45, or any fragment thereof, and/or KRAB is encoded by a polynucleotide comprising a sequence having at least 90% or greater identity to SEQ ID NO: 46, or any fragment thereof, and/or KRAB comprises an amino acid sequence having one, two, three, four, five or more changes selected from amino acid substitutions, insertions, or deletions, relative to SEQ ID NO: 45, or any fragment thereof, and/or KRAB is encoded by a polynucleotide comprising a sequence having one, two, three, four, five or more changes selected from nucleotide substitutions, insertions, or deletions, relative to SEQ ID NO: 46, or any fragment thereof. In some embodiments, the fusion protein comprises the amino acid sequence of SEQ ID NO: 47 or 49, or any fragment thereof, and/or the fusion protein is encoded by a polynucleotide comprising the sequence of SEQ ID NO: 48 or 50, and/or the fusion protein comprises an amino acid sequence having at least 90% or greater identity to SEQ ID NO: 47 or 49, or any fragment thereof, and/or the fusion protein is encoded by a polynucleotide comprising a sequence having at least 90% or greater identity to SEQ ID NO: 48 or 50, or any fragment thereof, and/or the fusion protein comprises an amino acid sequence having one, two, three, four, five or more changes selected from amino acid substitutions, insertions, or deletions, relative to SEQ ID NO: 47 or 49, or any fragment thereof, and/or the fusion protein is encoded by a polynucleotide having one, two, three, four, five or more changes selected from nucleotide substitutions, insertions, or deletions, relative to SEQ ID NO: 48 or 50. In some embodiments, the second polypeptide domain has transcription activation activity. In some embodiments, the gene is FOXO1, MDB2, YY1, DNMT1, IRF1, CTCF, GTF2B, or HIC1. In some embodiments, the second polypeptide domain comprises a polypeptide selected from VP16, VP64, p65, TET1, VPR, VPH, Rta, and p300, or a fragment thereof. In some embodiments, the second polypeptide domain comprises VP64, p300, VPH, or VPR, or a fragment thereof. In some embodiments, the second polypeptide domain comprises the amino acid sequence of SEQ ID NO: 41, 42, 53, or 55, or any fragment thereof, and/or the second polypeptide domain is encoded by a polynucleotide comprising the sequence of SEQ ID NO: 54 or 56, and/or the second polypeptide domain comprises an amino acid sequence having at least 90% or greater identity to SEQ ID NO: 41, 42, 53, or 55, or any fragment thereof, and/or the second polypeptide domain is encoded by a polynucleotide comprising a sequence having at least 90% or greater identity to SEQ ID NO: 54 or 56, or any fragment thereof, and/or the second polypeptide domain comprises an amino acid sequence having one, two, three, four, five or more changes selected from amino acid substitutions, insertions, or deletions, relative to SEQ ID NO: 41, 42, 53, or 55, or any fragment thereof, and/or the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof. In some embodiments, the fusion protein comprises the amino acid sequence of SEQ ID NO: 43, or any fragment thereof, and/or the fusion protein is encoded by a polynucleotide comprising the sequence of SEQ ID NO: 44, and/or the fusion protein comprises an amino acid sequence having at least 90% or greater identity to SEQ ID NO: 43, or any fragment thereof, and/or the fusion protein is encoded by a polynucleotide comprising a sequence having at least 90% or greater identity to SEQ ID NO: 44, or any fragment thereof, and/or the fusion protein comprises an amino acid sequence having one, two, three, four, five or more changes selected from amino acid substitutions, insertions, or deletions, relative to SEQ ID NO: 43, or any fragment thereof, and/or the fusion protein is encoded by a polynucleotide comprising a sequence having one, two, three, four, five or more changes selected from nucleotide substitutions, insertions, or deletions, relative to SEQ ID NO: 44.

Another aspect of the disclosure provides a composition for increasing T cells, the composition comprising an inhibitor of a gene selected from ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1. In some embodiments, the inhibitor comprises a shRNA or siRNA targeting the gene or a fragment thereof. In some embodiments, the inhibitor comprises a DNA targeting composition as detailed herein, and the second polypeptide domain has transcription repression activity.

Another aspect of the disclosure provides a composition for increasing T cells, the composition comprising an activator of a gene selected from FOXO1, MDB2, YY1, DNMT1, IRF1, CTCF, GTF2B, or HIC1. In some embodiments, the activator comprises a polynucleotide encoding the gene, or a polypeptide encoded by the gene, or a combination thereof or a fragment thereof. In some embodiments, the activator comprises a polynucleotide selected from SEQ ID NOs: 337-369 or a fragment thereof, or a polypeptide selected from SEQ ID NOs: 370-402 or a fragment thereof. In some embodiments, the activator comprises a DNA targeting composition as detailed herein, and the second polypeptide domain has transcription activation activity.

In some embodiments, the composition further comprises an activator of the BATF3 gene. In some embodiments, the activator of the BATF3 gene comprises a polynucleotide encoding BATF3, or a BATF3 polypeptide, or a DNA targeting composition as detailed herein, wherein the second polypeptide domain has transcription activation activity, or a combination thereof. In some embodiments, the composition further includes at least one cancer therapy.

Another aspect of the disclosure provides an isolated polynucleotide sequence as detailed herein. Another aspect of the disclosure provides a vector comprising an isolated polynucleotide sequence as detailed herein. Another aspect of the disclosure provides a cell comprising a composition as detailed herein, or an isolated polynucleotide sequence as detailed herein, or a vector as detailed herein, or a combination thereof. In some embodiments, the cell is a CD8+ T cell. Another aspect of the disclosure provides a pharmaceutical composition comprising a composition as detailed herein, or an isolated polynucleotide sequence as detailed herein, or a vector as detailed herein, or a combination thereof.

Another aspect of the disclosure provides a method of modulating T cells. The method may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, modulating T cells comprises increasing T cells, or increasing memory T cells, or preventing T cell exhaustions, or reversing T cell exhaustions, or a combination thereof. In some embodiments, the composition or isolated polynucleotide sequence or vector is administered to a T cell, and the T cell thereby increases expression of IL7RA, or decreases expression of CCR7, or a combination thereof.

Another aspect of the disclosure provides a method of increasing T cells. The method may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof.

Another aspect of the disclosure provides a method of enhancing adoptive T cell therapy (ACT) in a subject. The method may include administering to the subject a composition as detailed herein, or an isolated polynucleotide sequence as detailed herein, or a vector as detailed herein, or a cell as detailed herein, or a pharmaceutical composition as detailed herein, or a combination thereof.

Another aspect of the disclosure provides a method of treating cancer in a subject. The method may include administering to the subject a composition as detailed herein, or an isolated polynucleotide sequence as detailed herein, or a vector as detailed herein, or a cell as detailed herein, or a pharmaceutical composition as detailed herein, or a combination thereof. In some embodiments, the method further includes administering an additional cancer therapy.

The disclosure provides for other aspects and embodiments that will be apparent in light of the following detailed description and accompanying figures.

### BRIEF DESCRIPTION OF THE DRAWINGS

FIGS. 1A-1K show compact and efficient dSaCas9 epigenome editors for targeted gene silencing and activation. FIG. 1A is a schematic showing an all-in-one lentiviral plasmid encoding for dSaCas9^{KRAB} and a gRNA cassette. FIG. 1B is a schematic showing CD2 and B2M promoter tiling CRISPRi screens in human CD8+ T cells. FIG. 1C is a volcano plot of significance (P_{adj}) versus fold change in gRNA abundance between CD2-high and CD2-low populations for the CD2 CRISPRi screen. Dark gray data points indicate CD2 gRNA hits with a P_{adj}<0.05 or log₂(fc)<−1. Black data points indicate non-significant CD2 gRNAs and light gray data points indicate the 250 non-targeting (NT) gRNAs. FIG. 1D is a scatter plot showing CD2 gRNA fold change versus gRNA position relative to the transcriptional start site (TSS). Dashed lines represent the previously defined optimal window (−50 to +300 bp of TSS) for CRISPRi. FIG. 1E is a scatter plot showing CD2 gRNA fold change as a function of the final base pair of the PAM (5′-NNGRRN-3′). x represents the number of gRNA hits and y represents the total number of gRNAs in the library for each PAM variant. A one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 1F is a graph showing validation of CD2 gRNA hits. Percentage of CD2 positive cells on day 9 post-transduction plotted in rank order based on the mean gRNA activity (n=3 replicates of CD8+ T cells from pooled PBMC donors, error bars represent SEM). A one-way ANOVA with Dunnett's post hoc test was used to compare the mean percentage of CD2 positive cells for each gRNA to NT. The final base pair of the PAM for each gRNA is indicated beneath the gRNA label. FIG. 1G is Relationship between CD2 gRNA activity and fold enrichment in screen. Relative CD2 mean fluorescent intensity (MFI) was calculating by normalizing the MFI of each gRNA to the MFI of the NT population.

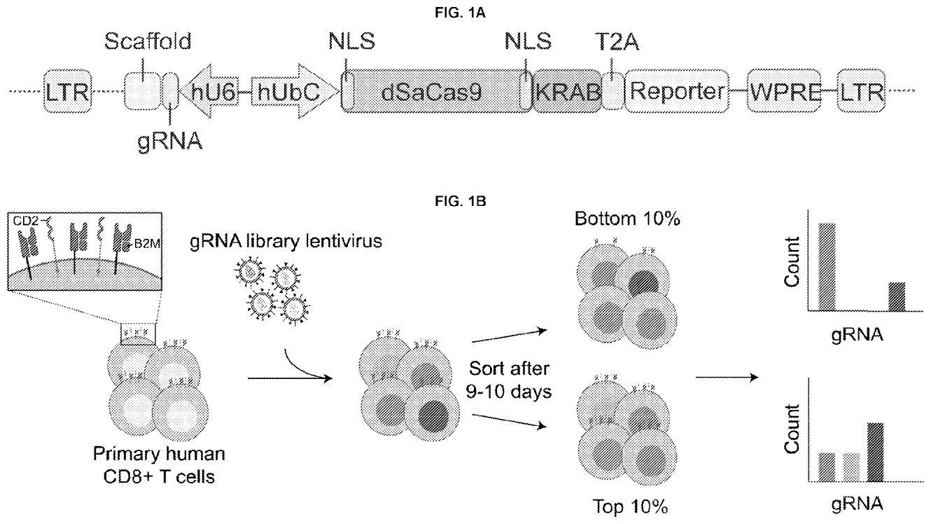

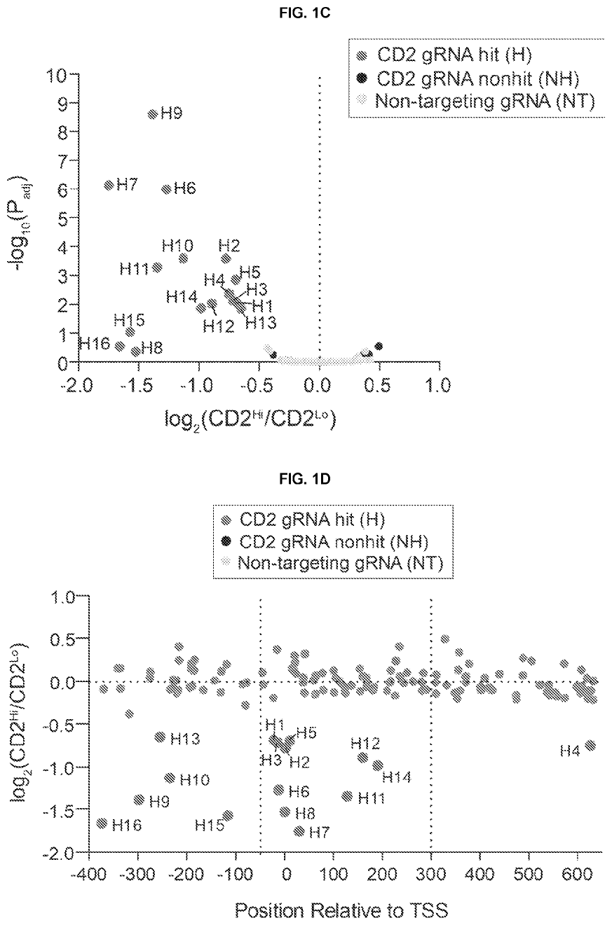

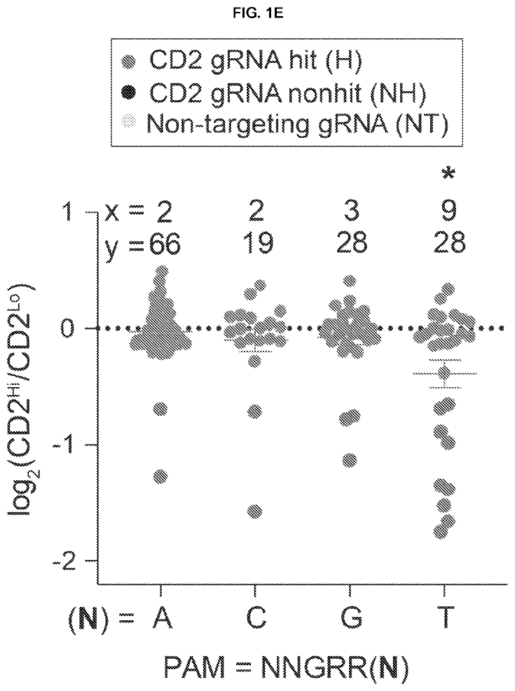

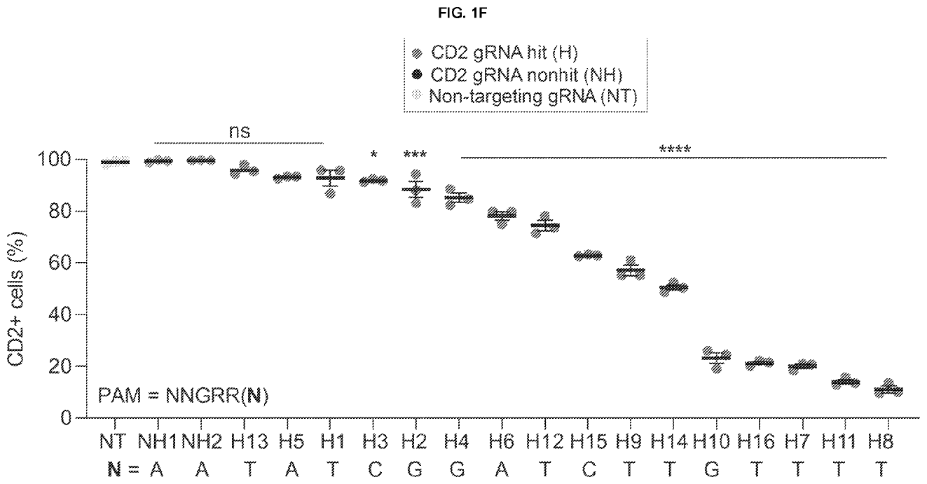

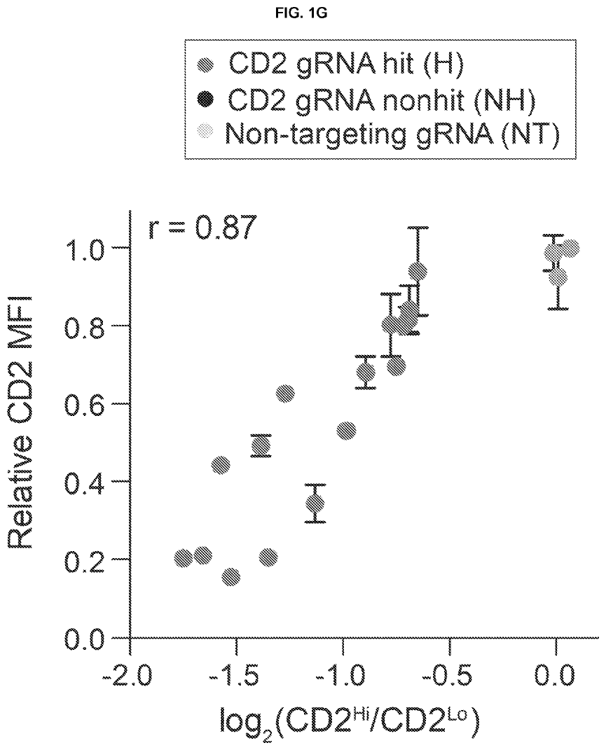

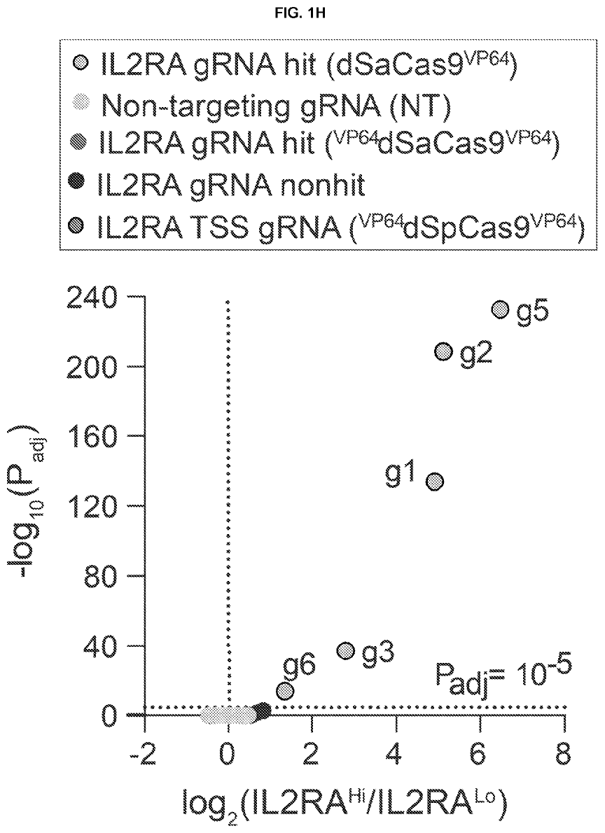

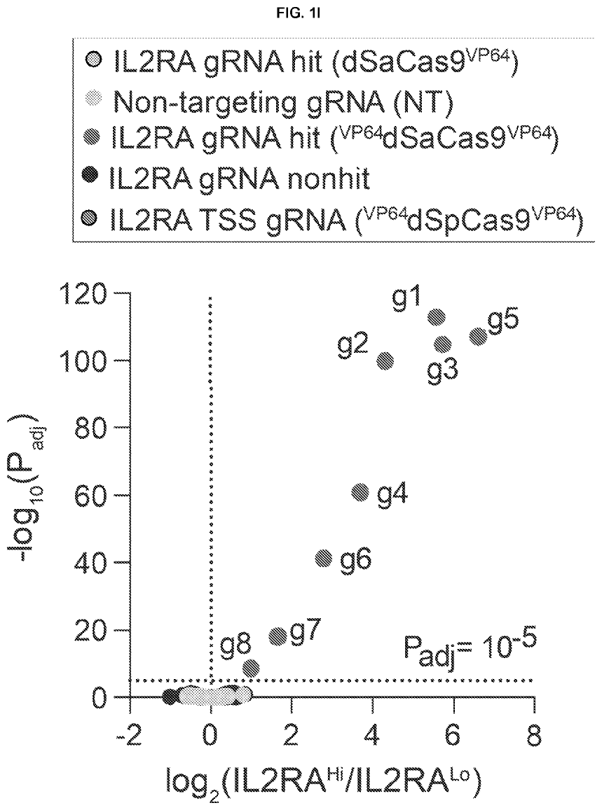

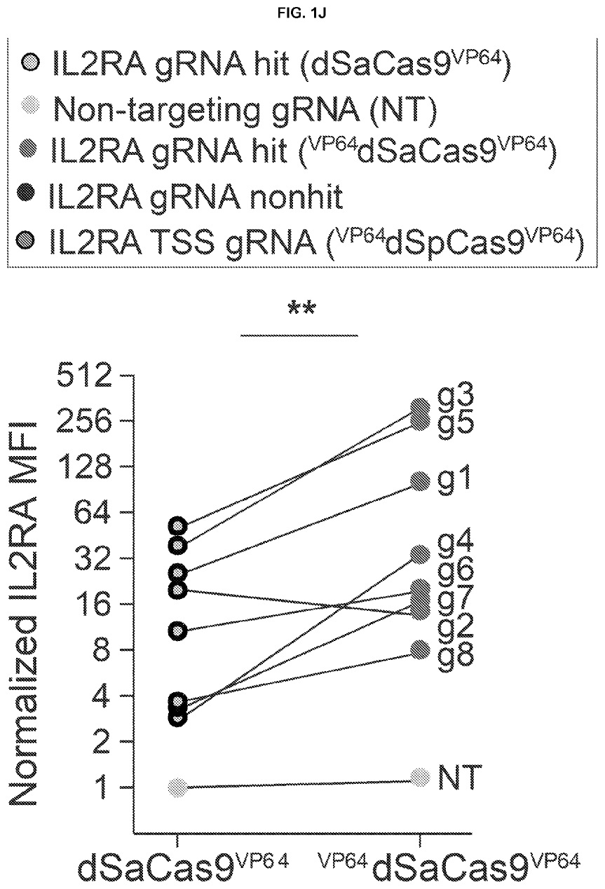

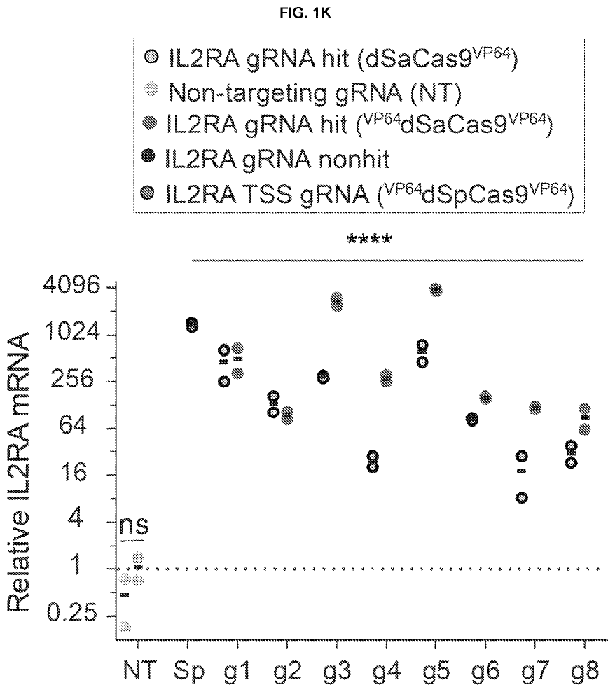

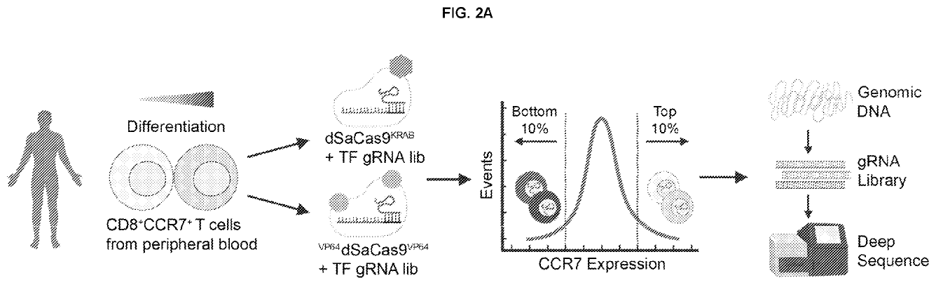

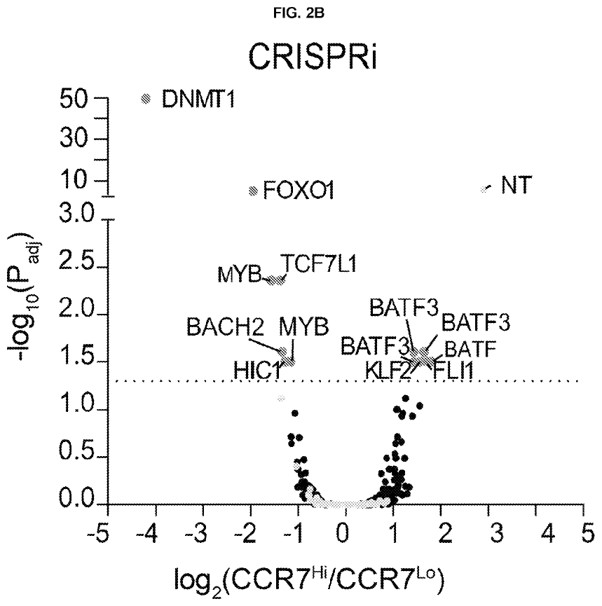

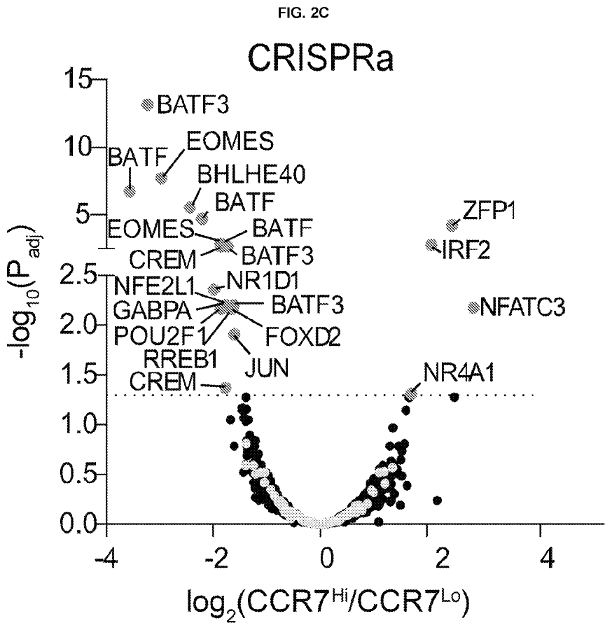

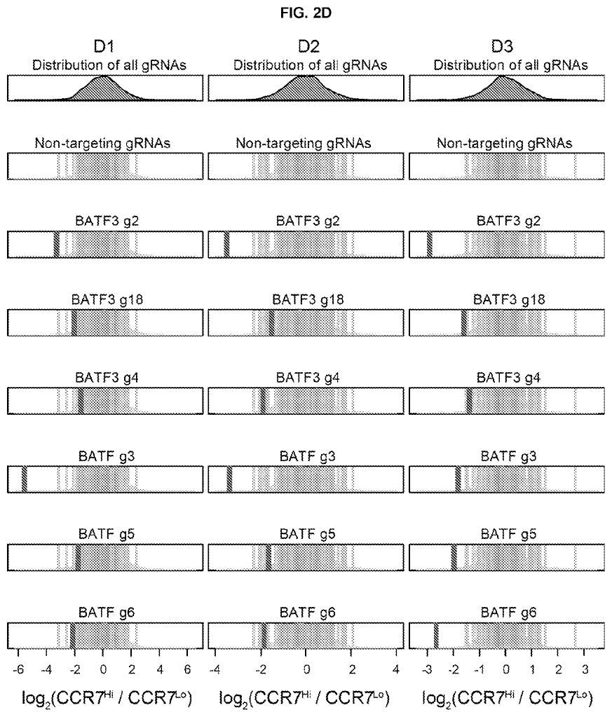

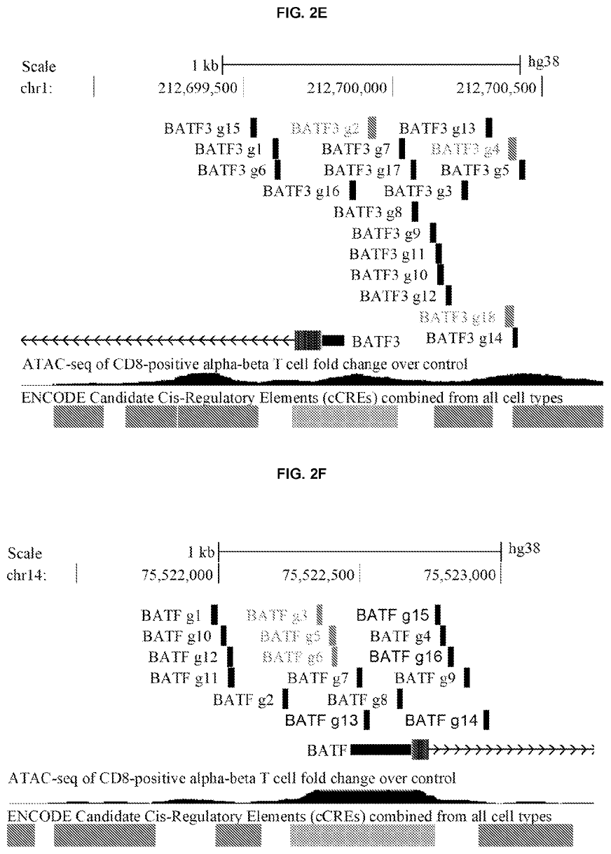

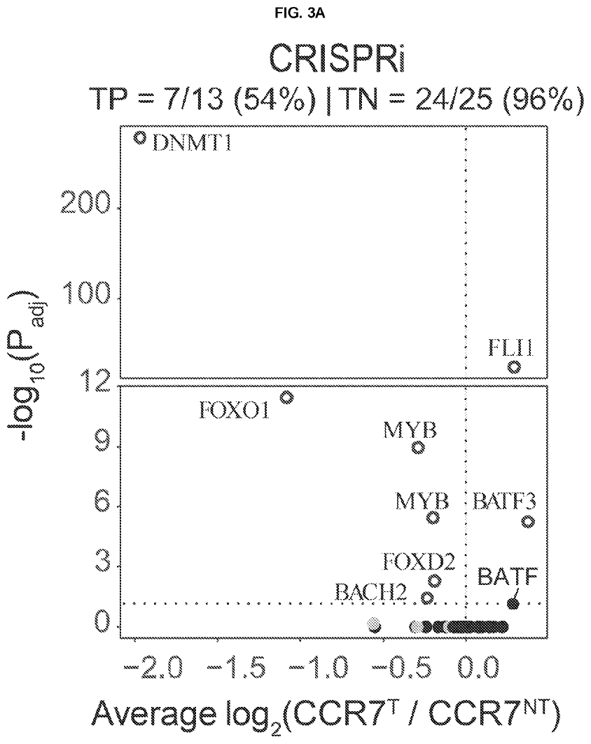

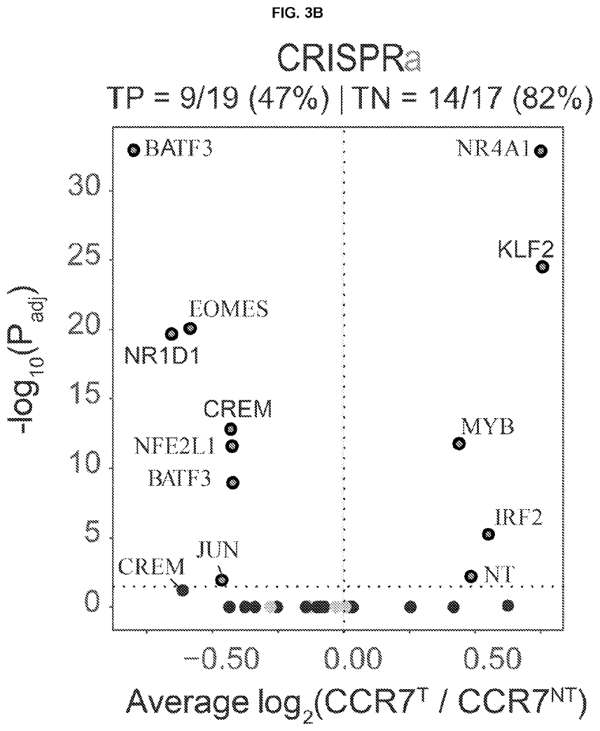

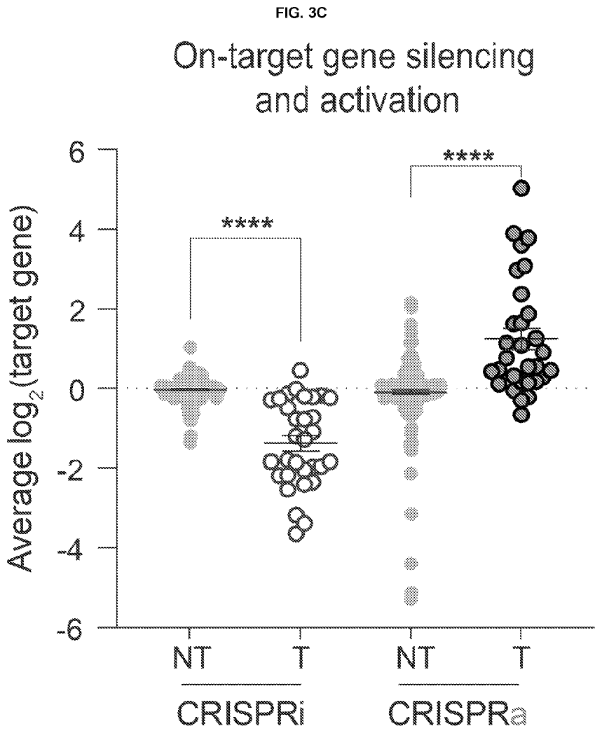

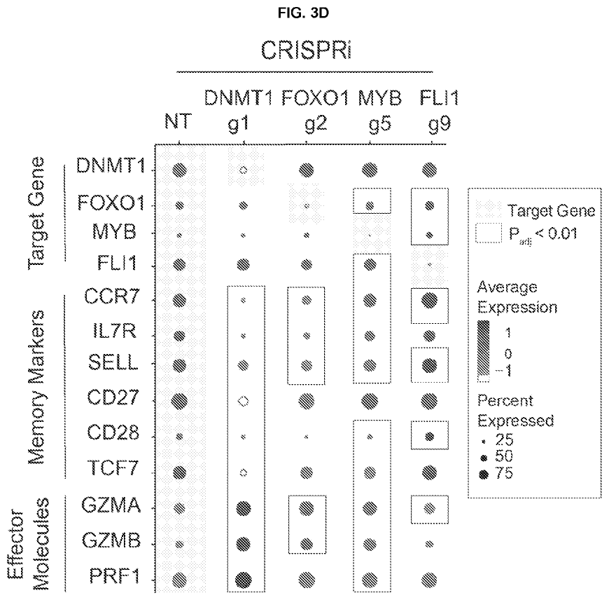

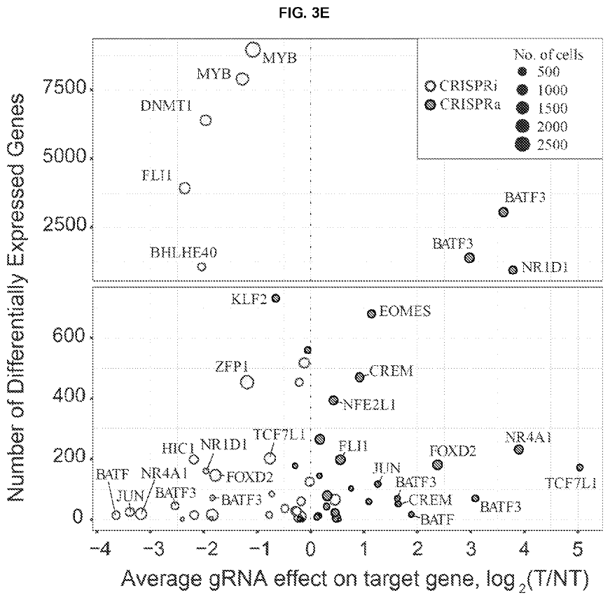

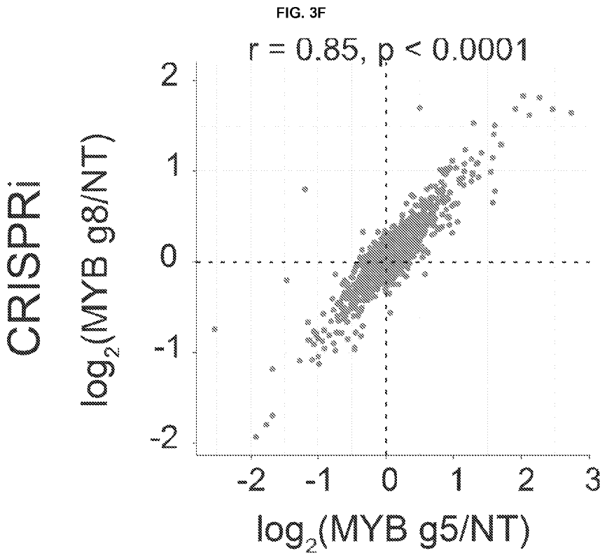

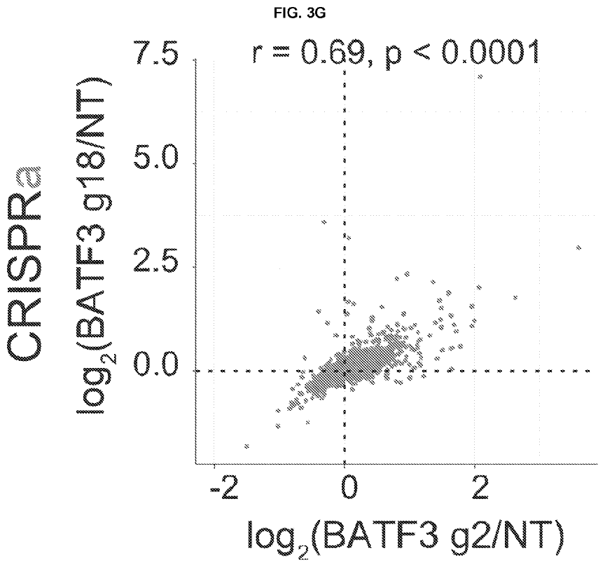

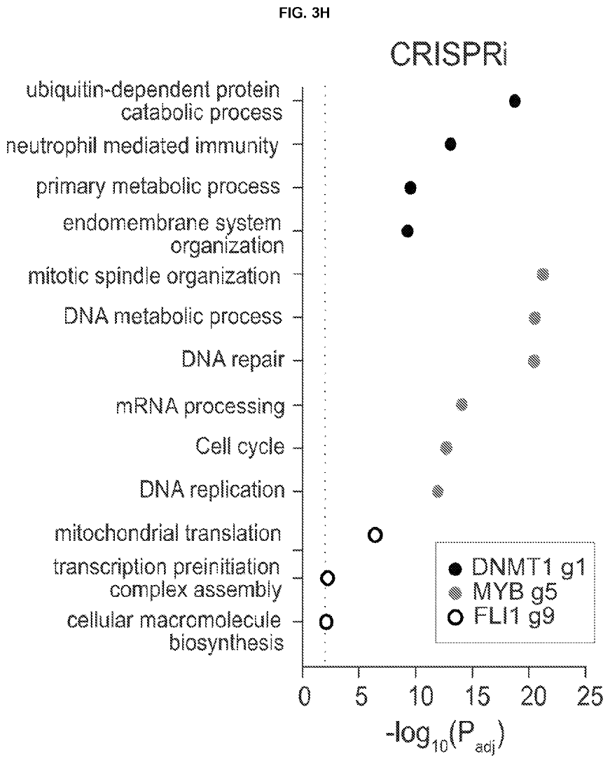

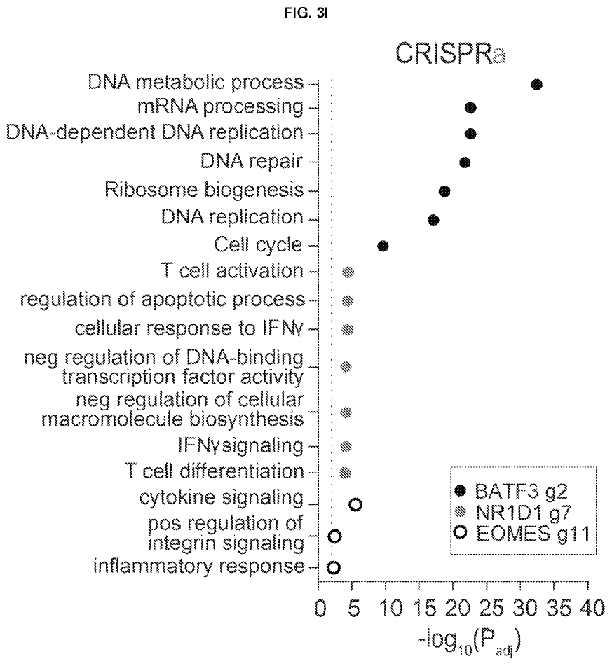

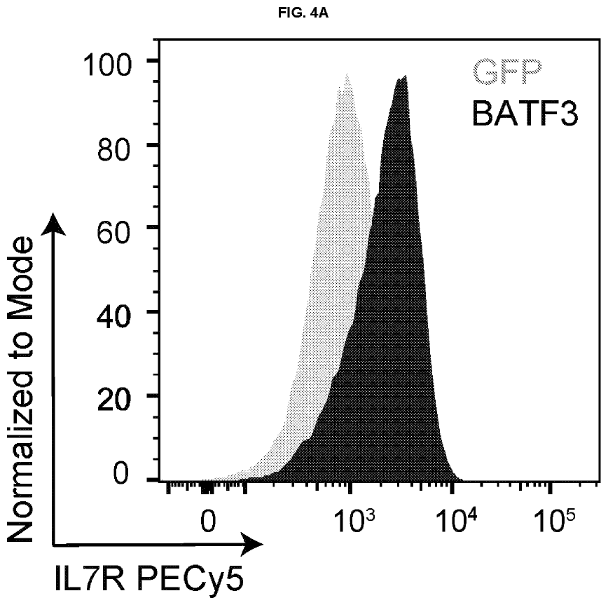

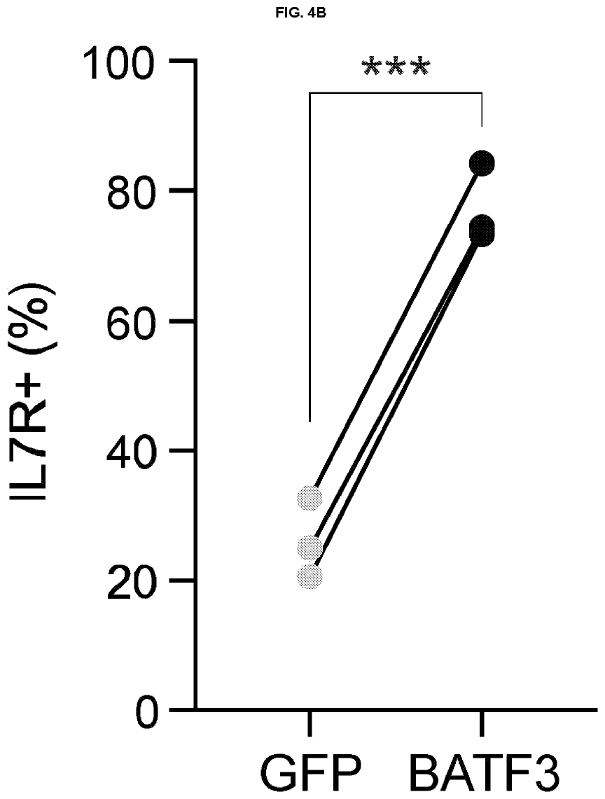

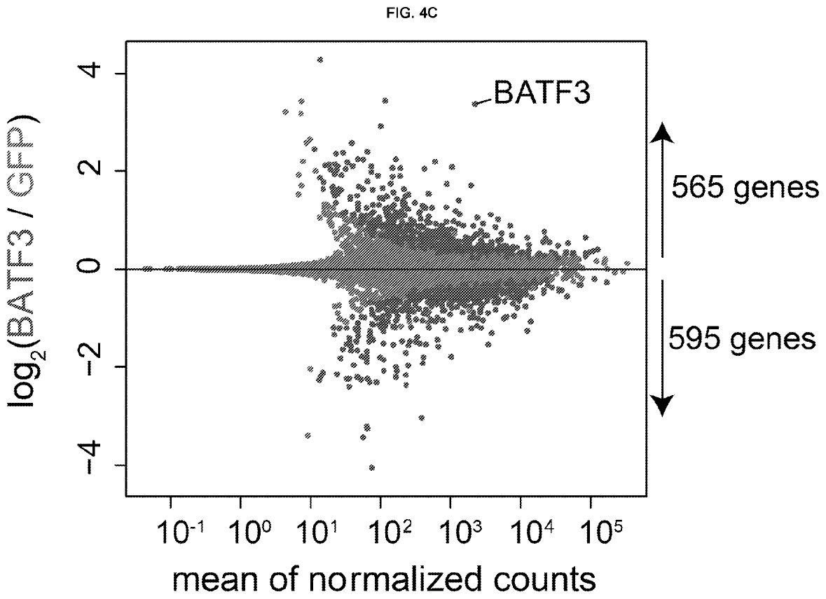

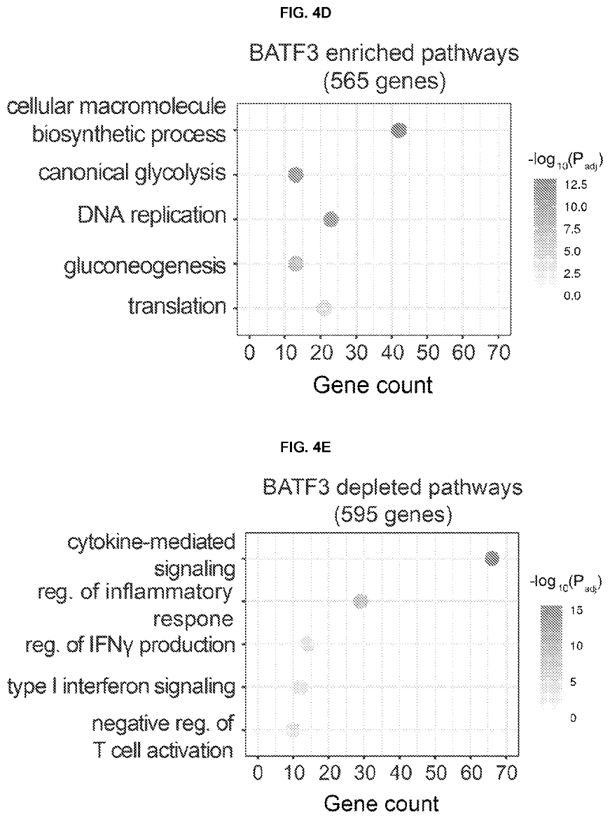

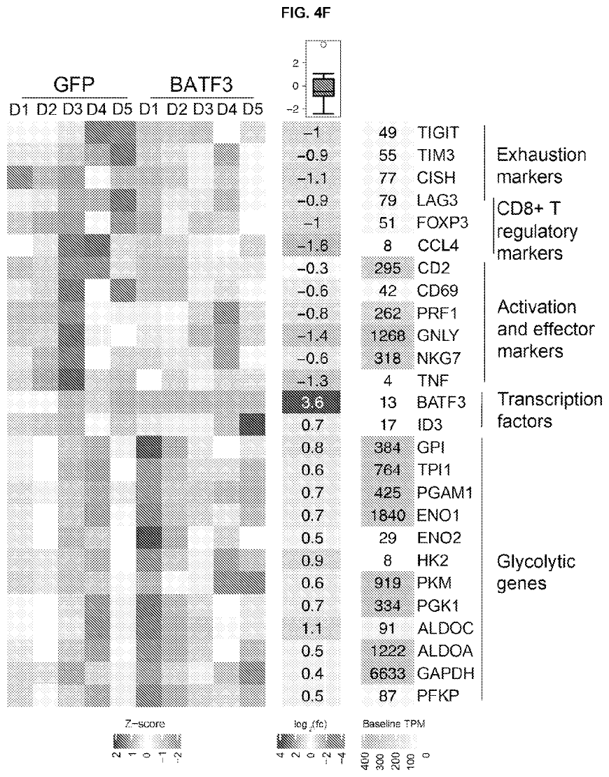

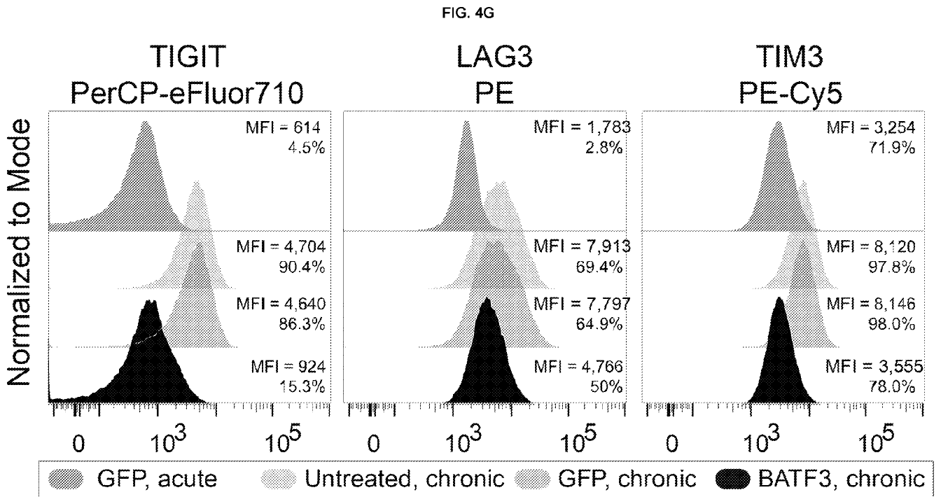

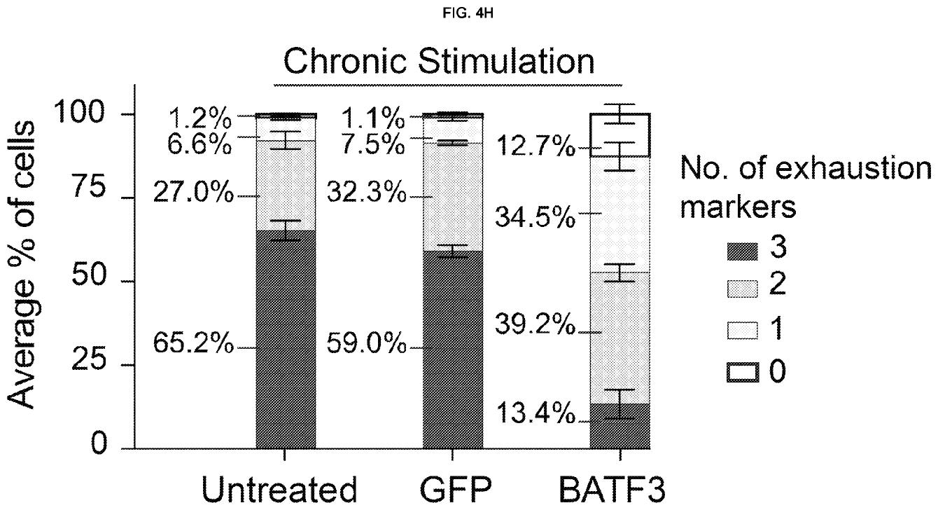

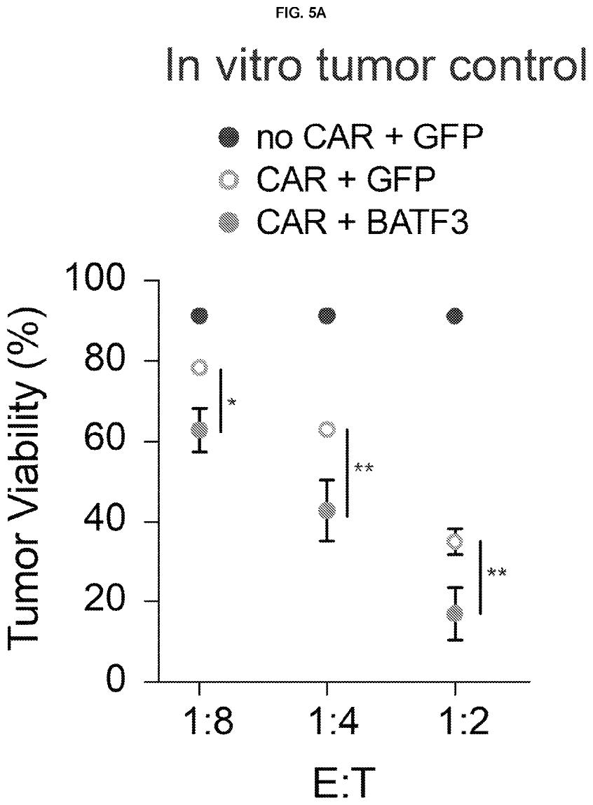

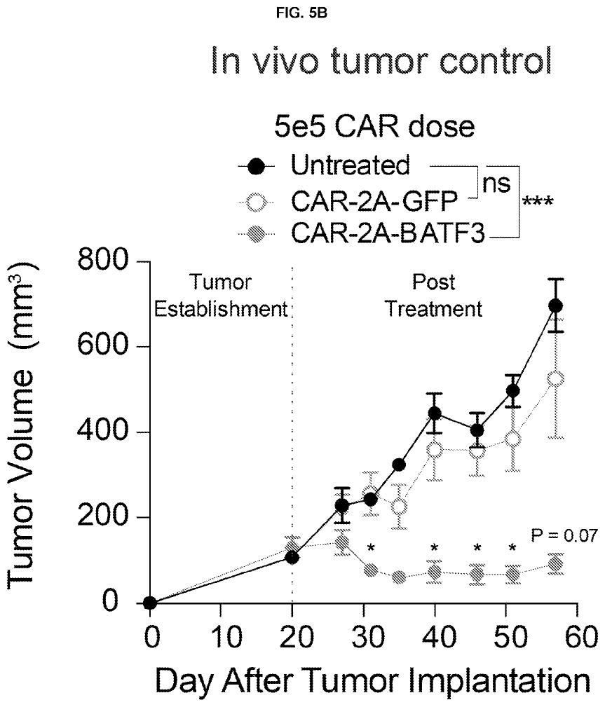

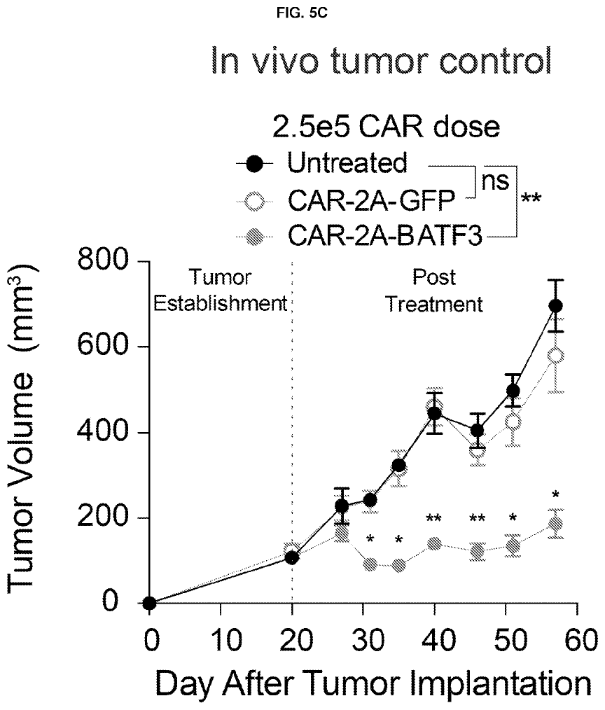

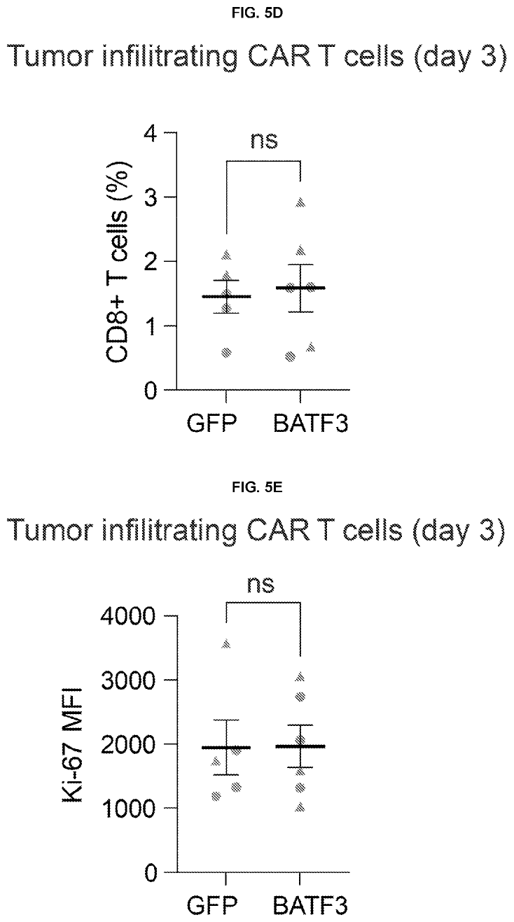

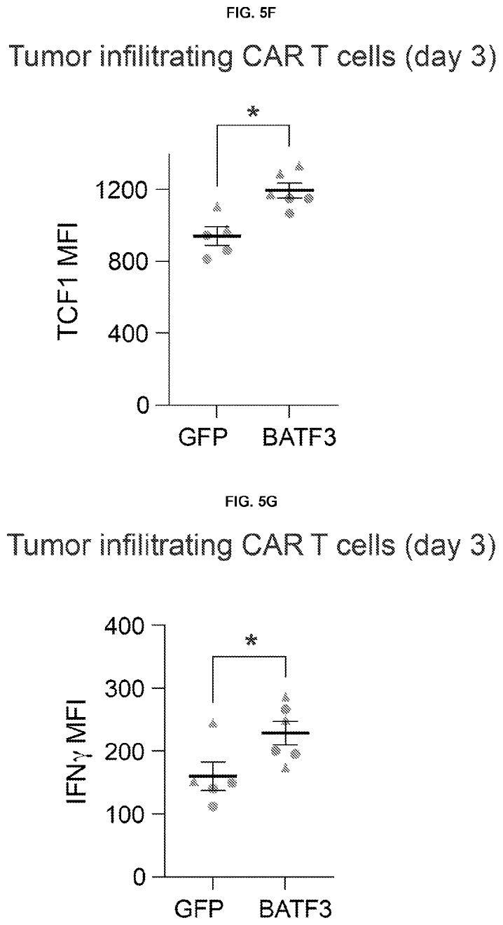

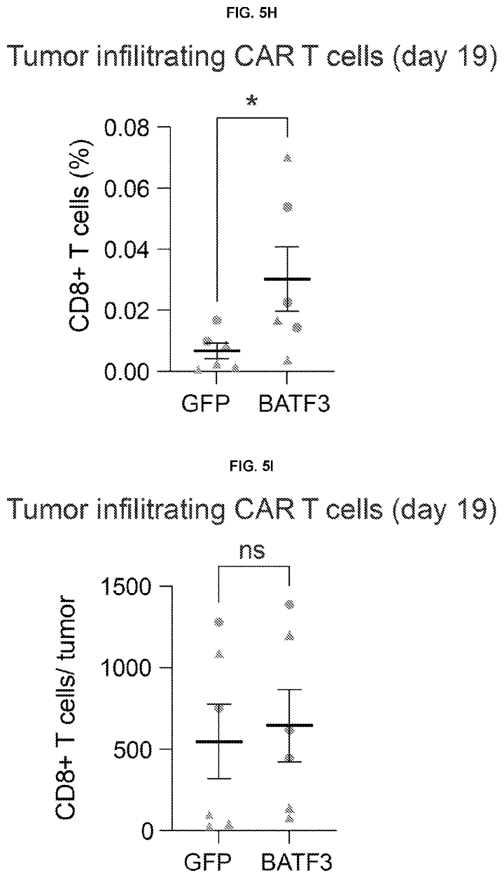

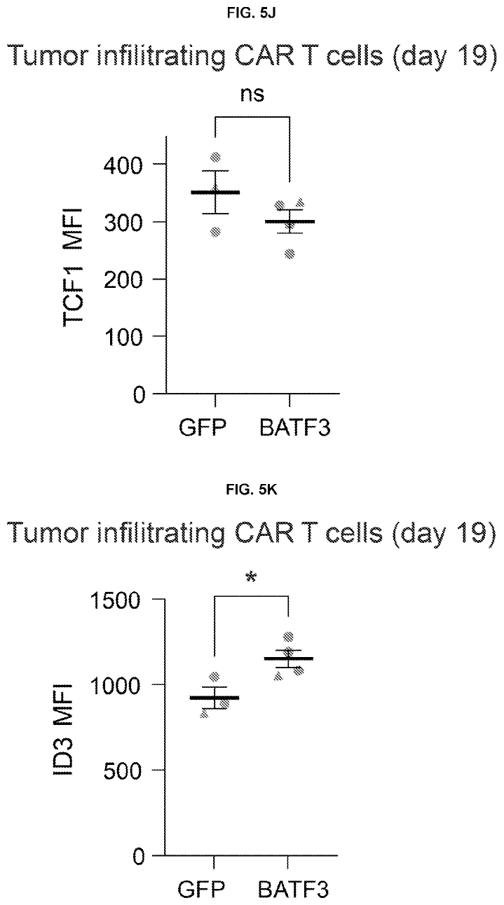

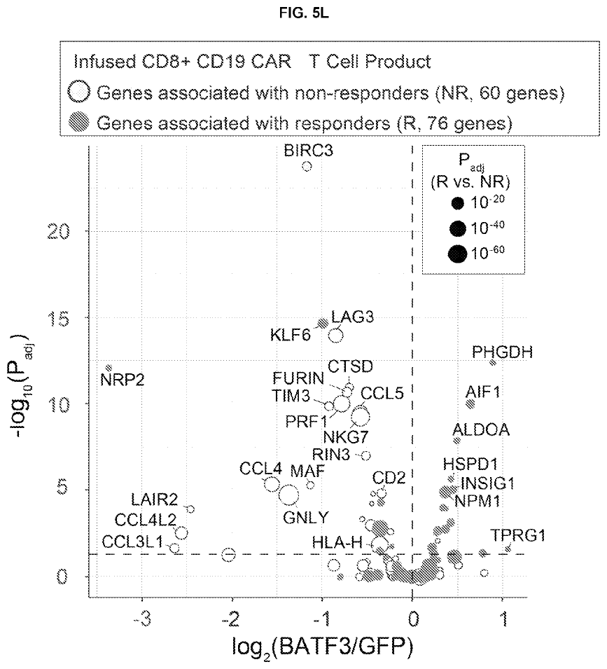

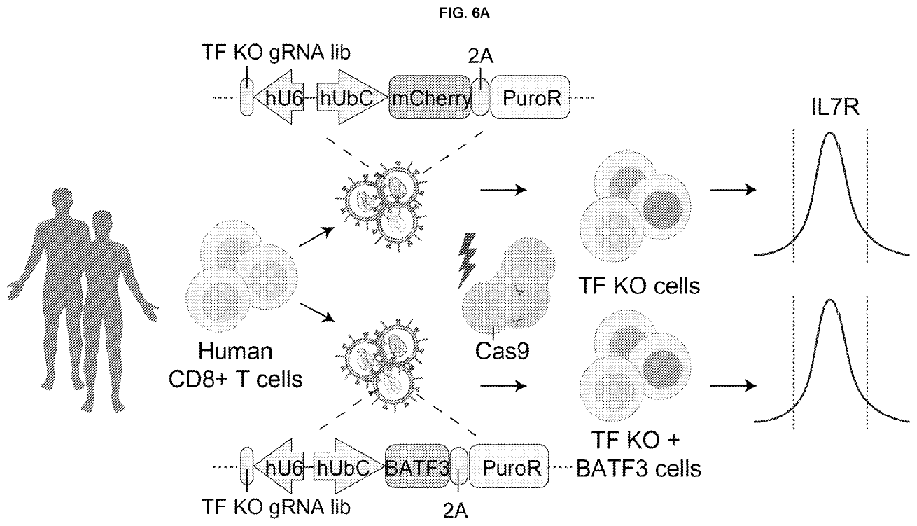

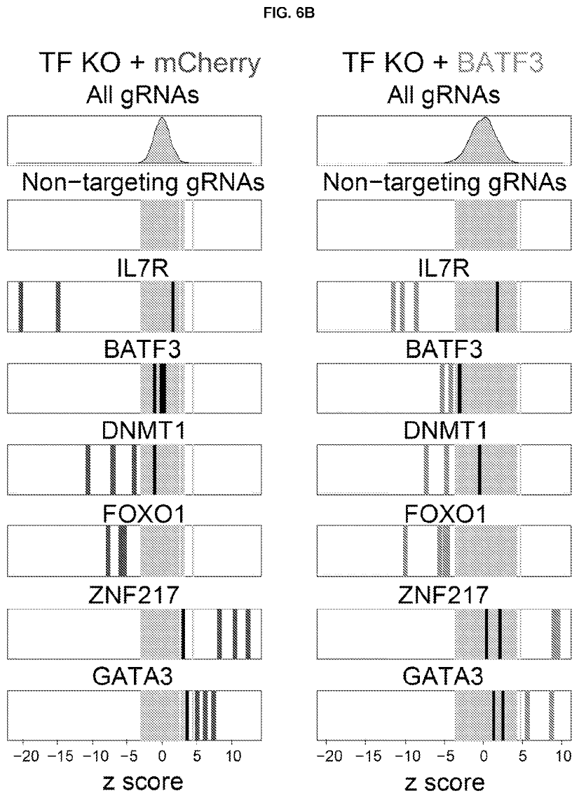

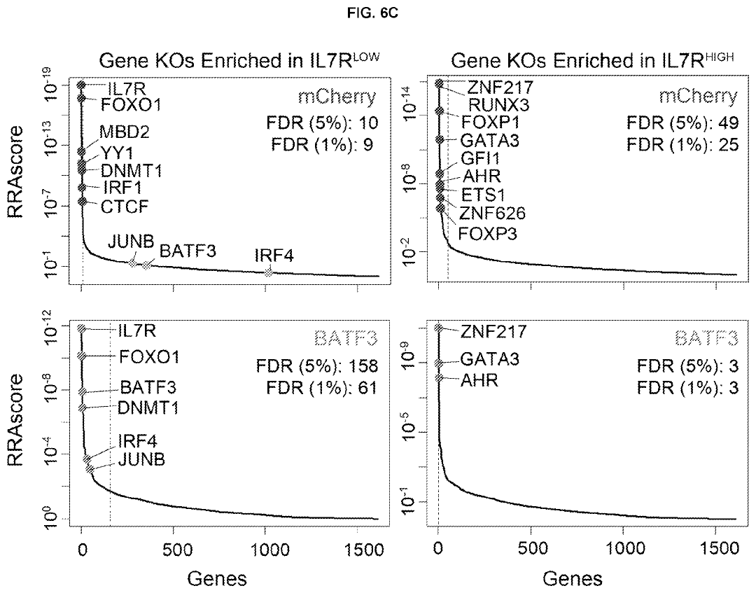

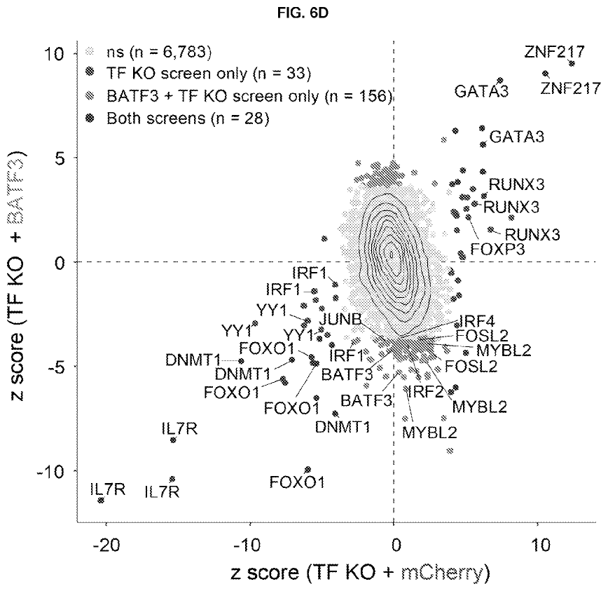

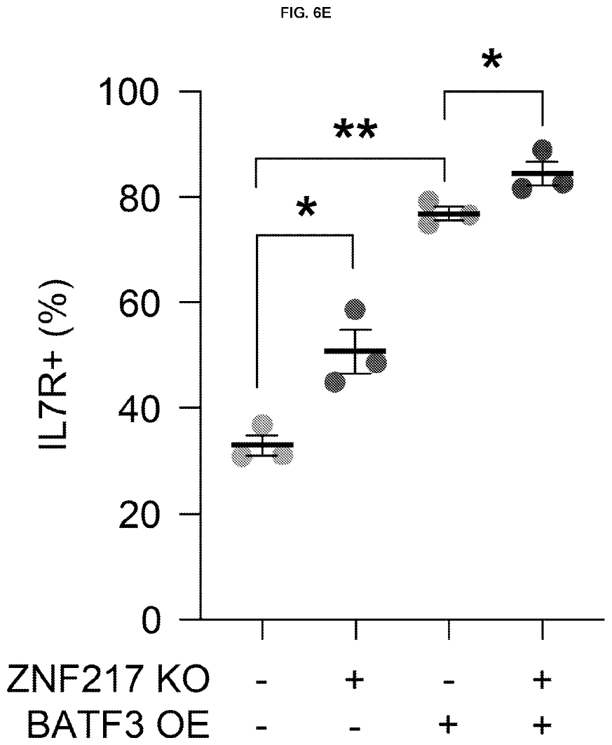

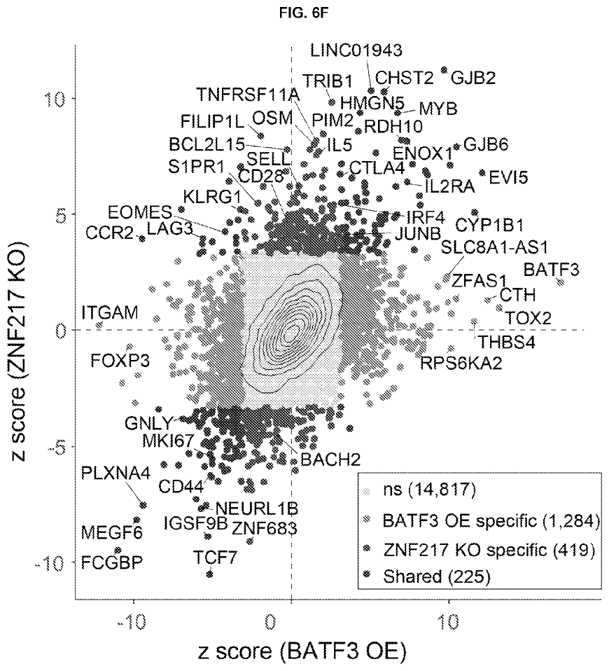

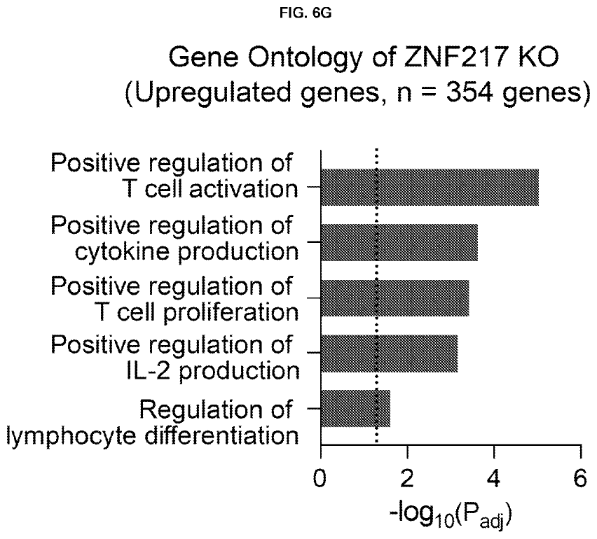

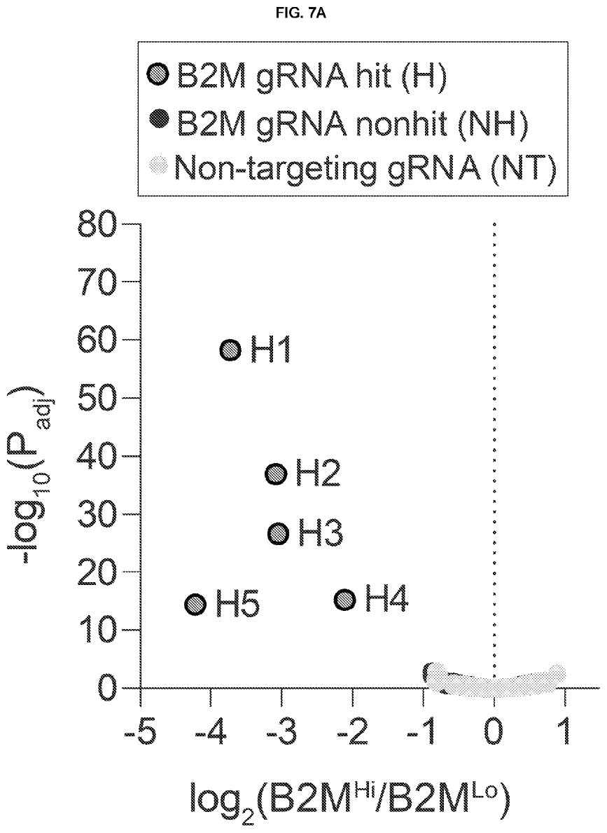

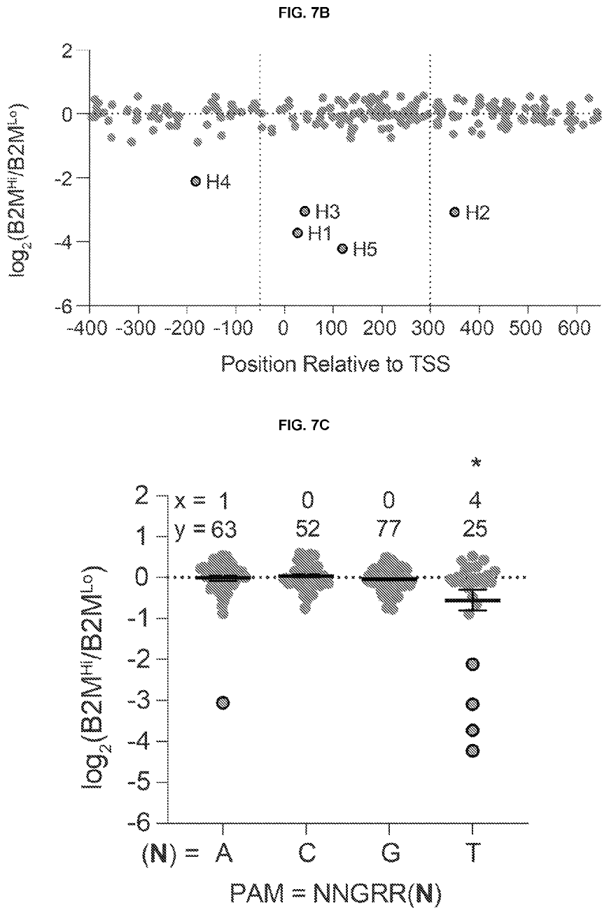

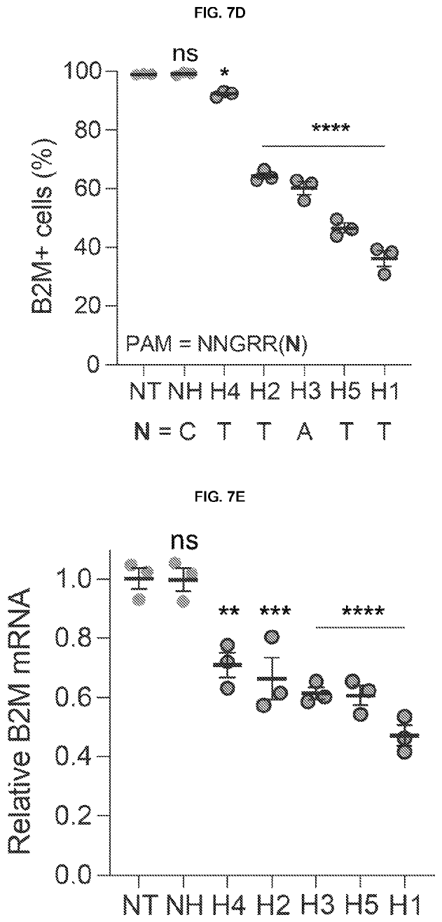

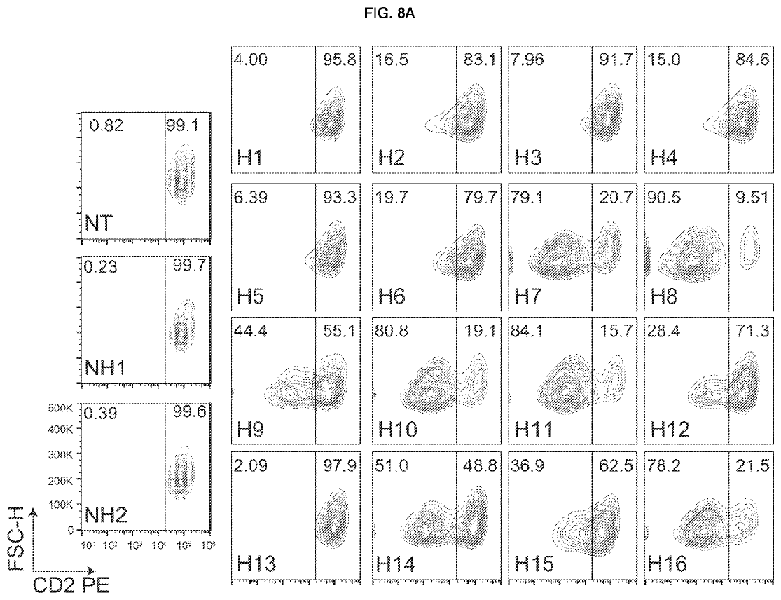

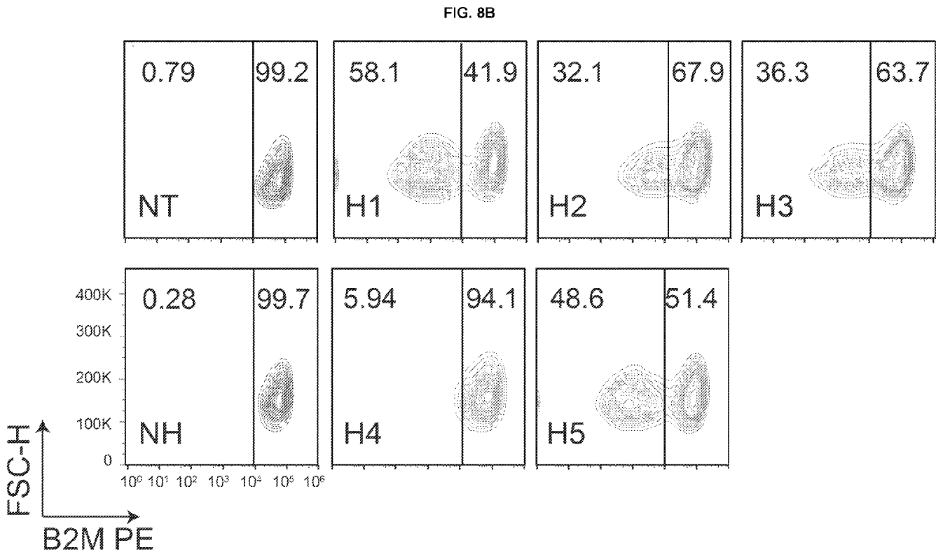

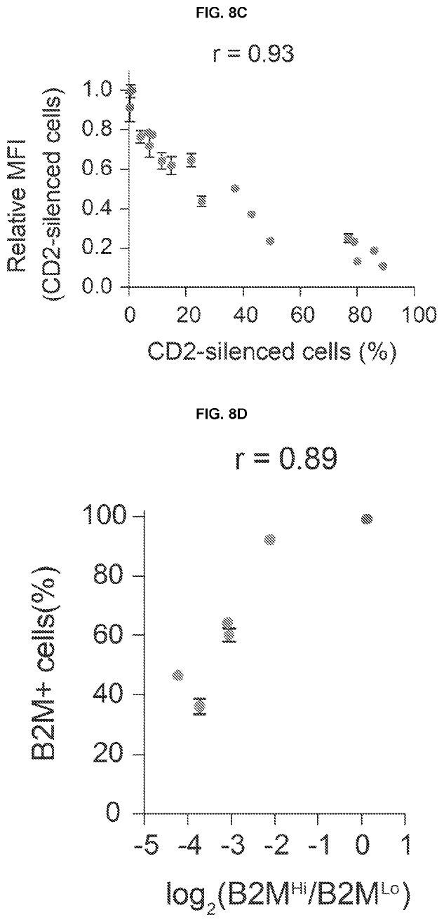

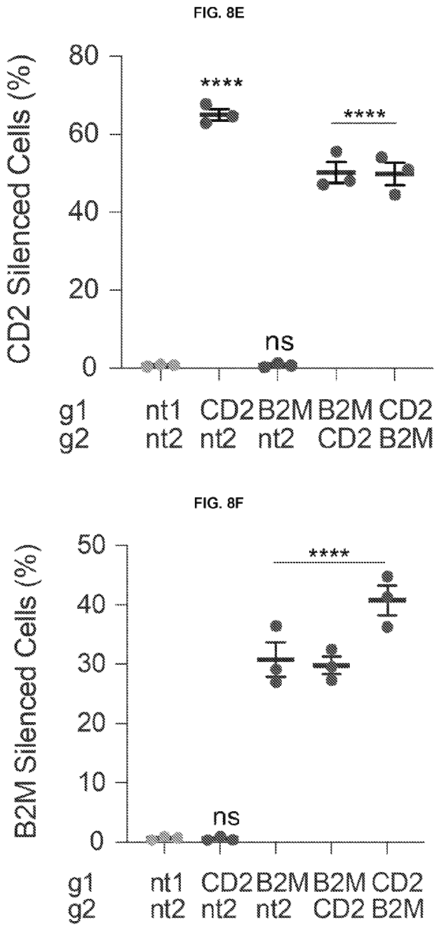

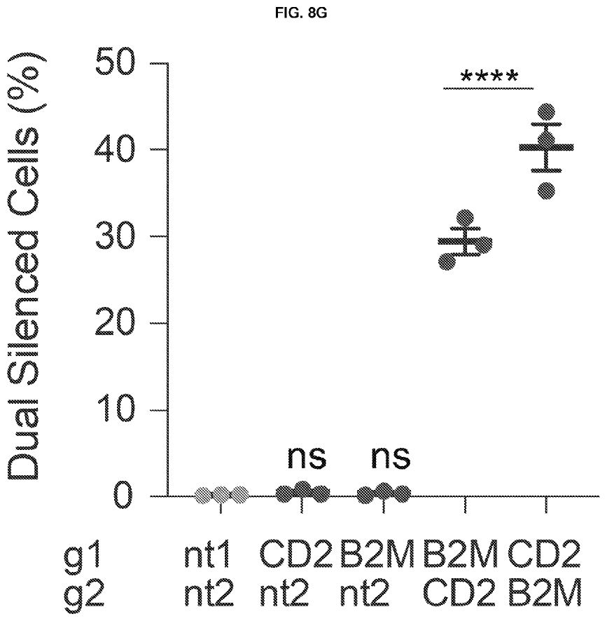

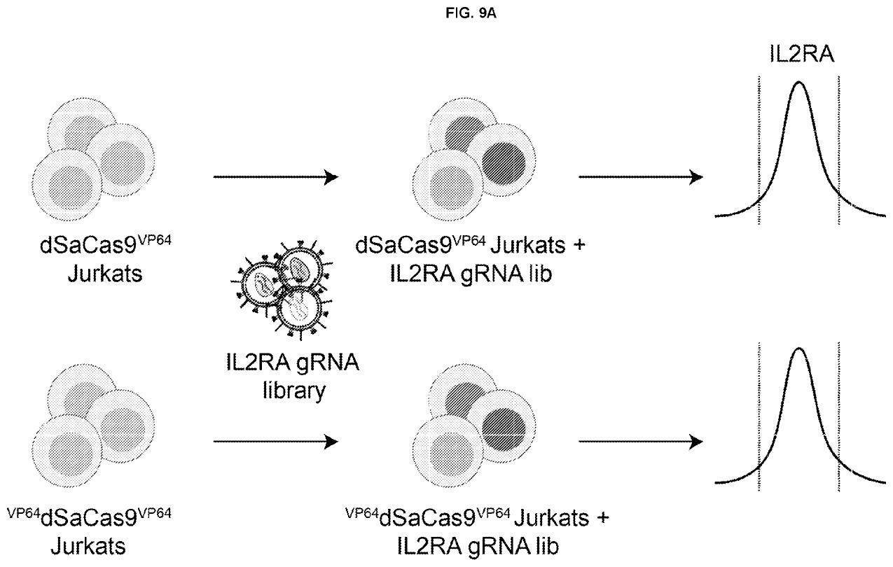

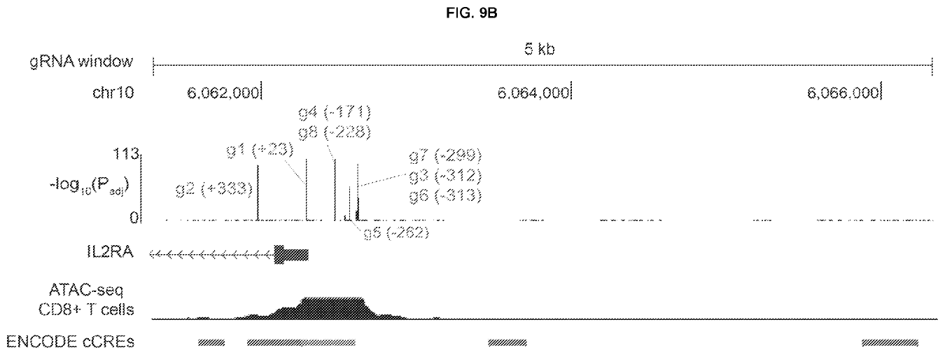

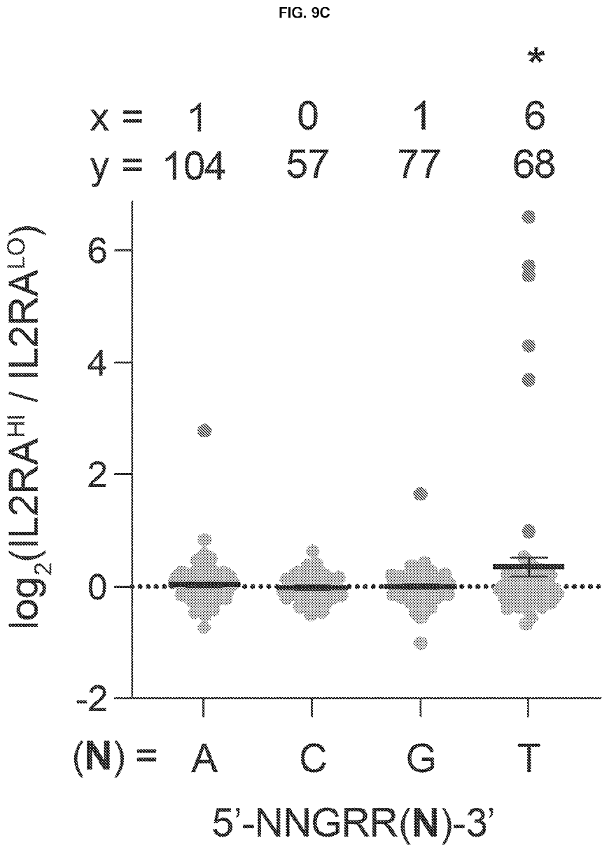

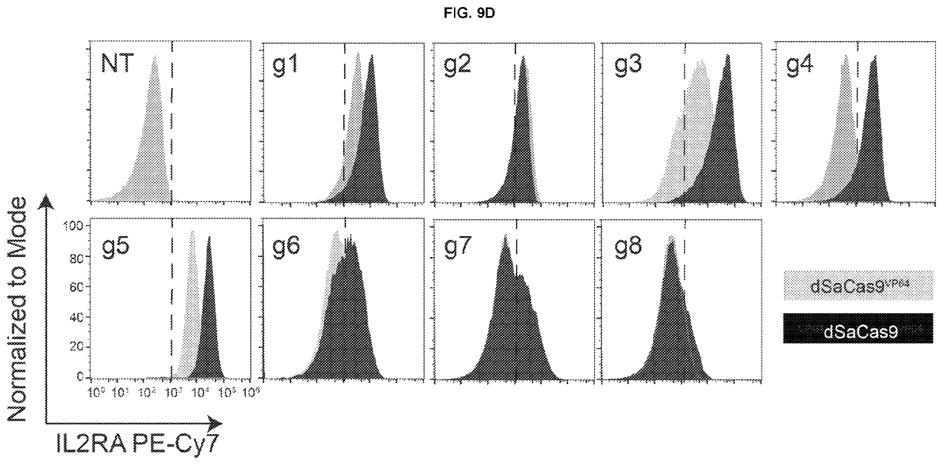

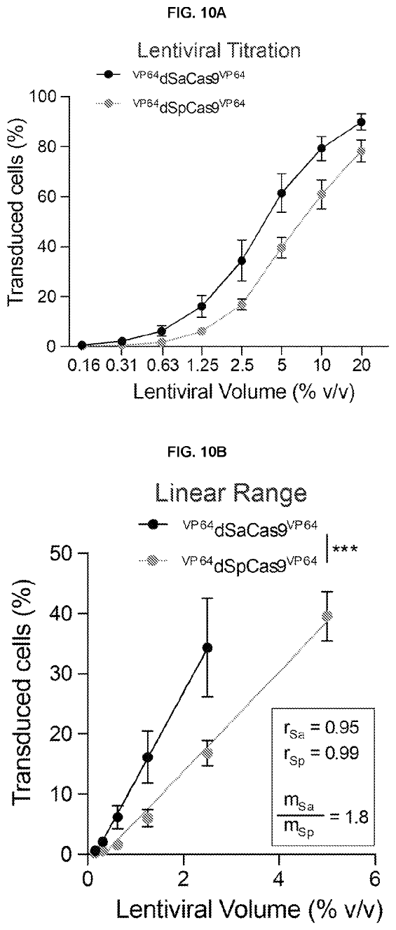

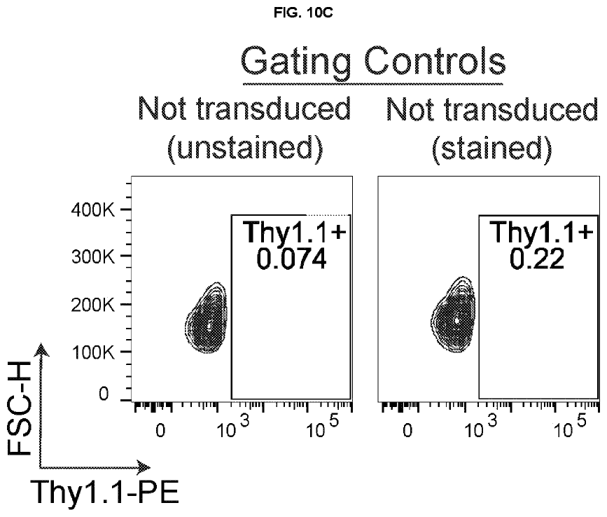

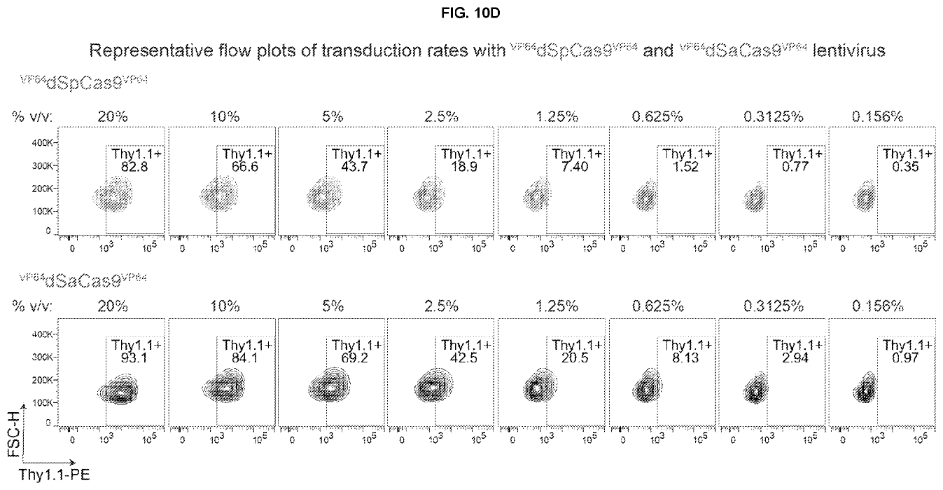

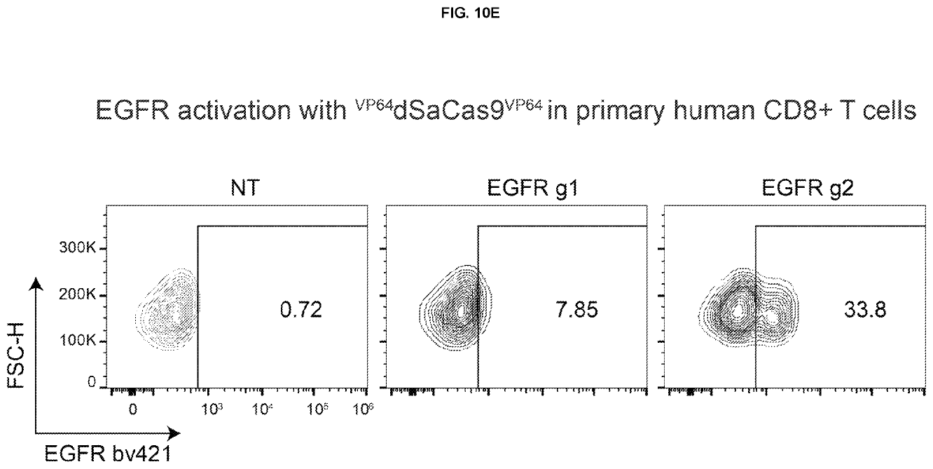

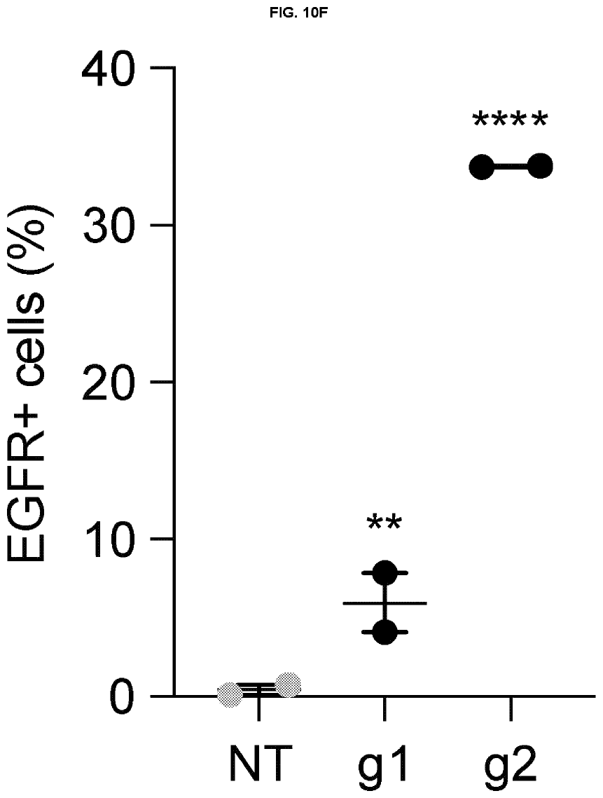

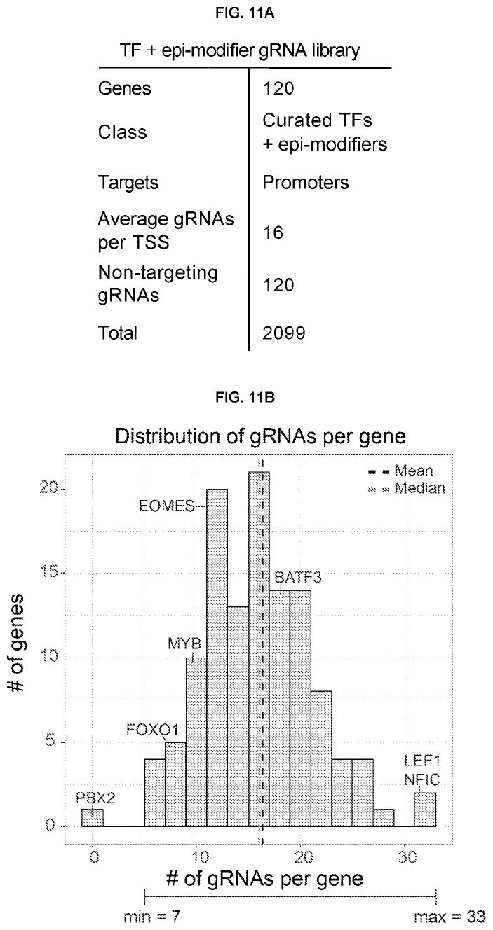

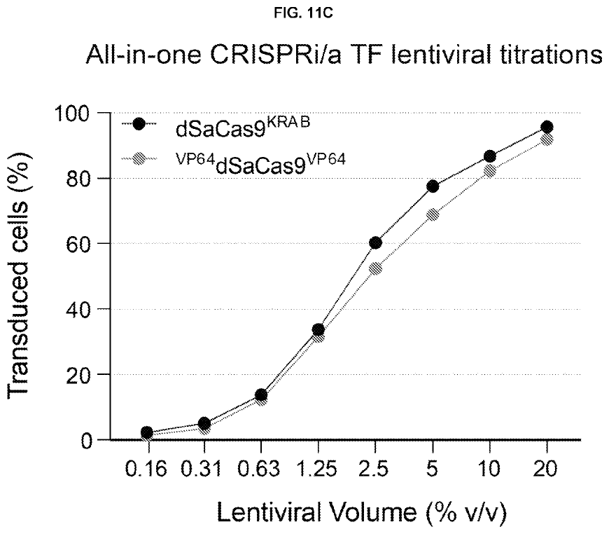

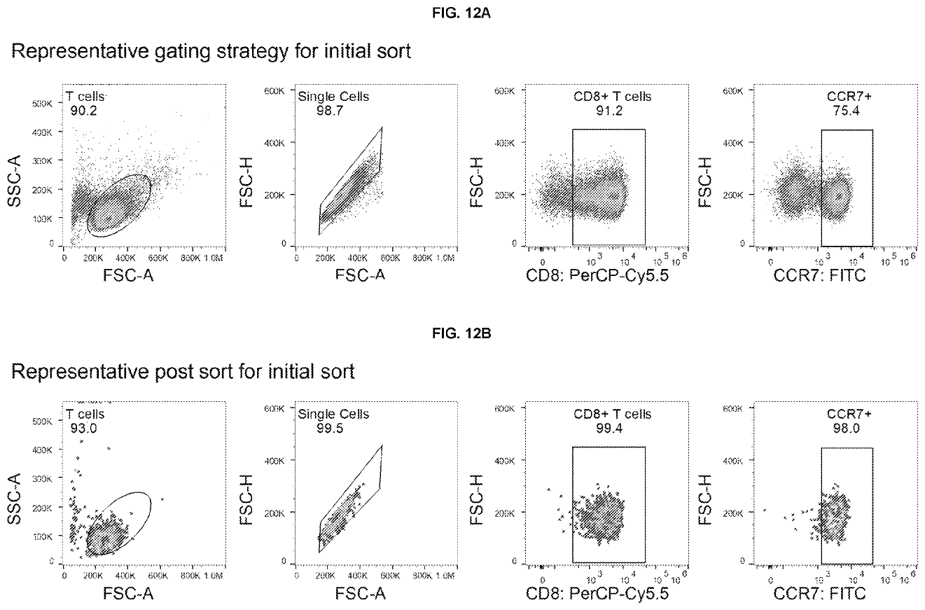

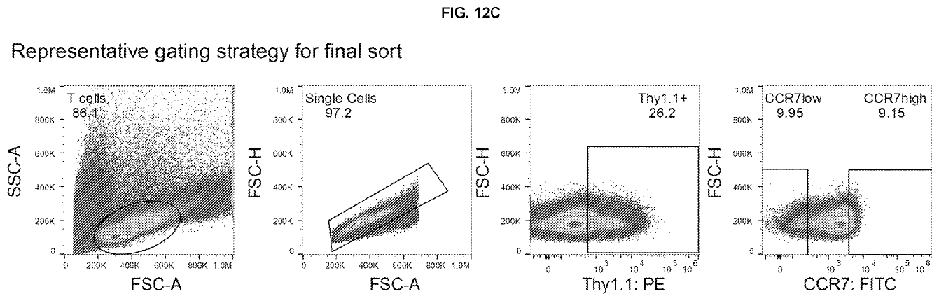

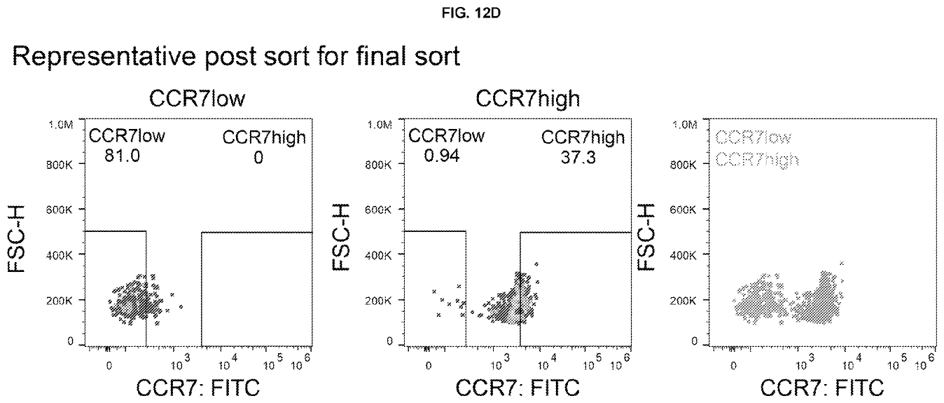

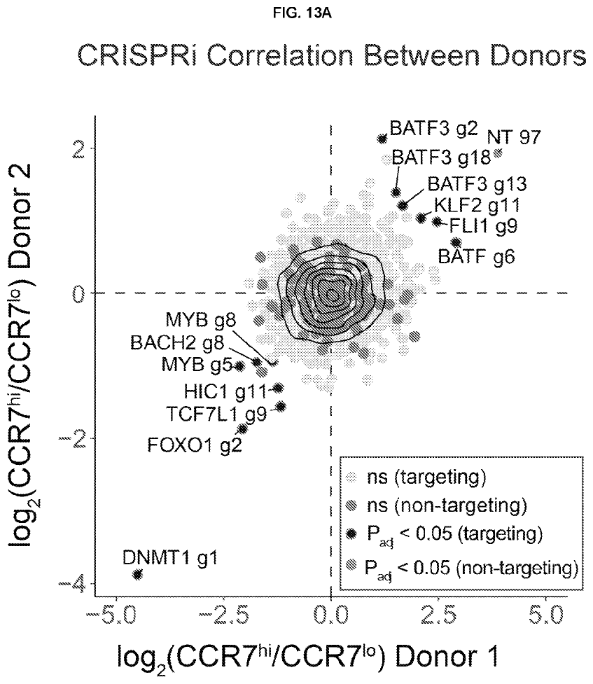

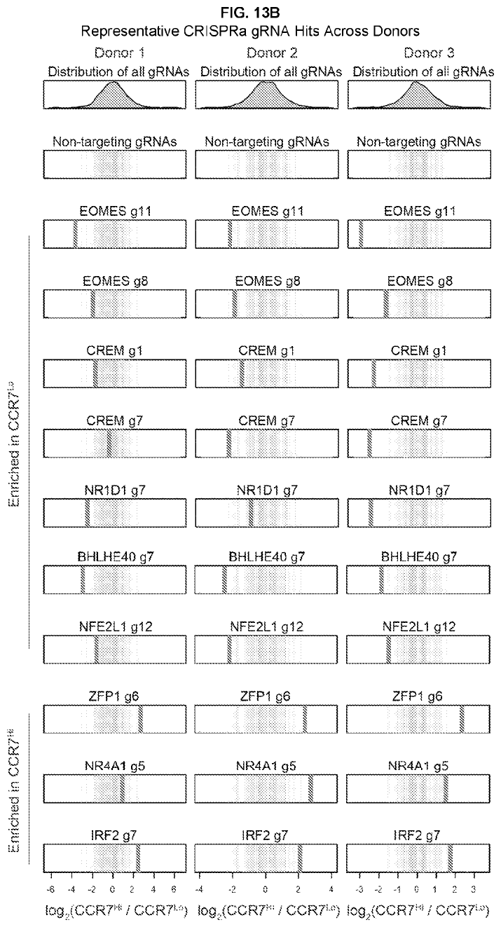

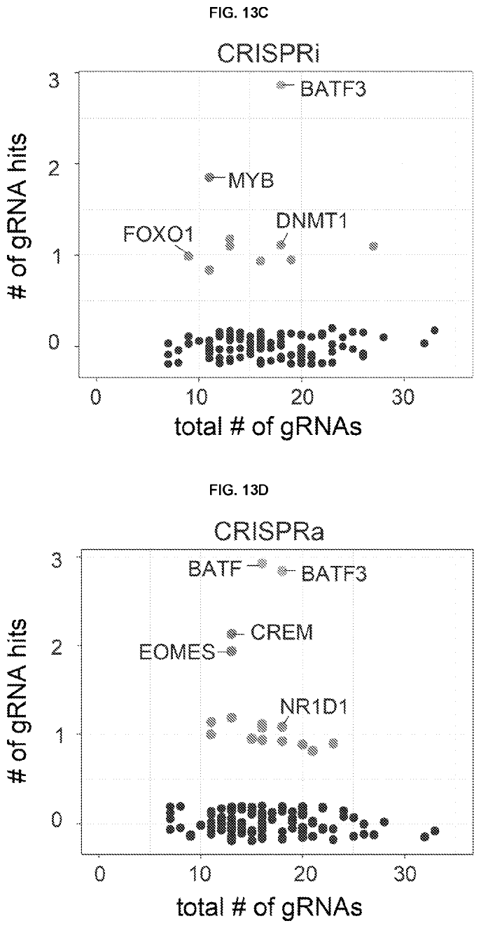

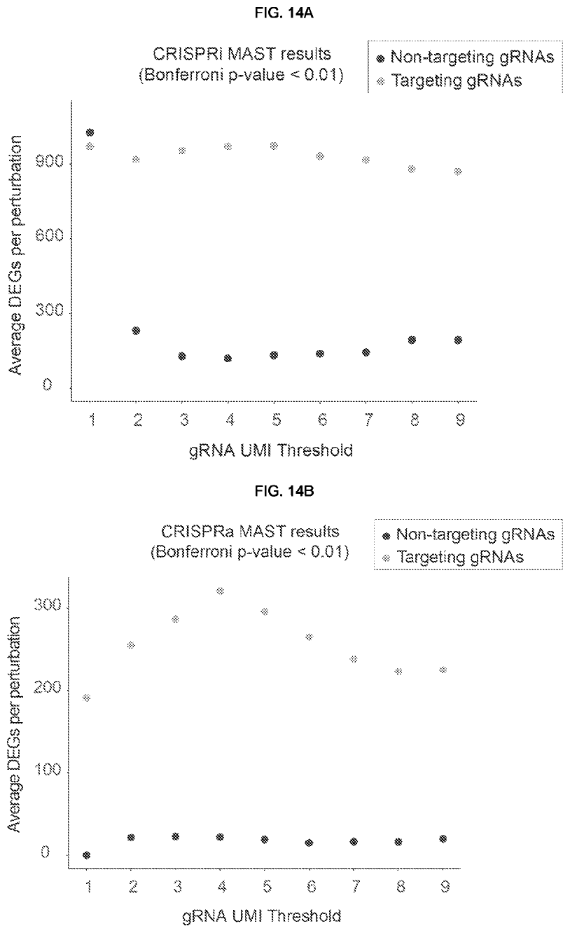

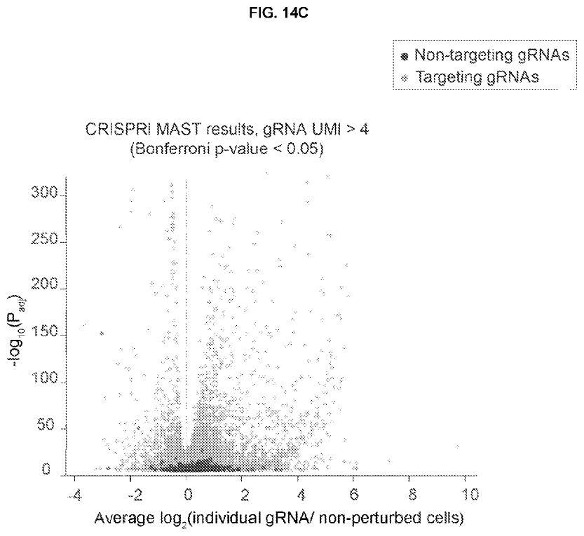

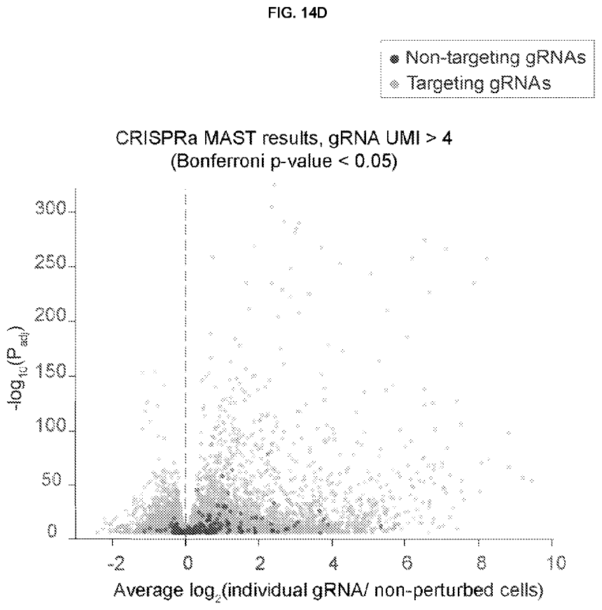

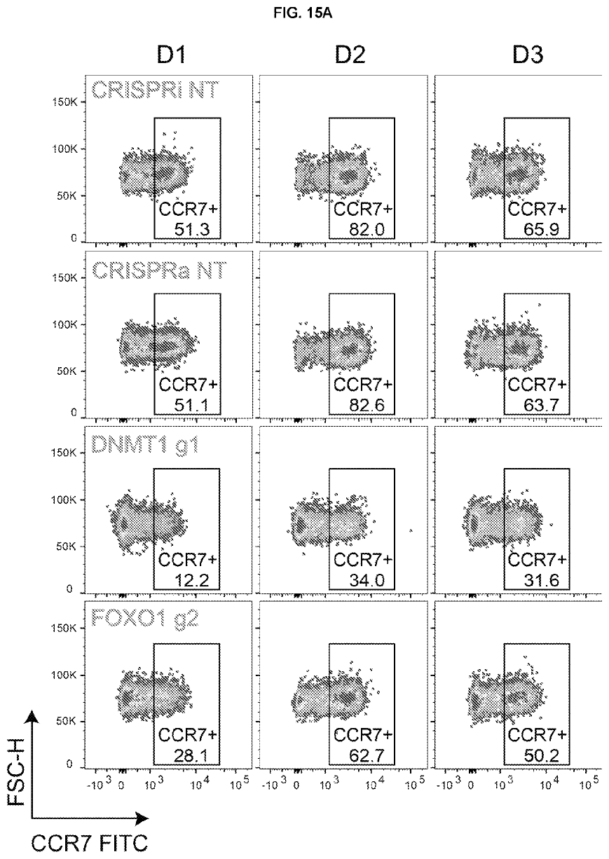

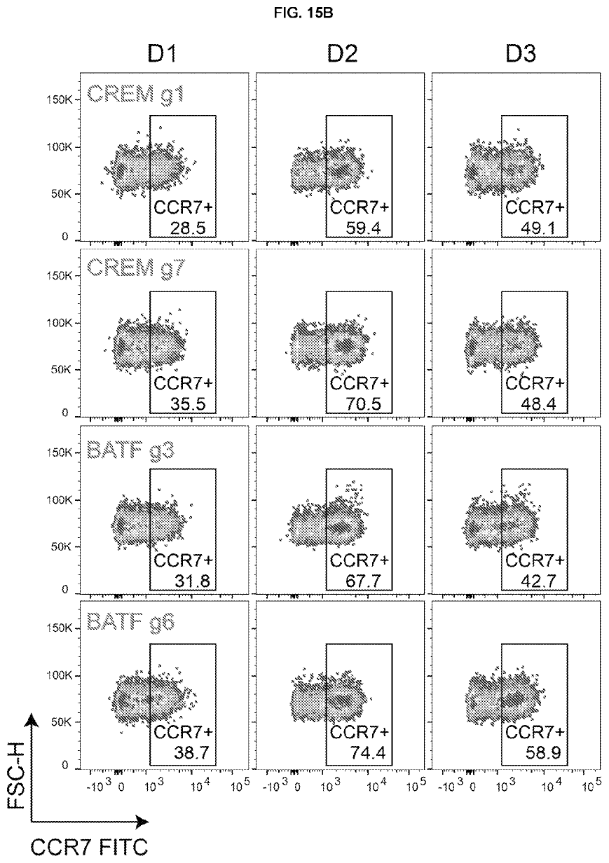

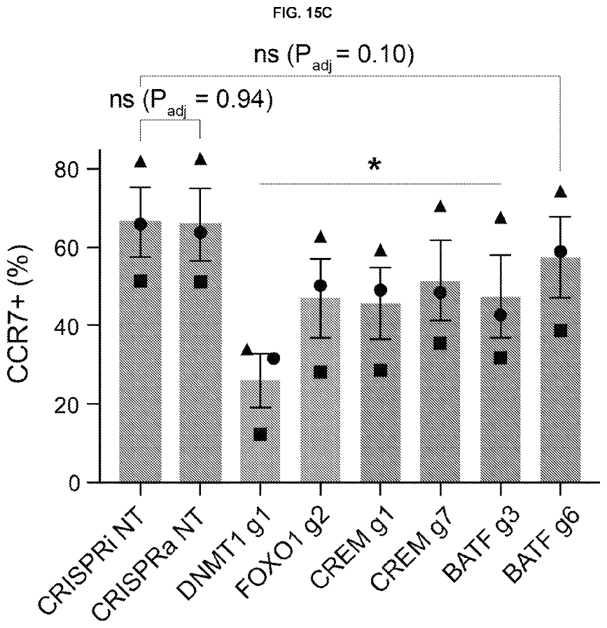

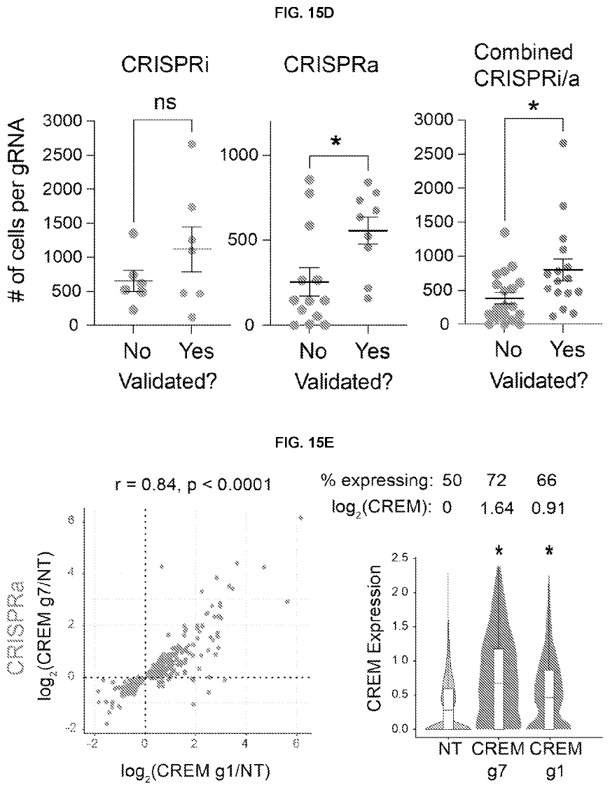

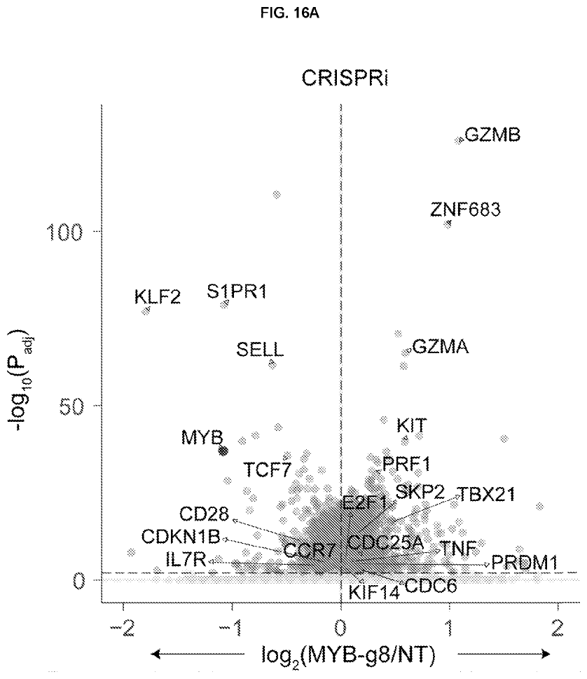

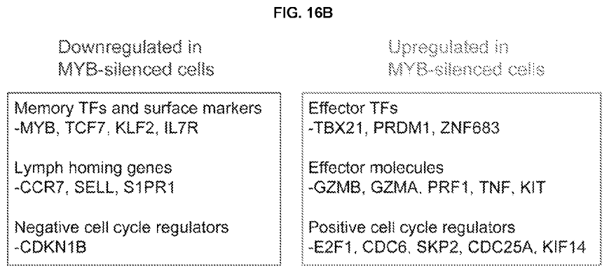

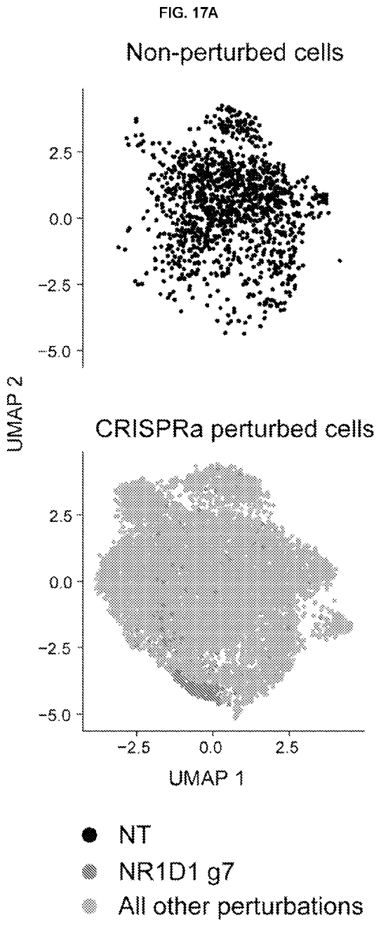

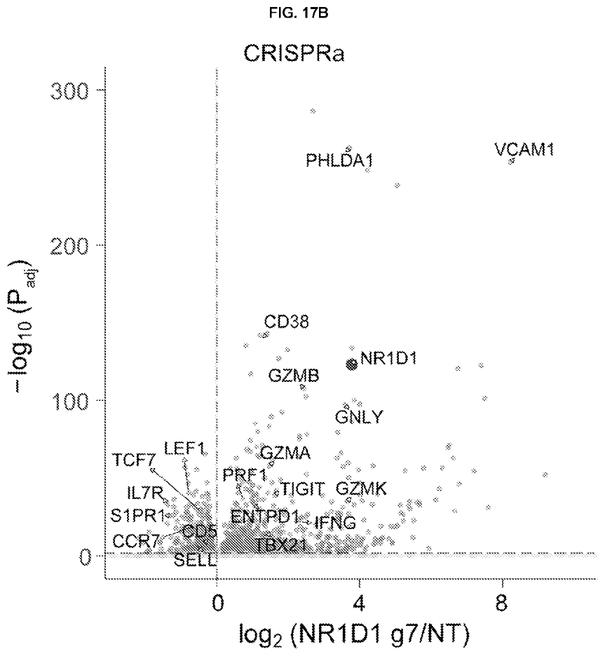

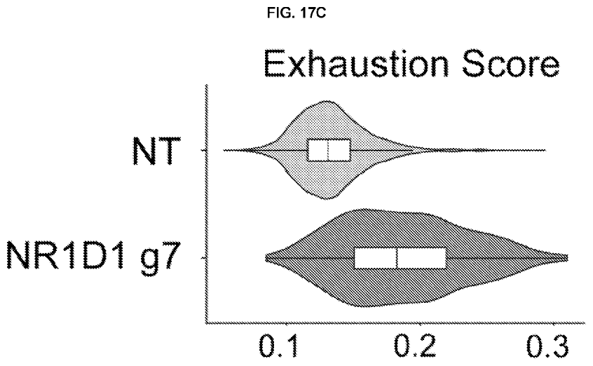

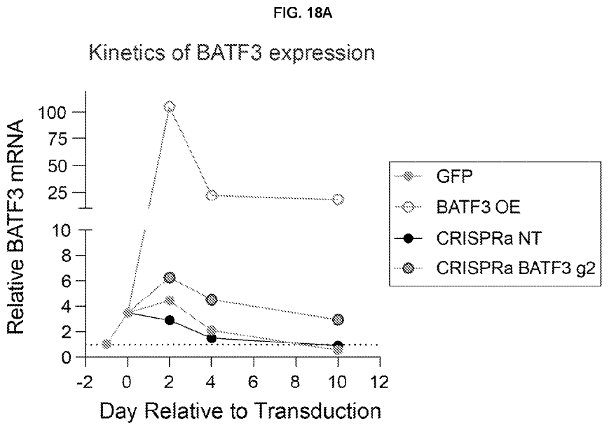

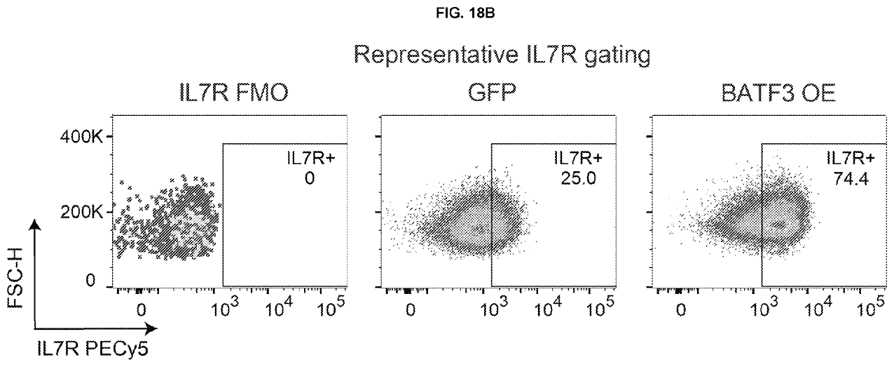

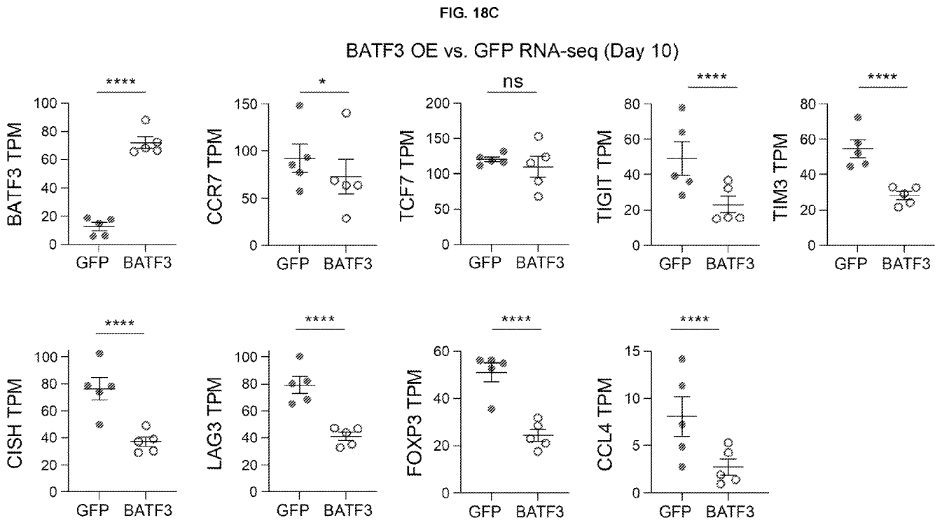

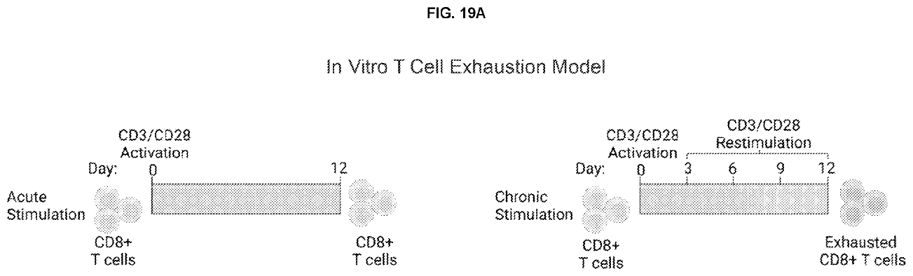

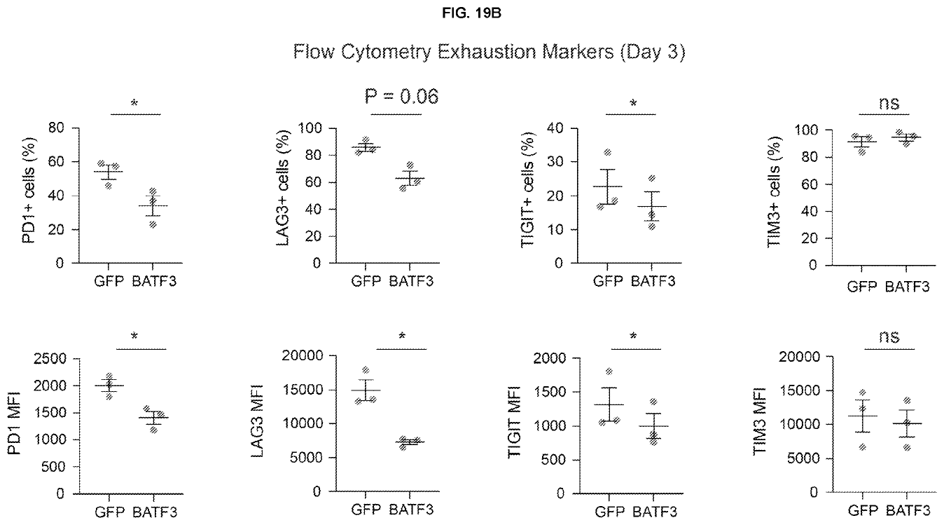

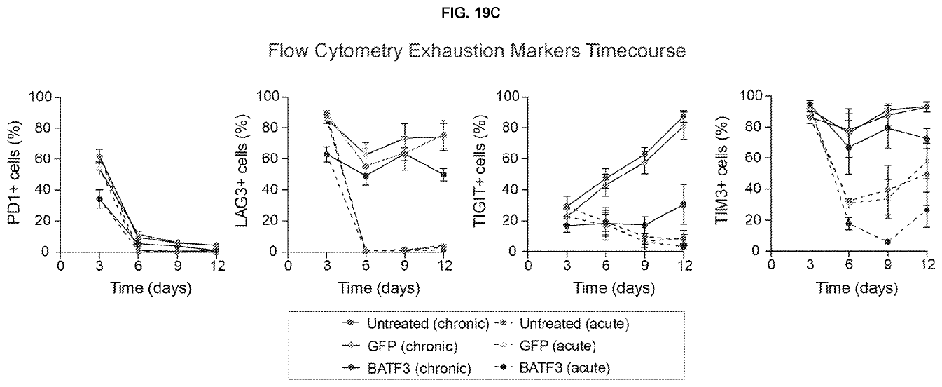

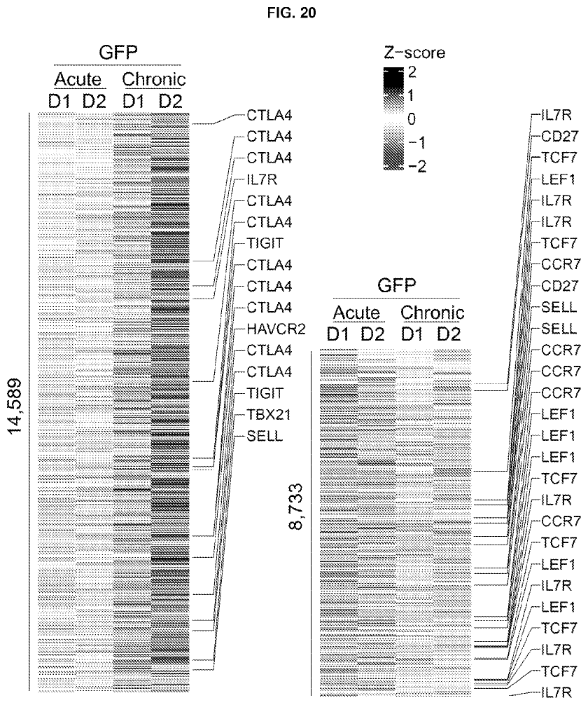

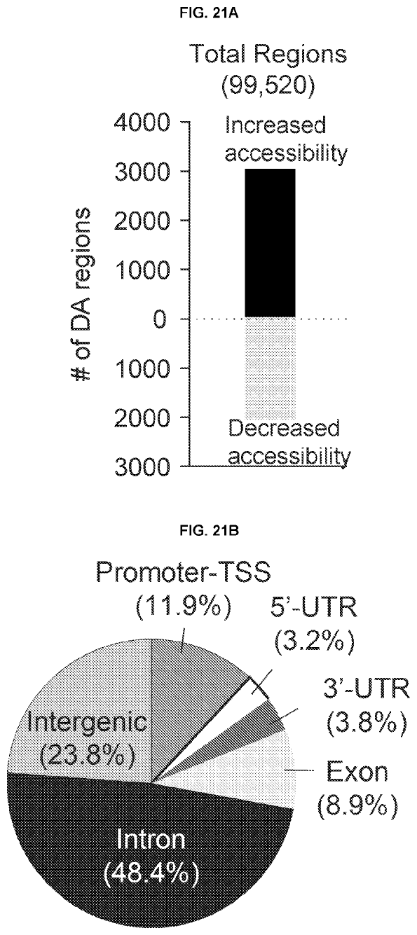

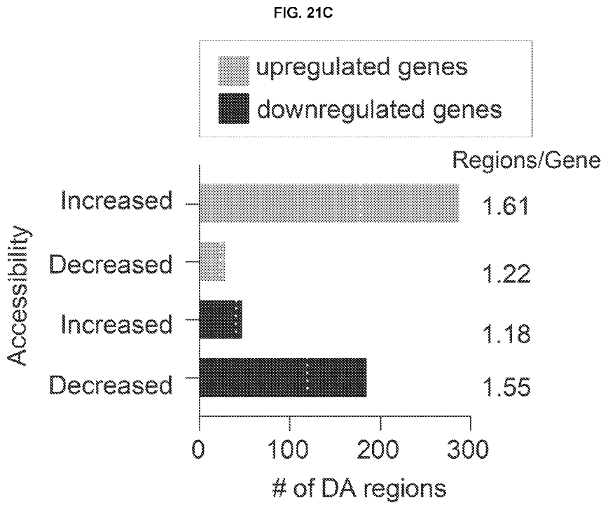

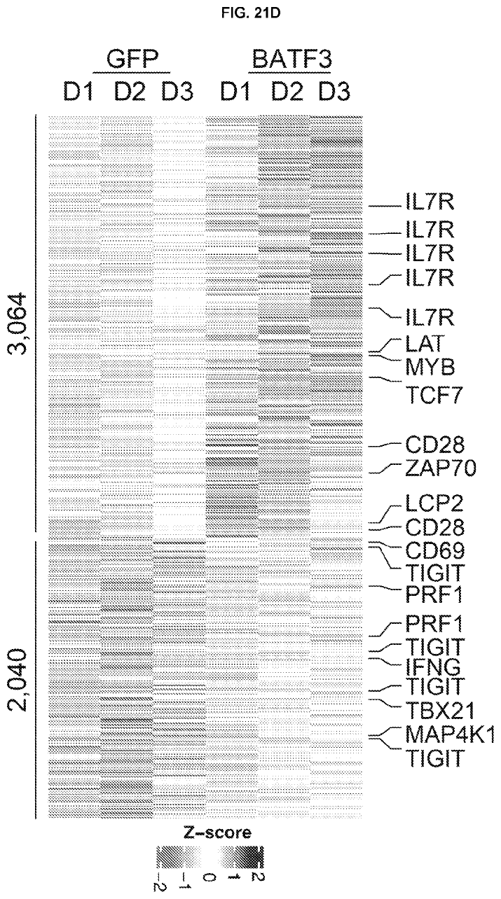

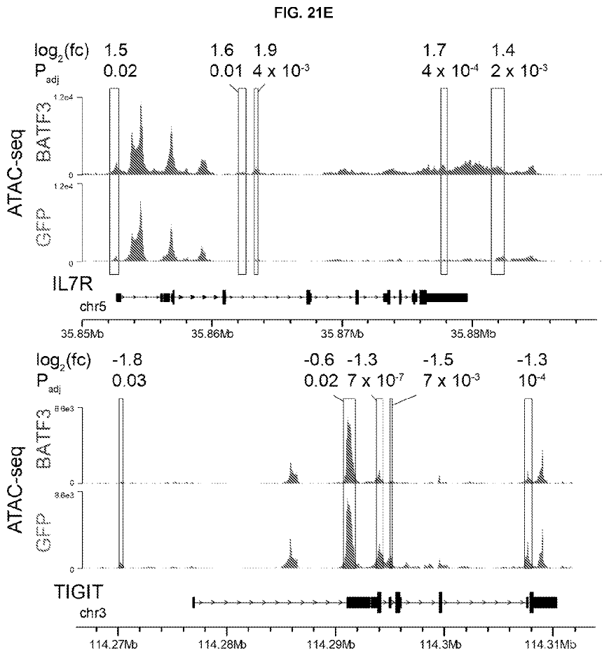

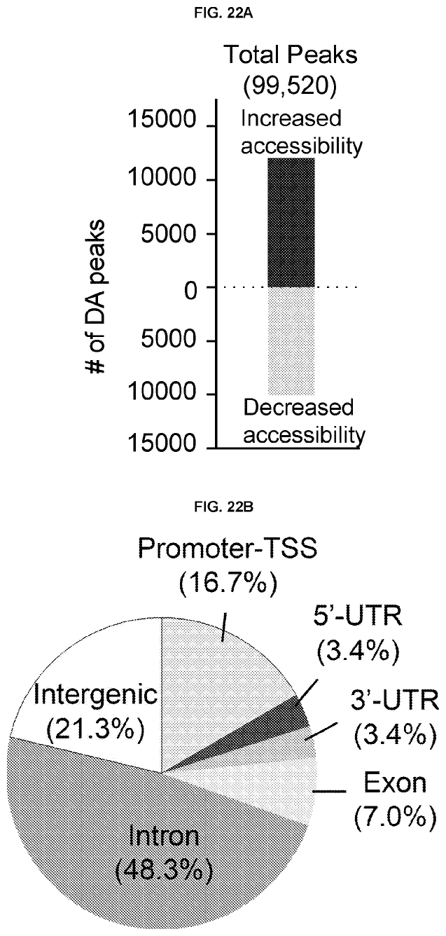

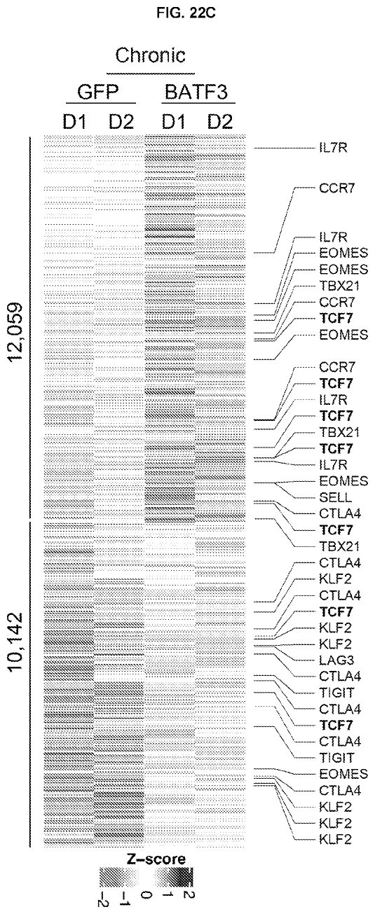

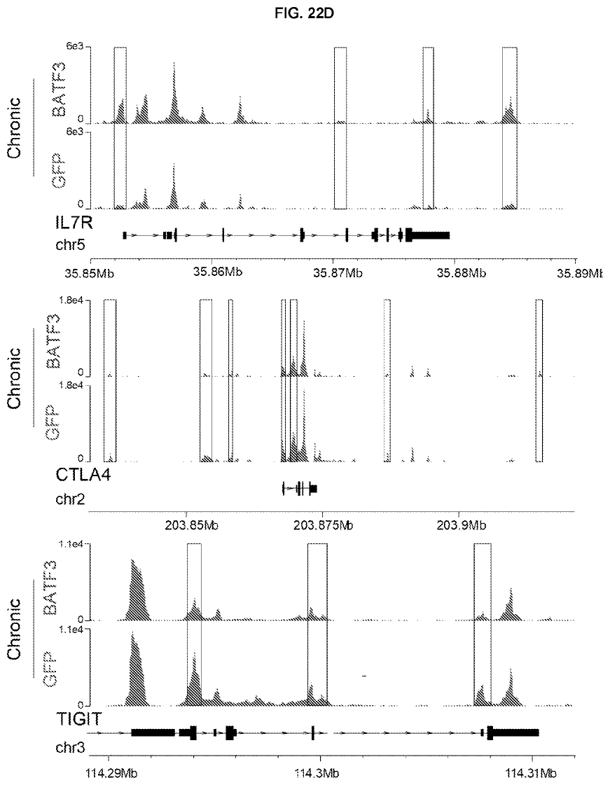

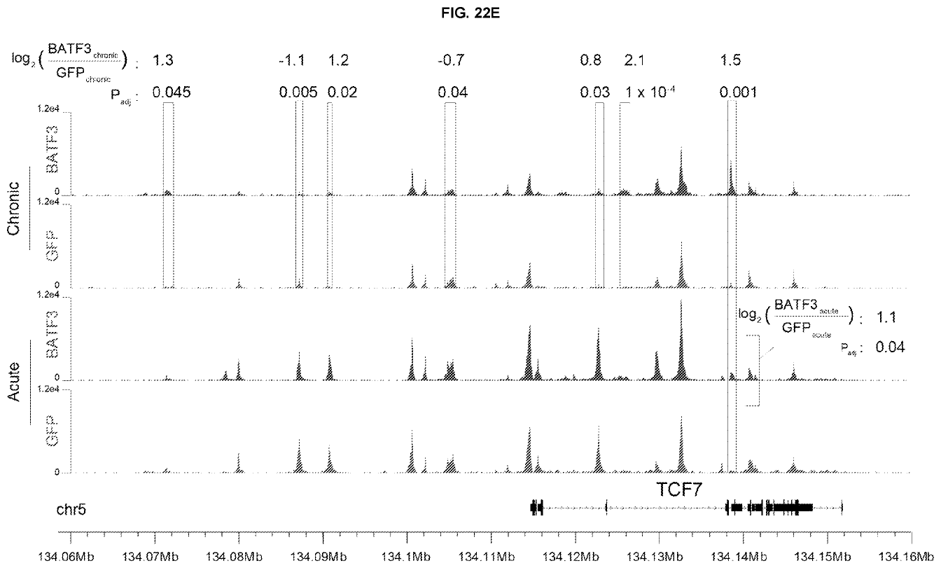

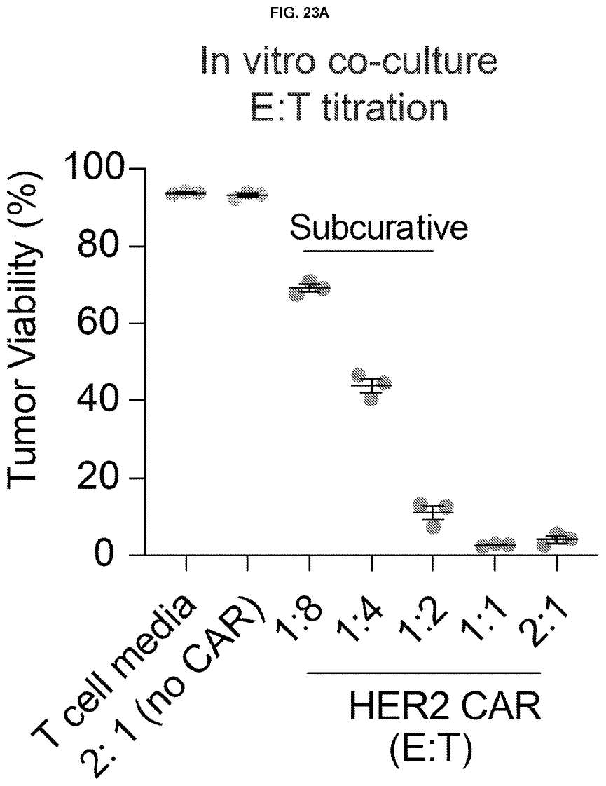

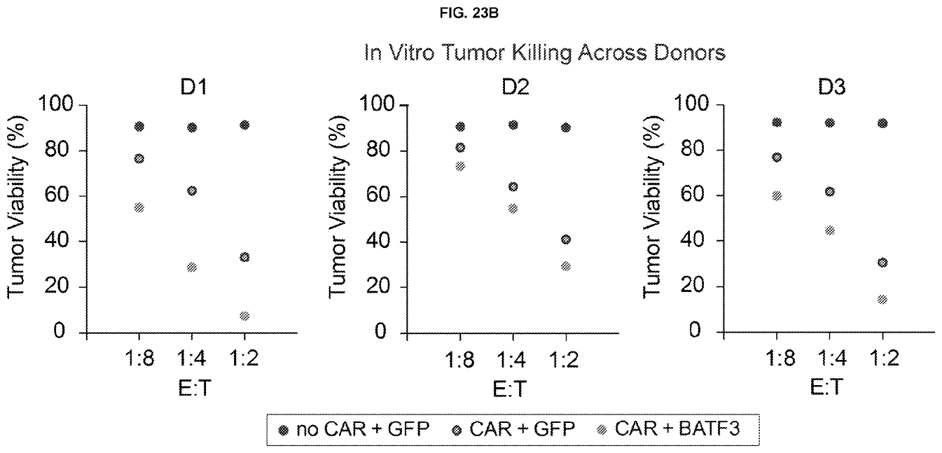

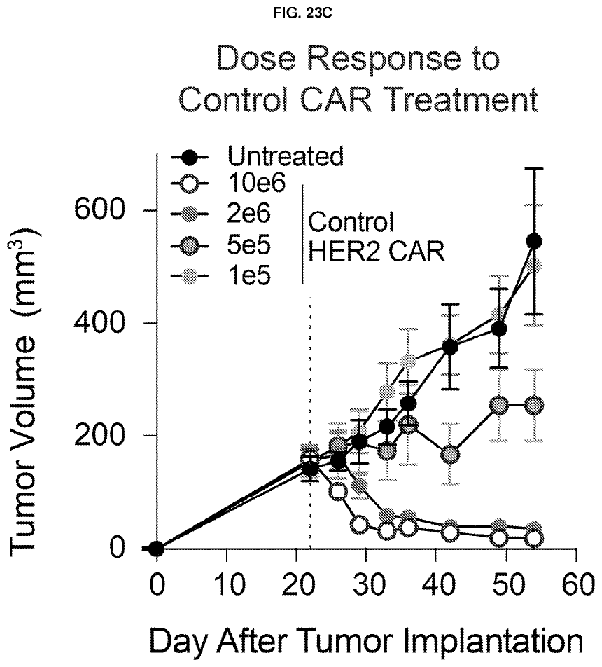

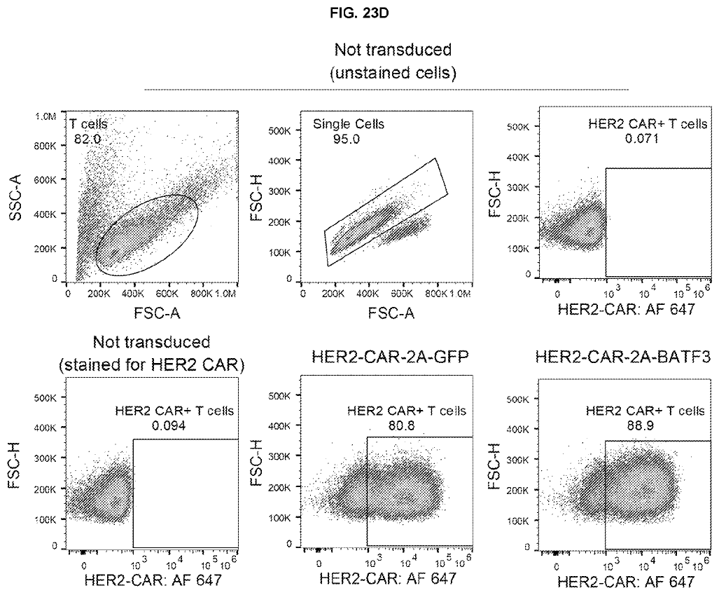

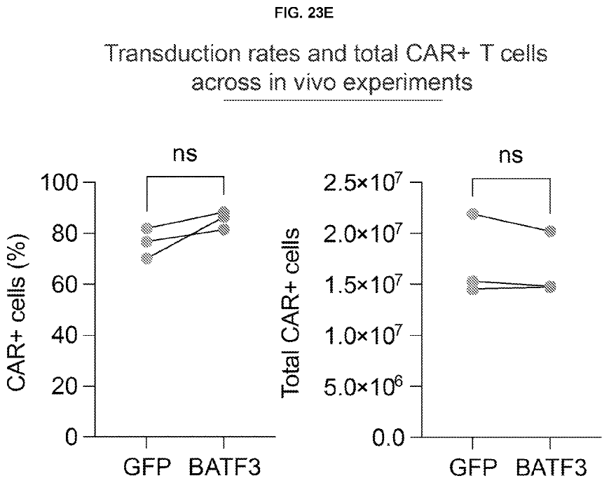

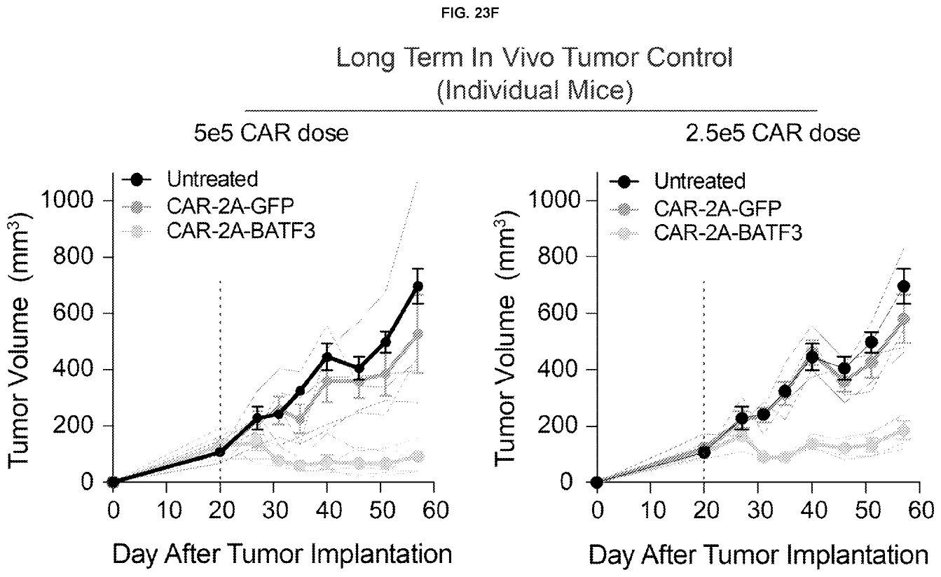

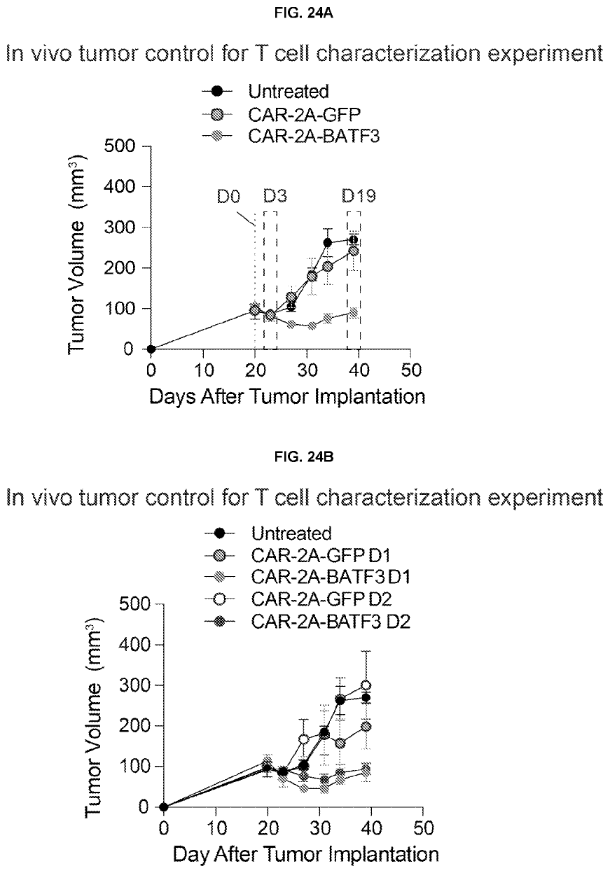

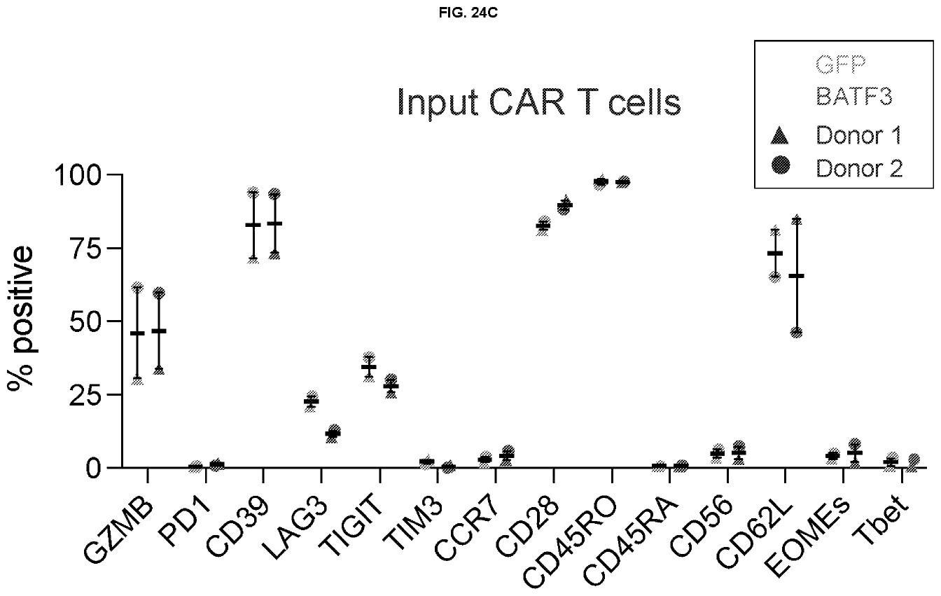

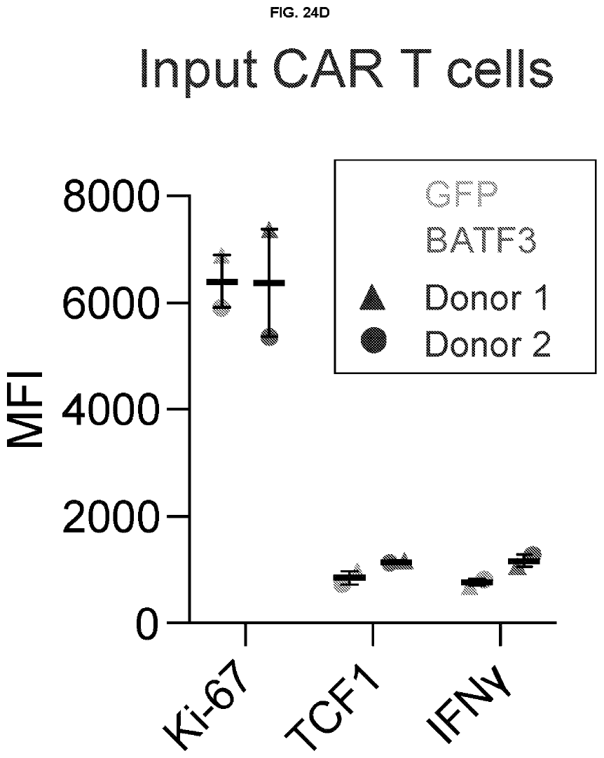

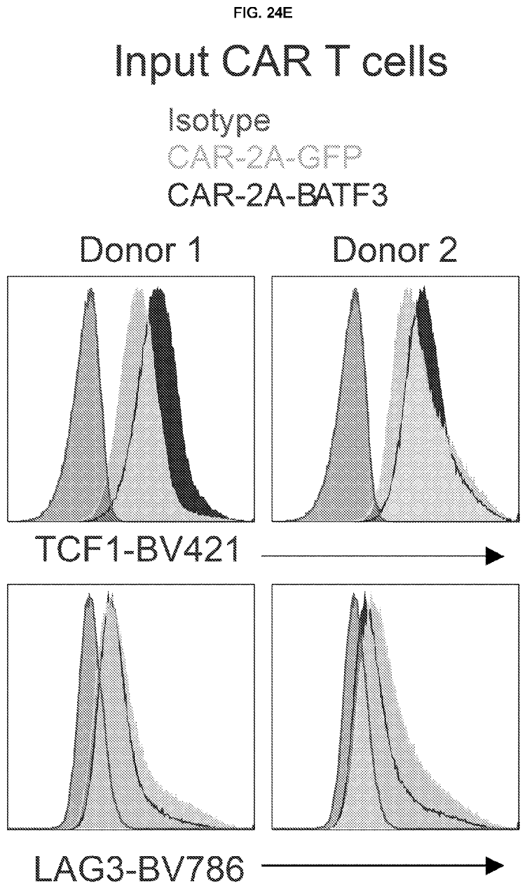

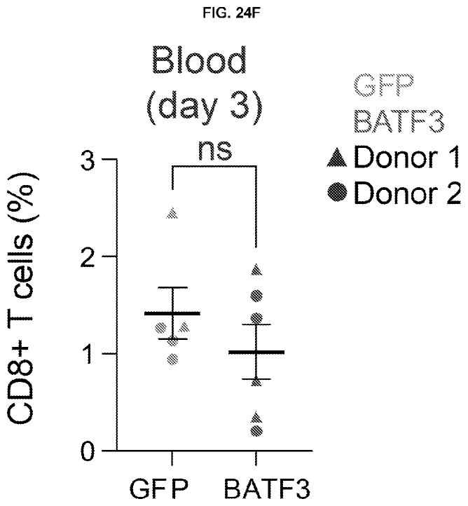

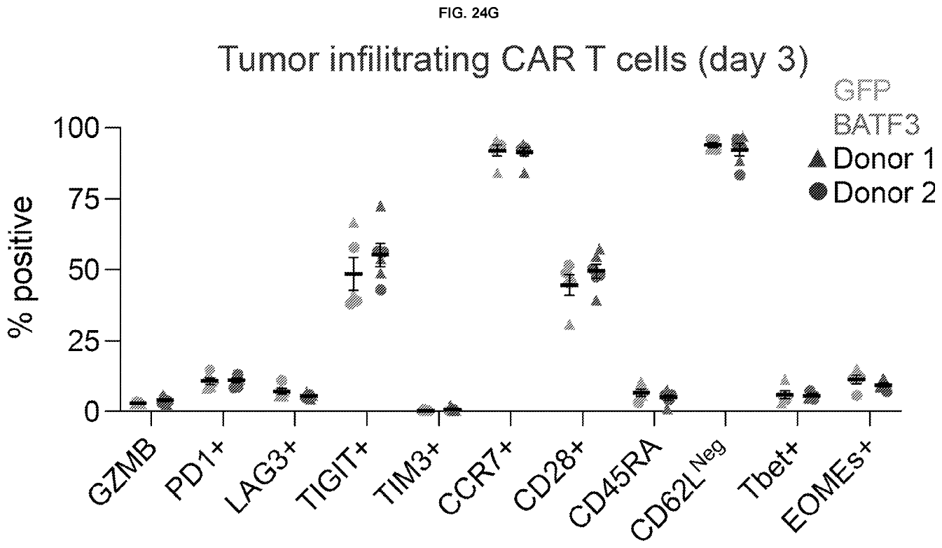

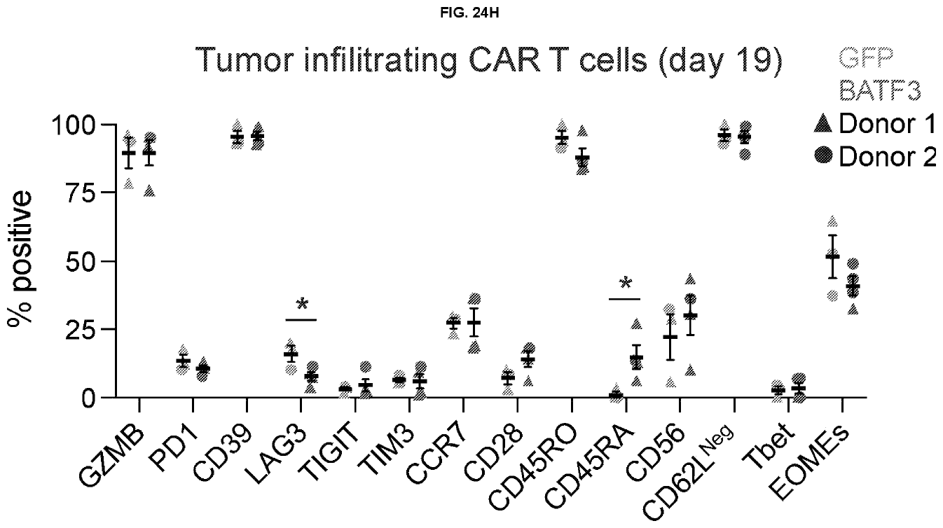

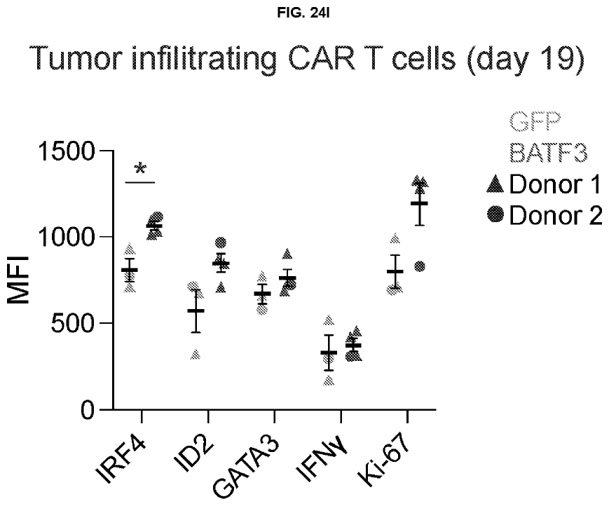

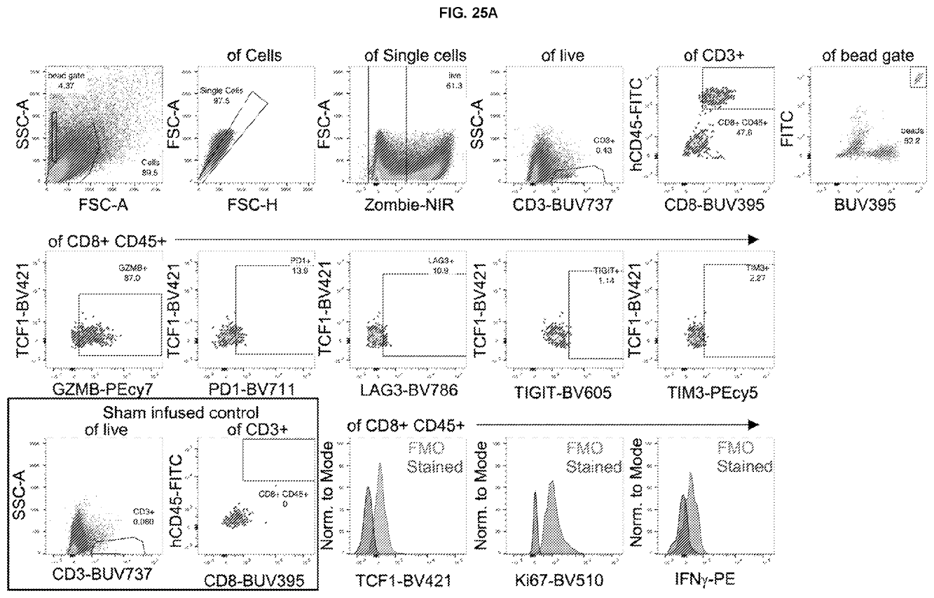

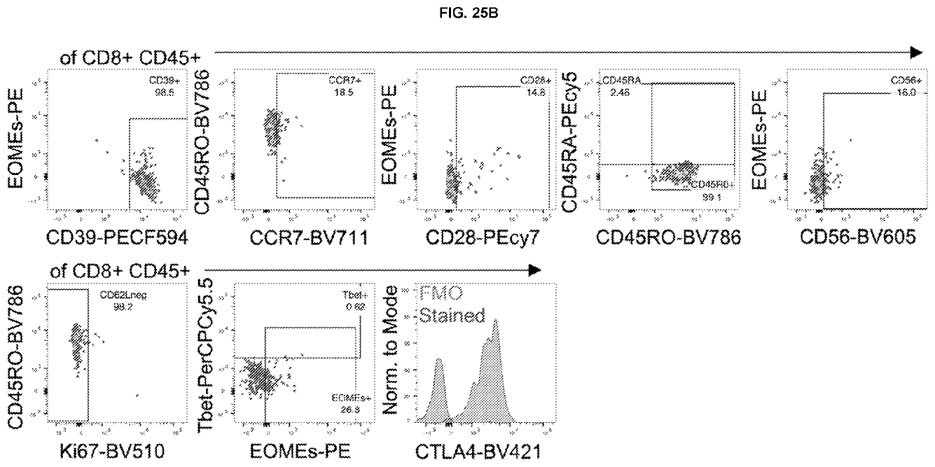

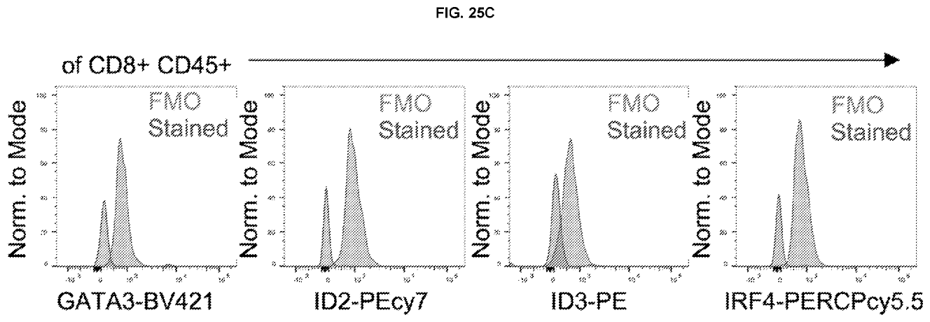

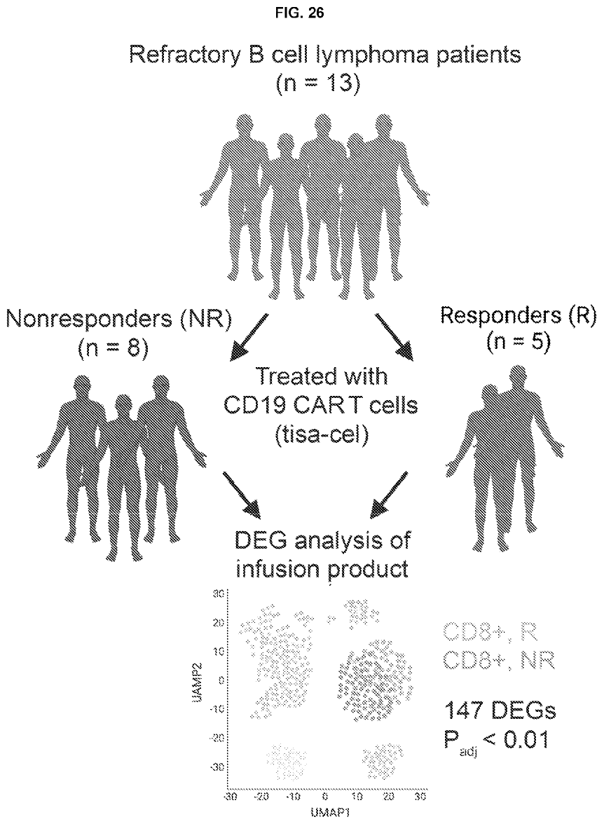

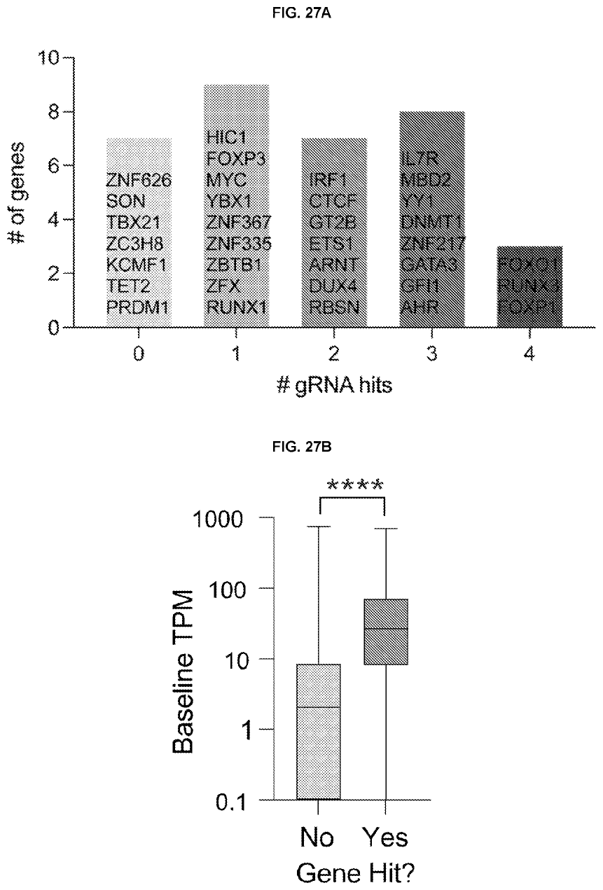

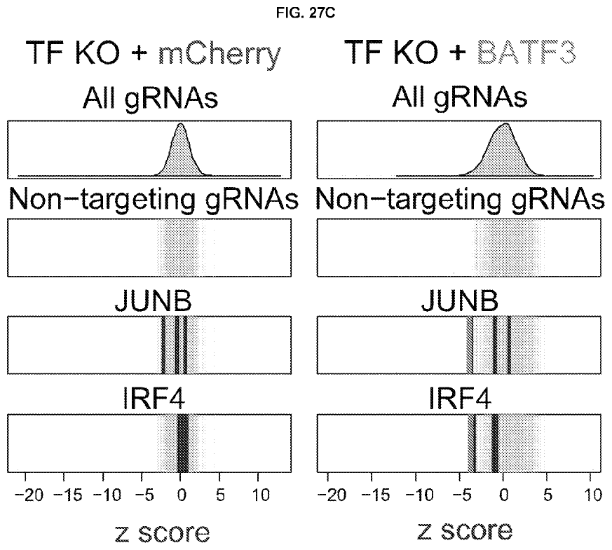

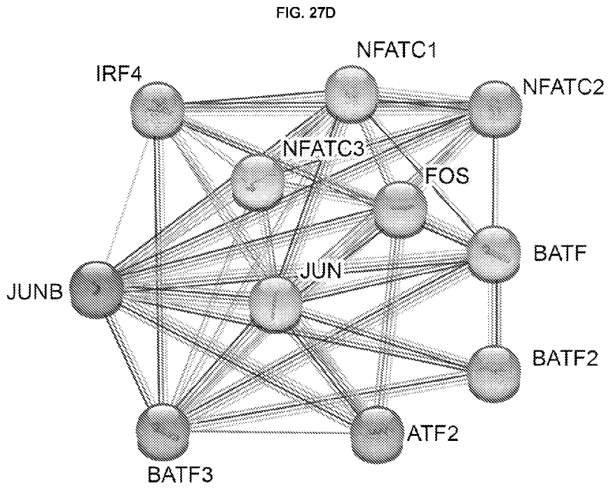

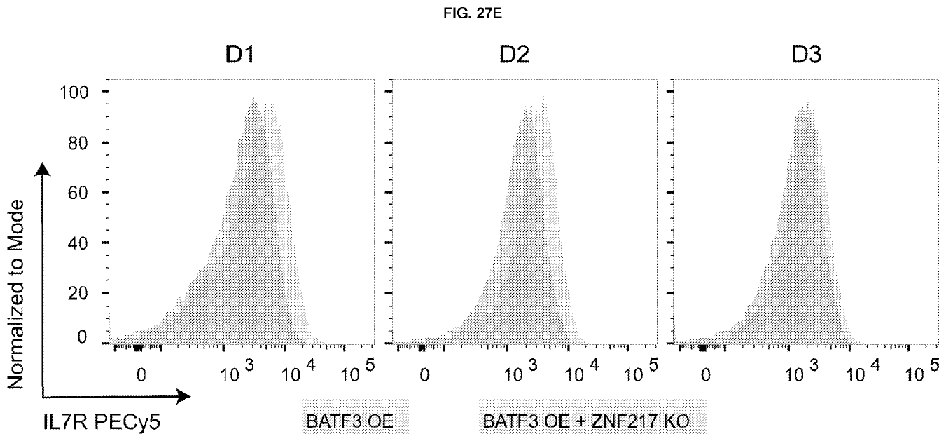

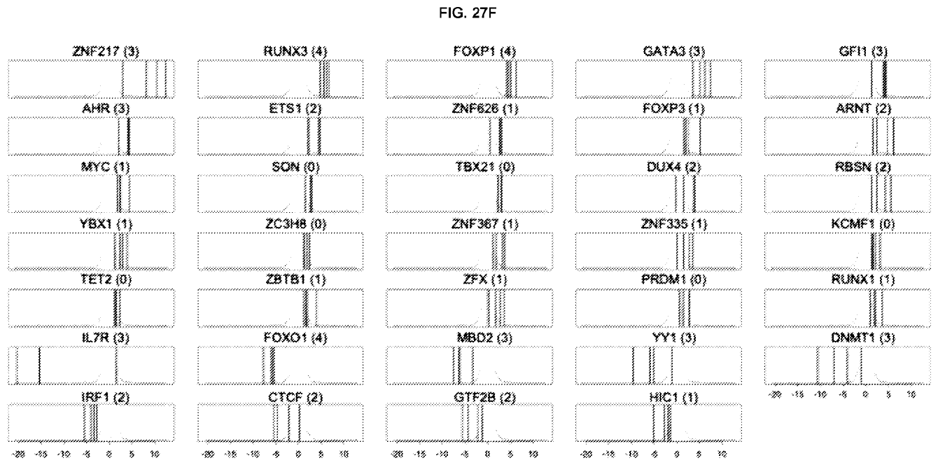

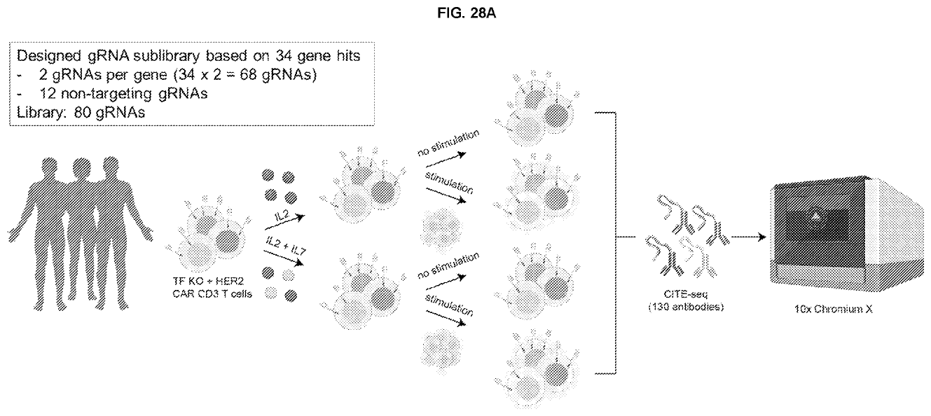

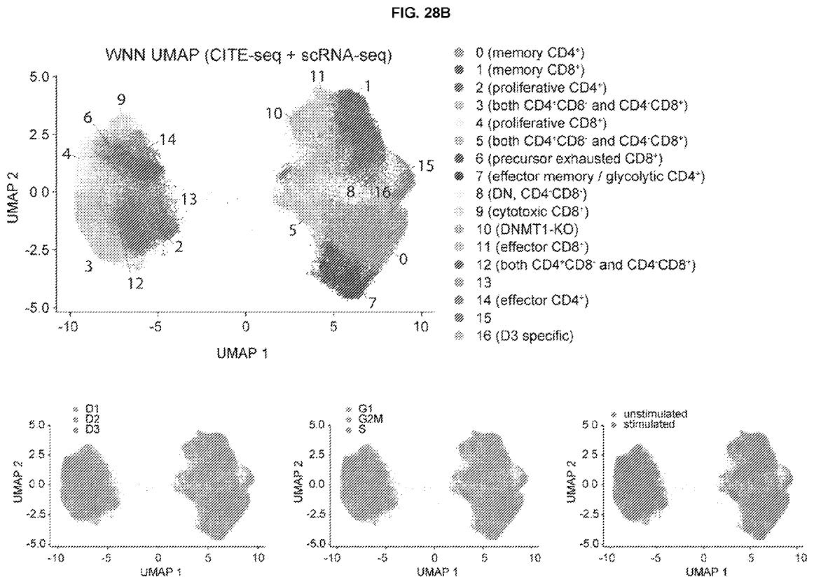

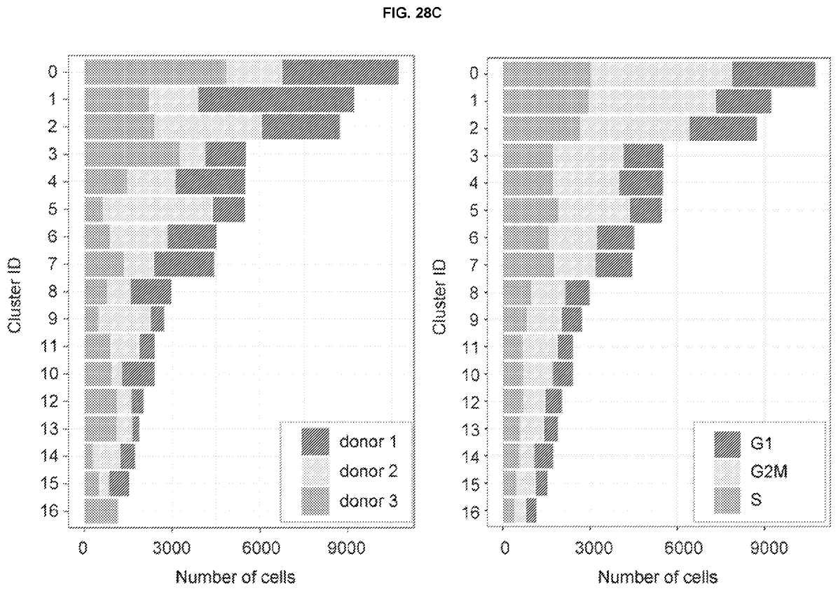

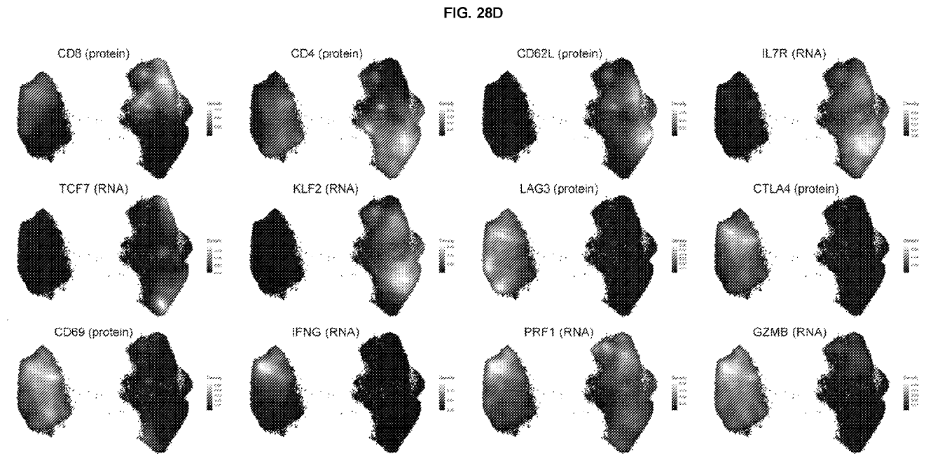

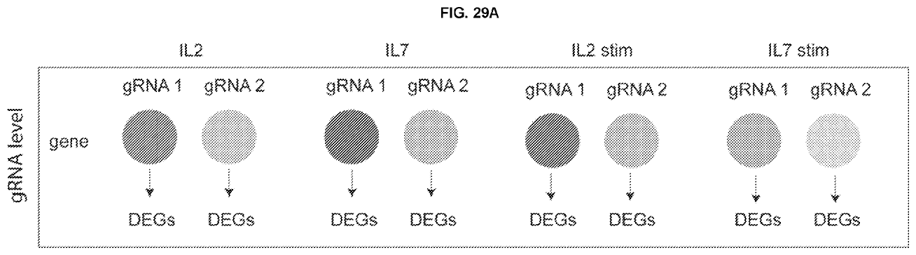

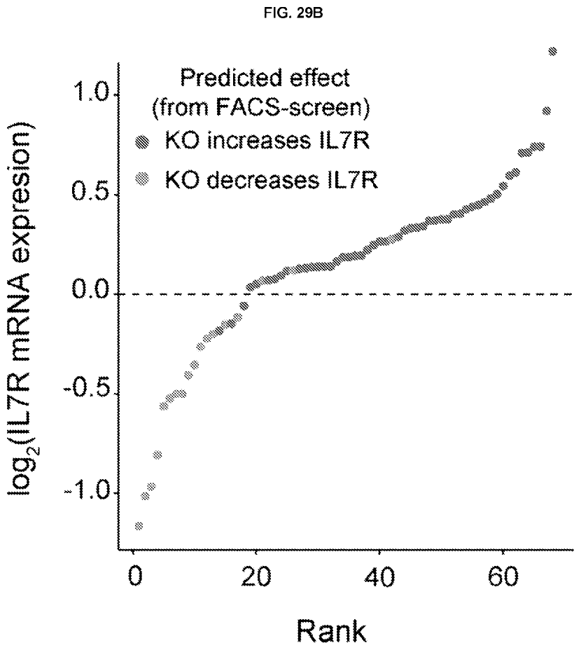

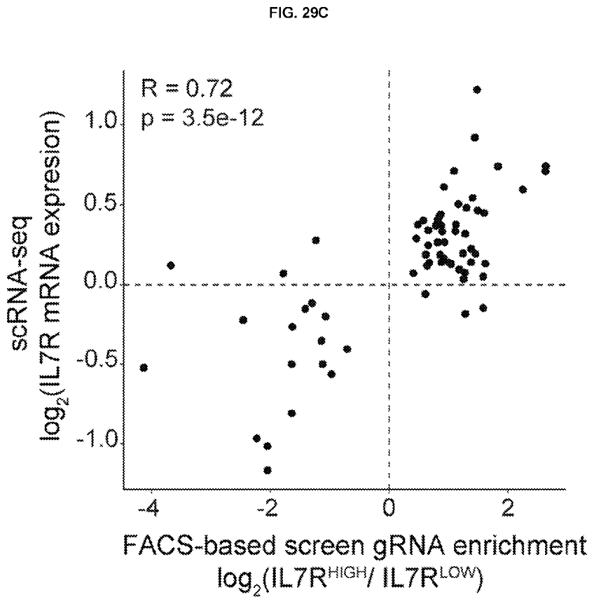

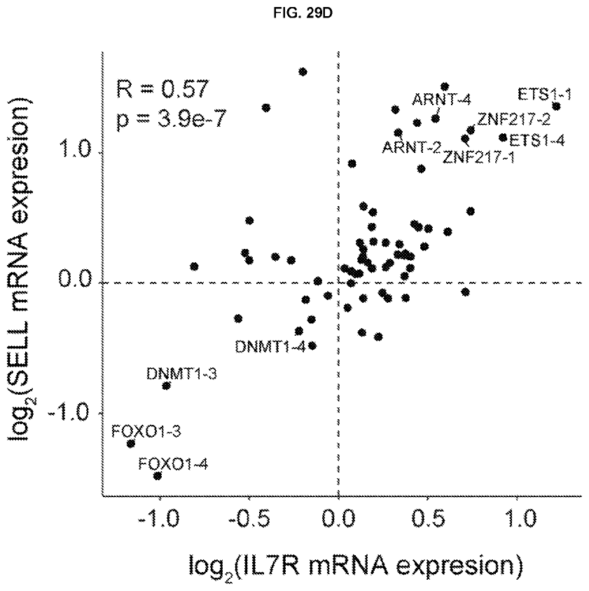

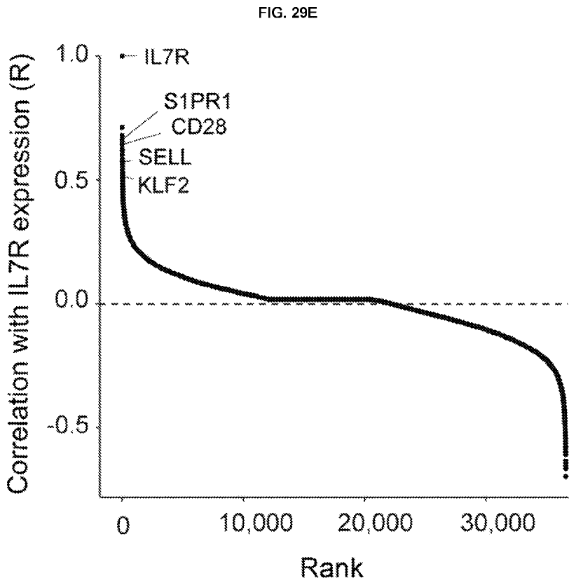

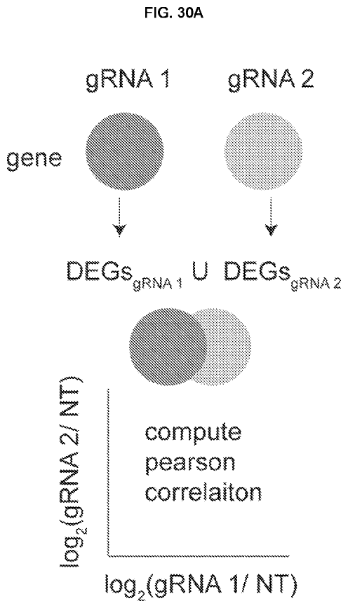

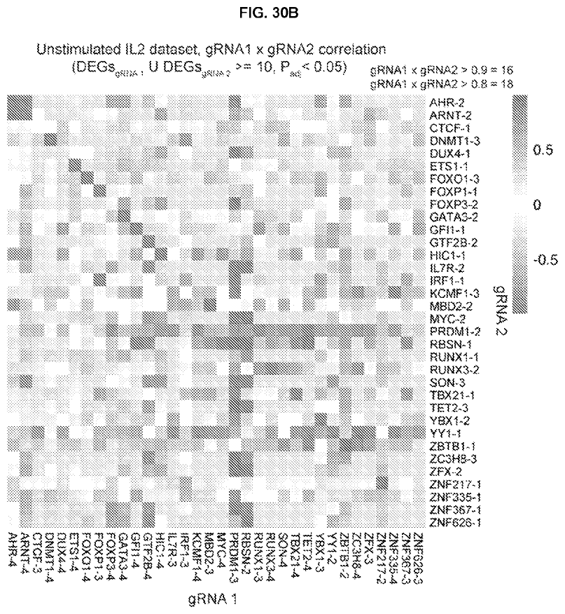

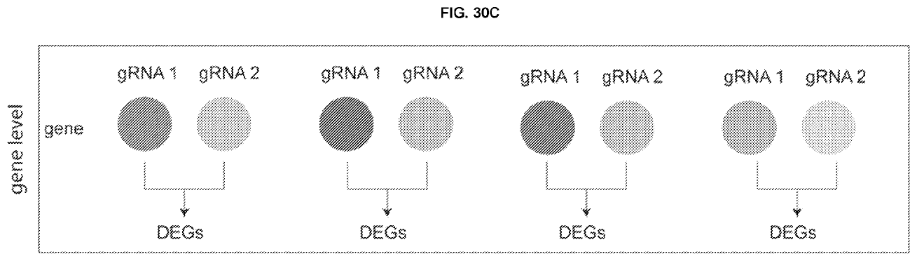

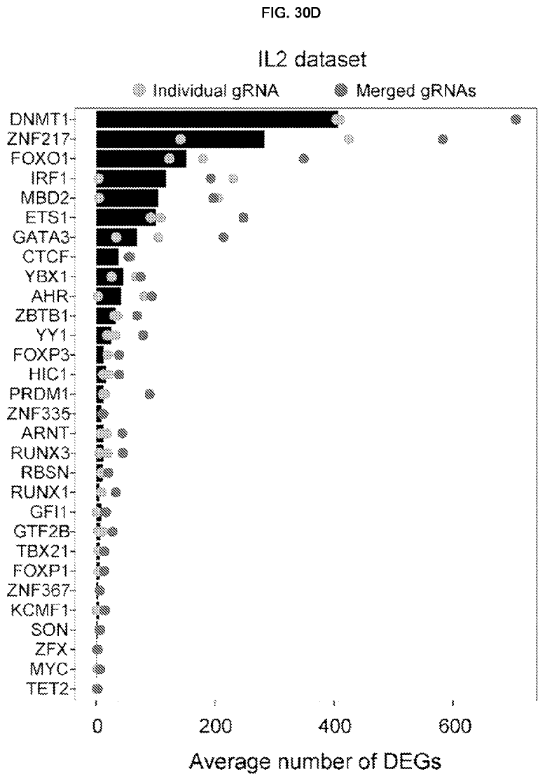

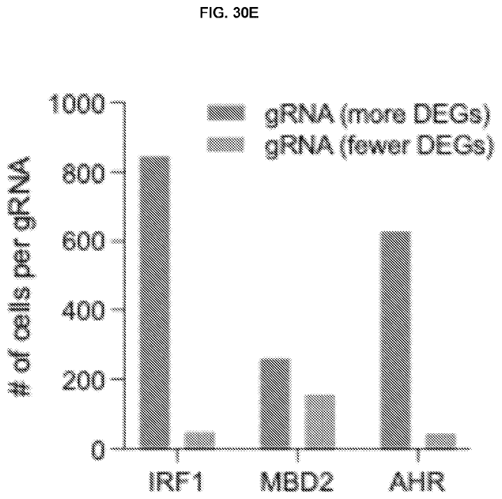

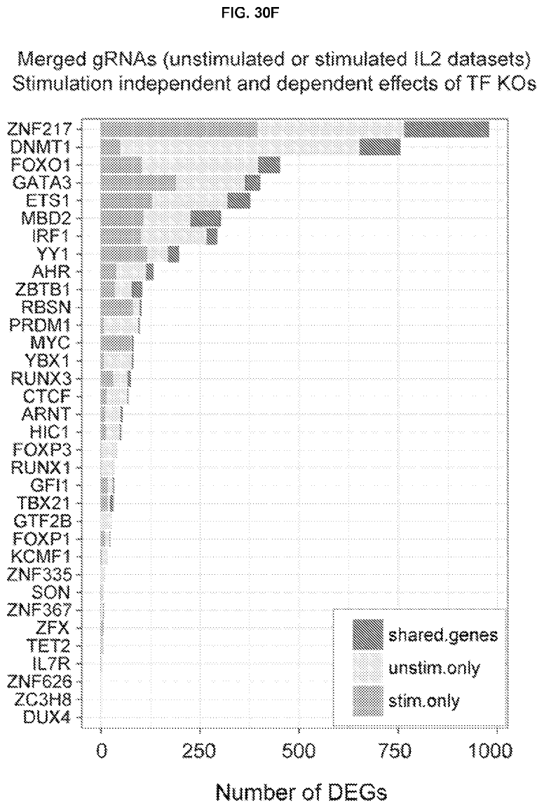

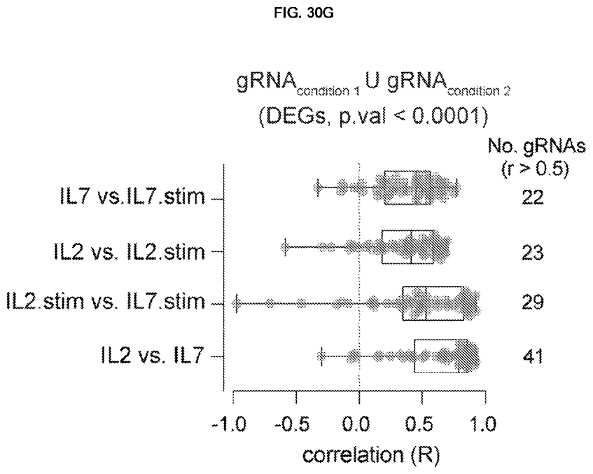

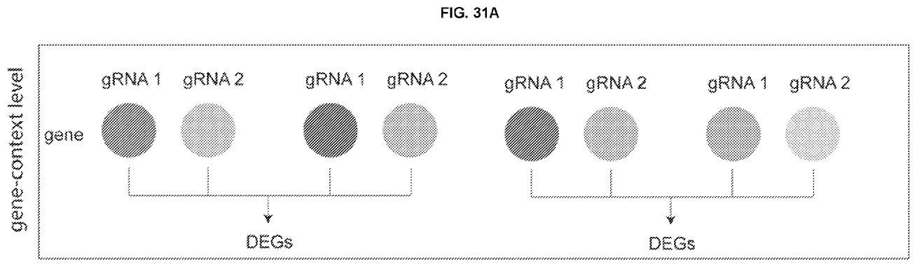

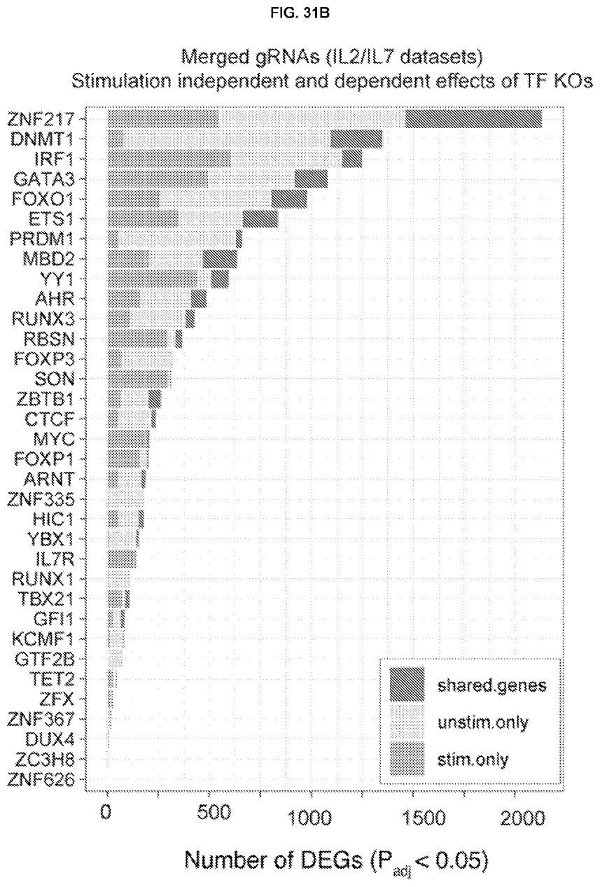

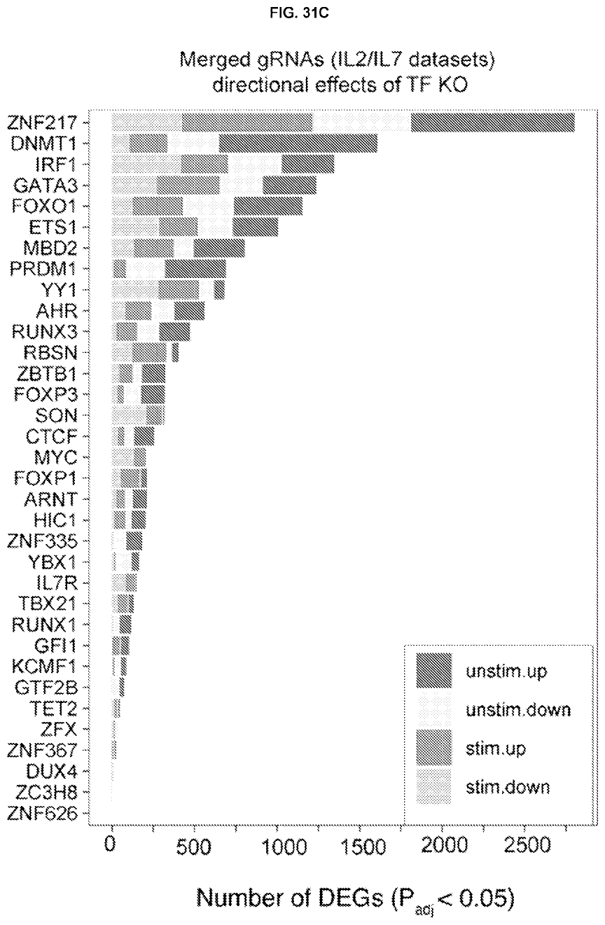

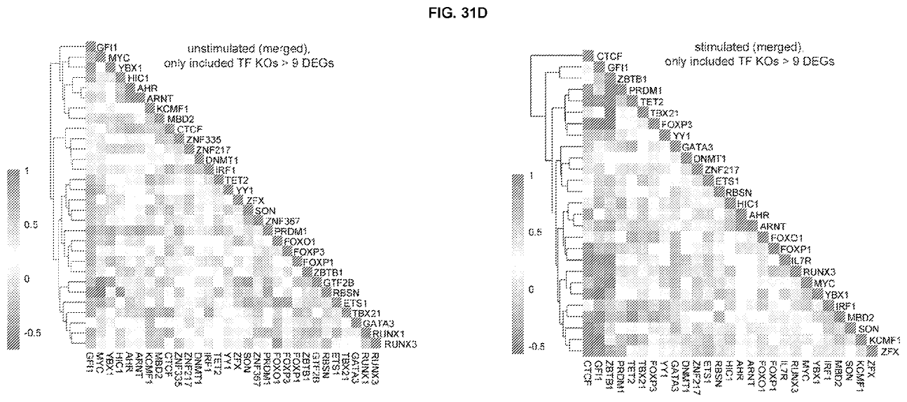

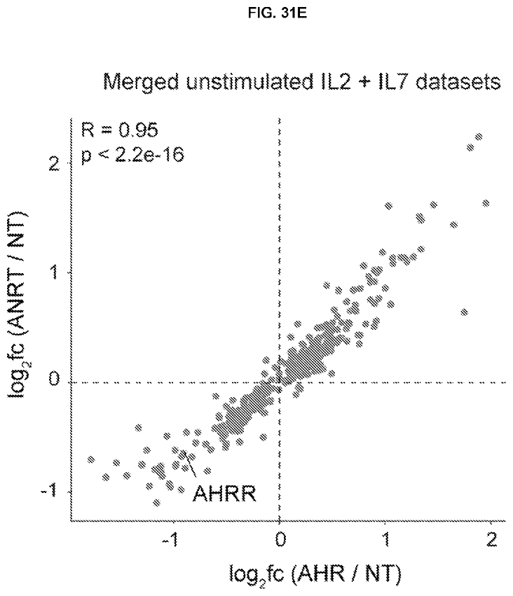

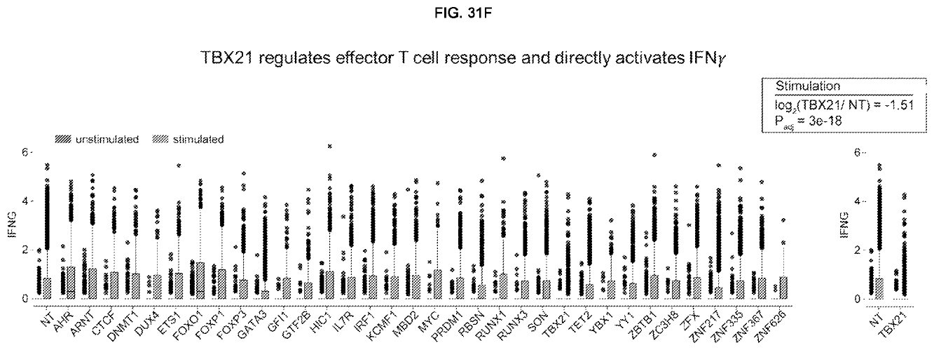

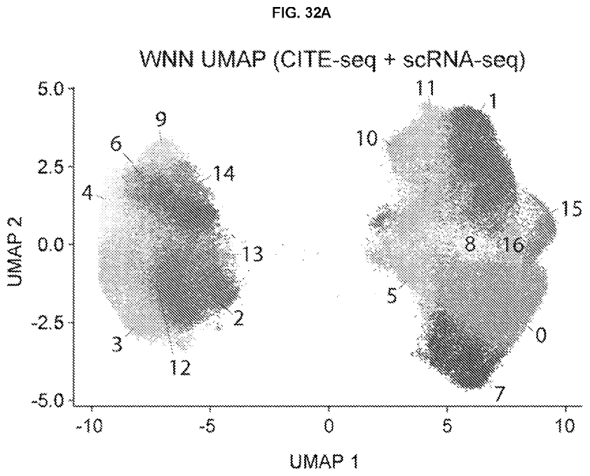

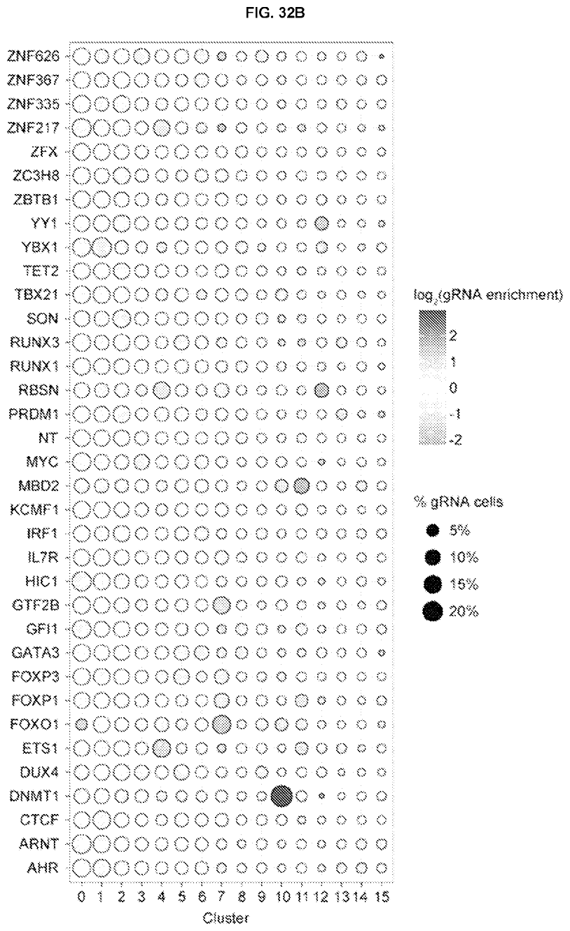

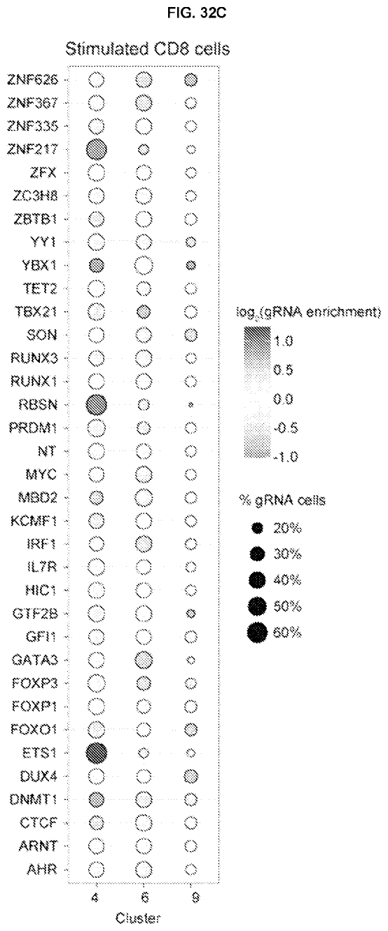

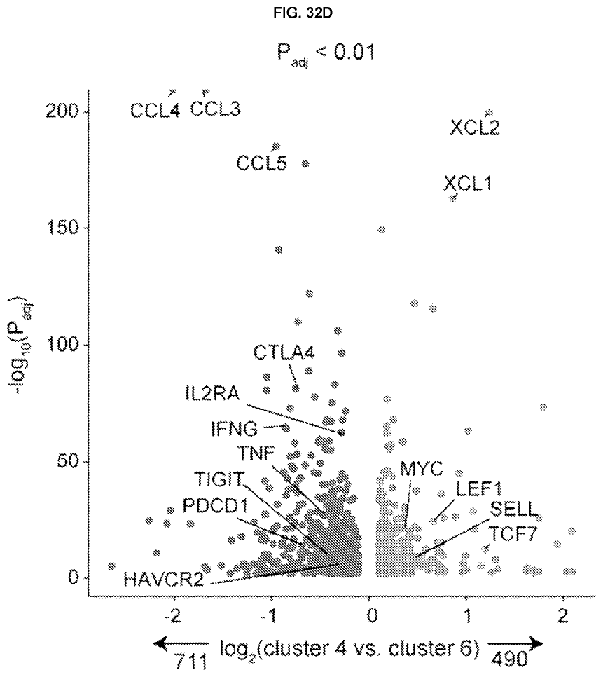

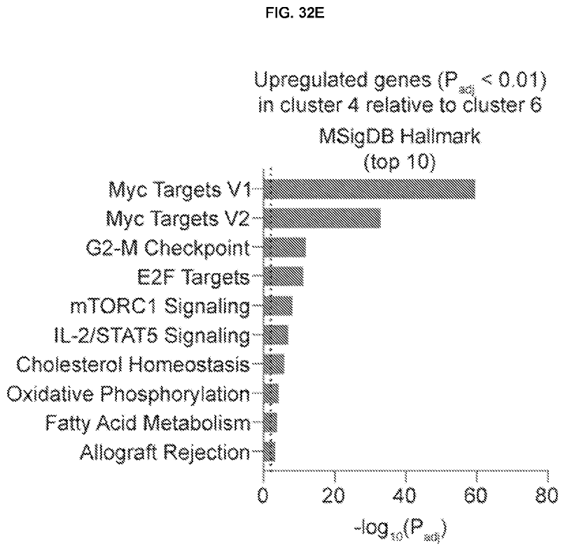

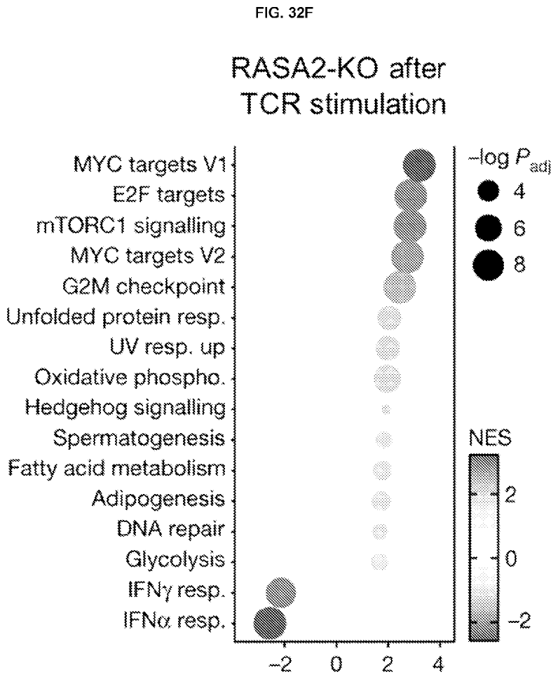

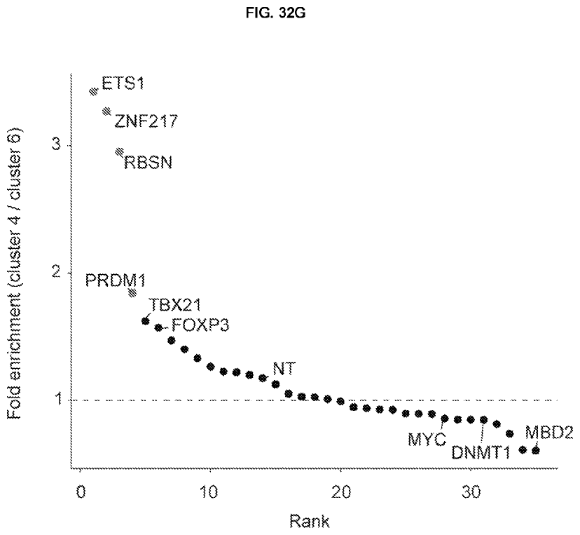

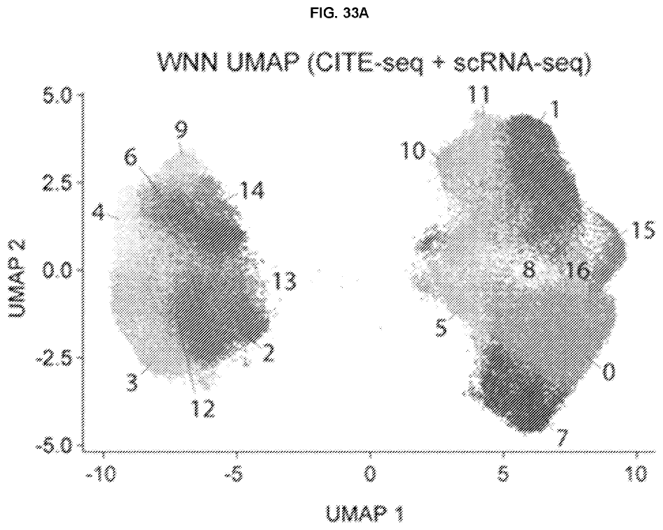

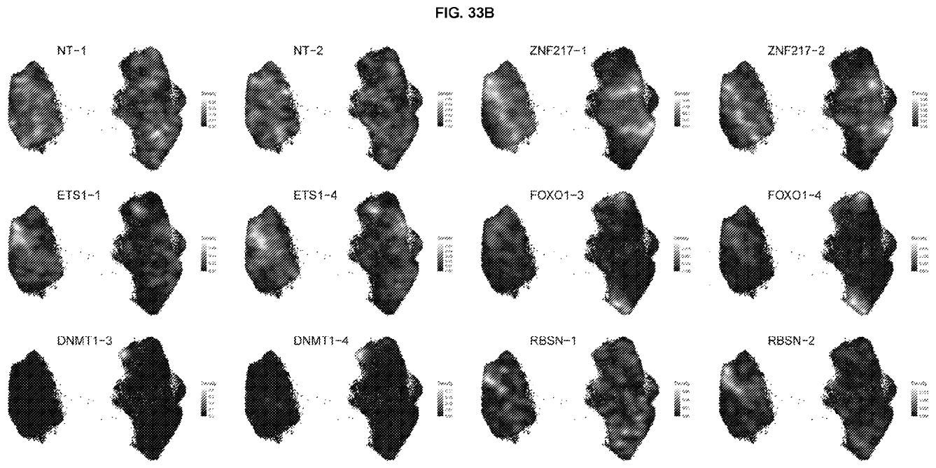

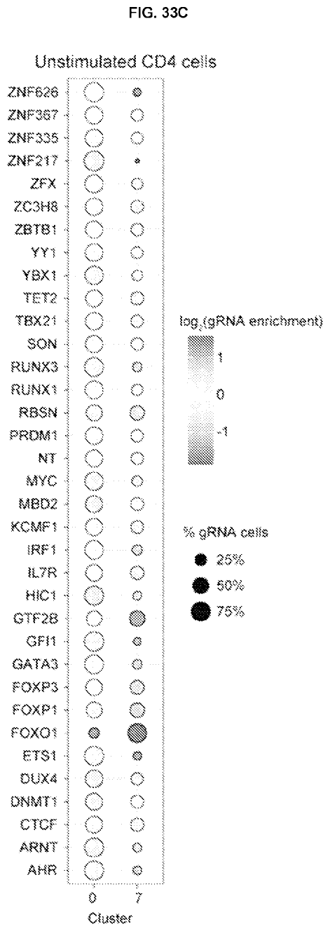

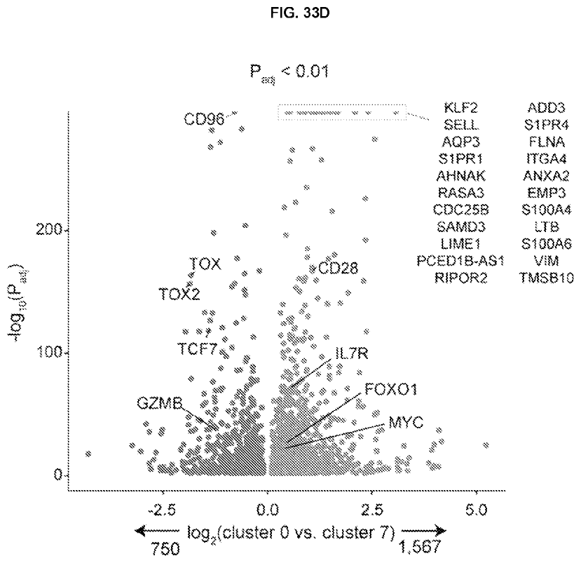

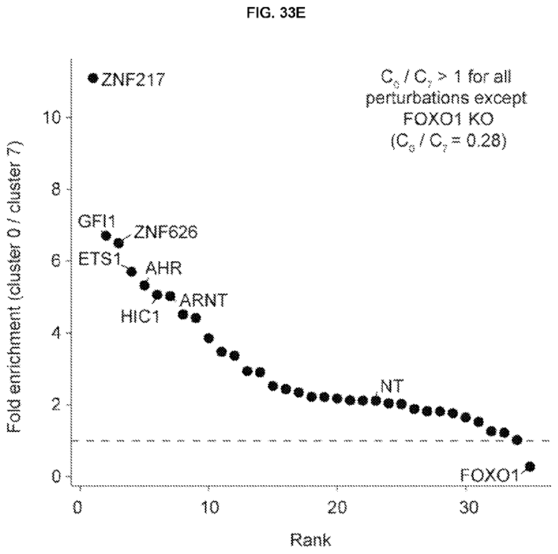

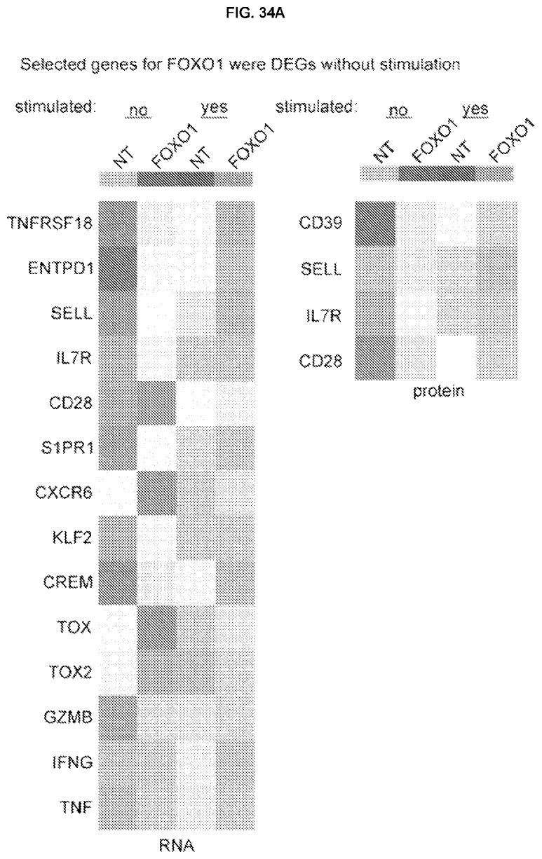

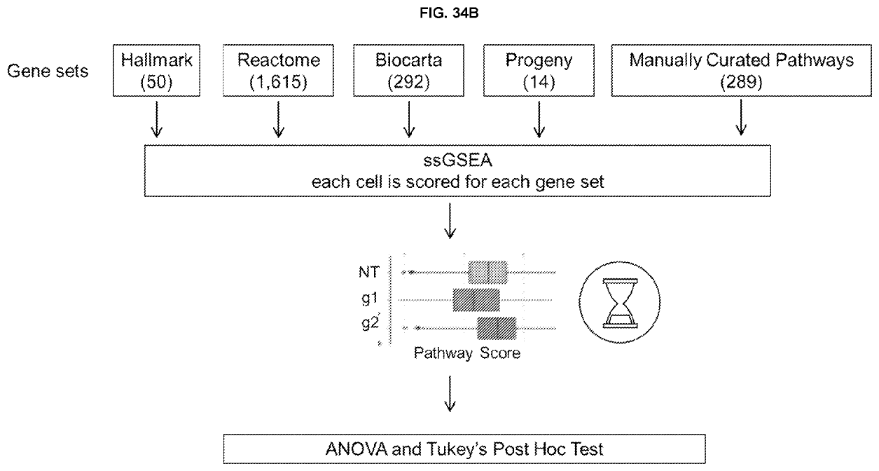

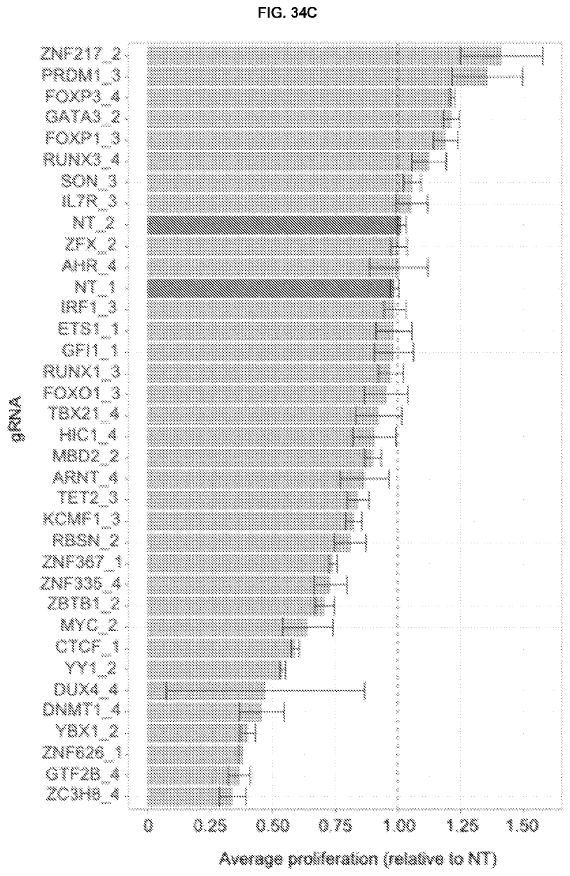

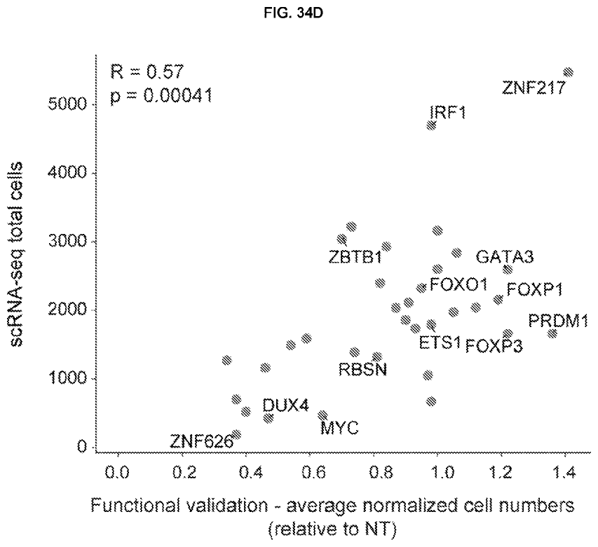

Pearson's correlation coefficient (r) is indicated in the upper left. FIGS. 1H-1I are volcano plots showing significance (P_{adj}) versus fold change in gRNA abundance between IL2RA-high and IL2RA-low populations for the IL2RA CRISPRa Jurkat screens (n=3 replicates) with (FIG. 1H) dSaCas9^{VP64} and (FIG. 11) ^{VP64}dSaCas9^{VP64}. Light gray with black outline and dark gray data points indicate respective gRNA hits (P_{adj}<10⁻⁵). Black data points indicate non-significant IL2RA gRNAs and light gray data points indicate the 94 NT gRNAs. FIG. 1J is a graph showing normalized IL2RA MFI of dSaCas9^{VP64} and ^{VP64}dSaCas9^{VP64} Jurkat lines transduced with indicated gRNAs (n=2 replicates). Each gRNA was normalized to the IL2RA MFI of the dSaCas9^{VP64} Jurkat line transduced with NT. A paired ratio t-test was used to compare gRNA activity between dSaCas9^{VP64} and ^{VP64}dSaCas9^{VP64} Jurkat lines. FIG. 1K is a graph showing relative IL2RA mRNA expression of Jurkat CRISPRa lines transduced with indicated gRNA on day 9 post-transduction (n=2, error bars represent SEM). A one-way ANOVA with Dunnett's post hoc test was used to compare each gRNA to the NT.

FIGS. 2A-2F show that CRISPR interference and activation gene screens identify transcriptional and epigenetic regulators of human CD8+ T cell state. FIG. 2A is a schematic of CRISPRi/a TF screens. FIGS. 2B-2C are volcano plots showing significance (P_{adj}) versus fold change in gRNA abundance between CCR7-high and CCR7-low populations for the (FIG. 2B) CRISPRi and (FIG. 2C) CRISPRa screens. Dark gray data points indicate gRNA hits (P_{adj}<0.05) and are annotated with their target gene. Black and light gray data points represent non-significant gRNAs and non-targeting gRNAs. FIG. 2D are graphs showing fold change of BATF3 and BATF CRISPRa gRNA hits for each donor. Dark grey vertical lines represent BATF3 or BATF gRNAs and light gray vertical lines represent the distribution of 120 non-targeting gRNAs. FIGS. 2E-2F are diagrams showing all BATF3 (FIG. 2E) and BATF (FIG. 2F) CRISPRa gRNAs in gRNA library relative to TSS, chromatin accessibility, and cCREs. Gray and black vertical lines represent gRNA hits and non-significant gRNAs, respectively.

FIGS. 3A-3I show single cell RNA-sequencing characterization of gene candidates. FIGS. 3A-B are volcano plots showing significance (P_{adj}) versus average fold change of CCR7 expression for each gRNA compared to non-perturbed cells for CRISPRi (FIG. 3A) and CRISPRa (FIG. 3B) perturbations. Open circle and gray with black outline data points indicate gRNA hits (P_{adj}<0.05). Light gray data points indicate NT gRNAs. True positive and negative rates are displayed above each volcano plot. FIG. 3C is a plot showing the average fold change in target gene expression for non-targeting gRNAs and targeting gRNAs across CRISPRi and CRISPRa perturbations. A two-way ANOVA with Tukey's post hoc test was used to compare the fold change in target gene expression between groups. FIG. 3D is a dot plot depicting the average expression and percent of cells expressing target genes, memory markers, and effector molecules for the indicated CRISPRi perturbations. FIG. 3E is a scatter plot showing the number of differentially expressed genes (DEGs defined as P_{adj}<0.01) associated with each gRNA versus the gRNA effect on the target gene for both CRISPRi and CRISPRa perturbations. FIG. 3F is a scatter plot showing the correlation of the union set of DEGs between the top CRISPRi MYB gRNAs. FIG. 3G is a scatter plot showing the correlation of the union set of DEGs between the top CRISPRa BATF3 gRNAs. FIGS. 3H-3I are plots showing representative enriched pathways for the top three (FIG. 3H) CRISPRi and (FIG. 3I) CRISPRa gRNAs.

FIGS. 4A-4H show that BATF3 overexpression promotes specific features of memory T cells and counters exhaustion and cytotoxic gene signatures. FIG. 4A is a representative histogram of IL7R expression in CD8+ T cells with or without BATF3 overexpression on day 8 post transduction. FIG. 4B is a graph showing the summary statistics of IL7R expression with or without BATF3 overexpression (n=3 individual donors, paired t test was used to compare IL7R expression between groups, lines connect the same donor). FIG. 4C is a graph showing differential gene expression analysis between CD8+ T cells with or without BATF3 overexpression on day 10 post transduction. Dark grey data points indicate differentially expressed genes (DEGs, P_{adj}<0.01, n=5 donors). FIGS. 4D-4E are graphs showing selected enriched (FIG. 4D) and depleted (FIG. 4E) biological processes from BATF3 overexpression. FIG. 4F is a heatmap of DEGs related to T cell exhaustion, regulatory function, cytotoxicity, transcriptional activity, and glycolysis. FIG. 4G is representative histograms of exhaustion markers (TIGIT, LAG3, and TIM3) on day 12 after acute or chronic stimulation across groups. FIG. 4H is a stacked bar chart with average percentage of CD8+ T cells positive for 0, 1, 2, or 3 exhaustion markers (TIGIT, LAG3, TIM3) on day 12 after chronic stimulation across groups (n=3 independent donors, error bars represent SEM).

FIGS. 5A-5L show that BATF3 OE enhances tumor control in vivo and programs a transcriptional signature associated with clinical response to ACT. FIG. 5A is a graph showing tumor viability after 24 hours of co-culture with GFP CAR^{null}, GFP CAR+, and BATF3 OE CAR+ CD8 T cells at indicated effector to target (E:T) cell ratios (n=3 individual donors, error bars represent SEM). A two-way ANOVA with Dunnett's post hoc test was used to compare tumor viability between GFP+CAR+ and BATF3+ CAR+ T cells at each E:T ratio. FIGS. 5B-5C are graphs showing tumor volume over time for untreated mice and mice treated with (FIG. 5B) 5×10⁵ or (FIG. 5C) 2.5×10⁵ CAR T cells with or without BATF3 overexpression (n=1 donor, 4-5 mice per treatment, error bars represent SEM). A two-way ANOVA was used to compare the tumor volumes at each time point across treatments. Tumor volumes were not statistically different between untreated and control CAR groups at any time point. Tumor volumes were significantly different between untreated and BATF3 OE CAR groups from day 31 onward. The asterisks above indicate significant differences in tumor volumes between mice treated with control and BATF3 OE CAR T cells at each time point. FIG. 5D is a graph showing the average percentage of CD8+ T cells within each resected, dissociated tumor on day 3 post-treatment (n=2 donors, 2-3 mice per donor, error bars represent SEM). A Mann—Whitney test was used to compare the percentage of CD8+ cells between groups. FIGS. 5E-5G are graphs showing Ki-67 (FIG. 5E), TCF1 (FIG. 5F), and IFNy (FIG. 5G) MFI of tumor infiltrating CAR T cells on day 3 across groups (n=2 donors, 2-3 mice per donor, error bars represent SEM). Unpaired t tests were used to compare MFI between groups. FIG. 5H is a graph showing the average percentage and FIG. 5I is a graph showing the total number of CD8+ T cells within each resected, dissociated tumor on day 19 post-treatment across groups. A Mann-Whitney test was used to compare the percentage and total number of CD8+ cells between the two groups. FIGS. 5J-5K are graphs showing TCF1 (FIG. 5J) and ID3 (FIG. 5K) MFI of tumor infiltrating CAR T cells on day 19 across groups (n=2 donors, 1-3 mice per donor, error bars represent SEM).

Unpaired t tests were used to compare MFI between groups. FIG. 5L is a volcano plot of significance (P_{ad}j) versus fold change between BATF3 OE and control CD8+ T cells for a subset of 144 genes that were negatively (open circle data points) or positively (gray data points) associated with clinical outcome to CD19 CAR T cell treatment. The size of each data point corresponds to the strength of association between gene expression and clinical response.

FIGS. 6A-6G show that CRISPRko screens reveal co-factors of BATF3 and novel targets for cancer immunotherapy. FIG. 6A is a schematic of CRISPRko screens. FIG. 6B is graphs showing z scores of gRNAs for selected genes in mCherry (left) and BATF3 (right) screens. Enriched gRNAs (P_{adj}<0.01) are labeled in dark gray or medium gray for each screen. Non-targeting gRNAs are labeled in light gray. FIG. 6C is graphs showing each gene target in the mCherry (top) and BATF3 (bottom) screens ranked based on the MAGeCK robust ranking aggregation (RRA) score in both IL7R^{LOW} (left) and IL7R^{HIGH} (right) populations. Dark gray and light gray data points represent enriched genes in each screen. Dashed lines indicate an FDR <0.05 cutoff. FIG. 6D is a scatter plot of z scores for each gRNA in CRISPR-ko screens with mCherry and BATF3 with enriched gRNAs (P_{adj}<0.01) colored medium gray, dark gray, or black. FIG. 6E is a graph showing the average percentage IL7R+ (left) and relative IL7R MFI (right) in CD8+ T cells with mCherry or BATF3 across gRNAs. Relative IL7R MFI was calculated by dividing the IL7R MFI of each targeting gRNA by the IL7R MFI of the non-targeting gRNA for each donor within the treatment group (n=3 donors, error bars represent SEM). FIG. 6F is a scatter plot of z score of BATF3 overexpression and ZNF217 knockout. FIG. 6G is a graph showing the gene ontology of ZNF217 knockout

FIGS. 7A-7E show a proof-of-principle CRISPRi B2M promoter tiling screen in primary human CDS+ T cells. FIG. 7A is a volcano plot of significance (P_{adj}) versus fold change in gRNA abundance between B2M-high and B2M-low populations for the B2M CRISPRi screen. Black outlined gray data points indicate B2M gRNA hits with a P_{adj}<10⁻¹⁰. Black data points indicate non-significant B2M gRNAs. Gray data points indicate the 250 NT gRNAs. FIG. 7B is B2M gRNA fold change versus gRNA position relative to the transcriptional start site (TSS). Dashed lines represent the previously defined optimal window (−50 to +300 bp of TSS) for CRISPRi. FIG. 7C is B2M gRNA fold change as a function of the final base pair of the PAM (5′-NNGRRN-3′). x represents the number of gRNA hits and y represents the total number of gRNAs in the library for each PAM variant. A global one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 7D is Validation of B2M gRNA hits. Percentage of B2M positive cells on day 9 post-transduction plotted in rank order based on the mean gRNA activity (n=3 replicates of CDS+ T cells from pooled PBMC donors, error bars represent SEM). A one-way ANOVA with Dunnett's post hoc test was used to compare the mean percentage of B2M positive cells for each gRNA to NT. The final base pair of the PAM for each gRNA is indicated beneath the gRNA label. FIG. 7E Relative B2M mRNA expression of CD8+ cells transduced with indicated gRNA on day 9 post-transduction (n=3, error bars represent SEM) using RT-qPCR. A one-way ANOVA with Dunnett's post hoc test was used to compare each gRNA to the NT.

FIGS. 8A-8G show flow cytometry validation of CD2 and B2M gRNA hits and multiplex gene silencing. Representative contour plots of (FIG. 8A) CD2 and (FIG. 8B) B2M expression in CD8+ T cells across non-targeting (NT), non-hit (NH), and gRNA hits (H) measured on day 9 post transduction. FIG. 8C is a graph showing the relationship between relative CD2 mean fluorescence intensity (MFI) of CD2 silenced cells and the percentage of CD2 silenced cells. Pearson's correlation coefficient (r) is indicated above the graph. FIG. 8D is a graph showing the relationship between B2M gRNA activity and fold change enrichment in screen. Pearson's correlation coefficient (r) is indicated above the graph. Average percentage of (FIG. 8E) CD2 silenced, (FIG. 8F) B2M silenced, and (FIG. 8G) dual CD2 and B2M silenced CD8+ T cells on day 10 post transduction with the indicated pairs of non-targeting, CD2, and B2M gRNAs (n=3 replicates of CD8+ T cells from pooled P8MC donors, error bars represent SEM). g1 is driven by a human U6 promoter and g2 is driven by a mouse U6 promoter. CD2 HS and B2M H1 gRNAs were used for multiplex gene silencing experiments.

FIGS. 9A-9D show dSaCas9^{VP64} and ^{VP64}dSaCas9^{VP64} IL2RA promoter tiling CRISPRa screens in Jurkats. FIG. 9A is a schematic of dSaCas9^{VP64} and ^{VP64}dSaCas9^{VP64} IL2RA promoter tiling CRISPRa screens in Jurkats. FIG. 9B is an UCSC genome browser track of IL2RA locus with statistical significance displayed for each gRNA in ^{VPS4}dSaCas9^{VP64} CRISPRa screen. gRNA hits are annotated and labeled in dark gray. ATAC-seq and ENCODE candidate cis regulatory elements (cCREs) tracks are overlayed for visualization of chromatin accessibility and annotations. cCREs in medium gray are defined as promoter-like elements and cCREs in light gray are defined as enhancer-like elements. FIG. 9C is a graph showing fold change in gRNA abundance as a function of the final base pair of the PAM (5′-NNGRRN-3′) for IL2RA gRNAs. x represents the number of gRNA hits for each PAM and y represents the total number of gRNAs for each PAM. A global one-way ANOVA with Dunnett's post hoc test was used to compare the average fold change of gRNAs for each PAM variant to NNGRRT (*<0.05 denotes that the fold change of gRNAs targeting NNGRRT PAMs was significantly different than all other PAM variants). FIG. 9D is a representative overlayed histograms of IL2RA expression for dSaCas9^{VP64} and VP64dSaCas9^{VP64} Jurkat lines on day 9 post-transduction across gRNAs.

FIGS. 10A-10F show a head-to-head comparison of ^{VP64}dSaCas^{VP64} and ^{VP64}dSpCas9^{VP64} lentiviral titers and ^{VP64}dSaCas9^{VP64} activity in primary human CD8+ T cells. FIG. 10A is a graph showing the transduction rate of primary human CD8+ T cells as a function of lentiviral volume for all-in-one ^{VP64}dSaCas9^{VP64} and ^{VP64}dSpCas9^{VP64} plasmids on day 9 post-transduction (n=2 donors, error bars represent SEM). FIG. 10B is a graph showing the linear range of transduction rate as a function of lentiviral volume. Pearson's correlation coefficient (r) and ratio of slopes were calculated using simple linear regression. (n=2 donors, error bars represent SEM, ***<0.001 denotes the slopes of the two lines are significantly different). FIG. 10C is a plot showing flow cytometry controls used to set the Thy1.1+ gate for the lentiviral titer experiment. FIG. 10D is representative contour plots of Thy1.1 expression in CD8+ T cells transduced with serial titrations of ^{VP64}dSaCas9^{VP64} and ^{VP64}dSpCas9^{VP64} lentivirus. FIG. 10E is representative contour plots of EGFR expression in primary human CD8+ T cells on day 8 post-transduction with all-in-one lentiviruses encoding for ^{VP64}dSaCas9^{VP64} and either a non-targeting (NT) or an EGFR gRNA. FIG. 10F is summary statistics of EGFR activation (n=2 replicates of CD8+ T cells from pooled PBMC donors, error bars represent SEM). A global one-way ANOVA with Dunnett's post hoc test was used to compare the effect of EGFR gRNAs to the NT gRNA.

FIGS. 11A-11C show TF and epigenetic modifier gRNA library design. FIG. 11A is details of gRNA library targeting curated list of TFs and epigenetic modifiers. FIG. 11B is a histogram of gRNA representation across 121 candidate genes. FIG. 11C is a graph showing the transduction rate of primary human CD8+ T cells as a function of lentiviral volume for all-in-one CRISPRi and CRISPRa TF gRNA plasmids on day 9 post-transduction.

FIGS. 12A-12D show representative gating and post sorts for initial and final sorts for CRISPRi/a TF screens. FIG. 12A is a representative initial gating for CD8+CCR7+ T cell population. FIG. 12B is a representative post sort of CD8+CCR7+ T cell population. FIG. 12C is a representative final gating strategy for transduced (Thy1.1+) cells in the lower and upper 10% tails of CCR7 expression on day 9 post-transduction. FIG. 12D is a representative post sorts of Thy1.1+CCR7-low (left) and Thy1.1+CCR7-high (middle) populations. Overlay of sorted populations (right) shows clear separation of CCR7-low and CCR7-high populations.

FIGS. 13A-13D show effects of CRISPRi/a gRNA hits across donors. FIG. 13A is a scatter plot of fold change in gRNA abundances between CCR7-high and CCR7-low populations across two donors in CRISPRi screens. FIG. 13B is a distribution of fold change in gRNA abundances between CCR7-high and CCR7-low populations for representative gRNA hits in the CRISPRa screen across three donors. Blue vertical lines represent gRNA hits and gray vertical lines represent the distribution of 120 non-targeting gRNAs. FIGS. 13C-13D are scatter plots of number of gRNA hits versus total number of gRNAs for each gene in (FIG. 13C) CRISPRi and (FIG. 13D) CRISPRa TF screens. A slight vertical stagger was implemented for visualization purposes, but there are only discrete values as denoted by the colors (0 gRNA hits=black, 1 gRNA hit=medium gray, 2 gRNA hits=dark gray, 3 gRNA hits=light gray).

FIGS. 14A-14D show quality control of differential gene expression analyses for CRISPRi and CRISPRa TF scRNA-seq characterization. FIGS. 14A-14B are graphs showing the average number of differentially expressed genes (DEGs) for targeting and non-targeting gRNAs as a function of gRNA UMI threshold used for gRNA assignment to cells in (FIG. 14A) CRISPRi and (FIG. 14B) CRISPRa scRNA-seq screens. FIGS. 14C-14D are volcano plots of (FIG. 14C) CRISPRi and (FIG. 14D) CRISPRa scRNA-seq screens with the statistical significance (P_{adj}) of each significant gRNA-gene pair plotted versus the fold change in gene expression relative to non-perturbed cells.

FIGS. 15A-15E show individual validation of subset of CRISPRi/a gRNAs on CCR7 expression. FIGS. 15A-15B are flow cytometry plots of CCR7 expression in CD8+ T cells across three donors with the indicated CRISPRi/a perturbations. FIG. 15C is a graph showing the summary statistics of percent CCR7+ T cells across CRISPRi/a perturbations. A paired, one-way ANOVA test was used to compare the percentage of CCR7+ T cells for each perturbation to the CRISPRi NT treatment group (n=3 donors, error bars represent SEM, different shapes are used to denote each donor). FIG. 15D is graphs showing the number of cells assigned to each gRNA across CRISPRi (left), CRISPRa (middle), and joint CRISPRi/a (right) scRNA-seq datasets with gRNAs stratified based on whether they affected CCR7 expression as predicted by the flow-based screen. A Mann-Whitney test was used to determine statistical significance for each group. FIG. 15E is graphs showing the correlation of the union set of DEGs between CREM gRNAs from CRISPRa scRNA- seq characterization (left) and violin plots of CREM expression across cells assigned to indicated gRNA with the fold change in target gene expression relative to NT and the percent of cells expressing the target gene indicated above (right).

FIGS. 16A-16B show that MYB silencing drives T cells towards an effector phenotype. FIG. 16A is a volcano plot of statistical significance (P_{ad}j) for each gene versus the fold change in gene expression in MYB CRISPRi-perturbed cells relative to non-perturbed cells. All DEGs (P_{ad}, <0.01) are labeled blue apart from MYB, which is labeled dark red. Selected DEGs are annotated. FIG. 16B is a classification of annotated DEGs based on their functional role.

FIGS. 17A-17C show that NR1D1 synthetically induces exhaustion phenotype. FIG. 17A is a UMAP plot of CRISPRa scRNA-seq characterization with cells split by perturbation status: non-perturbed (top) and perturbed (bottom). Dark gray data points indicate cells with a NR1D1 gRNA. Cells were clustered using Seurat's CalcPerturbSig function to mitigate confounding sources of variation such as the donor and phase of cell cycle. FIG. 17B is a volcano plot of statistical significance (P_{ad}j) for each gene versus the fold change in gene expression in NR1 D1 CRISPRa-perturbed cells relative to non-perturbed cells. All DEGs (P_{ad}, <0.01) are labeled blue apart from NR1D1, which is labeled black. Selected DEGs are annotated. FIG. 17C is a violin plot of exhaustion gene signature score across all non-perturbed and NR1D1-perturbed cells in the CRISPRa scRNA-seq screen. UCell gene signature scores are based on the Mann-Whitney U statistic.

FIGS. 18A-18C show kinetics of BATF3 expression and effects of BATF3 OE. FIG. 18A is a graph showing the median BATF3 expression as a function of time relative to BATF3 expression before T cell activation across four treatment groups (n=3 donors, fold change in BATF3 expression was calculated using 2^{-dCT} method relative to baseline BATF3 expression, internal householding control was excluded because T cell stimulation dramatically alters expression of householding genes such as GAPDH and TBP, input mass of RNA into the reverse transcription reaction was the same for all samples). FIG. 18B is plots showing an IL7R fluorescent minus one (FMO, left) control was used to set the IL7R+ gate. Representative IL7R expression of CD8+ T cells from a donor transduced with either GFP (middle) or BATF3 OE (right) on day 8 post-transduction. FIG. 18C is graphs showing transcripts per million (TPM) of selected genes across n=5 donors with either GFP or BATF3 OE on day 10 post transduction. Statistical significance for each gene was determined using paired DESeq2 analysis between treatment groups.

FIGS. 19A-19C show that BATF3 OE attenuates expression of T cell exhaustion markers in chronically stimulated CD8+ T cells. FIG. 19A is a schematic of acute (left) and chronic stimulation (right) with CD3/CD28 dynabeads. FIG. 19B is graphs showing the average percentage of positive cells (top panel) and mean fluorescence intensity (MFI, bottom panel) of exhaustion markers: PD1, LAG3, TIGIT, and TIM3 on day 3 post-transduction with GFP or BATF3 OE (n=3 individual donors, error bars represent SEM, paired t tests used to determine statistical significance). FIG. 19C is graphs showing the time course of PD1, LAG3, TIGIT, and TIM3 expression post-transduction with GFP or BATF3 OE under acute or chronic stimulation (n=3 individual donors, error bars represent SEM).

FIG. 20 shows that chronic antigen stimulation drives extensive chromatin remodeling in control CD8+ T cells. FIG. 20 is a heatmap of differentially accessible regions with selected regions annotated with their nearest gene.

FIGS. 21A-21E show that BATF3 remodels the chromatin landscape of CD8+ T cells under acute stimulation. FIG. 21A is a graph showing the number of ATAC-seq regions with increased or decreased accessibility in CD8+ T cells (n=3 individual donors, differential accessible regions defined as P_{adj}<0.05) with BATF3 OE on day 14 post-transduction. FIG. 21B is a pie graph showing the proportion of differentially accessible (DA) regions based on genomic feature classification. FIG. 21C is a graph showing joint analysis of RNA-seq and ATAC-seq datasets and number of differentially accessible regions near upregulated and downregulated genes. Dashed lines represent the number of unique DEGs associated with DA regions. FIG. 21D is a heatmap of DA regions with selected regions annotated with their nearest gene. FIG. 21E is a representative ATAC-seq tracks at IL7R and TIGIT loci with overlayed rectangles indicating DA regions.

FIGS. 22A-22E show that BATF3 remodels the chromatin landscape of CDB+ T cells under chronic stimulation. FIG. 22A is a graph showing the number of ATAC-seq regions with increased or decreased accessibility in HER2-CAR-2A-BATF3 CD8+ T cells compared to HER2-CAR-2A-GFP CD8+ T cells on day 14 post-transduction after repeated rounds of tumor restimulation (n=2 individual donors, differential accessible regions defined as P_{adj}<0.05). FIG. 22B is a pie graph showing the proportion of differentially accessible (DA) regions based on genomic feature classification. FIG. 22C is a heatmap of DA regions with selected regions annotated with their nearest gene. FIG. 22D is a representative ATAC-seq tracks at IL7R, CTLA4, TIGIT loci with overlayed rectangles indicating DA regions. FIG. 22E is a representative ATAC-seq tracks of the TCF7 locus under acute and chronic stimulation with and without BATF3 OE.

FIGS. 23A-23F show that BATF3 OE in human CD8+ T cells enhances in vitro and in vivo tumor control. FIG. 23A is a graph showing tumor viability after 24 hours of culture in T cell media, co-culture with CAR^{null} T cells, or co-culture with a titration of CAR+ T cell doses ranging from 1:8 to 2:1 E:T (n=3 donors, error bars represent SEM). FIG. 23B is graphs showing tumor viability after 24 hours of co-culture with GFP CAR^{null}, GFP CAR+, and BATF3 OE CAR+ CD8 T cells at indicated effector to target (E:T) cell ratios for each donor. FIG. 23C is a graph showing tumor volume over time as a function of the dose of control HER2 CAR T cells (n=5 mice per treatment, error bars represent SEM). Mice were intravenously injected with CAR T cells on day 21. FIG. 23D is a representative flow plots of CAR expression in CD8+ T cells with control and BATF3 OE CAR lentiviral plasmids on day 9 post-transduction (the same day that the mice were intravenously injected with CAR T cells). FIG. 23E is graphs showing the summary statistics of transduction rates and total CAR+ T cells with control and BATF3 OE CAR lentiviral plasmids on day 9 post-transduction (n=3 donors, lines connect donors across treatments). FIG. 23F is graphs showing tumor volumes of individual mice treated with 5×10⁵ (left panel, n=5 mice per treatment group) or 2.5×10⁵ (right panel, n=4 mice per treatment group) CAR T cells with or without BATF3 overexpression. Thinner lines represent individual mice and thicker lines represent the average tumor volume across mice in a treatment group.

FIGS. 24A-24I show characterization of CAR T cells with or without BATF3 overexpression at multiple time points during in vivo tumor control experiment. FIG. 24A is a graph showing tumor volume over time for untreated mice and mice treated with 5×10⁵ CAR T cells with or without BATF3 overexpression (n=2 donors, 3-4 mice per donor, error bars represent SEM). Flow analysis on input CAR T cells and tumor infiltrating CAR T cells at day 3 and day 19 post-treatment. FIG. 24B is a graph showing the same as FIG. 24A except stratified based on donor. FIG. 24C is a graph showing the percentage of positive cells for indicated markers of input CAR T cells across groups. FIG. 24D is a graph showing MFI for indicated markers of input CAR T cells across groups. FIG. 24E is histograms of TCF1 and LAG3 expression for input CAR T cells. FIG. 24F is a graph showing the average percentage of CD8+ T cells in peripheral blood on day 3 post-treatment across groups. A Mann-Whitney test was used to compare the percentage of CD8+ cells between the two groups. FIG. 24G is a graph showing the percentage of positive cells for indicated markers of tumor infiltrating CAR T cells on day 3 post-treatment across groups. FIG. 24H is a graph showing the percentage of positive cells for indicated markers of tumor infiltrating CAR T cells on day 19 post-treatment across groups. An unpaired t test was used to compare expression of each marker between groups. FIG. 24I is a graph showing MFI for indicated markers of tumor infiltrating CAR T cells on day 19 post-treatment across groups. An unpaired t test was used to compare expression of each marker between groups. Error bars represent SEM for all panels in this figure.

FIGS. 25A-25C show representative gating strategies for data in FIGS. 5A-K and FIGS. 24A-1. FIGS. 25A-25C are representative plots showing dissociated single cell suspensions from tumors at day 19 post treatment were gated for live singlets, followed by identification of CD3+, CD8+, and human (h)CD45+ to identify CAR T cells as shown. Identified T cells were then stained with three panels [see methods: panels 1 (FIG. 25A), 2 (FIG. 25B) and 3 (FIG. 25C) for indicated targets]. Fluorescence minus one (FMO) controls were used to confirm appropriate compensation and determine positivity for each marker, relevant markers are shown in histograms comparing FMO vs antibody staining.

FIG. 26 shows that BATF3 programs a transcriptional response associated with positive clinical outcome to CAR T cell therapy. FIG. 26 is a schematic of transcriptomic comparison between infused CD8+ CAR T cells of non-responders (NR, dark gray) and responders (R, medium gray).

FIGS. 27A-27F show that CRISPRko screens reveal co-factors of BATF3 and novel targets for cancer immunotherapy. FIG. 27A is a graph showing the number of gRNA hits (P_{adj}<0.01) per gene in the CRISPRko screen without BATF3 OE. Genes with at least 1 enriched gRNA were included in this plot. FIG. 27B is a boxplot of baseline expression of genes stratified based on whether they were hits in the CRISPRko screen without BATF3 OE. Genes with an FDR<0.01 based on mageck gene-level analysis were classified as hits. FIG. 27C is plots of z scores of gRNAs for JUNB and IRF4 in mCherry (left) and BATF3 (right) screens. Enriched gRNAs (P_{adj}<0.01) are labeled in dark gray and non-targeting gRNAs are labeled in light gray. FIG. 27D is a schematic of predicted functional protein association network of BATF3 using STRING. FIG. 27E is plots showing the effect of ZNF217 knockout on IL7R expression in CD8+ T cells across three donors with BATF3 OE. FIG. 27F is plots of z scores of gRNAs.

FIG. 28A is a schematic showing design of gRNA library. FIG. 28B is UMAPs plot. FIG. 28C is graphs showing clustering of donors (left) and cell cycle phase (right). FIG. 28D is maps showing protein and RNA density.

FIG. 29A is a schematic for gRNA levels for IL2, IL7, IL2 sim, and IL7 sim. FIG. 29B is a graph showing predicted effect from FACS screen on IL7R mRNA expression. FIG. 29C is a graph showing IL7R mRNA expression from scRNA-seq data. FIG. 29D is a graph showing SELL mRNA expression as compared to IL7R mRNA expression. FIG. 29E is a graph showing a correlation with IL7R expression.

FIG. 30A is a schematic for a Pearson correlation. FIG. 30B is a heatmap showing gRNA1 and gRNA2 correlation. FIG. 30C is a schematic for gRNA at the gene level. FIG. 30D is a graph showing the average number of DEGs for individual and merged gRNAs. FIG. 30E is a graph showing the number of cells per gRNA. FIG. 30F is a graph showing the number of DEGs for merged gRNA for unstimulated and stimulated IL-2. FIG. 30G is a boxplot showing the correlation between IL-2 and IL-7 conditions.

FIG. 31A is a schematic for gRNA at the gene-context level. FIG. 31B is a graph showing the number of DEGs for merged IL-2 and IL-7 gRNAs for stimulation independent and dependent effects. FIG. 31C is a graph showing the number of DEGs for merged IL-2 and IL-7 gRNAs for directional effects of TF KO. FIG. 31D is heatmaps for stimulated and unstimulated conditions. FIG. 31E is a graph showing ARNT/NT compared to AHR/NT for merged unstimulated IL-2 and IL-7 datasets. FIG. 31F is a graph showing that TBX21 regulates effector T cell response and directly actives IFNγ.

FIG. 32A is an UMAP for WNN. FIG. 32B is a heatmap showing the enrichment of gRNAs in each cluster. FIG. 32C is a heatmap showing the enrichment of gRNAs in stimulated CD8+ T cells. FIG. 32D is a graph showing modulated genes for cluster 4 versus cluster 6. FIG. 32E is a graph showing the upregulated genes in cluster 4 relative to cluster 6. FIG. 32F is a heatmap showing NES score for RASA-2 KO after TCR stimulation. FIG. 32G is a graph showing fold enrichment for cluster 4/cluster 6.

FIG. 33A is a UMAP for WNN. FIG. 33B is heatmaps showing density of genes. FIG. 33C is a heatmap showing gRNA enrichment in unstimulated CD4+ T cells. FIG. 33D is a graph showing cluster 0 vs. cluster 7. FIG. 33E is a graph showing the fold enrichment for cluster 0/cluster 7.

FIG. 34A is a heatmap showing selected genes for FOXO1 were DEGs without stimulation. FIG. 34B is a schematic for scoring targets from genes sets. FIG. 34C is a graph showing average proliferation relative to NT for gRNAs. FIG. 34D is a graph showing average normalized cell numbers relative to NT.

### DETAILED DESCRIPTION

Provided herein are compositions and methods for increasing or enhancing T cells, which may be used to enhance ACT. The compositions and methods may include increasing or decreasing a gene or gene product such as transcription factors. The gene may be selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. Transcriptions factors (TFs) are central mediators of cellular reprogramming and differentiation. As described herein, a Staphylococcus aureus Cas9 (SaCas9)-based epigenome editors were used for targeted gene silencing and activation in primary human T cells. These tools were leveraged to profile the effects of 120 genes with complementary CRISPR interference (CRISPRi) and activation (CRISPRa) screens on human CD8+ T cell state. These screens and subsequent validation revealed that BATF3 overexpression could be harnessed to support specific features of memory T cells, counter T cell exhaustion, and improve tumor control. By conducting parallel pooled CRISPR knockout (CRISPRko) screens of all human transcription factor genes (TFome) with or without BATF3 overexpression, co-factors and downstream targets of BATF3 were defined. BATF3 overexpression was found to promote specific features of memory T cells such as increased IL7R expression and glycolytic capacity, while attenuating gene programs associated with cytotoxicity, regulatory T cell function, and T cell exhaustion. In the context of chronic antigen stimulation, BATF3 overexpression countered phenotypic and epigenetic signatures of T cell exhaustion. CAR T cells overexpressing BATF3 significantly outperformed control CAR T cells in both in vitro and in vivo tumor models. Moreover, it was found that BATF3 programmed a transcriptional profile that correlated with positive clinical response to adoptive T cell therapy. Orthogonal CRISPR-based screening approaches were developed to systematically discover regulators of complex T cell phenotypes, which may be used to engineer T cells with enhanced durability and therapeutic potential. BATF3 may interact with JUNB and IRF4 to regulate gene expression, and the results detailed herein illuminated several other novel targets for enhancing T cells for use in cancer therapies. Collectively, the gene targets identified and described herein may be used to improve the efficacy of ACT.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Allogeneic” refers to any material derived from another subject of the same species. Allogeneic cells are genetically distinct and immunologically incompatible yet belong to the same species. Typically, “allogeneic” is used to define cells, such as stem cells, that are transplanted from a donor to a recipient of the same species.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medullablastoma, non-small cell lung cancer, and/or mesothelioma. In embodiments detailed herein, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides.

Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

The term “shRNA” stands for short hairpin RNA or small hairpin RNA. A shRNA is an artificial RNA molecule with a tight hairpin turn that can be used to silence target gene expression via RNA interference (RNAi). Expression of shRNA in cells may be facilitated by delivery of plasmids or viral or bacterial vectors. The shRNA is processed by Dicer into siRNA.

The term “siRNA” stands for small interfering RNA siRNA, sometimes also known as short interfering RNA or silencing RNA. A siRNA is a class of double-stranded RNA molecule. The siRNA may be natural or artificial. The siRNA forms a complex with the RNA-induced silencing complex (RISC). The antisense (guide) strand of siRNA directs RISC to mRNA that has a complementary sequence, and then the mRNA is cleaved by RISC or its translation is repressed.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment. The subject may have a disease or condition. In some embodiments, the subject has cancer. In some embodiments, the subject is human.

“Substantially identical” can mean that a first and second amino acid or polynucleotide sequence are at least 60%, 65%, 70%, 75%, 80%, 85%, 90%, 95%, 96%, 97%, 98%, or 99%, or less than 100% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is a gene detailed herein as a modulator of T cells.

“Target region” as used herein refers to the region of the target gene to which the CRISPR/Cas9-based gene editing or targeting system is designed to bind.

“T cells” are a type of white blood cell of the immune system and play a central role in the adaptive immune response. T cells express a T-cell receptor (TCR) on their cell surface. The T cell receptor (TCR) of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. T cells may differentiate into different types of T cells. T cells may include, for example, CD8+ T cells (“killer T cells” or “cytotoxic T cells”) and CD4+ T cells (“helper T cells”). CD8+ T cells and CD4+ T cells may further differentiate into other types of T cells including, for example, regulatory T cells (“suppressor T cells”) and memory T cells. In some embodiments herein, the T cell is a memory T cell. An antigen-naïve T cell expands and differentiates into a memory T cell after encountering the cognate antigen within the context of a major histocompatibility complex (MHC) molecule on the surface of an antigen presenting cell. Memory T cells may be CD8+ or CD4+. Memory T cells are long-lived and can quickly expand to large numbers of effector T cells upon re-exposure to their cognate antigen.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Modulators of T Cells

Provided herein are modulators of T cells. Modifying or modulating may include increasing or decreasing, for example. In some embodiments, the compositions and methods comprise an agent that increases T cells. Increasing T cells may include increasing the number of T cells and/or increasing the number of memory T cells and/or increasing the lifetime of a T cell and/or preventing T cell exhaustion and/or reducing T cell exhaustion and/or reversing T cell exhaustion and/or enhancing the therapeutic potential of T cells. Modifying a T cell may include modifying the expression of a target gene within the T cell. The compositions and methods detailed herein may engineer or modify the gene expression programs within T cells by engineering the T cells directly. In some embodiments, the compositions and methods comprise an agent that decreases expression of CCR7 and/or increases expression of IL7RA in T cells. Expression of a marker such as CCR7 or IL7RA may be done by any suitable means in the art, including, for example, ELISA, immunohistochemistry, flow cytometry, FACS, DNA or RNA sequencing, and hybridization of reporters or probes to RNA transcripts.

The agent, or the composition or the method comprising the agent, may target a gene or a regulatory element thereof. Regulatory elements include, for example, promoters and enhancers. Regulatory elements may be within 1000 base pairs of the transcription start site. Regulatory elements may be within 600 base pairs of the transcription start site. The agent, or the composition or the method comprising the agent, may modify the expression of a gene. For example, the agent, or the composition or the method comprising the agent, may reduce, inhibit, decrease, activate, increase, or enhance the expression or activity of a gene or its gene protein product. The agent, or the composition or the method comprising the agent, may directly or indirectly modulate the activity of the gene's protein product. For example, the agent, or the composition or the method comprising the agent, may increase or decrease the binding or enzymatic activity of the gene's protein product, or inhibit the binding of the gene's protein product to another molecule or ligand, or increase the binding of the gene's protein product to another molecule or ligand, or increase or decrease the degradation of the gene's protein product, or a combination thereof.

The targeted gene may be selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory element thereof, or a region thereof, or a combination thereof. In some embodiments, the gene is ZNF217 or ETS.1. In some embodiments, the gene is RBSN, PRDM1, GATA3, or RUNX3. Genes are also detailed in McCutcheon et al., Nat. Genet. 2023; 55(12): 2211-2223, which is incorporated herein by reference in its entirety.

In some embodiments, the agent is an inhibitor, and the agent may inhibit or reduce or decrease expression or activity of a gene or gene protein product to increase T cells. In such embodiments, the gene may be ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1, or a combination thereof.

In some embodiments, the agent is an activator. As an activator, the agent may activate or enhance expression or activity of a gene or gene protein product to increase T cells. In such embodiments, the gene may be FOXO1, MDB2, YY1, DNMT1, IRF1, CTCF, GTF2B, or HIC1, or a combination thereof.

The agent may comprise, for example, a polynucleotide, a polypeptide, a small molecule, a lipid, a carbohydrate, or a combination thereof. In some embodiments, the agent comprises a protein. In some embodiments, the agent comprises an antibody. The antibody may bind to a protein encoded by a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. In some embodiments, the agent comprises a polynucleotide. The agent may comprise a polynucleotide encoding the gene or a fragment thereof or a polynucleotide comprising a cDNA of the gene or a fragment thereof selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. For example, the polynucleotide may comprise a sequence selected from SEQ ID NOs: 337-369, or a fragment thereof. The agent may comprise a polypeptide comprising a protein product of the gene or a fragment thereof selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. For example, the polypeptide may comprise a sequence selected from SEQ ID NOs: 370-402, or a fragment thereof. In some embodiments, the agent comprises a DNA targeting composition as detailed herein or at least one component thereof.

Examples of genes for modulating T cells are shown in TABLE 1.

TABLE 1
Exemplary genes and sequences for modulating T cells.

| Gene | DNA | Protein |
|---|---|---|
| AHR | ATGAACAGCAGCAGCGCCAACATCACCTACGCCAGTC | MNSSSANITYASR |
| | GCAAGCGGCGGAAGCCGGTGCAGAAAACAGTAAAGC | KRRKPVQKTVKPIP |
| | CAATCCCAGCTGAAGGAATCAAGTCAAATCCTTCCAAG | AEGIKSNPSKRHR |
| | CGGCATAGAGACCGACTTAATACAGAGTTGGACCGTTT | DRLNTELDRLASLL |
| | GGCTAGCCTGCTGCCTTTCCCACAAGATGTTATTAATA | PFPQDVINKLDKLS |
| | AGTTGGACAAACTTTCAGTTCTTAGGCTCAGCGTCAGT | VLRLSVSYLRAKSF |
| | TACCTGAGAGCCAAGAGCTTCTTTGATGTTGCATTAAA | FDVALKSSPTERN |
| | ATCCTCCCCTACTGAAAGAAACGGAGGCCAGGATAAC | GGQDNCRAANFR |
| | TGTAGAGCAGCAAATTTCAGAGAAGGCCTGAACTTACA | EGLNLQEGEFLLQ |
| | AGAAGGAGAATTCTTATTACAGGCTCTGAATGGCTTTG | ALNGFVLVVTTDAL |
| | TATTAGTTGTCACTACAGATGCTTTGGTCTTTTATGCTT | VFYASSTIQDYLGF |
| | CTTCTACTATACAAGATTATCTAGGGTTTCAGCAGTCT | QQSDVIHQSVYELI |
| | GATGTCATACATCAGAGTGTATATGAACTTATCCATAC | HTEDRAEFQRQLH |
| | CGAAGACCGAGCTGAATTTCAGCGTCAGCTACACTGG | WALNPSQCTESG |
| | GCATTAAATCCTTCTCAGTGTACAGAGTCTGGACAAGG | QGIEEATGLPQTVV |
| | AATTGAAGAAGCCACTGGTCTCCCCCAGACAGTAGTCT | CYNPDQIPPENSPL |
| | GTTATAACCCAGACCAGATTCCTCCAGAAAACTCTCCT | MERCFICRLRCLLD |
| | TTAATGGAGAGGTGCTTCATATGTCGTCTAAGGTGTCT | NSSGFLAMNFQGK |
| | GCTGGATAATTCATCTGGTTTTCTGGCAATGAATTTCC | LKYLHGQKKKGKD |
| | AAGGGAAGTTAAAGTATCTTCATGGACAGAAAAAGAAA | GSILPPQLALFAIAT |
| | GGGAAAGATGGATCAATACTTCCACCTCAGTTGGCTTT | PLQPPSILEIRTKNF |
| | GTTTGCGATAGCTACTCCACTTCAGCCACCATCCATAC | IFRTKHKLDFTPIGC |
| | TTGAAATCCGGACCAAAAATTTTATCTTTAGAACCAAAC | DAKGRIVLGYTEAE |
| | ACAAACTAGACTTCACACCTATTGGTTGTGATGCCAAA | LCTRGSGYQFIHA |
| | GGAAGAATTGTTTTAGGATATACTGAAGCAGAGCTGTG | ADMLYCAESHIRMI |
| | CACGAGAGGCTCAGGTTATCAGTTTATTCATGCAGCTG | KTGESGMIVFRLLT |
| | ATATGCTTTATTGTGCCGAGTCCCATATCCGAATGATT | KNNRWTWVQSNA |
| | AAGACTGGAGAAAGTGGCATGATAGTTTTCCGGCTTCT | RLLYKNGRPDYIIV |
| | TACAAAAAACAACCGATGGACTTGGGTCCAGTCTAATG | TQRPLTDEEGTEH |
| | CACGCCTGCTTTATAAAAATGGAAGACCAGATTATATC | LRKRNTKLPFMFTT |
| | ATTGTAACTCAGAGACCACTAACAGATGAGGAAGGAAC | GEAVLYEATNPFP |
| | AGAGCATTTACGAAAACGAAATACGAAGTTGCCTTTTA | AIMDPLPLRTKNGT |
| | TGTTTACCACTGGAGAAGCTGTGTTGTATGAGGCAACC | SGKDSATTSTLSK |
| | AACCCTTTTCCTGCCATAATGGATCCCTTACCACTAAG | DSLNPSSLLAAMM |
| | GACTAAAAATGGCACTAGTGGAAAAGACTCTGCTACCA | QQDESIYLYPASST |
| | CATCCACTCTAAGCAAGGACTCTCTCAATCCTAGTTCC | SSTAPFENNFFNE |
| | CTCCTGGCTGCCATGATGCAACAAGATGAGTCTATTTA | SMNECRNWQDNT |
| | TCTCTATCCTGCTTCAAGTACTTCAAGTACTGCACCTTT | APMGNDTILKHEQI |
| | TGAAAACAACTTTTTCAACGAATCTATGAATGAATGCAG | DQPQDVNSFAGG |
| | AAATTGGCAAGATAATACTGCACCGATGGGAAATGATA | HPGLFQDSKNSDL |
| | CTATCCTGAAACATGAGCAAATTGACCAGCCTCAGGAT | YSIMKNLGIDFEDIR |
| | GTGAACTCATTTGCTGGAGGTCACCCAGGGCTCTTTCA | HMQNEKFFRNDFS |
| | AGATAGTAAAAACAGTGACTTGTACAGCATAATGAAAA | GEVDFRDIDLTDEI |
| | ACCTAGGCATTGATTTTGAAGACATCAGACACATGCAG | LTYVQDSLSKSPFI |
| | AATGAAAAATTTTTCAGAAATGATTTTTCTGGTGAGGTT | PSDYQQQQSLALN |
| | GACTTCAGAGACATTGACTTAACGGATGAAATCCTGAC | SSCMVQEHLHLEQ |
| | GTATGTCCAAGATTCTTTAAGTAAGTCTCCCTTCATACC | QQQHHQKQVVVE |
| | TTCAGATTATCAACAGCAACAGTCCTTGGCTCTGAACT | PQQQLCQKMKHM |
| | CAAGCTGTATGGTACAGGAACACCTACATCTAGAACAG | QVNGMFENWNSN |
| | CAACAGCAACATCACCAAAAGCAAGTAGTAGTGGAGC | QFVPFNCPQQDPQ |
| | CACAGCAACAGCTGTGTCAGAAGATGAAGCACATGCA | QYNVFTDLHGISQ |
| | AGTTAATGGCATGTTTGAAAATTGGAACTCTAACCAATT | EFPYKSEMDSMPY |
| | CGTGCCTTTCAATTGTCCACAGCAAGACCCACAACAAT | TQNFISCNQPVLP |
| | ATAATGTCTTTACAGACTTACATGGGATCAGTCAAGAG | QHSKCTELDYPMG |
| | TTCCCCTACAAATCTGAAATGGATTCTATGCCTTATACA | SFEPSPYPTTSSLE |
| | CAGAACTTTATTTCCTGTAATCAGCCTGTATTACCACAA | DFVTCLQLPENQK |
| | CATTCCAAATGTACAGAGCTGGACTACCCTATGGGGA | HGLNPQSAIITPQT |
| | GTTTTGAACCATCCCCATACCCCACTACTTCTAGTTTA | CYAGAVSMYQCQ |
| | GAAGATTTTGTCACTTGTTTACAACTTCCTGAAAACCAA | PEPQHTHVGQMQ |
| | AAGCATGGATTAAATCCACAGTCAGCCATAATAACTCC | YNPVLPGQQAFLN |
| | TCAGACATGTTATGCTGGGGCCGTGTCGATGTATCAGT | KFQNGVLNETYPA |
| | GCCAGCCAGAACCTCAGCACACCCACGTGGGTCAGAT | ELNNINNTQTTTHL |
| | GCAGTACAATCCAGTACTGCCAGGCCAACAGGCATTTT | QPLHHPSEARPFP |
| | TAAACAAGTTTCAGAATGGAGTTTTAAATGAAACATATC | DLTSSGFL |
| | CAGCTGAATTAAATAACATAAATAACACTCAGACTACCA | (SEQ ID NO: 370) |
| | CACATCTTCAGCCACTTCATCATCCGTCAGAAGCCAGA | |
| | CCTTTTCCTGATTTGACATCCAGTGGATTCCTGTAA | |
| | (SEQ ID NO: 337) | |
| ARNT | ATGGCGGCGACTACTGCCAACCCCGAAATGACATCAG | MAATTANPEMTSD |
| | ATGTACCATCACTGGGTCCAGCCATTGCCTCTGGAAAC | VPSLGPAIASGNS |
| | TCTGGACCTGGAATTCAAGGTGGAGGAGCCATTGTCC | GPGIQGGGAIVQR |
| | AGAGGGCTATTAAGCGGCGACCAGGGCTGGATTTTGA | AIKRRPGLDFDDD |
| | TGATGATGGAGAAGGGAACAGTAAATTTTTGAGGTGTG | GEGNSKFLRCDDD |
| | ATGATGATCAGATGTCTAACGATAAGGAGCGGTTTGCC | QMSNDKERFARSD |
| | AGGTCGGATGATGAGCAGAGCTCTGCGGATAAAGAGA | DEQSSADKERLAR |
| | GACTTGCCAGGGAAAATCACAGTGAAATTGAACGGCG | ENHSEIERRRRNK |
| | GCGACGGAACAAGATGACAGCCTACATCACAGAACTG | MTAYITELSDMVPT |
| | TCAGATATGGTACCCACCTGTAGTGCCCTGGCTCGAA | CSALARKPDKLTIL |
| | AACCAGACAAGCTAACCATCTTACGCATGGCAGTTTCT | RMAVSHMKSLRGT |
| | CACATGAAGTCCTTGCGGGGAACTGGCAACACATCCA | GNTSTDGSYKPSF |
| | CTGATGGCTCCTATAAGCCGTCTTTCCTCACTGATCAG | LTDQELKHLILEAA |
| | GAACTGAAACATTTGATCTTGGAGGCAGCAGATGGCTT | DGFLFIVSCETGRV |
| | TCTGTTTATTGTCTCATGTGAGACAGGCAGGGTGGTGT | VYVSDSVTPVLNQ |
| | ATGTGTCTGACTCCGTGACTCCTGTTTTGAACCAGCCA | PQSEWFGSTLYDQ |
| | CAGTCTGAATGGTTTGGCAGCACACTCTATGATCAGGT | VHPDDVDKLREQL |
| | GCACCCAGATGATGTGGATAAACTTCGTGAGCAGCTTT | STSENALTGRILDL |
| | CCACTTCAGAAAATGCCCTGACAGGGCGTATCCTGGA | KTGTVKKEGQQSS |
| | TCTAAAGACTGGAACAGTGAAAAAGGAAGGTCAGCAG | MRMCMGSRRSFIC |
| | TCTTCCATGAGAATGTGTATGGGCTCAAGGAGATCGTT | RMRCGSSSVDPVS |
| | TATTTGCCGAATGAGGTGTGGCAGTAGCTCTGTGGAC | VNRLSFVRNRCRN |
| | CCAGTTTCTGTGAATAGGCTGAGCTTTGTGAGGAACAG | GLGSVKDGEPHFV |
| | ATGCAGGAATGGACTTGGCTCTGTAAAGGATGGGGAA | WVHCTGYIKAWPP |
| | CCTCACTTCGTGGTGGTCCACTGCACAGGCTACATCA | AGVSLPDDDPEAG |
| | AGGCCTGGCCCCCAGCAGGTGTTTCCCTCCCAGATGA | QGSKFCLVAIGRL |
| | TGACCCAGAGGCTGGCCAGGGAAGCAAGTTTTGCCTA | QVTSSPNCTDMSN |
| | GTGGCCATTGGCAGATTGCAGGTAACTAGTTCTCCCAA | VCQPTEFISRHNIE |
| | CTGTACAGACATGAGTAATGTTTGTCAACCAACAGAGT | GIFTFVDHRCVATV |
| | TCATCTCCCGACACAACATTGAGGGTATCTTCACTTTT | GYQPQELLGKNIV |
| | GTGGATCACCGCTGTGTGGCTACTGTTGGCTACCAGC | EFCHPEDQQLLRD |
| | CACAGGAACTCTTAGGAAAGAATATTGTAGAATTCTGT | SFQQVVKLKGQVL |
| | CATCCTGAAGACCAGCAGCTTCTAAGAGACAGCTTCCA | SVMFRFRSKNQE |
| | ACAGGTAGTGAAATTAAAAGGCCAAGTGCTGTCTGTCA | WLWMRTSSFTFQ |
| | TGTTCCGGTTCCGGTCTAAGAACCAAGAATGGCTCTG | NPYSDEIEYIICTNT |
| | GATGAGAACCAGCTCCTTTACTTTCCAGAACCCTTACT | NVKNSSQEPRPTL |
| | CAGATGAAATTGAGTACATCATCTGTACCAACACCAAT | SNTIQRPQLGPTA |
| | GTGAAGAACTCTAGCCAAGAACCACGGCCTACACTCT | NLPLEMGSGQLAP |
| | CCAACACAATCCAGAGGCCACAACTAGGTCCCACAGC | RQQQQQTELDMV |
| | TAATTTACCCCTGGAGATGGGCTCAGGACAGCTGGCA | PGRDGLASYNHSQ |
| | CCCAGGCAGCAGCAACAGCAAACAGAATTGGACATGG | VVQPVTTTGPEHS |
| | TACCAGGAAGAGATGGACTGGCCAGCTACAATCATTC | KPLEKSDGLFAQD |
| | CCAGGTGGTTCAGCCTGTGACAACCACAGGACCAGAA | RDPRFSEIYHNINA |
| | CACAGCAAGCCCCTTGAGAAGTCAGATGGTTTATTTGC | DQSKGISSSTVPAT |
| | CCAGGATAGAGATCCAAGATTTTCAGAAATCTATCACA | QQLFSQGNTFPPT |
| | ACATCAATGCGGATCAGAGTAAAGGCATCTCCTCCAG | PRPAENFRNSGLA |
| | CACTGTCCCTGCCACCCAACAGCTATTCTCCCAGGGC | PPVTIVQPSASAG |
| | AACACATTCCCTCCTACCCCCCGGCCGGCAGAGAATT | QMLAQISRHSNPT |
| | TCAGGAATAGTGGCCTAGCCCCTCCTGTAACCATTGTC | QGATPTWTPTTRS |
| | CAGCCATCAGCTTCTGCAGGACAGATGTTGGCCCAGA | GFSAQQVATQATA |
| | TTTCCCGCCACTCCAACCCCACCCAAGGAGCAACCCC | KTRTSQFGVGSFQ |
| | AACTTGGACCCCTACTACCCGCTCAGGCTTTTCTGCCC | TPSSFSSMSLPGA |
| | AGCAGGTGGCTACCCAGGCTACTGCTAAGACTCGTAC | PTASPGAAAYPSL |
| | TTCCCAGTTTGGTGTGGGCAGCTTTCAGACTCCATCCT | TNRGSNFAPETGQ |
| | CCTTCAGCTCCATGTCCCTCCCTGGTGCCCCAACTGC | TAGQFQTRTAEGV |
| | ATCGCCTGGTGCTGCTGCCTACCCTAGTCTCACCAATC | GVWPQWQGQQP |
| | GTGGATCTAACTTTGCTCCTGAGACTGGACAGACTGCA | HHRSSSSEQHVQ |
| | GGACAATTCCAGACACGGACAGCAGAGGGTGTGGGT | QPPAQQPGQPEVF |
| | GTCTGGCCACAGTGGCAGGGCCAGCAGCCTCATCATC | QEMLSMLGDQSN |
| | GTTCAAGTTCTAGTGAGCAACATGTTCAACAACCGCCA | SYNNEEFPDLTMF |
| | GCACAGCAACCTGGCCAGCCTGAGGTCTTCCAGGAGA | PPFSE |
| | TGCTGTCCATGCTGGGAGATCAGAGCAACAGCTACAA | (SEQ ID NO: 371) |
| | CAATGAAGAATTCCCTGATCTAACTATGTTTCCCCCCTT | |
| | TTCAGAATAG | |
| | (SEQ ID NO: 338) | |
| CTCF | ATGGAAGGTGATGCAGTCGAAGCCATTGTGGAGGAGT | MEGDAVEAIVEES |
| | CCGAAACTTTTATTAAAGGAAAGGAGAGAAAGACTTAC | ETFIKGKERKTYQR |
| | CAGAGACGCCGGGAAGGGGGCCAGGAAGAAGATGCC | RREGGQEEDACHL |
| | TGCCACTTACCCCAGAACCAGACGGATGGGGGTGAGG | PQNQTDGGEVVQ |
| | TGGTCCAGGATGTCAACAGCAGTGTACAGATGGTGAT | DVNSSVQMVMME |
| | GATGGAACAGCTGGACCCCACCCTTCTTCAGATGAAG | QLDPTLLQMKTEV |
| | ACTGAAGTAATGGAGGGCACAGTGGCTCCAGAAGCAG | MEGTVAPEAEAAV |
| | AGGCTGCTGTGGACGATACCCAGATTATAACTTTACAG | DDTQIITLQVVNME |
| | GTTGTAAATATGGAGGAACAGCCCATAAACATAGGAGA | EQPINIGELQLVQV |
| | ACTTCAGCTTGTTCAAGTACCTGTTCCTGTGACTGTAC | PVPVTVPVATTSVE |
| | CTGTTGCTACCACTTCAGTAGAAGAACTTCAGGGGGCT | ELQGAYENEVSKE |
| | TATGAAAATGAAGTGTCTAAAGAGGGCCTTGCGGAAA | GLAESEPMICHTLP |
| | GTGAACCCATGATATGCCACACCCTACCTTTGCCTGAA | LPEGFQVVKVGAN |
| | GGGTTTCAGGTGGTTAAAGTGGGGGCCAATGGAGAGG | GEVETLEQGELPP |
| | TGGAGACACTAGAACAAGGGGAACTTCCACCCCAGGA | QEDPSWQKDPDY |
| | AGATCCTAGTTGGCAAAAAGACCCAGACTATCAGCCAC | QPPAKKTKKTKKS |
| | CAGCCAAAAAAACAAAGAAAACCAAAAAGAGCAAACTG | KLRYTEEGKDVDV |
| | CGTTATACAGAGGAGGGCAAAGATGTAGATGTGTCTG | SVYDFEEEQQEGL |
| | TCTACGATTTTGAGGAAGAACAGCAGGAGGGTCTGCT | LSEVNAEKVVGNM |
| | ATCAGAGGTTAATGCAGAGAAAGTGGTTGGTAATATGA | KPPKPTKIKKKGVK |
| | AGCCTCCAAAGCCAACAAAAATTAAAAAGAAAGGTGTA | KTFQCELCSYTCP |
| | AAGAAGACATTCCAGTGTGAGCTTTGCAGTTACACGTG | RRSNLDRHMKSHT |
| | TCCACGGCGTTCAAATTTGGATCGTCACATGAAAAGCC | DERPHKCHLCGRA |
| | ACACTGATGAGAGACCACACAAGTGCCATCTCTGTGG | FRTVTLLRNHLNTH |
| | CAGGGCATTCAGAACAGTCACCCTCCTGAGGAATCAC | TGTRPHKCPDCDM |
| | CTTAACACACACACAGGTACTCGTCCTCACAAGTGCCC | AFVTSGELVRHRR |
| | AGACTGCGACATGGCCTTTGTGACCAGTGGAGAATTG | YKHTHEKPFKCSM |
| | GTTCGGCATCGTCGTTACAAACACACCCACGAGAAGC | CDYASVEVSKLKR |
| | CATTCAAGTGTTCCATGTGCGATTACGCCAGTGTAGAA | HIRSHTGERPFQC |
| | GTCAGCAAATTAAAACGTCACATTCGCTCTCATACTGG | SLCSYASRDTYKL |
| | AGAGCGTCCGTTTCAGTGCAGTTTGTGCAGTTATGCCA | KRHMRTHSGEKPY |
| | GCAGGGACACATACAAGCTGAAAAGGCACATGAGAAC | ECYICHARFTQSGT |
| | CCATTCAGGGGAAAAGCCTTATGAATGTTATATTTGTC | MKMHILQKHTENV |
| | ATGCTCGGTTTACCCAAAGTGGTACCATGAAGATGCAC | AKFHCPHCDTVIAR |
| | ATTTTACAGAAGCACACAGAAAATGTGGCCAAATTTCA | KSDLGVHLRKQHS |
| | CTGTCCCCACTGTGACACAGTCATAGCCCGAAAAAGT | YIEQGKKCRYCDA |
| | GATTTGGGTGTCCACTTGCGAAAGCAGCATTCCTATAT | VFHERYALIQHQKS |
| | TGAGCAAGGCAAGAAATGCCGTTACTGTGATGCTGTG | HKNEKRFKCDQCD |
| | TTTCATGAGCGCTATGCCCTCATCCAGCATCAGAAGTC | YACRQERHMIMHK |
| | ACACAAGAATGAGAAGCGCTTTAAGTGTGACCAGTGT | RTHTGEKPYACSH |
| | GATTACGCTTGTAGACAGGAGAGGCACATGATCATGC | CDKTFRQKQLLDM |
| | ACAAGCGCACCCACACCGGGGAGAAGCCTTACGCCTG | HFKRYHDPNFVPA |
| | CAGCCACTGCGATAAGACCTTCCGCCAGAAGCAGCTT | AFVCSKCGKTFTR |
| | CTCGACATGCACTTCAAGCGCTATCACGACCCCAACTT | RNTMARHADNCA |
| | CGTCCCTGCGGCTTTTGTCTGTTCTAAGTGTGGGAAAA | GPDGVEGENGGE |
| | CATTTACACGTCGGAATACCATGGCAAGACATGCTGAT | TKKSKRGRKRKMR |
| | AATTGTGCTGGCCCAGATGGCGTAGAGGGGGAAAATG | SKKEDSSDSENAE |
| | GAGGAGAAACGAAGAAGAGTAAACGTGGAAGAAAAAG | PDLDDNEDEEEPA |
| | AAAGATGCGCTCTAAGAAAGAAGATTCCTCTGACAGTG | VEIEPEPEPQPVTP |
| | AAAATGCTGAACCAGATCTGGACGACAATGAGGATGA | APPPAKKRRGRPP |
| | GGAGGAGCCTGCCGTAGAAATTGAACCTGAGCCAGAG | GRTNQPKQNQPTA |
| | CCTCAGCCTGTGACCCCAGCCCCACCACCCGCCAAGA | IIQVEDQNTGAIENII |
| | AGCGGAGAGGACGACCCCCTGGCAGAACCAACCAGC | VEVKKEPDAEPAE |
| | CCAAACAGAACCAGCCAACAGCTATCATTCAGGTTGAA | GEEEEAQPAATDA |
| | GACCAGAATACAGGTGCAATTGAGAACATTATAGTTGA | PNGDLTPEMILSM |
| | AGTAAAAAAAGAGCCAGATGCTGAGCCCGCAGAGGGA | MDR |
| | GAGGAAGAGGAGGCCCAGCCAGCTGCCACAGATGCC | (SEQ ID NO: 372) |
| | CCCAACGGAGACCTCACGCCCGAGATGATCCTCAGCA | |
| | TGATGGACCGGTGA | |
| | (SEQ ID NO: 339) | |
| DNMT1 | ATGCCGGCGCGTACCGCCCCAGCCCGGGTGCCCACA | MPARTAPARVPTL |
| | CTGGCCGTCCCGGCCATCTCGCTGCCCGACGATGTCC | AVPAISLPDDVRRR |
| | GCAGGCGGCTCAAAGATTTGGAAAGAGACAGCTTAAC | LKDLERDSLTEKEC |
| | AGAAAAGGAATGTGTGAAGGAGAAATTGAATCTCTTGC | VKEKLNLLHEFLQT |
| | ACGAATTTCTGCAAACAGAAATAAAGAATCAGTTATGT | EIKNQLCDLETKLR |
| | GACTTGGAAACCAAATTACGTAAAGAAGAATTATCCGA | KEELSEEGYLAKV |
| | GGAGGGCTACCTGGCTAAAGTCAAATCCCTTTTAAATA | KSLLNKDLSLENGA |
| | AAGATTTGTCCTTGGAGAACGGTGCTCATGCTTACAAC | HAYNREVNGRLEN |
| | CGGGAAGTGAATGGACGTCTAGAAAACGGGAACCAAG | GNQARSEARRVG |
| | CAAGAAGTGAAGCCCGTAGAGTGGGAATGGCAGATGC | MADANSPPKPLSK |
| | CAACAGCCCCCCCAAACCCCTTTCCAAACCTCGCACG | PRTPRRSKSDGEA |
| | CCCAGGAGGAGCAAGTCCGATGGAGAGGCTAAGCCT | KPEPSPSPRITRKS |
| | GAACCTTCACCTAGCCCCAGGATTACAAGGAAAAGCA | TRQTTITSHFAKGP |
| | CCAGGCAAACCACCATCACATCTCATTTTGCAAAGGGC | AKRKPQEESERAK |
| | CCTGCCAAACGGAAACCTCAGGAAGAGTCTGAAAGAG | SDESIKEEDKDQD |
| | CCAAATCGGATGAGTCCATCAAGGAAGAAGACAAAGA | EKRRRVTSRERVA |
| | CCAGGATGAGAAGAGACGTAGAGTTACATCCAGAGAA | RPLPAEEPERAKS |
| | CGAGTTGCTAGACCGCTTCCTGCAGAAGAACCTGAAA | GTRTEKEEERDEK |
| | GAGCAAAATCAGGAACGCGCACTGAAAAGGAAGAAGA | EEKRLRSQTKEPT |
| | AAGAGATGAAAAAGAAGAAAAGAGACTCCGAAGTCAAA | PKQKLKEEPDREA |
| | CCAAAGAACCAACACCCAAACAGAAACTGAAGGAGGA | RAGVQADEDEDG |
| | GCCGGACAGAGAAGCCAGGGCAGGCGTGCAGGCTGA | DEKDEKKHRSQPK |
| | CGAGGACGAAGATGGAGACGAGAAAGATGAGAAGAAG | DLAAKRRPEEKEP |
| | CACAGAAGTCAACCCAAAGATCTAGCTGCCAAACGGA | EKVNPQISDEKDE |
| | GGCCCGAAGAAAAAGAACCTGAAAAAGTAAATCCACA | DEKEEKRRKTTPK |
| | GATTTCTGATGAAAAAGACGAGGATGAAAAGGAGGAG | EPTEKKMARAKTV |
| | AAGAGACGCAAAACGACCCCCAAAGAACCAACGGAGA | MNSKTHPPKCIQC |
| | AAAAAATGGCTCGCGCCAAAACAGTCATGAACTCCAAG | GQYLDDPDLKYGQ |
| | ACCCACCCTCCCAAGTGCATTCAGTGCGGGCAGTACC | HPPDAVDEPQMLT |
| | TGGACGACCCTGACCTCAAATATGGGCAGCACCCACC | NEKLSIFDANESGF |
| | AGACGCGGTGGATGAGCCACAGATGCTGACAAATGAG | ESYEALPQHKLTC |
| | AAGCTGTCCATCTTTGATGCCAACGAGTCTGGCTTTGA | FSVYCKHGHLCPID |
| | GAGTTATGAGGCGCTTCCCCAGCACAAACTGACCTGC | TGLIEKNIELFFSGS |
| | TTCAGTGTGTACTGTAAGCACGGTCACCTGTGTCCCAT | AKPIYDDDPSLEG |
| | CGACACCGGCCTCATCGAGAAGAATATCGAACTCTTCT | GVNGKNLGPINEW |
| | TTTCTGGTTCAGCAAAACCAATCTATGATGATGACCCA | WITGFDGGEKALIG |
| | TCTCTTGAAGGTGGTGTTAATGGCAAAAATCTTGGCCC | FSTSFAEYILMDPS |
| | CATAAATGAATGGTGGATCACTGGCTTTGATGGAGGTG | PEYAPIFGLMQEKI |
| | AAAAGGCCCTCATCGGCTTCAGCACCTCATTTGCCGAA | YISKIVVEFLQSNS |
| | TACATTCTGATGGATCCCAGTCCCGAGTATGCGCCCAT | DSTYEDLINKIETTV |
| | ATTTGGGCTGATGCAGGAGAAGATCTACATCAGCAAG | PPSGLNLNRFTED |
| | ATTGTGGTGGAGTTCCTGCAGAGCAATTCCGACTCGA | SLLRHAQFVVEQV |
| | CCTATGAGGACCTGATCAACAAGATCGAGACCACGGT | ESYDEAGDSDEQP |
| | TCCTCCTTCTGGCCTCAACTTGAACCGCTTCACAGAGG | IFLTPCMRDLIKLA |
| | ACTCCCTCCTGCGACACGCGCAGTTTGTGGTGGAGCA | GVTLGQRRAQARR |
| | GGTGGAGAGTTATGACGAGGCCGGGGACAGTGATGA | QTIRHSTREKDRG |
| | GCAGCCCATCTTCCTGACACCCTGCATGCGGGACCTG | PTKATTTKLVYQIF |
| | ATCAAGCTGGCTGGGGTCACGCTGGGACAGAGGCGA | DTFFAEQIEKDDRE |
| | GCCCAGGCGAGGCGGCAGACCATCAGGCATTCTACCA | DKENAFKRRRCGV |
| | GGGAGAAGGACAGGGGACCCACGAAAGCCACCACCA | CEVCQQPECGKC |
| | CCAAGCTGGTCTACCAGATCTTCGATACTTTCTTCGCA | KACKDMVKFGGS |
| | GAGCAAATTGAAAAGGATGACAGAGAAGACAAGGAGA | GRSKQACQERRC |
| | ACGCCTTTAAGCGCCGGCGATGTGGCGTCTGTGAGGT | PNMAMKEADDDE |
| | GTGTCAGCAGCCTGAGTGTGGGAAATGTAAAGCCTGC | EVDDNIPEMPSPK |
| | AAGGACATGGTTAAATTTGGTGGCAGTGGACGGAGCA | KMHQGKKKKQNK |
| | AGCAGGCTTGCCAAGAGCGGAGGTGTCCCAATATGGC | NRISWVGEAVKTD |
| | CATGAAGGAGGCAGATGACGATGAGGAAGTCGATGAT | GKKSYYKKVCIDAE |
| | AACATCCCAGAGATGCCGTCACCCAAAAAAATGCACCA | TLEVGDCVSVIPDD |
| | GGGGAAGAAGAAGAAACAGAACAAGAATCGCATCTCT | SSKPLYLARVTAL |
| | TGGGTCGGAGAAGCCGTCAAGACTGATGGGAAGAAGA | WEDSSNGQMFHA |
| | GTTACTATAAGAAGGTGTGCATTGATGCGGAAACCCTG | HWFCAGTDTVLGA |
| | GAAGTGGGGGACTGTGTCTCTGTTATTCCAGATGATTC | TSDPLELFLVDECE |
| | CTCAAAACCGCTGTATCTAGCAAGGGTCACGGCGCTG | DMQLSYIHSKVKVI |
| | TGGGAGGACAGCAGCAACGGGCAGATGTTTCACGCCC | YKAPSENWAMEG |
| | ACTGGTTCTGCGCTGGGACAGACACAGTCCTCGGGGC | GMDPESLLEGDDG |
| | CACGTCGGACCCTCTGGAGCTGTTCTTGGTGGATGAA | KTYFYQLWYDQDY |
| | TGTGAGGACATGCAGCTTTCATATATCCACAGCAAAGT | ARFESPPKTQPTE |
| | GAAAGTCATCTACAAAGCCCCCTCCGAAAACTGGGCC | DNKFKFCVSCARL |
| | ATGGAGGGAGGCATGGATCCCGAGTCCCTGCTGGAG | AEMRQKEIPRVLE |
| | GGGGACGACGGGAAGACCTACTTCTACCAGCTGTGGT | QLEDLDSRVLYYS |
| | ATGATCAAGACTACGCGAGATTCGAGTCCCCTCCAAAA | ATKNGILYRVGDG |
| | ACCCAGCCAACAGAGGACAACAAGTTCAAATTCTGTGT | VYLPPEAFTFNIKL |
| | GAGCTGTGCCCGTCTGGCTGAGATGAGGCAAAAAGAA | SSPVKRPRKEPVD |
| | ATCCCCAGGGTCCTGGAGCAGCTCGAGGACCTGGATA | EDLYPEHYRKYSD |
| | GCCGGGTCCTCTACTACTCAGCCACCAAGAACGGCAT | YIKGSNLDAPEPYR |
| | CCTGTACCGAGTTGGTGATGGTGTGTACCTGCCCCCT | IGRIKEIFCPKKSNG |
| | GAGGCCTTCACGTTCAACATCAAGCTGTCCAGTCCCG | RPNETDIKIRVNKF |
| | TGAAACGCCCACGGAAGGAGCCCGTGGATGAGGACC | YRPENTHKSTPAS |
| | TGTACCCAGAGCACTACCGGAAATACTCCGACTACATC | YHADINLLYWSDE |
| | AAAGGCAGCAACCTGGATGCCCCTGAGCCCTACCGAA | EAVVDFKAVQGRC |
| | TTGGCCGGATCAAAGAGATCTTCTGTCCCAAGAAGAG | TVEYGEDLPECVQ |
| | CAACGGCAGGCCCAATGAGACTGACATCAAAATCCGG | VYSMGGPNRFYFL |
| | GTCAACAAGTTCTACAGGCCTGAGAACACCCACAAGT | EAYNAKSKSFEDP |
| | CCACTCCAGCGAGCTACCACGCAGACATCAACCTGCT | PNHARSPGNKGK |
| | CTACTGGAGCGACGAGGAGGCCGTGGTGGACTTCAA | GKGKGKGKPKSQ |
| | GGCTGTGCAGGGCCGCTGCACCGTGGAGTATGGGGA | ACEPSEPEIEIKLPK |
| | GGACCTGCCCGAGTGCGTCCAGGTGTACTCCATGGGC | LRTLDVFSGCGGL |
| | GGCCCCAACCGCTTCTACTTCCTCGAGGCCTATAATG | SEGFHQAGISDTL |
| | CAAAGAGCAAAAGCTTTGAAGATCCTCCCAACCATGCC | WAIEMWDPAAQAF |
| | CGTAGCCCTGGAAACAAAGGGAAGGGCAAGGGAAAA | RLNNPGSTVFTED |
| | GGGAAGGGCAAGCCCAAGTCCCAAGCCTGTGAGCCG | CNILLKLVMAGETT |
| | AGCGAGCCAGAGATAGAGATCAAGCTGCCCAAGCTGC | NSRGQRLPQKGD |
| | GGACCCTGGATGTGTTTTCTGGCTGCGGGGGGTTGTC | VEMLCGGPPCQG |
| | GGAGGGATTCCACCAAGCAGGCATCTCTGACACGCTG | FSGMNRFNSRTYS |
| | TGGGCCATCGAGATGTGGGACCCTGCGGCCCAGGCG | KFKNSLVVSFLSYC |
| | TTCCGGCTGAACAACCCCGGCTCCACAGTGTTCACAG | DYYRPRFFLLENV |
| | AGGACTGCAACATCCTGCTGAAGCTGGTCATGGCTGG | RNFVSFKRSMVLK |
| | GGAGACCACCAACTCCCGCGGCCAGCGGCTGCCCCA | LTLRCLVRMGYQC |
| | GAAGGGAGACGTGGAGATGCTGTGCGGCGGGCCGCC | TFGVLQAGQYGVA |
| | CTGCCAGGGCTTCAGCGGCATGAACCGCTTCAATTCG | QTRRRAIILAAAPG |
| | CGCACCTACTCCAAGTTCAAAAACTCTCTGGTGGTTTC | EKLPLFPEPLHVFA |
| | CTTCCTCAGCTACTGCGACTACTACCGGCCCCGGTTC | PRACQLSVVVDDK |
| | TTCCTCCTGGAGAATGTCAGGAACTTTGTCTCCTTCAA | KFVSNITRLSSGPF |
| | GCGCTCCATGGTCCTGAAGCTCACCCTCCGCTGCCTG | RTITVRDTMSDLPE |
| | GTCCGCATGGGCTATCAGTGCACCTTCGGCGTGCTGC | VRNGASALEISYN |
| | AGGCCGGTCAGTACGGCGTGGCCCAGACTAGGAGGC | GEPQSWFQRQLR |
| | GGGCCATCATCCTGGCCGCGGCCCCTGGAGAGAAGC | GAQYQPILRDHICK |
| | TCCCTCTGTTCCCGGAGCCACTGCACGTGTTTGCTCC | DMSALVAARMRHI |
| | CCGGGCCTGCCAGCTGAGCGTGGTGGTGGATGACAA | PLAPGSDWRDLPN |
| | GAAGTTTGTGAGCAACATAACCAGGTTGAGCTCGGGT | IEVRLSDGTMARKL |
| | CCTTTCCGGACCATCACGGTGCGAGACACGATGTCCG | RYTHHDRKNGRSS |
| | ACCTGCCGGAGGTGCGGAATGGAGCCTCGGCACTGG | SGALRGVCSCVEA |
| | AGATCTCCTACAACGGGGAGCCTCAGTCCTGGTTCCA | GKACDPAARQFNT |
| | GAGGCAGCTCCGGGGCGCACAGTACCAGCCCATCCT | LIPWCLPHTGNRH |
| | CAGGGACCACATCTGTAAGGACATGAGTGCATTGGTG | NHWAGLYGRLEW |
| | GCTGCCCGCATGCGGCACATCCCCTTGGCCCCAGGG | DGFFSTTVTNPEP |
| | TCAGACTGGCGCGATCTGCCCAACATCGAGGTGCGGC | MGKQGRVLHPEQ |
| | TCTCAGACGGCACCATGGCCAGGAAGCTGCGGTATAC | HRVVSVRECARSQ |
| | CCACCATGACAGGAAGAACGGCCGCAGCAGCTCTGG | GFPDTYRLFGNILD |
| | GGCCCTCCGTGGGGTCTGCTCCTGCGTGGAAGCCGG | KHRQVGNAVPPPL |
| | CAAAGCCTGCGACCCCGCAGCCAGGCAGTTCAACACC | AKAIGLEIKLCMLA |
| | CTCATCCCCTGGTGCCTGCCCCACACCGGGAACCGGC | KARESASAKIKEEE |
| | ACAACCACTGGGCTGGCCTCTATGGAAGGCTCGAGTG | AAKD |
| | GGACGGCTTCTTCAGCACAACCGTCACCAACCCCGAG | (SEQ ID NO: 373) |
| | CCCATGGGCAAGCAGGGCCGCGTGCTCCACCCAGAG | |
| | CAGCACCGTGTGGTGAGCGTGCGGGAGTGTGCCCGC | |
| | TCCCAGGGCTTCCCTGACACCTACCGGCTCTTCGGCA | |
| | ACATCCTGGACAAGCACCGGCAGGTGGGCAATGCCGT | |
| | GCCACCGCCCCTGGCCAAAGCCATTGGCTTGGAGATC | |
| | AAGCTTTGTATGTTGGCCAAAGCCCGAGAGAGTGCCT | |
| | CAGCTAAAATAAAGGAGGAGGAAGCTGCTAAGGACTA | |
| | G | |
| | (SEQ ID NO: 340) | |
| DUX4 | ATGGCCCTCCCGACACCCTCGGACAGCACCCTCCCCG | MALPTPSDSTLPA |
| | CGGAAGCCCGGGGACGAGGACGGCGACGGAGACTCG | EARGRGRRRRLV |
| | TTTGGACCCCGAGCCAAAGCGAGGCCCTGCGAGCCT | WTPSQSEALRACF |
| | GCTTTGAGCGGAACCCGTACCCGGGCATCGCCACCAG | ERNPYPGIATRERL |
| | AGAACGGCTGGCCCAGGCCATCGGCATTCCGGAGCC | AQAIGIPEPRVQIW |
| | CAGGGTCCAGATTTGGTTTCAGAATGAGAGGTCACGC | FQNERSRQLRQHR |
| | CAGCTGAGGCAGCACCGGCGGGAATCTCGGCCCTGG | RESRPWPGRRGP |
| | CCCGGGAGACGCGGCCCGCCAGAAGGCCGGCGAAAG | PEGRRKRTAVTGS |
| | CGGACCGCCGTCACCGGATCCCAGACCGCCCTGCTC | QTALLLRAFEKDRF |
| | CTCCGAGCCTTTGAGAAGGATCGCTTTCCAGGCATCG | PGIAAREELARETG |
| | CCGCCCGGGAGGAGCTGGCCAGAGAGACGGGCCTCC | LPESRIQIWFQNRR |
| | CGGAGTCCAGGATTCAGATCTGGTTTCAGAATCGAAG | ARHPGQGGRAPA |
| | GGCCAGGCACCCGGGACAGGGTGGCAGGGCGCCCG | QAGGLCSAAPGG |
| | CGCAGGCAGGCGGCCTGTGCAGCGCGGCCCCCGGC | GHPAPSWVAFAHT |
| | GGGGGTCACCCTGCTCCCTCGTGGGTCGCCTTCGCC | GAWGTGLPAPHVP |
| | CACACCGGCGCGTGGGGAACGGGGCTTCCCGCACCC | CAPGALPQGAFVS |
| | CACGTGCCCTGCGCGCCTGGGGCTCTCCCACAGGGG | QAARAAPALQPSQ |
| | GCTTTCGTGAGCCAGGCAGCGAGGGCCGCCCCCGCG | AAPAEGISQPAPA |
| | CTGCAGCCCAGCCAGGCCGCGCCGGCAGAGGGGATC | RGDFAYAAPAPPD |
| | TCCCAACCTGCCCCGGCGCGCGGGGATTTCGCCTAC | GALSHPQAPRWPP |
| | GCCGCCCCGGCTCCTCCGGACGGGGCGCTCTCCCAC | HPGKSREDRDPQ |
| | CCTCAGGCTCCTCGGTGGCCTCCGCACCCGGGCAAAA | RDGLPGPCAVAQP |
| | GCCGGGAGGACCGGGACCCGCAGCGCGACGGCCTG | GPAQAGPQGQGV |
| | CCGGGCCCCTGCGCGGTGGCACAGCCTGGGCCCGCT | LAPPTSQGSPWW |
| | CAAGCGGGGCCGCAGGGCCAAGGGGTGCTTGCGCCA | GWGRGPQVAGAA |
| | CCCACGTCCCAGGGGAGTCCGTGGTGGGGCTGGGGC | WEPQAGAAPPPQ |
| | CGGGGTCCCCAGGTCGCCGGGGCGGCGTGGGAACC | PAPPDASASARQG |
| | CCAAGCCGGGGCAGCTCCACCTCCCCAGCCCGCGCC | QMQGIPAPSQALQ |
| | CCCGGACGCCTCCGCCTCCGCGCGGCAGGGGCAGAT | EPAPWSALPCGLL |
| | GCAAGGCATCCCGGCGCCCTCCCAGGCGCTCCAGGA | LDELLASPEFLQQA |
| | GCCGGCGCCCTGGTCTGCACTCCCCTGCGGCCTGCT | QPLLETEAPGELEA |
| | GCTGGATGAGCTCCTGGCGAGCCCGGAGTTTCTGCAG | SEEAASLEAPLSEE |
| | CAGGCGCAACCTCTCCTAGAAACGGAGGCCCCGGGG | EYRALLEEL |
| | GAGCTGGAGGCCTCGGAAGAGGCCGCCTCGCTGGAA | (SEQ ID NO: 374) |
| | GCACCCCTCAGCGAGGAAGAATACCGGGCTCTGCTGG | |
| | AGGAGCTTTAG | |
| | (SEQ ID NO: 341) | |
| ETS1 | ATGAAGGCGGCCGTCGATCTCAAGCCGACTCTCACCA | MKAAVDLKPTLTIIK |
| | TCATCAAGACGGAAAAAGTCGATCTGGAGCTTTTCCCC | TEKVDLELFPSPD |
| | TCCCCGGATATGGAATGTGCAGATGTCCCACTATTAAC | MECADVPLLTPSS |
| | TCCAAGCAGCAAAGAAATGATGTCTCAAGCATTAAAAG | KEMMSQALKATFS |
| | CTACTTTCAGTGGTTTCACTAAAGAACAGCAACGACTG | GFTKEQQRLGIPK |
| | GGGATCCCAAAAGACCCCCGGCAGTGGACAGAAACCC | DPRQWTETHVRD |
| | ATGTTCGGGACTGGGTGATGTGGGCTGTGAATGAATT | WVMWAVNEFSLK |
| | CAGCCTGAAAGGTGTAGACTTCCAGAAGTTCTGTATGA | GVDFQKFCMNGAA |
| | ATGGAGCAGCCCTCTGCGCCCTGGGTAAAGACTGCTT | LCALGKDCFLELAP |
| | TCTCGAGCTGGCCCCAGACTTTGTTGGGGACATCTTAT | DFVGDILWEHLEIL |
| | GGGAACATCTAGAGATCCTGCAGAAAGAGGATGTGAA | QKEDVKPYQVNGV |
| | ACCATATCAAGTTAATGGAGTCAACCCAGCCTATCCAG | NPAYPESRYTSDY |
| | AATCCCGCTATACCTCGGATTACTTCATTAGCTATGGT | FISYGIEHAQCVPP |
| | ATTGAGCATGCCCAGTGTGTTCCACCATCGGAGTTCTC | SEFSEPSFITESYQ |
| | AGAGCCCAGCTTCATCACAGAGTCCTATCAGACGCTC | TLHPISSEELLSLKY |
| | CATCCCATCAGCTCGGAAGAGCTCCTCTCCCTCAAGTA | ENDYPSVILRDPLQ |
| | TGAGAATGACTACCCCTCGGTCATTCTCCGAGACCCTC | TDTLQNDYFAIKQE |
| | TCCAGACAGACACCTTGCAGAATGACTACTTTGCTATC | VVTPDNMCMGRTS |
| | AAACAAGAAGTCGTCACCCCAGACAACATGTGCATGG | RGKLGGQDSFESI |
| | GGAGGACCAGTCGTGGTAAACTCGGGGGCCAGGACT | ESYDSCDRLTQSW |
| | CTTTTGAAAGCATAGAGAGCTACGATAGTTGTGATCGC | SSQSSFNSLQRVP |
| | CTCACCCAGTCCTGGAGCAGCCAGTCATCTTTCAACA | SYDSFDSEDYPAA |
| | GCCTGCAGCGTGTTCCCTCCTATGACAGCTTCGACTC | LPNHKPKGTFKDY |
| | AGAGGACTATCCGGCTGCCCTGCCCAACCACAAGCCC | VRDRADLNKDKPVI |
| | AAGGGCACCTTCAAGGACTATGTGCGGGACCGTGCTG | PAAALAGYTGSGPI |
| | ACCTCAATAAGGACAAGCCTGTCATTCCTGCTGCTGCC | QLWQFLLELLTDK |
| | CTAGCTGGCTACACAGGCAGTGGACCAATCCAGCTAT | SCQSFISWTGDGW |
| | GGCAGTTTCTTCTGGAATTACTCACTGATAAATCCTGT | EFKLSDPDEVARR |
| | CAGTCTTTTATCAGCTGGACAGGAGATGGCTGGGAATT | WGKRKNKPKMNY |
| | CAAACTTTCTGACCCAGATGAGGTGGCCAGGAGATGG | EKLSRGLRYYYDK |
| | GGAAAGAGGAAAAACAAACCTAAGATGAATTATGAGAA | NIIHKTAGKRYVYR |
| | ACTGAGCCGTGGCCTACGCTACTATTACGACAAAAACA | FVCDLQSLLGYTP |
| | TCATCCACAAGACAGCGGGGAAACGCTACGTGTACCG | EELHAMLDVKPDA |
| | CTTTGTGTGTGACCTGCAGAGCCTGCTGGGGTACACC | DE |
| | CCTGAGGAGCTGCACGCCATGCTGGACGTCAAGCCAG | (SEQ ID NO: 375) |
| | ATGCCGACGAGTGA | |
| | (SEQ ID NO: 342) | |
| FOXO1 | ATGGCCGAGGCGCCTCAGGTGGTGGAGATCGACCCG | MAEAPQWVEIDPD |
| | GACTTCGAGCCGCTGCCCCGGCCGCGCTCGTGCACC | FEPLPRPRSCTWP |
| | TGGCCGCTGCCCAGGCCGGAGTTTAGCCAGTCCAACT | LPRPEFSQSNSAT |
| | CGGCCACCTCCAGCCCGGCGCCGTCGGGCAGCGCGG | SSPAPSGSAAANP |
| | CTGCCAACCCCGACGCCGCGGCGGGCCTGCCCTCGG | DAAAGLPSASAAA |
| | CCTCGGCTGCCGCTGTCAGCGCCGACTTCATGAGCAA | VSADFMSNLSLLE |
| | CCTGAGCTTGCTGGAGGAGAGCGAGGACTTCCCGCA | ESEDFPQAPGSVA |
| | GGCGCCCGGCTCCGTGGCGGCGGCGGTGGCGGCGG | AAVAAAAAAAATG |
| | CGGCCGCCGCGGCCGCCACCGGGGGGCTGTGCGGG | GLCGDFQGPEAG |
| | GACTTCCAGGGCCCGGAGGCGGGCTGCCTGCACCCA | CLHPAPPQPPPPG |
| | GCGCCACCGCAGCCCCCGCCGCCCGGGCCGCTGTCG | PLSQHPPVPPAAA |
| | CAGCACCCGCCGGTGCCCCCCGCCGCCGCTGGGCCG | GPLAGQPRKSSSS |
| | CTCGCGGGGCAGCCGCGCAAGAGCAGCTCGTCCCGC | RRNAWGNLSYADL |
| | CGCAACGCGTGGGGCAACCTGTCCTACGCCGACCTCA | ITKAIESSAEKRLTL |
| | TCACCAAGGCCATCGAGAGCTCGGCGGAGAAGCGGC | SQIYEWMVKSVPY |
| | TCACGCTGTCGCAGATCTACGAGTGGATGGTCAAGAG | FKDKGDSNSSAG |
| | CGTGCCCTACTTCAAGGATAAGGGTGACAGCAACAGC | WKNSIRHNLSLHS |
| | TCGGCGGGCTGGAAGAATTCAATTCGTCATAATCTGTC | KFIRVQNEGTGKS |
| | CCTACACAGCAAGTTCATTCGTGTGCAGAATGAAGGAA | SWWMLNPEGGKS |
| | CTGGAAAAAGTTCTTGGTGGATGCTCAATCCAGAGGG | GKSPRRRAASMD |
| | TGGCAAGAGCGGGAAATCTCCTAGGAGAAGAGCTGCA | NNSKFAKSRSRAA |
| | TCCATGGACAACAACAGTAAATTTGCTAAGAGCCGAAG | KKKASLQSGQEGA |
| | CCGAGCTGCCAAGAAGAAAGCATCTCTCCAGTCTGGC | GDSPGSQFSKWP |
| | CAGGAGGGTGCTGGGGACAGCCCTGGATCACAGTTTT | ASPGSHSNDDFDN |
| | CCAAATGGCCTGCAAGCCCTGGCTCTCACAGCAATGA | WSTFRPRTSSNAS |
| | TGACTTTGATAACTGGAGTACATTTCGCCCTCGAACTA | TISGRLSPIMTEQD |
| | GCTCAAATGCTAGTACTATTAGTGGGAGACTCTCACCC | DLGEGDVHSMVYP |
| | ATTATGACCGAACAGGATGATCTTGGAGAAGGGGATG | PSAAKMASTLPSL |
| | TGCATTCTATGGTGTACCCGCCATCTGCCGCAAAGATG | SEISNPENMENLLD |
| | GCCTCTACTTTACCCAGTCTGTCTGAGATAAGCAATCC | NLNLLSSPTSLTVS |
| | CGAAAACATGGAAAATCTTTTGGATAATCTCAACCTTCT | TQSSPGTMMQQT |
| | CTCATCACCAACATCATTAACTGTTTCGACCCAGTCCT | PCYSFAPPNTSLN |
| | CACCTGGCACCATGATGCAGCAGACGCCGTGCTACTC | SPSPNYQKYTYGQ |
| | GTTTGCGCCACCAAACACCAGTTTGAATTCACCCAGCC | SSMSPLPQMPIQT |
| | CAAACTACCAAAAATATACATATGGCCAATCCAGCATG | LQDNKSSYGGMS |
| | AGCCCTTTGCCCCAGATGCCTATACAAACACTTCAGGA | QYNCAPGLLKELLT |
| | CAATAAGTCGAGTTATGGAGGTATGAGTCAGTATAACT | SDSPPHNDIMTPV |
| | GTGCGCCTGGACTCTTGAAGGAGTTGCTGACTTCTGA | DPGVAQPNSRVLG |
| | CTCTCCTCCCCATAATGACATTATGACACCAGTTGATC | QNVMMGPNSVMS |
| | CTGGGGTAGCCCAGCCCAACAGCCGGGTTCTGGGCC | TYGSQASHNKMM |
| | AGAACGTCATGATGGGCCCTAATTCGGTCATGTCAACC | NPSSHTHPGHAQQ |
| | TATGGCAGCCAGGCATCTCATAACAAAATGATGAATCC | TSAVNGRPLPHTV |
| | CAGCTCCCATACCCACCCTGGACATGCTCAGCAGACA | STMPHTSGMNRLT |
| | TCTGCAGTTAACGGGCGTCCCCTGCCCCACACGGTAA | QVKTPVQVPLPHP |
| | GCACCATGCCCCACACCTCGGGTATGAACCGCCTGAC | MQMSALGGYSSV |
| | CCAAGTGAAGACACCTGTACAAGTGCCTCTGCCCCAC | SSCNGYGRMGLLH |
| | CCCATGCAGATGAGTGCCCTGGGGGGCTACTCCTCCG | QEKLPSDLDGMFIE |
| | TGAGCAGCTGCAATGGCTATGGCAGAATGGGCCTTCT | RLDCDMESIIRNDL |
| | CCACCAGGAGAAGCTCCCAAGTGACTTGGATGGCATG | MDGDTLDFNFDNV |
| | TTCATTGAGCGCTTAGACTGTGACATGGAATCCATCAT | LPNQSFPHSVKTT |
| | TCGGAATGACCTCATGGATGGAGATACATTGGATTTTA | THSWVSG |
| | ACTTTGACAATGTGTTGCCCAACCAAAGCTTCCCACAC | (SEQ ID NO: 376) |
| | AGTGTCAAGACAACGACACATAGCTGGGTGTCAGGCT | |
| | GA | |
| | (SEQ ID NO: 343) | |
| FOXP1 | ATGATGCAAGAATCTGGGACTGAGACAAAAAGTAACG | MMQESGTETKSN |
| | GTTCAGCCATCCAGAATGGGTCGGGCGGCAGCAACCA | GSAIQNGSGGSNH |
| | CTTACTAGAGTGCGGCGGTCTTCGGGAGGGGCGGTC | LLECGGLREGRSN |
| | CAACGGAGAGACGCCGGCCGTGGACATCGGGGCAGC | GETPAVDIGAADLA |
| | TGACCTCGCCCACGCCCAGCAGCAGCAGCAACAGGC | HAQQQQQQALQV |
| | ACTTCAGGTGGCAAGACAGCTCCTTCTTCAGCAGCAA | ARQLLLQQQQQQ |
| | CAGCAGCAGCAAGTTAGTGGATTAAAATCTCCCAAGAG | QVSGLKSPKRNDK |
| | GAATGACAAACAACCAGCTCTTCAGGTTCCCGTGTCAG | QPALQVPVSVAMM |
| | TGGCTATGATGACACCTCAAGTTATCACTCCCCAGCAA | TPQVITPQQMQQIL |
| | ATGCAGCAGATCCTCCAGCAACAAGTGCTGAGCCCTC | QQQVLSPQQLQVL |
| | AGCAGCTCCAGGTTCTCCTCCAGCAGCAGCAGGCCCT | LQQQQALMLQQQ |
| | CATGCTTCAACAGCAGCAGCTTCAAGAGTTTTATAAAA | QLQEFYKKQQEQL |
| | AACAACAGGAACAGTTGCAGCTTCAACTTTTACAACAA | QLQLLQQQHAGKQ |
| | CAACATGCTGGAAAACAGCCTAAAGAGCAACAGCAGG | PKEQQQVATQQLA |
| | TGGCTACCCAGCAGTTGGCTTTTCAGCAGCAGCTTTTA | FQQQLLQMQQLQ |
| | CAGATGCAGCAGTTACAGCAGCAGCACCTCCTGTCTTT | QQHLLSLQRQGLL |
| | GCAGCGCCAAGGCCTTCTGACAATTCAGCCCGGGCAG | TIQPGQPALPLQPL |
| | CCTGCCCTTCCCCTTCAACCTCTTGCTCAAGGCATGAT | AQGMIPTELQQLW |
| | TCCAACAGAACTGCAGCAGCTCTGGAAAGAAGTGACA | KEVTSAHTAEETT |
| | AGTGCTCATACTGCAGAAGAAACCACAGGCAACAATCA | GNNHSSLDLTTTC |
| | CAGCAGTTTGGATCTGACCACGACATGTGTCTCCTCCT | VSSSAPSKTSLIMN |
| | CTGCACCTTCCAAGACCTCCTTAATAATGAACCCACAT | PHASTNGQLSVHT |
| | GCCTCTACCAATGGACAGCTCTCAGTCCACACTCCCAA | PKRESLSHEEHPH |
| | AAGGGAAAGTTTGTCCCATGAGGAGCACCCCCATAGC | SHPLYGHGVCKW |
| | CATCCTCTCTATGGACATGGTGTATGCAAGTGGCCAG | PGCEAVCEDFQSF |
| | GCTGTGAAGCAGTGTGCGAAGATTTCCAATCATTTCTA | LKHLNSEHALDDR |
| | AAACATCTCAACAGTGAGCATGCGCTGGACGATAGAA | STAQCRVQMQWV |
| | GTACAGCCCAATGTAGAGTACAAATGCAGGTTGTACAG | QQLELQLAKDKER |
| | CAGTTAGAGCTACAGCTTGCAAAAGACAAAGAACGCCT | LQAMMTHLHVKST |
| | GCAAGCCATGATGACCCACCTGCATGTGAAGTCTACA | EPKAAPQPLNLVS |
| | GAACCCAAAGCCGCCCCTCAGCCCTTGAATCTGGTAT | SVTLSKSASEASP |
| | CAAGTGTCACTCTCTCCAAGTCCGCATCGGAGGCTTCT | QSLPHTPTTPTAPL |
| | CCACAGAGCTTACCTCATACTCCAACGACCCCAACCG | TPVTQGPSVITTTS |
| | CCCCCCTGACTCCCGTCACCCAAGGCCCCTCTGTCAT | MHTVGPIRRRYSD |
| | CACAACCACCAGCATGCACACGGTGGGACCCATCCGC | KYNVPISSADIAQN |
| | AGGCGGTACTCAGACAAATACAACGTGCCCATTTCGTC | QEFYKNAEVRPPF |
| | AGCAGATATTGCGCAGAACCAAGAATTTTATAAGAACG | TYASLIRQAILESPE |
| | CAGAAGTTAGACCACCATTTACATATGCATCTTTAATTA | KQLTLNEIYNWFTR |
| | GGCAGGCCATTCTCGAATCTCCAGAAAAGCAGCTAAC | MFAYFRRNAATWK |
| | ACTAAATGAGATCTATAACTGGTTCACACGAATGTTTG | NAVRHNLSLHKCF |
| | CTTACTTCCGACGCAACGCGGCCACGTGGAAGAATGC | VRVENVKGAVWTV |
| | AGTGCGTCATAATCTTAGTCTTCACAAGTGTTTTGTGC | DEVEFQKRRPQKI |
| | GAGTAGAAAACGTTAAAGGGGCAGTATGGACAGTGGA | SGNPSLIKNMQSS |
| | TGAAGTAGAATTCCAAAAACGAAGGCCACAAAAGATCA | HAYCTPLNAALQA |
| | GTGGTAACCCTTCCCTTATTAAAAACATGCAGAGCAGC | SMAENSIPLYTTAS |
| | CACGCCTACTGCACACCTCTCAATGCAGCTTTACAGGC | MGNPTLGNLASAI |
| | TTCAATGGCTGAGAATAGTATACCTCTATACACTACCG | REELNGAMEHTNS |
| | CTTCCATGGGAAATCCCACTCTGGGCAACTTAGCCAG | NESDSSPGRSPM |
| | CGCAATACGGGAAGAGCTGAACGGGGCAATGGAGCAT | QAVHPVHVKEEPL |
| | ACCAACAGCAACGAGAGTGACAGCAGTCCAGGCAGAT | DPEEAEGPLSLVTT |
| | CTCCTATGCAAGCCGTGCATCCTGTACACGTCAAAGAA | ANHSPDFDHDRDY |
| | GAGCCCCTCGATCCAGAGGAAGCTGAAGGGCCCCTGT | EDEPVNEDME |
| | CCTTAGTGACAACAGCCAACCACAGTCCAGATTTTGAC | (SEQ ID NO: 377) |
| | CATGACAGAGATTACGAAGATGAACCAGTAAACGAGG | |
| | ACATGGAGTGA | |
| | (SEQ ID NO: 344) | |
| FOXP3 | ATGCCCAACCCCAGGCCTGGCAAGCCCTCGGCCCCTT | MPNPRPGKPSAPS |
| | CCTTGGCCCTTGGCCCATCCCCAGGAGCCTCGCCCAG | LALGPSPGASPSW |
| | CTGGAGGGCTGCACCCAAAGCCTCAGACCTGCTGGG | RAAPKASDLLGAR |
| | GGCCCGGGGCCCAGGGGGAACCTTCCAGGGCCGAGA | GPGGTFQGRDLR |
| | TCTTCGAGGCGGGGCCCATGCCTCCTCTTCTTCCTTG | GGAHASSSSLNPM |
| | AACCCCATGCCACCATCGCAGCTGCAGCTGCCCACAC | PPSQLQLPTLPLV |
| | TGCCCCTAGTCATGGTGGCACCCTCCGGGGCACGGCT | MVAPSGARLGPLP |
| | GGGCCCCTTGCCCCACTTACAGGCACTCCTCCAGGAC | HLQALLQDRPHFM |
| | AGGCCACATTTCATGCACCAGCTCTCAACGGTGGATG | HQLSTVDAHARTP |
| | CCCACGCCCGGACCCCTGTGCTGCAGGTGCACCCCC | VLQVHPLESPAMIS |
| | TGGAGAGCCCAGCCATGATCAGCCTCACACCACCCAC | LTPPTTATGVFSLK |
| | CACCGCCACTGGGGTCTTCTCCCTCAAGGCCCGGCCT | ARPGLPPGINVASL |
| | GGCCTCCCACCTGGGATCAACGTGGCCAGCCTGGAAT | EWVSREPALLCTF |
| | GGGTGTCCAGGGAGCCGGCACTGCTCTGCACCTTCCC | PNPSAPRKDSTLS |
| | AAATCCCAGTGCACCCAGGAAGGACAGCACCCTTTCG | AVPQSSYPLLANG |
| | GCTGTGCCCCAGAGCTCCTACCCACTGCTGGCAAATG | VCKWPGCEKVFEE |
| | GTGTCTGCAAGTGGCCCGGATGTGAGAAGGTCTTCGA | PEDFLKHCQADHL |
| | AGAGCCAGAGGACTTCCTCAAGCACTGCCAGGCGGAC | LDEKGRAQCLLQR |
| | CATCTTCTGGATGAGAAGGGCAGGGCACAATGTCTCC | EMVQSLEQQLVLE |
| | TCCAGAGAGAGATGGTACAGTCTCTGGAGCAGCAGCT | KEKLSAMQAHLAG |
| | GGTGCTGGAGAAGGAGAAGCTGAGTGCCATGCAGGC | KMALTKASSVASS |
| | CCACCTGGCTGGGAAAATGGCACTGACCAAGGCTTCA | DKGSCCIVAAGSQ |
| | TCTGTGGCATCATCCGACAAGGGCTCCTGCTGCATCG | GPWPAWSGPRE |
| | TAGCTGCTGGCAGCCAAGGCCCTGTCGTCCCAGCCTG | APDSLFAVRRHLW |
| | GTCTGGCCCCCGGGAGGCCCCTGACAGCCTGTTTGCT | GSHGNSTFPEFLH |
| | GTCCGGAGGCACCTGTGGGGTAGCCATGGAAACAGC | NMDYFKFHNMRPP |
| | ACATTCCCAGAGTTCCTCCACAACATGGACTACTTCAA | FTYATLIRWAILEAP |
| | GTTCCACAACATGCGACCCCCTTTCACCTACGCCACG | EKQRTLNEIYHWFT |
| | CTCATCCGCTGGGCCATCCTGGAGGCTCCAGAGAAGC | RMFAFFRNHPATW |
| | AGCGGACACTCAATGAGATCTACCACTGGTTCACACG | KNAIRHNLSLHKCF |
| | CATGTTTGCCTTCTTCAGAAACCATCCTGCCACCTGGA | VRVESEKGAVWTV |
| | AGAACGCCATCCGCCACAACCTGAGTCTGCACAAGTG | DELEFRKKRSQRP |
| | CTTTGTGCGGGTGGAGAGCGAGAAGGGGGCTGTGTG | SRCSNPTPGP |
| | GACCGTGGATGAGCTGGAGTTCCGCAAGAAACGGAGC | (SEQ ID NO: 378) |
| | CAGAGGCCCAGCAGGTGTTCCAACCCTACACCTGGCC | |
| | CCTGA | |
| | (SEQ ID NO: 345) | |
| GATA3 | ATGGAGGTGACGGCGGACCAGCCGCGCTGGGTGAGC | MEVTADQPRWVS |
| | CACCACCACCCCGCCGTGCTCAACGGGCAGCACCCG | HHHPAVLNGQHPD |
| | GACACGCACCACCCGGGCCTCAGCCACTCCTACATGG | THHPGLSHSYMDA |
| | ACGCGGCGCAGTACCCGCTGCCGGAGGAGGTGGATG | AQYPLPEEVDVLF |
| | TGCTTTTTAACATCGACGGTCAAGGCAACCACGTCCCG | NIDGQGNHVPPYY |
| | CCCTACTACGGAAACTCGGTCAGGGCCACGGTGCAGA | GNSVRATVQRYPP |
| | GGTACCCTCCGACCCACCACGGGAGCCAGGTGTGCC | THHGSQVCRPPLL |
| | GCCCGCCTCTGCTTCATGGATCCCTACCCTGGCTGGA | HGSLPWLDGGKAL |
| | CGGCGGCAAAGCCCTGGGCAGCCACCACACCGCCTC | GSHHTASPWNLSP |
| | CCCCTGGAATCTCAGCCCCTTCTCCAAGACGTCCATC | FSKTSIHHGSPGPL |
| | CACCACGGCTCCCCGGGGCCCCTCTCCGTCTACCCCC | SVYPPASSSSLSG |
| | CGGCCTCGTCCTCCTCCTTGTCGGGGGGCCACGCCA | GHASPHLFTFPPT |
| | GCCCGCACCTCTTCACCTTCCCGCCCACCCCGCCGAA | PPKDVSPDPSLST |
| | GGACGTCTCCCCGGACCCATCGCTGTCCACCCCAGGC | PGSAGSARQDEKE |
| | TCGGCCGGCTCGGCCCGGCAGGACGAGAAAGAGTGC | CLKYQVPLPDSMK |
| | CTCAAGTACCAGGTGCCCCTGCCCGACAGCATGAAGC | LESSHSRGSMTAL |
| | TGGAGTCGTCCCACTCCCGTGGCAGCATGACCGCCCT | GGASSSTHHPITTY |
| | GGGTGGAGCCTCCTCGTCGACCCACCACCCCATCACC | PPYVPEYSSGLFP |
| | ACCTACCCGCCCTACGTGCCCGAGTACAGCTCCGGAC | PSSLLGGSPTGFG |
| | TCTTCCCCCCCAGCAGCCTGCTGGGCGGCTCCCCCAC | CKSRPKARSSTGR |
| | CGGCTTCGGATGCAAGTCCAGGCCCAAGGCCCGGTC | ECVNCGATSTPLW |
| | CAGCACAGGCAGGGAGTGTGTGAACTGTGGGGCAAC | RRDGTGHYLCNAC |
| | CTCGACCCCACTGTGGCGGCGAGATGGCACGGGACA | GLYHKMNGQNRPL |
| | CTACCTGTGCAACGCCTGCGGGCTCTATCACAAAATG | IKPKRRLSAARRAG |
| | AACGGACAGAACCGGCCCCTCATTAAGCCCAAGCGAA | TSCANCQTTTTTL |
| | GGCTGTCTGCAGCCAGGAGAGCAGGGACGTCCTGTG | WRRNANGDPVCN |
| | CGAACTGTCAGACCACCACAACCACACTCTGGAGGAG | ACGLYYKLHNINRP |
| | GAATGCCAATGGGGACCCTGTCTGCAATGCCTGTGGG | LTMKKEGIQTRNR |
| | CTCTACTACAAGCTTCACAATATTAACAGACCCCTGAC | KMSSKSKKCKKVH |
| | TATGAAGAAGGAAGGCATCCAGACCAGAAACCGAAAA | DSLEDFPKNSSFN |
| | ATGTCTAGCAAATCCAAAAAGTGCAAAAAAGTGCATGA | PAALSRHMSSLSHI |
| | CTCACTGGAGGACTTCCCCAAGAACAGCTCGTTTAACC | SPFSHSSHMLTTP |
| | CGGCCGCCCTCTCCAGACACATGTCCTCCCTGAGCCA | TPMHPPSSLSFGP |
| | CATCTCGCCCTTCAGCCACTCCAGCCACATGCTGACC | HHPSSMVTAMG |
| | ACGCCCACGCCGATGCACCCGCCATCCAGCCTGTCCT | (SEQ ID NO: 379) |
| | TTGGACCACACCACCCCTCCAGCATGGTCACCGCCAT | |
| | GGGTTAG | |
| | (SEQ ID NO: 346) | |
| GFI1 | ATGCCGCGCTCATTTCTCGTCAAAAGCAAGAAGGCTCA | MPRSFLVKSKKAH |
| | CAGCTACCACCAGCCGCGCTCCCCAGGACCAGACTAT | SYHQPRSPGPDYS |
| | TCCCTCCGTTTAGAGAATGTACCGGCGCCTAGCCGAG | LRLENVPAPSRAD |
| | CAGACAGCACTTCAAATGCAGGCGGGGCGAAGGCGG | STSNAGGAKAEPR |
| | AGCCCCGGGACCGTTTGTCCCCCGAATCGCAGCTGAC | DRLSPESQLTEAP |
| | CGAAGCCCCAGACAGAGCCTCCGCATCCCCAGACAGC | DRASASPDSCEGS |
| | TGCGAAGGCAGCGTCTGCGAACGGAGCTCGGAGTTTG | VCERSSEFEDFWR |
| | AGGACTTCTGGAGGCCCCCGTCACCCTCCGCGTCTCC | PPSPSASPASEKS |
| | AGCCTCGGAGAAGTCAATGTGCCCATCGCTGGACGAA | MCPSLDEAQPFPL |
| | GCCCAGCCCTTCCCCCTGCCTTTCAAACCGTACTCATG | PFKPYSWSGLAGS |
| | GAGCGGCCTGGCGGGTTCTGACCTGCGGCACCTGGT | DLRHLVQSYRPCG |
| | GCAGAGCTACCGACCGTGTGGGGCCCTGGAGCGTGG | ALERGAGLGLFCE |
| | CGCTGGCCTGGGCCTCTTCTGCGAACCCGCCCCGGA | PAPEPGHPAALYG |
| | GCCTGGCCACCCGGCCGCGCTGTACGGCCCGAAGCG | PKRAAGGAGAGAP |
| | GGCTGCCGGCGGCGCGGGGGCCGGGGCGCCAGGGA | GSCSAGAGATAGP |
| | GCTGCAGCGCAGGGGCCGGTGCCACCGCTGGCCCTG | GLGLYGDFGSAAA |
| | GCCTAGGGCTCTACGGCGACTTCGGGTCTGCGGCAG | GLYERPTAAAGLL |
| | CCGGGCTGTATGAGAGGCCCACGGCAGCGGCGGGCT | YPERGHGLHADKG |
| | TGCTGTACCCCGAGCGTGGCCACGGGCTGCACGCAG | AGVKVESELLCTRL |
| | ACAAGGGCGCTGGCGTCAAGGTGGAGTCGGAGCTGC | LLGGGSYKCIKCSK |
| | TGTGCACCCGCCTGCTGCTGGGCGGCGGCTCCTACAA | VFSTPHGLEVHVR |
| | GTGCATCAAGTGCAGCAAGGTGTTCTCCACGCCGCAC | RSHSGTRPFACEM |
| | GGGCTCGAGGTGCACGTGCGCAGGTCCCACAGCGGT | CGKTFGHAVSLEQ |
| | ACCAGACCCTTTGCCTGCGAGATGTGCGGCAAGACCT | HKAVHSQERSFDC |
| | TCGGGCACGCGGTGAGCCTGGAGCAGCACAAAGCCG | KICGKSFKRSSTLS |
| | TGCACTCGCAGGAACGGAGCTTTGACTGTAAGATCTG | THLLIHSDTRPYPC |
| | TGGGAAGAGCTTCAAGAGGTCATCCACACTGTCCACA | QYCGKRFHQKSD |
| | CACCTGCTTATCCACTCAGACACTCGGCCCTACCCCT | MKKHTFIHTGEKPH |
| | GTCAGTACTGTGGCAAGAGGTTCCACCAGAAGTCAGA | KCQVCGKAFSQSS |
| | CATGAAGAAACACACTTTCATCCACACTGGTGAGAAGC | NLITHSRKHTGFKP |
| | CTCACAAGTGCCAGGTGTGCGGCAAGGCATTCAGCCA | FGCDLCGKGFQRK |
| | GAGCTCCAACCTCATCACCCACAGCCGCAAACACACA | VDLRRHRETQHGL |
| | GGCTTCAAGCCCTTCGGCTGCGACCTCTGTGGGAAGG | K |
| | GTTTCCAGAGGAAGGTGGACCTCCGAAGGCACCGGG | (SEQ ID NO: 380) |
| | AGACGCAGCATGGGCTCAAATGA | |
| | (SEQ ID NO: 347) | |
| GTF2B | ATGGCGTCTACCAGCCGTTTGGATGCTCTTCCAAGAGT | MASTSRLDALPRV |
| | CACATGTCCAAACCATCCAGATGCGATTTTAGTGGAGG | TCPNHPDAILVEDY |
| | ACTACAGAGCCGGTGATATGATCTGTCCTGAATGTGG | RAGDMICPECGLV |
| | CTTGGTTGTAGGTGACCGGGTTATTGATGTGGGATCT | VGDRVIDVGSEWR |
| | GAATGGCGAACTTTCAGCAATGACAAAGCAACAAAAGA | TFSNDKATKDPSR |
| | TCCATCTCGAGTTGGAGATTCTCAGAATCCTCTTCTGA | VGDSQNPLLSDGD |
| | GTGATGGAGATTTGTCTACCATGATTGGCAAGGGCAC | LSTMIGKGTGAASF |
| | AGGAGCTGCAAGTTTTGACGAATTTGGCAATTCTAAGT | DEFGNSKYQNRRT |
| | ACCAGAATCGGAGAACAATGAGCAGTTCTGATCGGGC | MSSSDRAMMNAF |
| | AATGATGAATGCATTCAAAGAAATCACTACCATGGCAG | KEITTMADRINLPR |
| | ACAGAATCAATCTACCTCGAAATATAGTTGATCGAACA | NIVDRTNNLFKQVY |
| | AATAATTTATTCAAGCAAGTATATGAACAGAAGAGCCT | EQKSLKGRANDAI |
| | GAAGGGAAGAGCTAATGATGCTATAGCTTCTGCTTGTC | ASACLYIACRQEG |
| | TCTATATTGCCTGTAGACAAGAAGGGGTTCCTAGGACA | VPRTFKEICAVSRI |
| | TTTAAAGAAATATGTGCCGTATCACGAATTTCTAAGAAA | SKKEIGRCFKLILKA |
| | GAAATTGGTCGGTGTTTTAAACTTATTTTGAAAGCGCTA | LETSVDLITTGDFM |
| | GAAACCAGTGTGGATTTGATTACAACTGGGGACTTCAT | SRFCSNLCLPKQV |
| | GTCCAGGTTCTGTTCCAACCTTTGTCTTCCTAAACAAG | QMAATHIARKAVEL |
| | TACAGATGGCAGCTACACATATAGCCCGTAAAGCTGTG | DLVPGRSPISVAAA |
| | GAACTGGACTTGGTTCCTGGGAGGAGCCCCATCTCTG | AIYMASQASAEKR |
| | TGGCAGCGGCAGCTATTTACATGGCCTCACAGGCATC | TQKEIGDIAGVADV |
| | AGCTGAAAAGAGGACCCAAAAAGAAATTGGAGATATTG | TIRQSYRLIYPRAP |
| | CTGGTGTTGCTGATGTTACAATCAGACAGTCCTATAGA | DLFPTDFKFDTPVD |
| | CTGATCTATCCTCGAGCCCCAGATCTGTTTCCTACAGA | KLPQL |
| | CTTCAAATTTGACACCCCAGTGGACAAACTACCACAGC | (SEQ ID NO: 381) |
| | TATAA | |
| | (SEQ ID NO: 348) | |
| HIC1 | ATGCTGGACACGATGGAGGCGCCCGGCCACTCCAGG | MLDTMEAPGHSR |
| | CAGCTGCTGCTGCAGCTCAACAACCAGCGCACCAAGG | QLLLQLNNQRTKG |
| | GCTTCTTGTGCGACGTGATCATCGTGGTGCAGAACGC | FLCDVIIVVQNALF |
| | CCTCTTCCGCGCGCACAAGAACGTGCTGGCGGCCAG | RAHKNVLAASSAY |
| | CAGCGCCTACCTCAAGTCCCTGGTGGTGCATGACAAC | LKSLWVHDNLLNLD |
| | CTGCTCAACCTGGACCATGACATGGTGAGCCCGGCCG | HDMVSPAVFRLVL |
| | TGTTCCGCCTGGTGCTGGACTTCATCTACACCGGCCG | DFIYTGRLADGAEA |
| | CCTGGCTGACGGCGCAGAGGCGGCTGCGGCCGCGG | AAAAAVAPGAEPS |
| | CCGTGGCCCCGGGGGCTGAGCCGAGCCTGGGCGCC | LGAVLAAASYLQIP |
| | GTGCTGGCCGCCGCCAGCTACCTGCAGATCCCCGAC | DLVALCKKRLKRH |
| | CTCGTGGCGCTGTGCAAGAAACGCCTCAAGCGCCACG | GKYCHLRGGGGG |
| | GCAAGTACTGCCACCTGCGGGGCGGCGGCGGCGGCG | GGGYAPYGRPGR |
| | GCGGCGGCTACGCGCCCTATGGTCGGCCGGGCCGGG | GLRAATPVIQACYP |
| | GCCTGCGGGCCGCCACGCCGGTCATCCAGGCCTGCT | SPVGPPPPPAAEP |
| | ACCCGTCCCCAGTCGGGCCTCCGCCGCCGCCTGCCG | PSGPEAAVNTHCA |
| | CGGAGCCGCCCTCGGGCCCAGAGGCCGCGGTCAACA | ELYASGPGPAAAL |
| | CGCACTGCGCCGAGCTGTACGCGTCGGGACCCGGCC | CASERRCSPLCGL |
| | CGGCCGCCGCACTCTGTGCCTCGGAGCGCCGCTGCT | DLSKKSPPGSAAP |
| | CCCCTCTTTGTGGCCTGGACCTGTCCAAGAAGAGCCC | ERPLAERELPPRP |
| | GCCGGGCTCCGCGGCGCCAGAGCGGCCGCTGGCTGA | DSPPSAGPAAYKE |
| | GCGCGAGCTGCCCCCGCGCCCGGACAGCCCTCCCAG | PPLALPSLPPLPFQ |
| | CGCCGGCCCCGCCGCCTACAAGGAGCCGCCTCTCGC | KLEEAAPPSDPFR |
| | CCTGCCGTCGCTGCCGCCGCTGCCCTTCCAGAAGCTG | GGSGSPGPEPPG |
| | GAGGAGGCCGCACCGCCTTCCGACCCATTTCGCGGC | RPDGPSLLYRWMK |
| | GGCAGCGGCAGCCCGGGACCCGAGCCCCCCGGCCG | HEPGLGSYGDELG |
| | CCCCGACGGGCCTAGTCTCCTCTATCGCTGGATGAAG | RERGSPSERCEER |
| | CACGAGCCGGGCCTGGGTAGCTATGGCGACGAGCTG | GGDAAVSPGGPPL |
| | GGCCGGGAGCGCGGCTCCCCCAGCGAGCGCTGCGAA | GLAPPPRYPGSLD |
| | GAGCGTGGTGGGGACGCGGCCGTCTCGCCCGGGGG | GPGAGGDGDDYK |
| | GCCCCCGCTCGGCCTGGCGCCGCCGCCGCGCTACCC | SSSEETGSSEDPS |
| | TGGCAGCCTGGACGGGCCCGGCGCGGGCGGCGACG | PPGGHLEGYPCPH |
| | GCGACGACTACAAGAGCAGCAGCGAGGAGACCGGTA | LAYGEPESFGDNL |
| | GCAGCGAGGACCCCAGCCCGCCTGGCGGCCACCTCG | YVCIPCGKGFPSS |
| | AGGGCTACCCATGCCCGCACCTGGCCTATGGCGAGC | EQLNAHVEAHVEE |
| | CCGAGAGCTTCGGTGACAACCTGTACGTGTGCATTCC | EEALYGRAEAAEV |
| | GTGCGGCAAGGGCTTCCCCAGCTCTGAGCAGCTGAAC | AAGAAGLGPPFGG |
| | GCGCACGTGGAGGCTCACGTGGAGGAGGAGGAAGCG | GGDKVAGAPGGL |
| | CTGTACGGCAGGGCCGAGGCGGCCGAAGTGGCCGCT | GELLRPYRCASCD |
| | GGGGCCGCCGGCCTAGGGCCCCCTTTTGGAGGCGGC | KSYKDPATLRQHE |
| | GGGGACAAGGTCGCCGGGGCTCCGGGTGGCCTGGGA | KTHWLTRPYPCTIC |
| | GAGCTGCTGCGGCCCTACCGCTGCGCGTCGTGCGAC | GKKFTQRGTMTRH |
| | AAGAGCTACAAGGACCCGGCCACGCTGCGGCAGCAC | MRSHLGLKPFACD |
| | GAGAAGACGCACTGGCTGACCCGGCCCTACCCATGCA | ACGMRFTRQYRLT |
| | CCATCTGCGGGAAGAAGTTCACGCAGCGTGGGACCAT | EHMRIHSGEKPYE |
| | GACGCGCCACATGCGCAGCCACCTGGGCCTCAAGCC | CQVCGGKFAQQR |
| | CTTCGCGTGCGACGCGTGCGGCATGCGGTTCACGCG | NLISHMKMHAVGG |
| | CCAGTACCGCCTCACGGAGCACATGCGCATCCACTCG | AAGAAGALAGLGG |
| | GGCGAGAAGCCCTACGAGTGCCAGGTGTGCGGCGGC | LPGVPGPDGKGKL |
| | AAGTTCGCACAGCAACGCAACCTCATCAGCCACATGA | DFPEGVFAVARLT |
| | AGATGCACGCCGTGGGGGGCGCGGCCGGCGCGGCC | AEQLSLKQQDKAA |
| | GGGGCGCTGGCGGGCTTGGGGGGGCTCCCCGGCGT | AAELLAQTTHFLHD |
| | CCCCGGCCCCGACGGCAAGGGCAAGCTCGACTTCCC | PKVALESLYPLAKF |
| | CGAGGGCGTCTTTGCTGTGGCTCGCCTCACGGCCGA | TAELGLSPDKAAE |
| | GCAGCTGAGCCTGAAGCAGCAGGACAAGGCGGCCGC | VLSQGAHLAAGPD |
| | GGCCGAGCTGCTGGCGCAGACCACGCACTTCCTGCA | GRTIDRFSPT |
| | CGACCCCAAGGTGGCGCTGGAGAGCCTCTACCCGCT | (SEQ ID NO: 382) |
| | GGCCAAGTTCACGGCCGAGCTGGGCCTCAGCCCCGA | |
| | CAAGGCGGCCGAGGTGCTGAGCCAGGGCGCTCACCT | |
| | GGCGGCCGGGCCCGACGGCCGGACCATCGACCGTTT | |
| | CTCTCCCACCTAG | |
| | (SEQ ID NO: 349) | |
| IRF1 | ATGCCCATCACTCGGATGCGCATGAGACCCTGGCTAG | MPITRMRMRPWLE |
| | AGATGCAGATTAATTCCAACCAAATCCCGGGGCTCATC | MQINSNQIPGLIWI |
| | TGGATTAATAAAGAGGAGATGATCTTCCAGATCCCATG | NKEEMIFQIPWKHA |
| | GAAGCATGCTGCCAAGCATGGCTGGGACATCAACAAG | AKHGWDINKDACL |
| | GATGCCTGTTTGTTCCGGAGCTGGGCCATTCACACAG | FRSWAIHTGRYKA |
| | GCCGATACAAAGCAGGGGAAAAGGAGCCAGATCCCAA | GEKEPDPKTWKAN |
| | GACGTGGAAGGCCAACTTTCGCTGTGCCATGAACTCC | FRCAMNSLPDIEEV |
| | CTGCCAGATATCGAGGAGGTGAAAGACCAGAGCAGGA | KDQSRNKGSSAVR |
| | ACAAGGGCAGCTCAGCTGTGCGAGTGTACCGGATGCT | VYRMLPPLTKNQR |
| | TCCACCTCTCACCAAGAACCAGAGAAAAGAAAGAAAGT | KERKSKSSRDAKS |
| | CGAAGTCCAGCCGAGATGCTAAGAGCAAGGCCAAGAG | KAKRKSCGDSSPD |
| | GAAGTCATGTGGGGATTCCAGCCCTGATACCTTCTCTG | TFSDGLSSSTLPD |
| | ATGGACTCAGCAGCTCCACTCTGCCTGATGACCACAG | DHSSYTVPGYMQD |
| | CAGCTACACAGTTCCAGGCTACATGCAGGACTTGGAG | LEVEQALTPALSPC |
| | GTGGAGCAGGCCCTGACTCCAGCACTGTCGCCATGTG | AVSSTLPDWHIPVE |
| | CTGTCAGCAGCACTCTCCCCGACTGGCACATCCCAGT | WVPDSTSDLYNFQ |
| | GGAAGTTGTGCCGGACAGCACCAGTGATCTGTACAAC | VSPMPSTSEATTD |
| | TTCCAGGTGTCACCCATGCCCTCCACCTCTGAAGCTAC | EDEEGKLPEDIMKL |
| | AACAGATGAGGATGAGGAAGGGAAATTACCTGAGGAC | LEQSEWQPTNVD |
| | ATCATGAAGCTCTTGGAGCAGTCGGAGTGGCAGCCAA | GKGYLLNEPGVQP |
| | CAAACGTGGATGGGAAGGGGTACCTACTCAATGAACC | TSVYGDFSCKEEP |
| | TGGAGTCCAGCCCACCTCTGTCTATGGAGACTTTAGCT | EIDSPGGDIGLSLQ |
| | GTAAGGAGGAGCCAGAAATTGACAGCCCAGGGGGGG | RVFTDLKNMDATW |
| | ATATTGGGCTGAGTCTACAGCGTGTCTTCACAGATCTG | LDSLLTPVRLPSIQ |
| | AAGAACATGGATGCCACCTGGCTGGACAGCCTGCTGA | AIPCAP |
| | CCCCAGTCCGGTTGCCCTCCATCCAGGCCATTCCCTG | (SEQ ID NO: 383) |
| | TGCACCGTAG | |
| | (SEQ ID NO: 350) | |
| KCMF 1 | ATGTCCCGACATGAAGGTGTCAGCTGTGATGCATGTTT | MSRHEGVSCDACL |
| | AAAAGGAAATTTTCGAGGTCGCAGATATAAGTGTTTAA | KGNFRGRRYKCLI |
| | TTTGCTACGATTACGATCTTTGTGCATCTTGTTATGAAA | CYDYDLCASCYES |
| | GTGGTGCAACAACAACAAGGCATACAACTGACCACCC | GATTTRHTTDHPM |
| | AATGCAGTGCATATTAACAAGGGTAGATTTTGATTTATA | QCILTRVDFDLYYG |
| | CTATGGTGGGGAAGCTTTCTCTGTAGAGCAGCCACAG | GEAFSVEQPQSFT |
| | TCTTTTACTTGTCCCTATTGTGGAAAAATGGGCTATAC | CPYCGKMGYTETS |
| | GGAGACATCTCTTCAAGAACATGTTACTTCTGAACATG | LQEHVTSEHAETS |
| | CAGAAACATCAACAGAAGTGATTTGTCCAATATGTGCA | TEVICPICAALPGG |
| | GCGTTACCTGGAGGCGATCCTAATCATGTCACGGATG | DPNHVTDDFAAHL |
| | ACTTTGCAGCTCATCTTACACTTGAACACAGAGCCCCT | TLEHRAPRDLDES |
| | AGAGATTTAGATGAATCGAGTGGTGTTCGACATGTACG | SGVRHVRRMFHP |
| | TAGAATGTTTCACCCTGGCCGGGGATTAGGAGGTCCT | GRGLGGPRARRS |
| | CGTGCTCGTAGATCAAACATGCACTTTACTAGCAGTTC | NMHFTSSSTGGLS |
| | TACTGGTGGACTTTCTTCTTCTCAGAGTTCATATTCTCC | SSQSSYSPSNREA |
| | AAGCAATAGGGAAGCCATGGATCCTATAGCTGAGCTTT | MDPIAELLSQLSGV |
| | TATCTCAGTTATCAGGAGTGAGACGTTCTGCAGGAGG | RRSAGGQLNSSGP |
| | ACAGCTTAATTCCTCTGGCCCTTCCGCTTCTCAGTTAC | SASQLQQLQMQLQ |
| | AACAACTGCAGATGCAGCTGCAGCTAGAACGGCAGCA | LERQHAQAARQQL |
| | TGCCCAGGCAGCACGGCAACAACTGGAGACCGCACG | ETARNATRRTNTS |
| | CAACGCAACCCGGCGTACTAACACAAGCAGTGTCACC | SVTTTITQSTATTNI |
| | ACTACAATCACACAATCCACAGCAACAACCAACATAGC | ANTESSQQTLQNS |
| | TAATACAGAAAGCAGTCAGCAGACTCTACAGAATTCCC | QFLLTRLNDPKMS |
| | AGTTTCTTTTAACAAGGTTGAATGATCCTAAAATGTCTG | ETERQSMESERAD |
| | AAACGGAGCGCCAGTCCATGGAAAGCGAGCGTGCAG | RSLFVQELLLSTLV |
| | ACCGCAGCCTGTTTGTCCAAGAGCTCCTTCTGTCCACT | REESSSSDEDDRG |
| | TTAGTGCGTGAAGAGAGCTCATCCTCAGATGAGGATG | EMADFGAMGCVDI |
| | ATCGGGGGGAGATGGCAGATTTTGGTGCTATGGGCTG | MPLDVALENLNLK |
| | TGTAGATATTATGCCTTTAGATGTTGCTTTAGAAAACCT | ESNKGNEPPPPPL |
| | AAATTTAAAAGAGAGTAATAAAGGAAATGAGCCTCCAC | (SEQ ID NO: 384) |
| | CACCTCCTCTTTGA | |
| | (SEQ ID NO: 351) | |
| MBD2 | ATGCGCGCGCACCCGGGGGGAGGCCGCTGCTGCCCG | MRAHPGGGRCCP |
| | GAGCAGGAGGAGGGGGAGAGTGCGGCGGGCGGCAG | EQEEGESAAGGS |
| | CGGCGCTGGCGGCGACTCCGCCATAGAGCAGGGGGG | GAGGDSAIEQGGQ |
| | CCAGGGCAGCGCGCTCGCCCCGTCCCCGGTGAGCGG | GSALAPSPVSGVR |
| | CGTGCGCAGGGAAGGCGCTCGGGGCGGCGGCCGTG | REGARGGGRGRG |
| | GCCGGGGGCGGTGGAAGCAGGCGGGCCGGGGCGGC | RWKQAGRGGGVC |
| | GGCGTCTGTGGCCGTGGCCGGGGCCGGGGCCGTGG | GRGRGRGRGRGR |
| | CCGGGGACGGGGACGGGGCCGGGGCCGGGGCCGCG | GRGRGRGRGRPP |
| | GCCGTCCCCCGAGTGGCGGCAGCGGCCTTGGCGGCG | SGGSGLGGDGGG |
| | ACGGCGGCGGCTGCGGCGGCGGCGGCAGCGGTGGC | CGGGGSGGGGAP |
| | GGCGGCGCCCCCCGGCGGGAGCCGGTCCCTTTCCCG | RREPVPFPSGSAG |
| | TCGGGGAGCGCGGGGCCGGGGCCCAGGGGACCCCG | PGPRGPRATESGK |
| | GGCCACGGAGAGCGGGAAGAGGATGGATTGCCCGGC | RMDCPALPPGWK |
| | CCTCCCCCCCGGATGGAAGAAGGAGGAAGTGATCCGA | KEEVIRKSGLSAGK |
| | AAATCTGGGCTAAGTGCTGGCAAGAGCGATGTCTACT | SDVYYFSPSGKKF |
| | ACTTCAGTCCAAGTGGTAAGAAGTTCAGAAGCAAGCCT | RSKPQLARYLGNT |
| | CAGTTGGCAAGGTACCTGGGAAATACTGTTGATCTCAG | VDLSSFDFRTGKM |
| | CAGTTTTGACTTCAGAACTGGAAAGATGATGCCTAGTA | MPSKLQKNKQRLR |
| | AATTACAGAAGAACAAACAGAGACTGCGAAACGATCCT | NDPLNQNKGKPDL |
| | CTCAATCAAAATAAGGGTAAACCAGACTTGAATACAAC | NTTLPIRQTASIFK |
| | ATTGCCAATTAGACAAACAGCATCAATTTTCAAACAACC | QPVTKVTNHPSNK |
| | GGTAACCAAAGTCACAAATCATCCTAGTAATAAAGTGA | VKSDPQRMNEQP |
| | AATCAGACCCACAACGAATGAATGAACAGCCACGTCA | RQLFWEKRLQGLS |
| | GCTTTTCTGGGAGAAGAGGCTACAAGGACTTAGTGCA | ASDVTEQIIKTMEL |
| | TCAGATGTAACAGAACAAATTATAAAAACCATGGAACT | PKGLQGVGPGSN |
| | ACCCAAAGGTCTTCAAGGAGTTGGTCCAGGTAGCAAT | DETLLSAVASALHT |
| | GATGAGACCCTTTTATCTGCTGTTGCCAGTGCTTTGCA | SSAPITGQVSAAVE |
| | CACAAGCTCTGCGCCAATCACAGGGCAAGTCTCCGCT | KNPAVWLNTSQPL |
| | GCTGTGGAAAAGAACCCTGCTGTTTGGCTTAACACATC | CKAFIVTDEDIRKQ |
| | TCAACCCCTCTGCAAAGCTTTTATTGTCACAGATGAAG | EERVQQVRKKLEE |
| | ACATCAGGAAACAGGAAGAGCGAGTACAGCAAGTACG | ALMADILSRAADTE |
| | CAAGAAATTGGAAGAAGCACTGATGGCAGACATCTTGT | EMDIEMDSGDEA |
| | CGCGAGCTGCTGATACAGAAGAGATGGATATTGAAAT | (SEQ ID NO: 385) |
| | GGACAGTGGAGATGAAGCCTAA | |
| | (SEQ ID NO: 352) | |
| MYC | CTGGATTTTTTTCGGGTAGTGGAAAACCAGCCTCCCGC | MDFFRVVENQPPA |
| | GACGATGCCCCTCAACGTTAGCTTCACCAACAGGAAC | TMPLNVSFTNRNY |
| | TATGACCTCGACTACGACTCGGTGCAGCCGTATTTCTA | DLDYDSVQPYFYC |
| | CTGCGACGAGGAGGAGAACTTCTACCAGCAGCAGCAG | DEEENFYQQQQQ |
| | CAGAGCGAGCTGCAGCCCCCGGCGCCCAGCGAGGAT | SELQPPAPSEDIW |
| | ATCTGGAAGAAATTCGAGCTGCTGCCCACCCCGCCCC | KKFELLPTPPLSPS |
| | TGTCCCCTAGCCGCCGCTCCGGGCTCTGCTCGCCCTC | RRSGLCSPSYVAV |
| | CTACGTTGCGGTCACACCCTTCTCCCTTCGGGGAGAC | TPFSLRGDNDGGG |
| | AACGACGGCGGTGGCGGGAGCTTCTCCACGGCCGAC | GSFSTADQLEMVT |
| | CAGCTGGAGATGGTGACCGAGCTGCTGGGAGGAGAC | ELLGGDMVNQSFI |
| | ATGGTGAACCAGAGTTTCATCTGCGACCCGGACGACG | CDPDDETFIKNIIIQ |
| | AGACCTTCATCAAAAACATCATCATCCAGGACTGTATG | DCMWSGFSAAAKL |
| | TGGAGCGGCTTCTCGGCCGCCGCCAAGCTCGTCTCAG | VSEKLASYQAARK |
| | AGAAGCTGGCCTCCTACCAGGCTGCGCGCAAAGACAG | DSGSPNPARGHSV |
| | CGGCAGCCCGAACCCCGCCCGCGGCCACAGCGTCTG | CSTSSLYLQDLSAA |
| | CTCCACCTCCAGCTTGTACCTGCAGGATCTGAGCGCC | ASECIDPSVVFPYP |
| | GCCGCCTCAGAGTGCATCGACCCCTCGGTGGTCTTCC | LNDSSSPKSCASQ |
| | CCTACCCTCTCAACGACAGCAGCTCGCCCAAGTCCTG | DSSAFSPSSDSLLS |
| | CGCCTCGCAAGACTCCAGCGCCTTCTCTCCGTCCTCG | STESSPQGSPEPL |
| | GATTCTCTGCTCTCCTCGACGGAGTCCTCCCCGCAGG | VLHEETPPTTSSDS |
| | GCAGCCCCGAGCCCCTGGTGCTCCATGAGGAGACAC | EEEQEDEEEIDVVS |
| | CGCCCACCACCAGCAGCGACTCTGAGGAGGAACAAGA | VEKRQAPGKRSES |
| | AGATGAGGAAGAAATCGATGTTGTTTCTGTGGAAAAGA | GSPSAGGHSKPPH |
| | GGCAGGCTCCTGGCAAAAGGTCAGAGTCTGGATCACC | SPLVLKRCHVSTH |
| | TTCTGCTGGAGGCCACAGCAAACCTCCTCACAGCCCA | QHNYAAPPSTRKD |
| | CTGGTCCTCAAGAGGTGCCACGTCTCCACACATCAGC | YPAAKRVKLDSVR |
| | ACAACTACGCAGCGCCTCCCTCCACTCGGAAGGACTA | VLRQISNNRKCTSP |
| | TCCTGCTGCCAAGAGGGTCAAGTTGGACAGTGTCAGA | RSSDTEENVKRRT |
| | GTCCTGAGACAGATCAGCAACAACCGAAAATGCACCA | HNVLERQRRNELK |
| | GCCCCAGGTCCTCGGACACCGAGGAGAATGTCAAGAG | RSFFALRDQIPELE |
| | GCGAACACACAACGTCTTGGAGCGCCAGAGGAGGAAC | NNEKAPKVVILKKA |
| | GAGCTAAAACGGAGCTTTTTTGCCCTGCGTGACCAGAT | TAYILSVQAEEQKLI |
| | CCCGGAGTTGGAAAACAATGAAAAGGCCCCCAAGGTA | SEEDLLRKRREQL |
| | GTTATCCTTAAAAAAGCCACAGCATACATCCTGTCCGT | KHKLEQLRNSCA |
| | CCAAGCAGAGGAGCAAAAGCTCATTTCTGAAGAGGAC | (SEQ ID NO: 386) |
| | TTGTTGCGGAAACGACGAGAACAGTTGAAACACAAACT | |
| | TGAACAGCTACGGAACTCTTGTGCGTAA | |
| | (SEQ ID NO: 353) | |
| PRDM1 | ATGAAAATGGACATGGAGGATGCGGATATGACTCTGT | MKMDMEDADMTL |
| | GGACAGAGGCTGAGTTTGAAGAGAAGTGTACATACATT | WTEAEFEEKCTYIV |
| | GTGAACGACCACCCCTGGGATTCTGGTGCTGATGGCG | NDHPWDSGADGG |
| | GTACTTCGGTTCAGGCGGAGGCATCCTTACCAAGGAA | TSVQAEASLPRNLL |
| | TCTGCTTTTCAAGTATGCCACCAACAGTGAAGAGGTTA | FKYATNSEEVIGVM |
| | TTGGAGTGATGAGTAAAGAATACATACCAAAGGGCACA | SKEYIPKGTRFGPL |
| | CGTTTTGGACCCCTAATAGGTGAAATCTACACCAATGA | IGEIYTNDTVPKNA |
| | CACAGTTCCTAAGAACGCCAACAGGAAATATTTTTGGA | NRKYFWRIYSRGE |
| | GGATCTATTCCAGAGGGGAGCTTCACCACTTCATTGAC | LHHFIDGFNEEKSN |
| | GGCTTTAATGAAGAGAAAAGCAACTGGATGCGCTATGT | WMRYVNPAHSPR |
| | GAATCCAGCACACTCTCCCCGGGAGCAAAACCTGGCT | EQNLAACQNGMNI |
| | GCGTGTCAGAACGGGATGAACATCTACTTCTACACCAT | YFYTIKPIPANQELL |
| | TAAGCCCATCCCTGCCAACCAGGAACTTCTTGTGTGGT | VWYCRDFAERLHY |
| | ATTGTCGGGACTTTGCAGAAAGGCTTCACTACCCTTAT | PYPGELTMMNLTQ |
| | CCCGGAGAGCTGACAATGATGAATCTCACACAAACAC | TQSSLKQPSTEKN |
| | AGAGCAGTCTAAAGCAACCGAGCACTGAGAAAAATGA | ELCPKNVPKREYS |
| | ACTCTGCCCAAAGAATGTCCCAAAGAGAGAGTACAGC | VKEILKLDSNPSKG |
| | GTGAAAGAAATCCTAAAATTGGACTCCAACCCCTCCAA | KDLYRSNISPLTSE |
| | AGGAAAGGACCTCTACCGTTCTAACATTTCACCCCTCA | KDLDDFRRRGSPE |
| | CATCAGAAAAGGACCTCGATGACTTTAGAAGACGTGG | MPFYPRVVYPIRAP |
| | GAGCCCCGAAATGCCCTTCTACCCTCGGGTCGTTTAC | LPEDFLKASLAYGI |
| | CCCATCCGGGCCCCTCTGCCAGAAGACTTTTTGAAAG | ERPTYITRSPIPSST |
| | CTTCCCTGGCCTACGGGATCGAGAGACCCACGTACAT | TPSPSARSSPDQS |
| | CACTCGCTCCCCCATTCCATCCTCCACCACTCCAAGCC | LKSSSPHSSPGNT |
| | CCTCTGCAAGAAGCAGCCCCGACCAAAGCCTCAAGAG | VSPVGPGSQEHRD |
| | CTCCAGCCCTCACAGCAGCCCTGGGAATACGGTGTCC | SYAYLNASYGTEG |
| | CCTGTGGGCCCCGGCTCTCAAGAGCACCGGGACTCCT | LGSYPGYAPLPHL |
| | ACGCTTACTTGAACGCGTCCTACGGCACGGAAGGTTT | PPAFIPSYNAHYPK |
| | GGGCTCCTACCCTGGCTACGCACCCCTGCCCCACCTC | FLLPPYGMNCNGL |
| | CCGCCAGCTTTCATCCCCTCGTACAACGCTCACTACCC | SAVSSMNGINNFG |
| | CAAGTTCCTCTTGCCCCCCTACGGCATGAATTGTAATG | LFPRLCPVYSNLLG |
| | GCCTGAGCGCTGTGAGCAGCATGAATGGCATCAACAA | GGSLPHPMLNPTS |
| | CTTTGGCCTCTTCCCGAGGCTGTGCCCTGTCTACAGC | LPSSLPSDGARRLL |
| | AATCTCCTCGGTGGGGGCAGCCTGCCCCACCCCATGC | QPEHPREVLVPAP |
| | TCAACCCCACTTCTCTCCCGAGCTCGCTGCCCTCAGAT | HSAFSFTGAAASM |
| | GGAGCCCGGAGGTTGCTCCAGCCGGAGCATCCCAGG | KDKACSPTSGSPT |
| | GAGGTGCTTGTCCCGGCGCCCCACAGTGCCTTCTCCT | AGTAATAEHVVQP |
| | TTACCGGGGCCGCCGCCAGCATGAAGGACAAGGCCT | KATSAAMAAPSSD |
| | GTAGCCCCACAAGCGGGTCTCCCACGGCGGGAACAG | EAMNLIKNKRNMT |
| | CCGCCACGGCAGAACATGTGGTGCAGCCCAAAGCTAC | GYKTLPYPLKKQN |
| | CTCAGCAGCGATGGCAGCCCCCAGCAGCGACGAAGC | GKIKYECNVCAKTF |
| | CATGAATCTCATTAAAAACAAAAGAAACATGACCGGCT | GQLSNLKVHLRVH |
| | ACAAGACCCTTCCCTACCCGCTGAAGAAGCAGAACGG | SGERPFKCQTCNK |
| | CAAGATCAAGTACGAATGCAACGTTTGCGCCAAGACTT | GFTQLAHLQKHYL |
| | TCGGCCAGCTCTCCAATCTGAAGGTCCACCTGAGAGT | VHTGEKPHECQVC |
| | GCACAGTGGAGAACGGCCTTTCAAATGTCAGACTTGC | HKRFSSTSNLKTHL |
| | AACAAGGGCTTTACTCAGCTCGCCCACCTGCAGAAAC | RLHSGEKPYQCKV |
| | ACTACCTGGTACACACGGGAGAAAAGCCACATGAATG | CPAKFTQFVHLKL |
| | CCAGGTCTGCCACAAGAGATTTAGCAGCACCAGCAAT | HKRLHTRERPHKC |
| | CTCAAGACCCACCTGCGACTCCATTCTGGAGAGAAAC | SQCHKNYIHLCSLK |
| | CATACCAATGCAAGGTGTGCCCTGCCAAGTTCACCCA | VHLKGNCAAAPAP |
| | GTTTGTGCACCTGAAACTGCACAAGCGTCTGCACACC | GLPLEDLTRINEEIE |
| | CGGGAGCGGCCCCACAAGTGCTCCCAGTGCCACAAG | KFDISDNADRLEDV |
| | AACTACATCCATCTCTGTAGCCTCAAGGTTCACCTGAA | EDDISVISVVEKEIL |
| | AGGGAACTGCGCTGCGGCCCCGGCGCCTGGGCTGCC | AWVRKEKEETGLK |
| | CTTGGAAGATCTGACCCGAATCAATGAAGAAATCGAGA | VSLQRNMGNGLLS |
| | AGTTTGACATCAGTGACAATGCTGACCGGCTCGAGGA | SGCSLYESSDLPL |
| | CGTGGAGGATGACATCAGTGTGATCTCTGTAGTGGAG | MKLPPSNPLPLVP |
| | AAGGAAATTCTGGCCGTGGTCAGAAAAGAGAAAGAAG | VKVKQETVEPMDP |
| | AAACTGGCCTGAAAGTGTCTTTGCAAAGAAACATGGG | (SEQ ID NO: 387) |
| | GAATGGACTCCTCTCCTCAGGGTGCAGCCTTTATGAGT | |
| | CATCAGATCTACCCCTCATGAAGTTGCCTCCCAGCAAC | |
| | CCACTACCTCTGGTACCTGTAAAGGTCAAACAAGAAAC | |
| | AGTTGAACCAATGGATCCTTAA | |
| | (SEQ ID NO: 354) | |
| RBSN | ATGGCTTCTCTGGACGACCCAGGGGAAGTGAGGGAG | MASLDDPGEVREG |
| | GGCTTCCTCTGCCCTCTGTGCCTGAAGGATCTGCAGT | FLCPLCLKDLQSFY |
| | CTTTCTATCAGCTTCACTCACATTACGAGGAAGAACAC | QLHSHYEEEHSGE |
| | TCAGGGGAAGACCGTGATGTCAAAGGGCAAATTAAAA | DRDVKGQIKSLVQ |
| | GTCTTGTCCAGAAGGCTAAAAAAGCAAAGGACAGGTT | KAKKAKDRLLKRE |
| | GTTGAAACGAGAAGGGGATGATCGAGCAGAGTCAGGG | GDDRAESGTQGY |
| | ACCCAAGGATATGAGTCTTTCAGCTATGGAGGGGTTG | ESFSYGGVDPYM |
| | ATCCTTACATGTGGGAACCCCAGGAGCTTGGTGCTGT | WEPQELGAVRSHL |
| | GAGAAGCCATCTTTCCGACTTCAAAAAACACCGAGCTG | SDFKKHRAARIDH |
| | CTAGAATTGACCACTATGTTGTGGAAGTCAATAAACTA | YVVEVNKLIIRLEKL |
| | ATAATCAGGTTAGAGAAGCTCACTGCATTTGACAGAAC | TAFDRTNTESAKIR |
| | AAATACTGAGTCTGCAAAGATTCGAGCAATAGAAAAGT | AIEKSVVPWVNDQ |
| | CTGTGGTGCCTTGGGTCAACGACCAGGATGTCCCTTT | DVPFCPDCGNKFS |
| | CTGTCCAGACTGTGGGAATAAGTTCAGCATCCGGAAC | IRNRRHHCRLCGSI |
| | CGCCGCCACCACTGCCGCCTCTGCGGGTCTATTATGT | MCKKCMELISLPLA |
| | GCAAGAAGTGTATGGAGCTCATCAGCCTTCCCTTGGC | NKLTSASKESLSTH |
| | AAACAAGCTCACCAGTGCCAGCAAGGAGTCCCTGAGC | TSPSQSPNSVHGS |
| | ACCCACACCAGCCCCAGCCAGTCACCCAACAGTGTCC | RRGSISSMSSVSS |
| | ATGGCTCCCGCCGAGGCAGCATCAGCAGCATGAGCA | VLDEKDDDRIRCC |
| | GTGTCAGCTCGGTCCTGGATGAGAAGGACGATGACCG | THCKDTLLKREQQI |
| | GATCCGCTGCTGTACACACTGCAAGGACACGCTGCTC | DEKEHTPDIVKLYE |
| | AAGAGAGAGCAGCAGATTGATGAGAAGGAGCACACAC | KLRLCMEKVDQKA |
| | CTGACATCGTGAAGCTCTACGAGAAATTACGACTTTGC | PEYIRMAASLNAG |
| | ATGGAGAAAGTTGACCAGAAAGCTCCAGAATACATCAG | ETTYSLEHASDLRV |
| | GATGGCAGCATCATTAAATGCTGGGGAGACAACCTAC | EVQKVYELIDALSK |
| | AGTCTGGAACATGCCAGTGACCTTCGAGTGGAAGTGC | KILTLGLNQDPPPH |
| | AGAAAGTGTATGAGCTGATAGACGCTTTAAGTAAGAAG | PSNLRLQRMIRYS |
| | ATCTTAACCTTGGGCTTGAACCAGGACCCTCCACCACA | ATLFVQEKLLGLMS |
| | TCCAAGCAATTTGCGGCTGCAGAGAATGATCAGATACT | LPTKEQFEELKKKR |
| | CAGCTACACTTTTTGTGCAGGAAAAGTTGCTTGGTTTG | KEEMERKRAVERQ |
| | ATGTCACTGCCAACCAAAGAACAGTTTGAGGAACTGAA | AALESQRRLEERQ |
| | AAAGAAAAGGAAGGAGGAAATGGAGAGGAAGAGGGC | SGLASRAANGEVA |
| | CGTGGAGAGACAAGCTGCCCTGGAGTCCCAGCGAAG | SLRRGPAPLRKAE |
| | GCTTGAGGAAAGGCAGAGTGGCCTGGCTTCTCGAGCG | GWLPLSGGQGQS |
| | GCCAACGGGGAGGTGGCATCTCTCCGCAGGGGCCCT | EDSDPLLQQIHNIT |
| | GCCCCCTTGAGAAAGGCTGAGGGCTGGCTCCCACTGT | SFIRQAKAAGRMD |
| | CAGGAGGTCAGGGGCAGAGTGAGGACTCAGACCCGC | EVRTLQENLRQLQ |
| | TCCTCCAGCAGATCCACAACATCACATCATTCATCAGG | DEYDQQQTEKAIE |
| | CAGGCCAAGGCCGCGGGCCGCATGGATGAAGTGCGC | LSRRQAEEEDLQR |
| | ACTCTGCAGGAGAACCTGCGGCAGCTGCAGGACGAGT | EQLQMLRERELER |
| | ATGACCAGCAGCAGACAGAGAAGGCCATCGAGCTGTC | EREQFRVASLHTR |
| | CCGGAGGCAGGCTGAGGAGGAGGACCTGCAGCGGGA | TRSLDFREIGPFQL |
| | ACAGCTGCAGATGTTGCGTGAACGGGAGTTGGAACGA | EPSREPRTHLAYA |
| | GAAAGGGAGCAGTTTCGGGTGGCATCCCTGCACACAC | LDLGSSPVPSSTA |
| | GGACTCGGTCCCTGGACTTCAGAGAAATCGGCCCTTT | PKTPSLSSTQPTR |
| | TCAGCTGGAGCCCAGCAGAGAGCCTCGCACCCACCTT | VWSGPPAVGQER |
| | GCTTATGCTTTGGATCTAGGCTCTTCCCCAGTTCCAAG | LPQSSMPQQHEG |
| | CAGCACAGCTCCCAAGACCCCTTCACTTAGCTCAACTC | PSLNPFDEEDLSS |
| | AACCCACCAGAGTGTGGTCTGGGCCCCCAGCCGTTGG | PMEEATTGPPAAG |
| | CCAGGAGCGCTTACCCCAGAGCAGCATGCCACAGCAA | VSLDPSARILKEYN |
| | CATGAGGGGCCCTCCTTAAACCCCTTTGATGAGGAAG | PFEEEDEEEEAVA |
| | ACCTCTCCAGCCCCATGGAAGAGGCCACTACTGGTCC | GNPFIQPDSPAPN |
| | TCCTGCTGCAGGGGTTTCCTTAGACCCTTCAGCCCGC | PFSEEDEHPQQRL |
| | ATCCTGAAAGAGTACAATCCTTTCGAGGAAGAGGACG | SSPLVPGNPFEEP |
| | AGGAGGAGGAAGCAGTGGCAGGGAATCCATTCATTCA | TCINPFEMDSDSG |
| | GCCAGACAGCCCAGCTCCTAACCCCTTCAGTGAGGAA | PEAEEPIEEELLLQ |
| | GACGAACATCCCCAGCAGAGGCTCTCAAGCCCTCTGG | QIDNIKAYIFDAKQ |
| | TTCCTGGTAACCCCTTTGAGGAACCCACCTGTATCAAC | CGRLDEVEVLTEN |
| | CCCTTTGAGATGGACAGTGACAGTGGGCCAGAGGCTG | LRELKHTLAKQKG |
| | AGGAGCCCATAGAGGAAGAGCTCCTCCTGCAGCAGAT | GTD |
| | CGATAACATCAAGGCATACATCTTTGATGCCAAGCAGT | (SEQ ID NO: 388) |
| | GCGGCCGCCTGGATGAGGTAGAGGTGCTGACAGAGA | |
| | ATCTGCGGGAGCTGAAGCACACCCTGGCCAAGCAGAA | |
| | GGGGGGCACTGACTGA | |
| | (SEQ ID NO: 355) | |
| RUNX1 | ATGCGTATCCCCGTAGATGCCAGCACGAGCCGCCGCT | MRIPVDASTSRRFT |
| | TCACGCCGCCTTCCACCGCGCTGAGCCCAGGCAAGAT | PPSTALSPGKMSE |
| | GAGCGAGGCGTTGCCGCTGGGCGCCCCGGACGCCG | ALPLGAPDAGAAL |
| | GCGCTGCCCTGGCCGGCAAGCTGAGGAGCGGCGACC | AGKLRSGDRSMVE |
| | GCAGCATGGTGGAGGTGCTGGCCGACCACCCGGGCG | VLADHPGELVRTD |
| | AGCTGGTGCGCACCGACAGCCCCAACTTCCTCTGCTC | SPNFLCSVLPTHW |
| | CGTGCTGCCTACGCACTGGCGCTGCAACAAGACCCTG | RCNKTLPIAFKVVA |
| | CCCATCGCTTTCAAGGTGGTGGCCCTAGGGGATGTTC | LGDVPDGTLVTVM |
| | CAGATGGCACTCTGGTCACTGTGATGGCTGGCAATGA | AGNDENYSAELRN |
| | TGAAAACTACTCGGCTGAGCTGAGAAATGCTACCGCA | ATAAMKNQVARFN |
| | GCCATGAAGAACCAGGTTGCAAGATTTAATGACCTCAG | DLRFVGRSGRGKS |
| | GTTTGTCGGTCGAAGTGGAAGAGGGAAAAGCTTCACT | FTLTITVFTNPPQV |
| | CTGACCATCACTGTCTTCACAAACCCACCGCAAGTCGC | ATYHRAIKITVDGP |
| | CACCTACCACAGAGCCATCAAAATCACAGTGGATGGG | REPRRHRQKLDDQ |
| | CCCCGAGAACCTCGAAGACATCGGCAGAAACTAGATG | TKPGSLSFSERLSE |
| | ATCAGACCAAGCCCGGGAGCTTGTCCTTTTCCGAGCG | LEQLRRTAMRVSP |
| | GCTCAGTGAACTGGAGCAGCTGCGGCGCACAGCCAT | HHPAPTPNPRASL |
| | GAGGGTCAGCCCACACCACCCAGCCCCCACGCCCAA | NHSTAFNPQPQSQ |
| | CCCTCGTGCCTCCCTGAACCACTCCACTGCCTTTAACC | MQDTRQIQPSPPW |
| | CTCAGCCTCAGAGTCAGATGCAGGATACAAGGCAGAT | SYDQSYQYLGSIA |
| | CCAACCATCCCCACCGTGGTCCTACGATCAGTCCTAC | SPSVHPATPISPGR |
| | CAATACCTGGGATCCATTGCCTCTCCTTCTGTGCACCC | ASGMTTLSAELSS |
| | AGCAACGCCCATTTCACCTGGACGTGCCAGCGGCATG | RLSTAPDLTAFSDP |
| | ACAACCCTCTCTGCAGAACTTTCCAGTCGACTCTCAAC | RQFPALPSISDPR |
| | GGCACCCGACCTGACAGCGTTCAGCGACCCGCGCCA | MHYPGAFTYSPTP |
| | GTTCCCCGCGCTGCCCTCCATCTCCGACCCCCGCATG | VTSGIGIGMSAMG |
| | CACTATCCAGGCGCCTTCACCTACTCCCCGACGCCGG | SATRYHTYLPPPY |
| | TCACCTCGGGCATCGGCATCGGCATGTCGGCCATGGG | PGSSQAQGGPFQ |
| | CTCGGCCACGCGCTACCACACCTACCTGCCGCCGCCC | ASSPSYHLYYGAS |
| | TACCCCGGCTCGTCGCAAGCGCAGGGAGGCCCGTTC | AGSYQFSMVGGE |
| | CAAGCCAGCTCGCCCTCCTACCACCTGTACTACGGCG | RSPPRILPPCTNAS |
| | CCTCGGCCGGCTCCTACCAGTTCTCCATGGTGGGCGG | TGSALLNPSLPNQ |
| | CGAGCGCTCGCCGCCGCGCATCCTGCCGCCCTGCAC | SDVVEAEGSHSNS |
| | CAACGCCTCCACCGGCTCCGCGCTGCTCAACCCCAGC | PTNMAPSARLEEA |
| | CTCCCGAACCAGAGCGACGTGGTGGAGGCCGAGGGC | VWRPY |
| | AGCCACAGCAACTCCCCCACCAACATGGCGCCCTCCG | (SEQ ID NO: 389) |
| | CGCGCCTGGAGGAGGCCGTGTGGAGGCCCTACTGA | |
| | (SEQ ID NO: 356) | |
| RUNX3 | ATGGCATCGAACAGCATCTTCGACTCCTTCCCGACCTA | MASNSIFDSFPTYS |
| | CTCGCCGACCTTCATCCGCGACCCAAGCACCAGCCGC | PTFIRDPSTSRRFT |
| | CGCTTCACACCTCCCTCCCCGGCCTTCCCCTGCGGCG | PPSPAFPCGGGG |
| | GCGGCGGCGGCAAGATGGGCGAGAACAGCGGCGCG | GKMGENSGALSA |
| | CTGAGCGCGCAGGCGGCCGTGGGGCCCGGAGGGCG | QAAVGPGGRARP |
| | CGCCCGGCCCGAGGTGCGCTCGATGGTGGACGTGCT | EVRSMVDVLADHA |
| | GGCGGACCACGCAGGCGAGCTCGTGCGCACCGACAG | GELVRTDSPNFLC |
| | CCCCAACTTCCTCTGCTCCGTGCTGCCCTCGCACTGG | SVLPSHWRCNKTL |
| | CGCTGCAACAAGACGCTGCCCGTCGCCTTCAAGGTGG | PVAFKVVALGDVP |
| | TGGCATTGGGGGACGTGCCGGATGGTACGGTGGTGA | DGTVVTVMAGNDE |
| | CTGTGATGGCAGGCAATGACGAGAACTACTCCGCTGA | NYSAELRNASAVM |
| | GCTGCGCAATGCCTCGGCCGTCATGAAGAACCAGGTG | KNQVARFNDLRFV |
| | GCCAGGTTCAACGACCTTCGCTTCGTGGGCCGCAGTG | GRSGRGKSFTLTIT |
| | GGCGAGGGAAGAGTTTCACCCTGACCATCACTGTGTT | VFTNPTQVATYHR |
| | CACCAACCCCACCCAAGTGGCGACCTACCACCGAGCC | AIKVTVDGPREPR |
| | ATCAAGGTGACCGTGGACGGACCCCGGGAGCCCAGA | RHRQKLEDQTKPF |
| | CGGCACCGGCAGAAGCTGGAGGACCAGACCAAGCCG | PDRFGDLERLRMR |
| | TTCCCTGACCGCTTTGGGGACCTGGAACGGCTGCGCA | VTPSTPSPRGSLS |
| | TGCGGGTGACACCGAGCACACCCAGCCCCCGAGGCT | TTSHFSSQPQTPIQ |
| | CACTCAGCACCACAAGCCACTTCAGCAGCCAGCCCCA | GTSELNPFSDPRQ |
| | GACCCCAATCCAAGGCACCTCGGAACTGAACCCATTC | FDRSFPTLPTLTES |
| | TCCGACCCCCGCCAGTTTGACCGCTCCTTCCCCACGC | RFPDPRMHYPGA |
| | TGCCAACCCTCACGGAGAGCCGCTTCCCAGACCCCAG | MSAAFPYSATPSG |
| | GATGCATTATCCCGGGGCCATGTCAGCTGCCTTCCCC | TSISSLSVAGMPAT |
| | TACAGCGCCACGCCCTCGGGCACGAGCATCAGCAGC | SRFHHTYLPPPYP |
| | CTCAGCGTGGCGGGCATGCCGGCCACCAGCCGCTTC | GAPQNQSGPFQA |
| | CACCATACCTACCTCCCGCCACCCTACCCGGGGGCCC | NPSPYHLYYGTSS |
| | CGCAGAACCAGAGCGGGCCCTTCCAGGCCAACCCGT | GSYQFSMVAGSSS |
| | CCCCCTACCACCTCTACTACGGGACATCCTCTGGCTC | GGDRSPTRMLASC |
| | CTACCAGTTCTCCATGGTGGCCGGCAGCAGCAGTGGG | TSSAASVAAGNLM |
| | GGCGACCGCTCACCTACCCGCATGCTGGCCTCTTGCA | NPSLGGQSDGVEA |
| | CCAGCAGCGCTGCCTCTGTCGCCGCCGGCAACCTCAT | DGSHSNSPTALST |
| | GAACCCCAGCCTGGGCGGCCAGAGTGATGGCGTGGA | PGRMDEAVWRPY |
| | GGCCGACGGCAGCCACAGCAACTCACCCACGGCCCT | (SEQ ID NO: 390) |
| | GAGCACGCCAGGCCGCATGGATGAGGCCGTGTGGCG | |
| | GCCCTACTGA | |
| | (SEQ ID NO: 357) | |
| SON | ATGGCGACCAACATCGAGCAGATTTTTAGGTCTTTCGT | MATNIEQIFRSFVV |
| | GGTCAGTAAATTCCGGGAAATTCAACAGGAGCTTTCCA | SKFREIQQELSSG |
| | GTGGAAGGAATGAAGGCCAGCTGAATGGTGAAACAAA | RNEGQLNGETNTP |
| | TACACCCATTGAAGGAAACCAGGCGGGTGATGCAGCT | IEGNQAGDAAASA |
| | GCCTCTGCCAGGAGTCTACCAAATGAAGAAATAGTGC | RSLPNEEIVQKIEE |
| | AGAAGATAGAGGAAGTACTTTCTGGGGTCTTAGATACA | VLSGVLDTELRYKP |
| | GAACTACGATATAAGCCAGACTTGAAAGAGGGCTCCA | DLKEGSRKSRCVS |
| | GAAAAAGTAGATGCGTATCTGTACAAACAGATCCTACT | VQTDPTDEIPTKKS |
| | GATGAAATTCCCACTAAAAAGTCAAAGAAGCATAAAAA | KKHKKHKNKKKKK |
| | GCACAAAAACAAAAAGAAGAAAAAGAAGAAAGAAAAGG | KKEKEKKYKRQPE |
| | AAAAAAAATATAAAAGACAGCCAGAAGAATCTGAGTCA | ESESKTKSHDDGN |
| | AAGACGAAATCTCATGATGATGGGAACATAGATTTAGA | IDLESDSFLKFDSE |
| | ATCTGATTCCTTTTTAAAGTTTGATTCTGAACCTTCAGC | PSAVALELPTRAFG |
| | TGTGGCGCTGGAGCTTCCTACAAGAGCATTTGGCCCA | PSETNESPAVVLE |
| | TCTGAGACCAATGAATCCCCTGCAGTTGTGCTAGAACC | PPVVSMEVSEPHIL |
| | TCCTGTAGTATCAATGGAGGTATCAGAGCCACACATCT | ETLKPATKTAELSV |
| | TAGAAACTCTGAAGCCAGCTACAAAAACTGCAGAACTG | VSTSVISEQSEQSV |
| | TCAGTTGTATCTACATCAGTAATCTCAGAGCAGTCAGA | AVMPEPSMTKILDS |
| | GCAGTCTGTGGCAGTAATGCCAGAACCATCCATGACA | FAAAPVPTTTLVLK |
| | AAGATTCTGGATTCCTTTGCAGCAGCACCAGTGCCTAC | SSEPVVTMSVEYQ |
| | TACAACACTGGTGTTGAAGTCATCTGAGCCAGTTGTAA | MKSVLKSVESTSP |
| | CAATGTCAGTGGAGTATCAGATGAAGTCTGTGCTGAAA | EPSKIMLVEPPVAK |
| | TCTGTGGAGAGCACATCTCCAGAGCCATCAAAGATCAT | VLEPSETLVVSSET |
| | GTTGGTAGAGCCCCCAGTAGCAAAAGTGTTAGAGCCT | PTEVYPEPSTSTT |
| | TCAGAAACCCTTGTGGTATCATCAGAGACACCTACTGA | MDFPESSAIEALRL |
| | GGTGTACCCTGAGCCAAGCACATCAACAACAATGGATT | PEQPVDVPSEIADS |
| | TTCCAGAGTCATCTGCAATTGAAGCGCTAAGATTGCCA | SMTRPQELPELPK |
| | GAGCAGCCTGTAGACGTACCATCGGAGATTGCAGATT | TTALELQESSVASA |
| | CATCCATGACAAGACCGCAGGAGTTGCCGGAGCTGCC | MELPGPPATSMPE |
| | TAAGACCACAGCGTTGGAGCTGCAGGAGTCGTCGGTG | LQGPPVTPVLELP |
| | GCCTCAGCGATGGAGTTGCCGGGGCCACCTGCGACC | GPSATPVPELPGP |
| | TCCATGCCGGAGTTGCAGGGGCCCCCTGTGACTCCAG | LSTPVPELPGPPAT |
| | TGCTGGAGTTACCTGGGCCCTCTGCTACCCCGGTGCC | AVPELPGPSVTPV |
| | AGAGTTGCCAGGGCCCCTTTCTACCCCAGTGCCTGAG | PQLSQELPGLPAP |
| | TTGCCAGGGCCCCCTGCGACAGCAGTGCCTGAGTTGC | SMGLEPPQEVPEP |
| | CAGGGCCCTCTGTGACACCAGTGCCACAGTTGTCGCA | PVMAQELPGLPLV |
| | GGAATTGCCAGGGCTTCCAGCACCATCCATGGGGTTG | TAAVELPEQPAVT |
| | GAGCCACCACAGGAGGTACCAGAGCCACCTGTGATGG | VAMELTEQPVTTT |
| | CACAGGAGTTGCCAGGGCTGCCTTTGGTGACAGCAGC | ELEQPVGMTTVEH |
| | AGTAGAGTTGCCAGAGCAGCCTGCGGTAACAGTAGCA | PGHPEVTTATGLL |
| | ATGGAGTTGACCGAACAACCTGTGACGACGACAGAGT | GQPEATMVLELPG |
| | TGGAGCAGCCTGTGGGGATGACAACGGTGGAACATCC | QPVATTALELPGQ |
| | TGGGCATCCTGAGGTGACAACGGCAACAGGGTTGCTG | PSVTGVPELPGLP |
| | GGGCAGCCTGAGGCAACGATGGTGCTGGAGTTGCCA | SATRALELSGQPV |
| | GGACAGCCAGTGGCAACGACAGCGCTGGAGTTGCCG | ATGALELPGPLMA |
| | GGGCAGCCTTCGGTGACTGGGGTGCCAGAGTTGCCA | AGALEFSGQSGAA |
| | GGGCTGCCTTCGGCAACTAGGGCACTGGAGTTGTCGG | GALELLGQPLATG |
| | GGCAGCCTGTGGCAACTGGGGCACTAGAGTTGCCTG | VLELPGQPGAPEL |
| | GGCCGCTCATGGCAGCTGGGGCACTGGAGTTCTCGG | PGQPVATVALEISV |
| | GGCAGTCTGGGGCAGCTGGAGCACTGGAGCTTTTGG | QSVVTTSELSTMT |
| | GGCAGCCTCTGGCAACAGGGGTGCTGGAGTTGCCAG | VSQSLEVPSTTALE |
| | GGCAGCCTGGGGCGCCAGAGTTGCCTGGGCAGCCTG | SYNTVAQELPTTLV |
| | TGGCAACTGTGGCGCTGGAGATCTCTGTTCAGTCTGT | GETSVTVGVDPLM |
| | GGTGACAACATCGGAGCTGTCAACGATGACCGTGTCG | APESHILASNTMET |
| | CAGTCCCTGGAGGTGCCCTCGACGACAGCGCTGGAAT | HILASNTMDSQML |
| | CCTATAATACGGTAGCACAGGAGCTGCCTACTACATTA | ASNTMDSQMLASN |
| | GTGGGGGAGACTTCTGTAACAGTAGGAGTGGATCCCT | TMDSQMLASSTMD |
| | TGATGGCCCCAGAATCCCATATATTAGCTTCTAACACC | SQMLATSSMDSQ |
| | ATGGAGACCCATATATTAGCATCCAACACCATGGACTC | MLATSSMDSQMLA |
| | CCAAATGCTAGCGTCCAACACCATGGACTCCCAGATG | TSTMDSQMLATSS |
| | CTAGCATCCAACACCATGGACTCCCAGATGTTAGCGTC | MDSQMLATSSMD |
| | TAGCACCATGGACTCCCAGATGTTAGCAACTAGCTCCA | SQMLATSSMDSQ |
| | TGGACTCCCAGATGTTAGCAACTAGCTCCATGGACTCC | MLATSSMDSQMLA |
| | CAGATGTTAGCAACTAGCACTATGGACTCCCAGATGTT | TSTMDSQMLATST |
| | AGCAACCAGTTCCATGGACTCCCAGATGTTAGCAACCA | MDSQMLATSSMD |
| | GCTCCATGGACTCCCAGATGTTAGCAACCAGCTCCAT | SQMLASGTMDSQ |
| | GGACTCCCAGATGTTAGCAACCAGCTCCATGGACTCC | MLASGTMDAQMLA |
| | CAGATGTTAGCAACCAGCACCATGGATTCTCAGATGTT | SGTMDAQMLASST |
| | AGCAACCAGCACCATGGACTCCCAGATGTTAGCAACT | QDSAMLGSKSPDP |
| | AGCTCAATGGATTCCCAGATGTTAGCATCTGGCACTAT | YRLAQDPYRLAQD |
| | GGACTCTCAAATGTTAGCTTCTGGCACCATGGATGCTC | PYRLGHDPYRLGH |
| | AGATGTTAGCGTCTGGTACCATGGATGCCCAGATGTTA | DAYRLGQDPYRLG |
| | GCGTCTAGTACCCAAGATTCTGCTATGTTGGGTTCAAA | HDPYRLTPDPYRM |
| | ATCTCCTGATCCCTATAGGTTAGCTCAGGATCCTTACA | SPRPYRIAPRSYRI |
| | GGTTAGCTCAGGATCCCTATAGGTTGGGCCATGACCC | APRPYRLAPRPLM |
| | CTATAGATTAGGTCATGATGCTTACAGGTTAGGACAAG | LASRRSMMMSYAA |
| | ACCCTTATAGATTAGGCCATGATCCCTACAGACTAACT | ERSMMSSYERSM |
| | CCTGATCCCTATAGGATGTCACCTAGACCCTACAGGAT | MSYERSMMSPMA |
| | AGCACCCAGGTCCTATAGAATAGCACCCAGGCCATAT | ERSMMSAYERSM |
| | AGGTTAGCACCTAGACCCCTGATGTTAGCATCTAGACG | MSAYERSMMSPM |
| | TTCTATGATGATGTCCTATGCTGCAGAACGTTCCATGA | AERSMMSAYERS |
| | TGTCATCTTACGAACGCTCTATGATGTCTTATGAGCGG | MMSAYERSMMSP |
| | TCTATGATGTCCCCTATGGCTGAACGCTCTATGATGTC | MADRSMMSMGAD |
| | AGCCTACGAGCGCTCTATGATGTCAGCCTACGAGCGC | RSMMSSYSAADRS |
| | TCTATGATGTCCCCTATGGCTGAGCGCTCTATGATGTC | MMSSYSAADRSM |
| | AGCTTATGAACGCTCCATGATGTCAGCTTATGAACGCT | MSSYTADRSMMS |
| | CCATGATGTCCCCAATGGCTGATCGATCTATGATGTCC | MAADSYTDSYTDT |
| | ATGGGTGCTGACCGGTCTATGATGTCGTCATACTCTGC | YTEAYMVPPLPPE |
| | TGCTGACCGGTCTATGATGTCATCGTACTCTGCAGCTG | EPPTMPPLPPEEP |
| | ACCGATCTATGATGTCATCTTATACTGCTGATCGTTCAA | PMTPPLPPEEPPE |
| | TGATGTCTATGGCTGCTGATTCTTACACCGATTCTTAC | GPALPTEQSALTA |
| | ACTGACACATATACAGAGGCATATATGGTGCCACCTTT | ENTWPTEVPSSPS |
| | GCCTCCTGAAGAGCCCCCAACAATGCCACCGTTGCCA | EESVSQPEPPVSQ |
| | CCTGAGGAGCCACCAATGACACCACCATTGCCTCCTG | SEISEPSAVPTDYS |
| | AGGAACCACCAGAGGGTCCAGCATTGCCCACTGAGCA | VSASDPSVLVSEA |
| | GTCAGCATTAACAGCTGAAAATACTTGGCCTACAGAGG | AVTVPEPPPEPES |
| | TGCCATCATCACCATCTGAAGAGTCTGTATCGCAGCCT | SITLTPVESAVVAE |
| | GAGCCTCCTGTGAGTCAAAGTGAGATTTCGGAGCCTT | EHEVVPERPVTCM |
| | CAGCAGTGCCTACTGATTATTCAGTGTCAGCATCAGAT | VSETPAMSAEPTV |
| | CCCTCAGTTTTAGTATCAGAGGCTGCTGTGACTGTTCC | LASEPPVMSETAE |
| | AGAACCACCACCAGAGCCAGAATCTTCAATTACGTTAA | TFDSMRASGHVAS |
| | CACCTGTAGAGTCTGCAGTAGTAGCAGAAGAACATGA | EVSTSLLVPAVTTP |
| | AGTTGTTCCAGAGAGACCAGTGACTTGTATGGTATCTG | VLAESILEPPAMAA |
| | AAACTCCCGCCATGTCAGCTGAACCAACTGTGTTAGCA | PESSAMAVLESSA |
| | TCAGAGCCTCCTGTTATGTCAGAGACAGCAGAAACATT | VTVLESSTVTVLES |
| | TGATTCCATGAGAGCCTCAGGACATGTTGCCTCAGAA | STVTVLEPSVVTVP |
| | GTATCTACATCCTTGTTGGTTCCAGCAGTAACTACTCC | EPPVVAEPDYVTIP |
| | AGTGCTGGCAGAGAGCATTCTGGAGCCGCCAGCCATG | VPVVSALEPSVPVL |
| | GCTGCCCCAGAGTCTTCAGCTATGGCTGTCCTGGAGT | EPAVSVLQPSMIVS |
| | CTTCGGCTGTGACCGTCCTGGAGTCTTCGACTGTGAC | EPSVSVQESTVTV |
| | TGTCCTGGAGTCTTCGACTGTAACTGTCCTGGAGCCTT | SEPAVTVSEQTQVI |
| | CGGTTGTGACTGTCCCGGAGCCTCCTGTTGTGGCTGA | PTEVAIESTPMILES |
| | GCCAGACTATGTTACCATTCCTGTGCCAGTTGTTTCTG | SIMSSHVMKGINLS |
| | CGCTGGAGCCTTCTGTGCCTGTTCTGGAACCAGCGGT | SGDQNLAPEIGMQ |
| | GTCAGTCCTTCAACCTTCTATGATTGTTTCAGAACCATC | EIALHSGEEPHAEE |
| | TGTTTCTGTCCAGGAATCGACTGTGACAGTTTCAGAGC | HLKGDFYESEHGI |
| | CTGCTGTCACAGTCTCAGAGCAGACTCAAGTAATACCA | NIDLNINNHLIAKEM |
| | ACTGAGGTGGCTATAGAGTCCACACCAATGATACTGG | EHNTVCAAGTSPV |
| | AATCTAGTATCATGTCATCACATGTTATGAAAGGAATTA | GEIGEEKILPTSET |
| | ATCTATCCTCTGGTGATCAAAATCTTGCTCCAGAGATT | KQRTVLDTYPGVS |
| | GGCATGCAGGAGATTGCATTGCATTCAGGTGAAGAAC | EADAGETLSSTGP |
| | CACATGCTGAGGAACACCTGAAAGGTGACTTTTACGAA | FALEPDATGTSKGI |
| | AGTGAACATGGTATAAATATAGACCTTAATATAAATAAT | EFTTASTLSLVNKY |
| | CATTTAATTGCTAAAGAGATGGAACATAATACAGTGTG | DVDLSLTTQDTEH |
| | TGCTGCTGGTACTAGTCCTGTTGGGGAAATTGGTGAA | DMVISTSPSGGSE |
| | GAGAAAATTTTGCCCACCAGTGAGACTAAACAGCGCA | ADIEGPLPAKDIHL |
| | CAGTATTGGATACCTACCCTGGTGTTAGTGAAGCTGAT | DLPSNNNLVSKDT |
| | GCAGGAGAAACTCTATCTTCTACTGGTCCTTTTGCTCT | EEPLPVKESDQTL |
| | GGAACCTGATGCAACAGGAACTAGTAAGGGTATTGAAT | AALLSPKESSGGE |
| | TTACCACAGCATCTACTCTCAGTTTAGTTAATAAATATG | KEVPPPPKETLPD |
| | ATGTTGATTTATCTTTAACTACTCAAGATACTGAACATG | SGFSANIEDINEAD |
| | ACATGGTAATTTCCACCAGTCCTAGTGGTGGTAGTGAA | LVRPLLPKDMERLT |
| | GCTGACATTGAAGGGCCTTTGCCTGCTAAAGATATTCA | SLRAGIEGPLLASD |
| | TCTTGATTTACCATCTAATAATAACCTTGTTAGTAAGGA | VGRDRSAASPVVS |
| | TACAGAAGAACCATTACCTGTAAAAGAGAGTGACCAGA | SMPERASESSSEE |
| | CATTAGCAGCTCTGCTCAGCCCTAAAGAAAGTAGTGGA | KDDYEIFVKVKDTH |
| | GGAGAAAAAGAAGTACCTCCCCCTCCTAAAGAGACAC | EKSKKNKNRDKGE |
| | TGCCTGATTCAGGATTTTCTGCCAATATTGAGGATATTA | KEKKRDSSLRSRS |
| | ATGAAGCAGATTTAGTGAGACCGTTACTTCCTAAGGAC | KRSKSSEHKSRKR |
| | ATGGAACGTCTTACAAGCCTTAGAGCTGGCATTGAAG | TSESRSRARKRSS |
| | GACCTTTACTTGCAAGTGATGTTGGACGTGACAGATCT | KSKSHRSQTRSRS |
| | GCTGCCAGCCCGGTTGTAAGTAGTATGCCAGAAAGAG | RSRRRRRSSRSRS |
| | CTTCAGAGTCTTCTTCAGAGGAAAAAGATGATTATGAA | KSRGRRSVSKEKR |
| | ATTTTTGTAAAAGTTAAGGACACTCACGAAAAAAGCAA | KRSPKHRSKSRER |
| | GAAAAATAAGAACCGTGATAAGGGGGAGAAAGAGAAG | KRKRSSSRDNRKT |
| | AAAAGAGACTCTTCATTAAGATCTCGAAGTAAGCGTTC | VRARSRTPSRRSR |
| | CAAATCTTCTGAACACAAATCACGCAAGCGTACCAGTG | SHTPSRRRRSRSV |
| | AATCTCGTTCTAGGGCAAGAAAGAGATCATCTAAGTCC | GRRRSFSISPSRR |
| | AAGTCTCATCGCTCTCAGACACGTTCACGGTCACGTTC | SRTPSRRSRTPSR |
| | AAGACGCAGGAGGAGAAGCAGCAGATCAAGATCAAAG | RSRTPSRRSRTPS |
| | TCTAGAGGAAGAAGATCTGTATCAAAAGAGAAGCGCAA | RRSRTPSRRSRTP |
| | AAGATCTCCAAAGCACAGATCCAAGTCTAGGGAAAGAA | SRRRRSRSVVRRR |
| | AAAGAAAAAGATCAAGCTCCAGGGATAACCGAAAGAC | SFSISPVRLRRSRT |
| | AGTTAGAGCTCGAAGTCGAACCCCAAGTCGTCGGAGT | PLRRRFSRSPIRRK |
| | CGGAGTCATACTCCAAGTCGTCGACGAAGGTCTAGAT | RSRSSERGRSPKR |
| | CTGTGGGTAGAAGAAGGAGCTTTAGCATTTCCCCAAG | LTDLDKAQLLEIAK |
| | CCGCCGCAGCCGCACCCCCAGCCGCCGCAGCCGCAC | ANAAAMCAKAGVP |
| | CCCCAGCCGCCGCAGCCGCACCCCCAGCCGCCGCAG | LPPNLKPAPPPTIE |
| | CCGCACCCCCAGCCGCCGGAGCCGCACCCCTAGCCG | EKVAKKSGGATIEE |
| | TCGGAGCCGCACCCCAAGCCGCCGGAGAAGATCAAG | LTEKCKQIAQSKED |
| | GTCTGTGGTAAGAAGACGAAGCTTCAGTATCTCACCAG | DDVIVNKPHVSDE |
| | TCAGATTAAGGCGATCAAGAACACCCTTAAGAAGAAGG | EEEEPPFYHHPFK |
| | TTTAGCAGATCTCCCATCCGTCGTAAAAGATCCAGGTC | LSEPKPIFFNLNIAA |
| | TTCTGAACGAGGCAGATCACCCAAACGTCTGACAGATT | AKPTPPKSQVTLTK |
| | TGGATAAGGCTCAATTACTTGAAATAGCCAAAGCTAAT | EFPVSSGSQHRKK |
| | GCAGCTGCCATGTGTGCTAAGGCTGGTGTCCCTTTAC | EADSVYGEWVPVE |
| | CACCAAACCTAAAGCCTGCACCTCCACCTACTATAGAA | KNGEENKDDDNVF |
| | GAGAAAGTTGCTAAAAAGTCAGGAGGAGCTACTATAGA | SSNLPSEPVDISTA |
| | AGAACTAACTGAGAAATGTAAACAGATCGCACAGAGTA | MSERALAQKRLSE |
| | AAGAAGATGATGATGTAATAGTGAATAAACCTCATGTTT | NAFDLEAMSMLNR |
| | CGGATGAAGAGGAAGAAGAACCTCCTTTTTATCATCAT | AQERIDAWAQLNSI |
| | CCCTTTAAACTCAGTGAACCCAAACCTATTTTTTTCAAT | PGQFTGSTGVQVL |
| | CTGAATATTGCTGCAGCAAAACCAACTCCACCAAAAAG | TQEQLANTGAQA |
| | CCAGGTAACATTAACAAAAGAATTCCCTGTATCATCTG | WIKKDQFLRAAPV |
| | GATCTCAACATCGGAAAAAAGAAGCGGATAGTGTTTAT | TGGMGAVLMRKM |
| | GGAGAATGGGTTCCTGTGGAGAAAAATGGTGAAGAAA | GWREGEGLGKNK |
| | ACAAAGATGATGATAATGTTTTCAGCAGCAATTTGCCC | EGNKEPILVDFKTD |
| | TCAGAGCCTGTGGACATCTCTACAGCAATGAGTGAAC | RKGLVAVGERAQK |
| | GGGCACTTGCTCAGAAAAGACTCAGTGAGAATGCATTT | RSGNFSAAMKDLS |
| | GATCTTGAAGCCATGAGCATGTTAAATAGAGCTCAGGA | GKHPVSALMEICN |
| | AAGGATTGATGCCTGGGCTCAGCTGAACTCTATTCCTG | KRRWQPPEFLLVH |
| | GCCAGTTCACAGGAAGTACAGGAGTACAGGTTTTGAC | DSGPDHRKHFLFR |
| | ACAAGAACAGTTGGCCAATACTGGTGCCCAAGCCTGG | VLRNGALTRPNCM |
| | ATTAAAAAGGATCAGTTCTTAAGAGCAGCCCCGGTAAC | FFLNRY |
| | TGGAGAGAAGGAGAAGGATTAGGAAAAAACAAAGAAG | (SEQ ID NO: 391) |
| | TGGAGGAATGGGAGCCGTTTTGATGAGAAAAATGGGC | |
| | GCAATAAGGAACCCATCCTAGTTGATTTTAAGACAGAC | |
| | CGAAAAGGTCTTGTTGCAGTAGGAGAAAGAGCACAAA | |
| | AGAGGTCTGGGAACTTCTCTGCTGCAATGAAAGATCTG | |
| | TCAGGCAAACATCCTGTGTCTGCTTTGATGGAGATCTG | |
| | TAATAAAAGAAGGTGGCAACCACCTGAATTTCTATTGG | |
| | TCCATGATAGTGGCCCTGATCATCGCAAACATTTTCTC | |
| | TTTAGGGTATTGAGAAATGGAGCCCTTACCAGACCCAA | |
| | TTGTATGTTTTTCTTGAATAGGTATTGA | |
| | (SEQ ID NO: 358) | |
| TBX21 | ATGGGCATCGTGGAGCCGGGTTGCGGAGACATGCTG | MGIVEPGCGDMLT |
| | ACGGGCACCGAGCCGATGCCGGGGAGCGACGAGGG | GTEPMPGSDEGR |
| | CCGGGCGCCTGGCGCCGACCCGCAGCACCGCTACTT | APGADPQHRYFYP |
| | CTACCCGGAGCCGGGCGCGCAGGACGCGGACGAGC | EPGAQDADERRG |
| | GTCGCGGGGGCGGCAGCCTGGGGTCTCCCTACCCGG | GGSLGSPYPGGAL |
| | GGGGCGCCTTGGTGCCCGCCCCGCCGAGCCGCTTCC | VPAPPSRFLGAYA |
| | TTGGAGCCTACGCCTACCCGCCGCGACCCCAGGCGG | YPPRPQAAGFPGA |
| | CCGGCTTCCCCGGCGCGGGCGAGTCCTTCCCGCCGC | GESFPPPADAEGY |
| | CCGCGGACGCCGAGGGCTACCAGCCGGGCGAGGGCT | QPGEGYAAPDPRA |
| | ACGCCGCCCCGGACCCGCGCGCCGGGCTCTACCCGG | GLYPGPREDYALP |
| | GGCCGCGTGAGGACTACGCGCTACCCGCGGGACTGG | AGLEVSGKLRVAL |
| | AGGTGTCGGGGAAACTGAGGGTCGCGCTCAACAACCA | NNHLLWSKFNQHQ |
| | CCTGTTGTGGTCCAAGTTTAATCAGCACCAGACAGAGA | TEMIITKQGRRMFP |
| | TGATCATCACCAAGCAGGGACGGCGGATGTTCCCATT | FLSFTVAGLEPTSH |
| | CCTGTCATTTACTGTGGCCGGGCTGGAGCCCACCAGC | YRMFVDVVLVDQH |
| | CACTACAGGATGTTTGTGGACGTGGTCTTGGTGGACC | HWRYQSGKWVQC |
| | AGCACCACTGGCGGTACCAGAGCGGCAAGTGGGTGC | GKAEGSMPGNRLY |
| | AGTGTGGAAAGGCCGAGGGCAGCATGCCAGGAAACC | VHPDSPNTGAHW |
| | GCCTGTACGTCCACCCGGACTCCCCCAACACAGGAGC | MRQEVSFGKLKLT |
| | GCACTGGATGCGCCAGGAAGTTTCATTTGGGAAACTA | NNKGASNNVTQMI |
| | AAGCTCACAAACAACAAGGGGGCGTCCAACAATGTGA | VLQSLHKYQPRLHI |
| | CCCAGATGATTGTGCTCCAGTCCCTCCATAAGTACCAG | VEVNDGEPEAACN |
| | CCCCGGCTGCATATCGTTGAGGTGAACGACGGAGAGC | ASNTHIFTFQETQFI |
| | CAGAGGCAGCCTGCAACGCTTCCAACACGCATATCTTT | AVTAYQNAEITQLK |
| | ACTTTCCAAGAAACCCAGTTCATTGCCGTGACTGCCTA | IDNNPFAKGFRENF |
| | CCAGAATGCCGAGATTACTCAGCTGAAAATTGATAATA | ESMYTSVDTSIPSP |
| | ACCCCTTTGCCAAAGGATTCCGGGAGAACTTTGAGTC | PGPNCQFLGGDHY |
| | CATGTACACATCTGTTGACACCAGCATCCCCTCCCCGC | SPLLPNQYPVPSR |
| | CTGGACCCAACTGTCAATTCCTTGGGGGAGATCACTA | FYPDLPGQAKDVV |
| | CTCTCCTCTCCTACCCAACCAGTATCCTGTTCCCAGCC | PQAYWLGAPRDH |
| | GCTTCTACCCCGACCTTCCTGGCCAGGCGAAGGATGT | SYEAEFRAVSMKP |
| | GGTTCCCCAGGCTTACTGGCTGGGGGCCCCCCGGGA | AFLPSAPGPTMSY |
| | CCACAGCTATGAGGCTGAGTTTCGAGCAGTCAGCATG | YRGQEVLAPGAG |
| | AAGCCTGCATTCTTGCCCTCTGCCCCTGGGCCCACCA | WPVAPQYPPKMG |
| | TGTCCTACTACCGAGGCCAGGAGGTCCTGGCACCTGG | PASWFRPMRTLPM |
| | AGCTGGCTGGCCTGTGGCACCCCAGTACCCTCCCAAG | EPGPGGSEGRGP |
| | ATGGGCCCGGCCAGCTGGTTCCGCCCTATGCGGACTC | EDQGPPLVWTEIA |
| | TGCCCATGGAACCCGGCCCTGGAGGCTCAGAGGGAC | PIRPESSDSGLGE |
| | GGGGACCAGAGGACCAGGGTCCCCCCTTGGTGTGGA | GDSKRRRVSPYPS |
| | CTGAGATTGCCCCCATCCGGCCGGAATCCAGTGATTC | SGDSSSPAGAPSP |
| | AGGACTGGGCGAAGGAGACTCTAAGAGGAGGCGCGT | FDKEAEGQFYNYF |
| | GTCCCCCTATCCTTCCAGTGGTGACAGCTCCTCCCCT | PN |
| | GCTGGGGCCCCTTCTCCTTTTGATAAGGAAGCTGAAG | (SEQ ID NO: 392) |
| | GACAGTTTTATAACTATTTTCCCAACTGA | |
| | (SEQ ID NO: 359) | |
| TET2 | ATGATCCAGGAAGAGCAAATTCAACTAGAGGGCAGCC | MIQEEQIQLEGSLV |
| | TTGTGGATGGCCCCGAAGCAAGCCTGATGGAACAGGA | DGPEASLMEQDRT |
| | TAGAACCAACCATGTTGAGGGCAACAGACTAAGTCCAT | NHVEGNRLSPFLIP |
| | TCCTGATACCATCACCTCCCATTTGCCAGACAGAACCT | SPPICQTEPLATKL |
| | CTGGCTACAAAGCTCCAGAATGGAAGCCCACTGCCTG | QNGSPLPERAHPE |
| | AGAGAGCTCATCCAGAAGTAAATGGAGACACCAAGTG | VNGDTKWHSFKSY |
| | GCACTCTTTCAAAAGTTATTATGGAATACCCTGTATGAA | YGIPCMKGSQNSR |
| | GGGAAGCCAGAATAGTCGTGTGAGTCCTGACTTTACA | VSPDFTQESRGYS |
| | CAAGAAAGTAGAGGGTATTCCAAGTGTTTGCAAAATGG | KCLQNGGIKRTVS |
| | AGGAATAAAACGCACAGTTAGTGAACCTTCTCTCTCTG | EPSLSGLLQIKKLK |
| | GGCTCCTTCAGATCAAGAAATTGAAACAAGACCAAAAG | QDQKANGERRNF |
| | GCTAATGGAGAAAGACGTAACTTCGGGGTAAGCCAAG | GVSQERNPGESS |
| | AAAGAAATCCAGGTGAAAGCAGTCAACCAAATGTCTCC | QPNVSDLSDKKES |
| | GATTTGAGTGATAAGAAAGAATCTGTGAGTTCTGTAGC | VSSVAQENAVKDF |
| | CCAAGAAAATGCAGTTAAAGATTTCACCAGTTTTTCAAC | TSFSTHNCSGPEN |
| | ACATAACTGCAGTGGGCCTGAAAATCCAGAGCTTCAG | PELQILNEQEGKSA |
| | ATTCTGAATGAGCAGGAGGGGAAAAGTGCTAATTACCA | NYHDKNIVLLKNKA |
| | TGACAAGAACATTGTATTACTTAAAAACAAGGCAGTGC | VLMPNGATVSASS |
| | TAATGCCTAATGGTGCTACAGTTTCTGCCTCTTCCGTG | VEHTHGELLEKTLS |
| | GAACACACACATGGTGAACTCCTGGAAAAAACACTGTC | QYYPDCVSIAVQK |
| | TCAATATTATCCAGATTGTGTTTCCATTGCGGTGCAGA | TTSHINAINSQATN |
| | AAACCACATCTCACATAAATGCCATTAACAGTCAGGCT | ELSCEITHPSHTSG |
| | ACTAATGAGTTGTCCTGTGAGATCACTCACCCATCGCA | QINSAQTSNSELPP |
| | TACCTCAGGGCAGATCAATTCCGCACAGACCTCTAACT | KPAAVVSEACDAD |
| | CTGAGCTGCCTCCAAAGCCAGCTGCAGTGGTGAGTGA | DADNASKLAAMLN |
| | GGCCTGTGATGCTGATGATGCTGATAATGCCAGTAAAC | TCSFQKPEQLQQQ |
| | TAGCTGCAATGCTAAATACCTGTTCCTTTCAGAAACCA | KSVFEICPSPAENN |
| | GAACAACTACAACAACAAAAATCAGTTTTTGAGATATG | IQGTTKLASGEEFC |
| | CCCATCTCCTGCAGAAAATAACATCCAGGGAACCACAA | SGSSSNLQAPGGS |
| | AGCTAGCGTCTGGTGAAGAATTCTGTTCAGGTTCCAGC | SERYLKQNEMNGA |
| | AGCAATTTGCAAGCTCCTGGTGGCAGCTCTGAACGGT | YFKQSSVFTKDSF |
| | ATTTAAAACAAAATGAAATGAATGGTGCTTACTTCAAGC | SATTTPPPPSQLLL |
| | AAAGCTCAGTGTTCACTAAGGATTCCTTTTCTGCCACT | SPPPPLPQVPQLP |
| | ACCACACCACCACCACCATCACAATTGCTTCTTTCTCC | SEGKSTLNGGVLE |
| | CCCTCCTCCTCTTCCACAGGTTCCTCAGCTTCCTTCAG | EHHHYPNQSNTTL |
| | AAGGAAAAAGCACTCTGAATGGTGGAGTTTTAGAAGAA | LREVKIEGKPEAPP |
| | CACCACCACTACCCCAACCAAAGTAACACAACACTTTT | SQSPNPSTHVCSP |
| | AAGGGAAGTGAAAATAGAGGGTAAACCTGAGGCACCA | SPMLSERPQNNCV |
| | CCTTCCCAGAGTCCTAATCCATCTACACATGTATGCAG | NRNDIQTAGTMTV |
| | CCCTTCTCCGATGCTTTCTGAAAGGCCTCAGAATAATT | PLCSEKTRPMSEH |
| | GTGTGAACAGGAATGACATACAGACTGCAGGGACAAT | LKHNPPIFGSSGEL |
| | GACTGTTCCATTGTGTTCTGAGAAAACAAGACCAATGT | QDNCQQLMRNKE |
| | CAGAACACCTCAAGCATAACCCACCAATTTTTGGTAGC | QEILKGRDKEQTR |
| | AGTGGAGAGCTACAGGACAACTGCCAGCAGTTGATGA | DLVPPTQHYLKPG |
| | GAAACAAAGAGCAAGAGATTCTGAAGGGTCGAGACAA | WIELKAPRFHQAE |
| | GGAGCAAACACGAGATCTTGTGCCCCCAACACAGCAC | SHLKRNEASLPSIL |
| | TATCTGAAACCAGGATGGATTGAATTGAAGGCCCCTCG | QYQPNLSNQMTSK |
| | TTTTCACCAAGCGGAATCCCATCTAAAACGTAATGAGG | QYTGNSNMPGGL |
| | CATCACTGCCATCAATTCTTCAGTATCAACCCAATCTCT | PRQAYTQKTTQLE |
| | CCAATCAAATGACCTCCAAACAATACACTGGAAATTCC | HKSQMYQVEMNQ |
| | AACATGCCTGGGGGGCTCCCAAGGCAAGCTTACACCC | GQSQGTVDQHLQ |
| | AGAAAACAACACAGCTGGAGCACAAGTCACAAATGTAC | FQKPSHQVHFSKT |
| | CAAGTTGAAATGAATCAAGGGCAGTCCCAAGGTACAG | DHLPKAHVQSLCG |
| | TGGACCAACATCTCCAGTTCCAAAAACCCTCACACCAG | TRFHFQQRADSQT |
| | GTGCACTTCTCCAAAACAGACCATTTACCAAAAGCTCA | EKLMSPVLKQHLN |
| | TGTGCAGTCACTGTGTGGCACTAGATTTCATTTTCAAC | QQASETEPFSNSH |
| | AAAGAGCAGATTCCCAAACTGAAAAACTTATGTCCCCA | LLQHKPHKQAAQT |
| | GTGTTGAAACAGCACTTGAATCAACAGGCTTCAGAGAC | QPSQSSHLPQNQ |
| | TGAGCCATTTTCAAACTCACACCTTTTGCAACATAAGC | QQQQKLQIKNKEEI |
| | CTCATAAACAGGCAGCACAAACACAACCATCCCAGAGT | LQTFPHPQSNNDQ |
| | TCACATCTCCCTCAAAACCAGCAACAGCAGCAAAAATT | QREGSFFGQTKVE |
| | ACAAATAAAGAATAAAGAGGAAATACTCCAGACTTTTC | ECFHGENQYSKSS |
| | CTCACCCCCAAAGCAACAATGATCAGCAAAGAGAAGG | EFETHNVQMGLEE |
| | ATCATTCTTTGGCCAGACTAAAGTGGAAGAATGTTTTC | VQNINRRNSPYSQ |
| | ATGGTGAAAATCAGTATTCAAAATCAAGCGAGTTCGAG | TMKSSACKIQVSC |
| | ACTCATAATGTCCAAATGGGACTGGAGGAAGTACAGAA | SNNTHLVSENKEQ |
| | TATAAATCGTAGAAATTCCCCTTATAGTCAGACCATGAA | TTHPELFAGNKTQ |
| | ATCAAGTGCATGCAAAATACAGGTTTCTTGTTCAAACA | NLHHMQYFPNNVI |
| | ATACACACCTAGTTTCAGAGAATAAAGAACAGACTACA | PKQDLLHRCFQEQ |
| | CATCCTGAACTTTTTGCAGGAAACAAGACCCAAAACTT | EQKSQQASVLQGY |
| | GCATCACATGCAATATTTTCCAAATAATGTGATCCCAAA | KNRNQDMSGQQA |
| | GCAAGATCTTCTTCACAGGTGCTTTCAAGAACAGGAGC | AQLAQQRYLIHNH |
| | AGAAGTCACAACAAGCTTCAGTTCTACAGGGATATAAA | ANVFPVPDQGGSH |
| | AATAGAAACCAAGATATGTCTGGTCAACAAGCTGCGCA | TQTPPQKDTQKHA |
| | ACTTGCTCAGCAAAGGTACTTGATACATAACCATGCAA | ALRWHLLQKQEQQ |
| | ATGTTTTTCCTGTGCCTGACCAGGGAGGAAGTCACACT | QTQQPQTESCHS |
| | CAGACCCCTCCCCAGAAGGACACTCAAAAGCATGCTG | QMHRPIKVEPGCK |
| | CTCTAAGGTGGCATCTCTTACAGAAGCAAGAACAGCA | PHACMHTAPPENK |
| | GCAAACACAGCAACCCCAAACTGAGTCTTGCCATAGTC | TWKKVTKQENPPA |
| | AGATGCACAGGCCAATTAAGGTGGAACCTGGATGCAA | SCDNVQQKSIIETM |
| | GCCACATGCCTGTATGCACACAGCACCACCAGAAAAC | EQHLKQFHAKSLF |
| | AAAACATGGAAAAAGGTAACTAAGCAAGAGAATCCACC | DHKALTLKSQKQV |
| | TGCAAGCTGTGATAATGTGCAGCAAAAGAGCATCATTG | KVEMSGPVTVLTR |
| | AGACCATGGAGCAGCATCTGAAGCAGTTTCACGCCAA | QTTAAELDSHTPAL |
| | GTCGTTATTTGACCATAAGGCTCTTACTCTCAAATCACA | EQQTTSSEKTPTK |
| | GAAGCAAGTAAAAGTTGAAATGTCAGGGCCAGTCACA | RTAASVLNNFIESP |
| | GTTTTGACTAGACAAACCACTGCTGCAGAACTTGATAG | SKLLDTPIKNLLDT |
| | CCACACCCCAGCTTTAGAGCAGCAAACAACTTCTTCAG | PVKTQYDFPSCRC |
| | AAAAGACACCAACCAAAAGAACAGCTGCTTCTGTTCTC | VEQIIEKDEGPFYT |
| | AATAATTTTATAGAGTCACCTTCCAAATTACTAGATACT | HLGAGPNVAAIREI |
| | CCTATAAAAAATTTATTGGATACACCTGTCAAGACTCAA | MEERFGQKGKAIRI |
| | TATGATTTCCCATCTTGCAGATGTGTAGAGCAAATTATT | ERVIYTGKEGKSS |
| | GAAAAAGATGAAGGTCCTTTTTATACCCATCTAGGAGC | QGCPIAKWWVRRS |
| | AGGTCCTAATGTGGCAGCTATTAGAGAAATCATGGAAG | SSEEKLLCLVRERA |
| | AAAGGTTTGGACAGAAGGGTAAAGCTATTAGGATTGAA | GHTCEAAVIVILILV |
| | AGAGTCATCTATACTGGTAAAGAAGGCAAAAGTTCTCA | WEGIPLSLADKLYS |
| | GGGATGTCCTATTGCTAAGTGGGTGGTTCGCAGAAGC | ELTETLRKYGTLTN |
| | AGCAGTGAAGAGAAGCTACTGTGTTTGGTGCGGGAGC | RRCALNEERTCAC |
| | GAGCTGGCCACACCTGTGAGGCTGCAGTGATTGTGAT | QGLDPETCGASFS |
| | TCTCATCCTGGTGTGGGAAGGAATCCCGCTGTCTCTG | FGCSWSMYYNGC |
| | GCTGACAAACTCTACTCGGAGCTTACCGAGACGCTGA | KFARSKIPRKFKLL |
| | GGAAATACGGCACGCTCACCAATCGCCGGTGTGCCTT | GDDPKEEEKLESH |
| | GAATGAAGAGAGAACTTGCGCCTGTCAGGGGCTGGAT | LQNLSTLMAPTYK |
| | CCAGAAACCTGTGGTGCCTCCTTCTCTTTTGGTTGTTC | KLAPDAYNNQIEYE |
| | ATGGAGCATGTACTACAATGGATGTAAGTTTGCCAGAA | HRAPECRLGLKEG |
| | GCAAGATCCCAAGGAAGTTTAAGCTGCTTGGGGATGA | RPFSGVTACLDFC |
| | CCCAAAAGAGGAAGAGAAACTGGAGTCTCATTTGCAAA | AHAHRDLHNMQN |
| | ACCTGTCCACTCTTATGGCACCAACATATAAGAAACTT | GSTLVCTLTREDN |
| | GCACCTGATGCATATAATAATCAGATTGAATATGAACA | REFGGKPEDEQLH |
| | CAGAGCACCAGAGTGCCGTCTGGGTCTGAAGGAAGG | VLPLYKVSDVDEF |
| | CCGTCCATTCTCAGGGGTCACTGCATGTTTGGACTTCT | GSVEAQEEKKRSG |
| | GTGCTCATGCCCACAGAGACTTGCACAACATGCAGAA | AIQVLSSFRRKVR |
| | TGGCAGCACATTGGTATGCACTCTCACTAGAGAAGACA | MLAEPVKTCRQRK |
| | ATCGAGAATTTGGAGGAAAACCTGAGGATGAGCAGCT | LEAKKAAAEKLSSL |
| | TCACGTTCTGCCTTTATACAAAGTCTCTGACGTGGATG | ENSSNKNEKEKSA |
| | AGTTTGGGAGTGTGGAAGCTCAGGAGGAGAAAAAACG | PSRTKQTENASQA |
| | GAGTGGTGCCATTCAGGTACTGAGTTCTTTTCGGCGAA | KQLAELLRLSGPV |
| | AAGTCAGGATGTTAGCAGAGCCAGTCAAGACTTGCCG | MQQSQQPQPLQK |
| | ACAAAGGAAACTAGAAGCCAAGAAAGCTGCAGCTGAA | QPPQPQQQQRPQ |
| | AAGCTTTCCTCCCTGGAGAACAGCTCAAATAAAAATGA | QQQPHHPQTESV |
| | AAAGGAAAAGTCAGCCCCATCACGTACAAAACAAACTG | NSYSASGSTNPYM |
| | AAAACGCAAGCCAGGCTAAACAGTTGGCAGAACTTTTG | RRPNPVSPYPNSS |
| | CGACTTTCAGGACCAGTCATGCAGCAGTCCCAGCAGC | HTSDIYGSTSPMNF |
| | CCCAGCCTCTACAGAAGCAGCCACCACAGCCCCAGCA | YSTSSQAAGSYLN |
| | GCAGCAGAGACCCCAGCAGCAGCAGCCACATCACCCT | SSNPMNPYPGLLN |
| | CAGACAGAGTCTGTCAACTCTTATTCTGCTTCTGGATC | QNTQYPSYQCNG |
| | CACCAATCCATACATGAGACGGCCCAATCCAGTTAGTC | NLSVDNCSPYLGS |
| | CTTATCCAAACTCTTCACACACTTCAGATATCTATGGAA | YSPQSQPMDLYRY |
| | GCACCAGCCCTATGAACTTCTATTCCACCTCATCTCAA | PSQDPLSKLSLPPI |
| | GCTGCAGGTTCATATTTGAATTCTTCTAATCCCATGAAC | HTLYQPRFGNSQS |
| | CCTTACCCTGGGCTTTTGAATCAGAATACCCAATATCC | FTSKYLGYGNQNM |
| | ATCATATCAATGCAATGGAAACCTATCAGTGGACAACT | QGDGFSSCTIRPN |
| | GCTCCCCATATCTGGGTTCCTATTCTCCCCAGTCTCAG | VHHVGKLPPYPTH |
| | CCGATGGATCTGTATAGGTATCCAAGCCAAGACCCTCT | EMDGHFMGATSRL |
| | GTCTAAGCTCAGTCTACCACCCATCCATACACTTTACC | PPNLSNPNMDYKN |
| | AGCCAAGGTTTGGAAATAGCCAGAGTTTTACATCTAAA | GEHHSPSHIIHNYS |
| | TACTTAGGTTATGGAAACCAAAATATGCAGGGAGATGG | AAPGMFNSSLHAL |
| | TTTCAGCAGTTGTACCATTAGACCAAATGTACATCATGT | HLQNKENDMLSHT |
| | AGGGAAATTGCCTCCTTATCCCACTCATGAGATGGATG | ANGLSKMLPALNH |
| | GCCACTTCATGGGAGCCACCTCTAGATTACCACCCAAT | DRTACVQGGLHKL |
| | CTGAGCAATCCAAACATGGACTATAAAAATGGTGAACA | SDANGQEKQPLAL |
| | TCATTCACCTTCTCACATAATCCATAACTACAGTGCAG | VQGVASGAEDNDE |
| | CTCCGGGCATGTTCAACAGCTCTCTTCATGCCCTGCAT | VWSDSEQSFLDPD |
| | CTCCAAAACAAGGAGAATGACATGCTTTCCCACACAGC | IGGVAVAPTHGSILI |
| | TAATGGGTTATCAAAGATGCTTCCAGCTCTTAACCATG | ECAKRELHATTPLK |
| | ATAGAACTGCTTGTGTCCAAGGAGGCTTACACAAATTA | NPNRNHPTRISLVF |
| | AGTGATGCTAATGGTCAGGAAAAGCAGCCATTGGCAC | YQHKSMNEPKHGL |
| | TAGTCCAGGGTGTGGCTTCTGGTGCAGAGGACAACGA | ALWEAKMAEKARE |
| | TGAGGTCTGGTCAGACAGCGAGCAGAGCTTTCTGGAT | KEEECEKYGPDYV |
| | CCTGACATTGGGGGAGTGGCCGTGGCTCCAACTCATG | PQKSHGKKVKREP |
| | GGTCAATTCTCATTGAGTGTGCAAAGCGTGAGCTGCAT | AEPHETSEPTYLRF |
| | GCCACAACCCCTTTAAAGAATCCCAATAGGAATCACCC | IKSLAERTMSVTTD |
| | CACCAGGATCTCCCTCGTCTTTTACCAGCATAAGAGCA | STVTTSPYAFTRVT |
| | TGAATGAGCCAAAACATGGCTTGGCTCTTTGGGAAGC | GPYNRYI |
| | CAAAATGGCTGAAAAAGCCCGTGAGAAAGAGGAAGAG | (SEQ ID NO: 393) |
| | TGTGAAAAGTATGGCCCAGACTATGTGCCTCAGAAATC | |
| | CCATGGCAAAAAAGTGAAACGGGAGCCTGCTGAGCCA | |
| | CATGAAACTTCAGAGCCCACTTACCTGCGTTTCATCAA | |
| | GTCTCTTGCCGAAAGGACCATGTCCGTGACCACAGAC | |
| | TCCACAGTAACTACATCTCCATATGCCTTCACTCGGGT | |
| | CACAGGGCCTTACAACAGATATATATGA | |
| | (SEQ ID NO: 360) | |
| YBX1 | ATGAGCAGCGAGGCCGAGACCCAGCAGCCGCCCGCC | MSSEAETQQPPAA |
| | GCCCCCCCCGCCGCCCCCGCCCTCAGCGCCGCCGAC | PPAAPALSAADTK |
| | ACCAAGCCCGGCACTACGGGCAGCGGCGCAGGGAGC | PGTTGSGAGSGG |
| | GGTGGCCCGGGCGGCCTCACATCGGCGGCGCCTGCC | PGGLTSAAPAGGD |
| | GGCGGGGACAAGAAGGTCATCGCAACGAAGGTTTTGG | KKVIATKVLGTVKW |
| | GAACAGTAAAATGGTTCAATGTAAGGAACGGATATGGT | FNVRNGYGFINRN |
| | TTCATCAACAGGAATGACACCAAGGAAGATGTATTTGT | DTKEDVFVHQTAIK |
| | ACACCAGACTGCCATAAAGAAGAATAACCCCAGGAAG | KNNPRKYLRSVGD |
| | TACCTTCGCAGTGTAGGAGATGGAGAGACTGTGGAGT | GETVEFDVVEGEK |
| | TTGATGTTGTTGAAGGAGAAAAGGGTGCGGAGGCAGC | GAEAANVTGPGGV |
| | AAATGTTACAGGTCCTGGTGGTGTTCCAGTTCAAGGCA | PVQGSKYAADRNH |
| | GTAAATATGCAGCAGACCGTAACCATTATAGACGCTAT | YRRYPRRRGPPRN |
| | CCACGTCGTAGGGGTCCTCCACGCAATTACCAGCAAA | YQQNYQNSESGE |
| | ATTACCAGAATAGTGAGAGTGGGGAAAAGAACGAGGG | KNEGSESAPEGQA |
| | ATCGGAGAGTGCTCCCGAAGGCCAGGCCCAACAACG | QQRRPYRRRRFPP |
| | CCGGCCCTACCGCAGGCGAAGGTTCCCACCTTACTAC | YYMRRPYGRRPQ |
| | ATGCGGAGACCCTATGGGCGTCGACCACAGTATTCCA | YSNPPVQGEVME |
| | ACCCTCCTGTGCAGGGAGAAGTGATGGAGGGTGCTGA | GADNQGAGEQGR |
| | CAACCAGGGTGCAGGAGAACAAGGTAGACCAGTGAG | PVRQNMYRGYRP |
| | GCAGAATATGTATCGGGGATATAGACCACGATTCCGC | RFRRGPPRQRQP |
| | AGGGGCCCTCCTCGCCAAAGACAGCCTAGAGAGGAC | REDGNEEDKENQ |
| | GGCAATGAAGAAGATAAAGAAAATCAAGGAGATGAGA | GDETQGQQPPQR |
| | CCCAAGGTCAGCAGCCACCTCAACGTCGGTACCGCCG | RYRRNFNYRRRRP |
| | CAACTTCAATTACCGACGCAGACGCCCAGAAAACCCTA | ENPKPQDGKETKA |
| | AACCACAAGATGGCAAAGAGACAAAAGCAGCCGATCC | ADPPAENSSAPEA |
| | ACCAGCTGAGAATTCGTCCGCTCCCGAGGCTGAGCAG | EQGGAE |
| | GGCGGGGCTGAGTAA | (SEQ ID NO: 394) |
| | (SEQ ID NO: 361) | |
| YY1 | ATGGCCTCGGGCGACACCCTCTACATCGCCACGGACG | MASGDTLYIATDG |
| | GCTCGGAGATGCCGGCCGAGATCGTGGAGCTGCACG | SEMPAEIVELHEIE |
| | AGATCGAGGTGGAGACCATCCCGGTGGAGACCATCGA | VETIPVETIETTVVG |
| | GACCACAGTGGTGGGCGAGGAGGAGGAGGAGGACGA | EEEEEDDDDEDGG |
| | CGACGACGAGGACGGCGGCGGTGGCGACCACGGCG | GGDHGGGGGHGH |
| | GCGGGGGCGGCCACGGGCACGCCGGCCACCACCAC | AGHHHHHHHHHH |
| | CACCACCATCACCACCACCACCACCCGCCCATGATCG | HPPMIALQPLVTDD |
| | CTCTGCAGCCGCTGGTCACCGACGACCCGACCCAGGT | PTQVHHHQEVILV |
| | GCACCACCACCAGGAGGTGATCCTGGTGCAGACGCG | QTREEVVGGDDSD |
| | CGAGGAGGTGGTGGGCGGCGACGACTCGGACGGGCT | GLRAEDGFEDQILI |
| | GCGCGCCGAGGACGGCTTCGAGGATCAGATTCTCATC | PVPAPAGGDDDYI |
| | CCGGTGCCCGCGCCGGCCGGCGGCGACGACGACTAC | EQTLVTVAAAGKS |
| | ATTGAACAAACGCTGGTCACCGTGGCGGCGGCCGGC | GGGGSSSSGGGR |
| | AAGAGCGGCGGCGGCGGCTCGTCGTCGTCGGGAGGC | VKKGGGKKSGKKS |
| | GGCCGCGTCAAGAAGGGCGGCGGCAAGAAGAGCGGC | YLSGGAGAAGGG |
| | AAGAAGAGTTACCTCAGCGGCGGGGCCGGCGCGGCG | GADPGNKKWEQK |
| | GGCGGCGGCGGCGCCGACCCGGGCAACAAGAAGTG | QVQIKTLEGEFSVT |
| | GGAGCAGAAGCAGGTGCAGATCAAGACCCTGGAGGG | MWSSDEKKDIDHE |
| | CGAGTTCTCGGTCACCATGTGGTCCTCAGATGAAAAAA | TWEEQIIGENSPP |
| | AAGATATTGACCATGAGACAGTGGTTGAAGAACAGATC | DYSEYMTGKKLPP |
| | ATTGGAGAGAACTCACCTCCTGATTATTCAGAATATAT | GGIPGIDLSDPKQL |
| | GACAGGAAAGAAACTTCCTCCTGGAGGAATACCTGGC | AEFARMKPRKIKE |
| | ATTGACCTCTCAGATCCCAAACAACTGGCAGAATTTGC | DDAPRTIACPHKG |
| | TAGAATGAAGCCAAGAAAAATTAAAGAAGATGATGCTC | CTKMFRDNSAMRK |
| | CAAGAACAATAGCTTGCCCTCATAAAGGCTGCACAAAG | HLHTHGPRVHVCA |
| | ATGTTCAGGGATAACTCGGCCATGAGAAAACATCTGCA | ECGKAFVESSKLK |
| | CACCCACGGTCCCAGAGTCCACGTCTGTGCAGAATGT | RHQLVHTGEKPFQ |
| | GGCAAAGCTTTTGTTGAGAGTTCAAAACTAAAACGACA | CTFEGCGKRFSLD |
| | CCAACTGGTTCATACTGGAGAGAAGCCCTTTCAGTGCA | FNLRTHVRIHTGDR |
| | CGTTCGAAGGCTGTGGGAAACGCTTTTCACTGGACTT | PYVCPFDGCNKKF |
| | CAATTTGCGCACACATGTGCGAATCCATACCGGAGAC | AQSTNLKSHILTHA |
| | AGGCCCTATGTGTGCCCCTTCGATGGTTGTAATAAGAA | KAKNNQ |
| | GTTTGCTCAGTCAACTAACCTGAAATCTCACATCTTAAC | (SEQ ID NO: 395 |
| | ACATGCTAAGGCCAAAAACAACCAGTGA | |
| | (SEQ ID NO: 362) | |
| ZBTB1 | ATGGCAAAGCCCAGCCACAGCAGCTATGTCCTTCAGC | MAKPSHSSYVLQQ |
| | AGCTAAACAACCAAAGAGAATGGGGTTTTCTCTGTGAC | LNNQREWGFLCDC |
| | TGCTGTATTGCAATTGATGACATTTACTTTCAAGCACAC | CIAIDDIYFQAHKAV |
| | AAGGCAGTTCTAGCTGCCTGTAGCTCCTATTTTAGAAT | LAACSSYFRMFFM |
| | GTTTTTCATGAACCATCAGCATAGTACTGCACAACTGA | NHQHSTAQLNLSN |
| | ATCTCAGCAACATGAAAATTAGTGCAGAATGTTTTGAT | MKISAECFDLILQF |
| | CTCATTTTGCAGTTTATGTATTTAGGAAAAATTATGACA | MYLGKIMTAPSSFE |
| | GCTCCCTCCAGTTTTGAGCAGTTTAAAGTGGCAATGAA | QFKVAMNYLQLYN |
| | CTACCTACAGCTATACAATGTTCCTGACTGTTTAGAAG | VPDCLEDIQDADC |
| | ACATCCAGGATGCAGATTGTTCTAGTTCAAAATGTTCC | SSSKCSSSASSKQ |
| | TCTTCTGCTTCCAGCAAACAGAACAGCAAAATGATATT | NSKMIFGVRMYED |
| | TGGGGTAAGAATGTATGAAGATACTGTGGCTCGAAATG | TVARNGNEANRW |
| | GCAATGAAGCCAACAGGTGGTGTGCAGAGCCAAGTTC | CAEPSSTVNTPHN |
| | AACGGTAAATACACCACATAATAGAGAGGCTGATGAAG | READEESLQLGNF |
| | AGTCTTTACAATTAGGTAATTTTCCTGAGCCACTATTTG | PEPLFDVCKKSSV |
| | ATGTATGTAAAAAAAGTTCCGTGTCCAAATTATCTACTC | SKLSTPKERVSRR |
| | CAAAAGAACGTGTGTCAAGACGCTTTGGGCGGAGTTT | FGRSFTCDSCGFG |
| | TACCTGTGATAGCTGTGGATTTGGCTTTAGCTGTGAAA | FSCEKLLDEHVLTC |
| | AATTATTAGATGAGCATGTGCTAACCTGTACTAACAGA | TNRHLYQNTRSYH |
| | CATTTATACCAAAACACAAGATCTTACCATAGAATAGTA | RIVDIRDGKDSNIK |
| | GATATTAGAGATGGAAAAGACAGTAACATCAAAGCTGA | AEFGEKDSSKTFS |
| | ATTTGGTGAAAAAGATTCTTCCAAAACATTTTCTGCACA | AQTDKYRGDTSQA |
| | GACGGACAAATACAGAGGAGACACAAGCCAGGCTGCT | ADDSASTTGSRKS |
| | GATGATTCAGCTTCAACCACTGGAAGCAGAAAAAGTAG | STVESEIASEEKSR |
| | CACAGTGGAGTCTGAAATAGCAAGCGAAGAGAAAAGC | AAERKRITIKMEPED |
| | AGAGCTGCTGAGAGGAAAAGGATTATTATTAAGATGGA | IPTDELKDFNIIKVT |
| | GCCAGAAGATATTCCTACAGATGAACTGAAAGACTTTA | DKDCNESTDNDEL |
| | ACATTATTAAAGTTACTGATAAAGACTGTAATGAATCCA | EDEPEEPFYRYYV |
| | CTGACAATGATGAATTAGAAGATGAACCTGAAGAGCCA | EEDVSIKKSGRKTL |
| | TTTTATAGATACTATGTTGAAGAAGATGTCAGCATAAAA | KPRMSVSADERG |
| | AAAAGTGGTAGGAAAACTCTAAAACCTCGAATGTCAGT | GLENMRPPNNSSP |
| | AAGTGCTGATGAAAGAGGTGGTTTAGAGAATATGAGG | VQEDAENASCELC |
| | CCCCCTAACAACAGCAGTCCAGTACAAGAGGATGCTG | GLTITEEDLSSHYL |
| | AAAATGCATCTTGTGAGCTGTGTGGACTTACAATAACC | AKHIENICACGKCG |
| | GAGGAGGACCTGTCATCTCATTACTTAGCCAAACACAT | QILVKGRQLQEHA |
| | TGAAAATATCTGTGCATGTGGTAAATGTGGACAAATAC | QRCGEPQDLTMN |
| | TTGTAAAGGGTAGGCAGCTTCAGGAACATGCTCAACG | GLGNTEEKMDLEE |
| | ATGTGGCGAGCCCCAAGATCTGACCATGAATGGGTTA | NPDEQSEIRDMFV |
| | GGAAATACTGAGGAGAAAATGGACTTGGAAGAGAATC | EMLDDFRDNHYQI |
| | CTGATGAGCAGTCCGAAATAAGAGATATGTTTGTTGAA | NSIQKKQLFKHSAC |
| | ATGCTGGATGATTTTAGGGACAATCATTACCAGATAAA | PFRCPNCGQRFET |
| | CAGTATCCAAAAAAAGCAGTTATTTAAACATTCTGCCTG | ENLVVEHMSSCLD |
| | CCCTTTTCGATGTCCTAATTGTGGCCAGCGTTTTGAAA | QDMFKSAIMEENE |
| | CTGAAAATCTAGTGGTTGAACATATGTCTAGCTGCTTA | RDHRRKHFCNLCG |
| | GATCAAGATATGTTTAAGAGTGCCATCATGGAAGAAAA | KGFYQRCHLREHY |
| | TGAAAGAGATCACAGACGAAAGCATTTTTGTAATCTGT | TVHTKEKQFVCQT |
| | GTGGAAAAGGATTTTATCAGCGGTGTCACTTAAGAGAA | CGKQFLRERQLRL |
| | CACTATACTGTTCATACTAAGGAAAAACAGTTTGTTTGT | HNDMHKGMARYV |
| | CAAACATGTGGAAAGCAGTTTTTAAGAGAGCGTCAGTT | CSICDQGNFRKHD |
| | GCGACTGCACAATGATATGCACAAAGGCATGGCCAGG | HVRHMISHLSAGE |
| | TATGTCTGTTCCATTTGTGATCAAGGAAACTTCAGAAAA | TICQVCFQIFPNNE |
| | CATGACCATGTACGGCATATGATTTCTCATTTATCTGCT | QLEQHMDVHLYTC |
| | GGTGAGACTATATGCCAGGTCTGCTTTCAGATATTCCC | GICGAKFNLRKDM |
| | AAATAATGAACAGTTGGAGCAGCACATGGATGTTCATC | RSHYNAKHLKRT |
| | TGTATACATGTGGAATATGTGGAGCAAAATTTAATTTGA | (SEQ ID NO: 396) |
| | GGAAAGATATGAGATCACATTATAATGCCAAGCATTTG | |
| | AAAAGAACCTGA | |
| | (SEQ ID NO: 363 | |
| ZC3H8 | ATGGATTTTGAGAATCTTTTCTCAAAACCCCCCAACCC | MDFENLFSKPPNP |
| | GGCCCTCGGCAAAACGGCCACGGACTCTGACGAAAGA | ALGKTATDSDERID |
| | ATCGATGATGAAATAGATACAGAAGTTGAAGAAACACA | DEIDTEVEETQEEK |
| | AGAAGAGAAAATTAAACTGGAGTGCGAGCAAATTCCCA | IKLECEQIPKKFRH |
| | AAAAATTTAGACACTCTGCAATATCACCAAAAAGTTCG | SAISPKSSLHRKSR |
| | CTGCATAGAAAATCAAGAAGTAAGGACTATGATGTATA | SKDYDVYSDNDIC |
| | TAGTGATAATGATATCTGCAGTCAGGAATCAGAAGATA | SQESEDNFAKELQ |
| | ATTTTGCCAAAGAGCTTCAACAGTACATACAAGCCAGA | QYIQAREMANAAQ |
| | GAAATGGCAAATGCTGCTCAACCTGAAGAATCTACAAA | PEESTKKEGVKDT |
| | GAAAGAAGGAGTAAAAGATACCCCACAGGCTGCTAAA | PQAAKQKNKNLKA |
| | CAAAAAAATAAAAATCTTAAAGCTGGTCACAAGAATGG | GHKNGKQKKMKR |
| | CAAACAGAAGAAAATGAAGCGAAAATGGCCTGGCCCT | KWPGPGNKGSNA |
| | GGAAACAAAGGATCAAATGCTTTGCTGAGGAACAGCG | LLRNSGSQEEDGK |
| | GCTCACAGGAAGAGGATGGTAAACCTAAAGAGAAGCA | PKEKQQHLSQAFI |
| | GCAGCATTTGAGTCAGGCATTCATCAACCAACATACAG | NQHTVERKGKQIC |
| | TGGAACGCAAGGGAAAACAAATTTGTAAATATTTTCTT | KYFLERKCIKGDQ |
| | GAAAGGAAATGTATTAAGGGAGACCAGTGTAAATTTGA | CKFDHDAEIEKKKE |
| | TCATGATGCAGAGATAGAAAAGAAAAAGGAAATGTGTA | MCKFYVQGYCTRG |
| | AGTTTTATGTACAAGGATATTGTACCAGAGGTGAAAAC | ENCLYLHNEYPCK |
| | TGTCTGTATTTGCATAATGAATATCCTTGTAAGTTTTAC | FYHTGTKCYQGEY |
| | CATACAGGAACAAAATGTTATCAGGGAGAATACTGCAA | CKFSHAPLTPETQ |
| | GTTTTCTCATGCTCCACTGACTCCTGAAACACAAGAAT | ELLAKVLDTEKKSC |
| | TGTTGGCTAAAGTTTTGGATACTGAAAAGAAGTCATGT | K |
| | AAATAA | (SEQ ID NO: 397) |
| | (SEQ ID NO: 364) | |
| ZFX | ATGGATGAAGATGGGCTTGAATTACAACAAGAGCCAAA | MDEDGLELQQEPN |
| | CTCATTTTTTGATGCAACAGGAGCTGATGGTACACACA | SFFDATGADGTHM |
| | TGGATGGTGATCAAATTGTTGTGGAAGTACAAGAAACT | DGDQIVVEVQETV |
| | GTTTTTGTTTCAGATGTTGTGGATTCAGACATAACTGTG | FVSDVVDSDITVHN |
| | CATAACTTTGTTCCTGATGACCCAGATTCAGTTGTAATC | FVPDDPDSVVIQD |
| | CAAGATGTTATTGAGGACGTTGTTATAGAAGATGTTCA | VIEDVVIEDVQCPDI |
| | GTGCCCAGATATCATGGAAGAAGCAGATGTGTCTGAA | MEEADVSETVIIPE |
| | ACGGTCATCATTCCTGAGCAAGTGCTGGACTCAGATGT | QVLDSDVTEEVSL |
| | AACTGAAGAAGTTTCTTTAGCACATTGCACAGTCCCAG | AHCTVPDDVLASDI |
| | ATGATGTTTTAGCTTCTGACATTACTTCAGCCTCAATGT | TSASMSMPEHVLT |
| | CTATGCCAGAACACGTCTTGACGGGTGATTCTATACAT | GDSIHVSDVGHVG |
| | GTGTCTGACGTTGGACATGTTGGACATGTTGGACATGT | HVGHVEHVVHDSV |
| | TGAACATGTGGTTCATGATAGTGTAGTGGAAGCAGAAA | VEAEIVTDPLTTDV |
| | TTGTCACTGATCCTCTGACTACCGACGTAGTTTCAGAA | VSEEVLVADCASE |
| | GAAGTATTGGTAGCAGACTGTGCCTCTGAAGCAGTCAT | AVIDANGIPVDQQD |
| | AGATGCCAATGGGATCCCTGTGGACCAGCAGGATGAT | DDKGNCEDYLMIS |
| | GACAAAGGCAACTGTGAGGACTACCTTATGATTTCCTT | LDDAGKIEHDGSS |
| | GGATGATGCTGGCAAAATAGAACACGATGGTTCTTCTG | GMTMDTESEIDPC |
| | GAATGACCATGGACACAGAGTCGGAAATTGATCCTTGT | KVDGTCPEVIKVYI |
| | AAAGTGGATGGCACTTGCCCTGAGGTCATCAAGGTGT | FKADPGEDDLGGT |
| | ACATTTTTAAAGCTGACCCTGGAGAAGATGACTTAGGT | VDIVESEPENDHG |
| | GGAACTGTAGACATTGTGGAGAGTGAGCCTGAGAATG | VELLDQNSSIRVPR |
| | ATCATGGAGTTGAACTGCTTGATCAGAACAGCAGTATT | EKMVYMTVNDSQP |
| | CGTGTTCCCAGGGAAAAGATGGTTTATATGACTGTCAA | EDEDLNVAEIADEV |
| | TGACTCTCAGCCAGAAGATGAAGATTTAAATGTTGCTG | YMEVIVGEEDAAA |
| | AAATCGCTGACGAAGTTTATATGGAAGTGATCGTAGGA | AAAAAAVHEQQMD |
| | GAGGAGGATGCTGCAGCAGCAGCGGCAGCCGCCGCC | DNEIKTFMPIAWAA |
| | GTGCACGAGCAGCAAATGGATGACAATGAAATCAAAA | AYGNNSDGIENRN |
| | CCTTCATGCCGATTGCATGGGCAGCAGCTTATGGTAAT | GTASALLHIDESAG |
| | AATTCTGATGGAATTGAAAACCGGAATGGCACTGCAAG | LGRLAKQKPKKRR |
| | TGCCCTCTTGCACATAGATGAGTCTGCTGGCCTCGGC | RPDSRQYQTAIIIG |
| | AGACTGGCTAAACAAAAACCAAAGAAAAGGAGAAGAC | PDGHPLTVYPCMI |
| | CTGATTCCAGGCAGTACCAAACAGCAATAATTATTGGC | CGKKFKSRGFLKR |
| | CCTGATGGACATCCTTTGACTGTCTATCCTTGCATGAT | HMKNHPEHLAKKK |
| | TTGTGGGAAGAAGTTTAAGTCGAGAGGTTTTTTGAAAA | YRCTDCDYTTNKKI |
| | GGCACATGAAAAACCATCCCGAACACCTTGCCAAGAA | SLHNHLESHKLTSK |
| | GAAATACCGCTGTACTGACTGTGATTACACTACCAACA | AEKAIECDECGKH |
| | AGAAGATAAGTTTACACAACCACCTGGAGAGCCACAA | FSHAGALFTHKMV |
| | GCTGACCAGCAAGGCAGAGAAGGCCATTGAATGCGAT | HKEKGANKMHKCK |
| | GAGTGTGGGAAGCATTTCTCTCATGCAGGGGCTTTGTT | FCEYETAEQGLLN |
| | TACTCACAAAATGGTGCATAAGGAAAAAGGAGCCAACA | RHLLAVHSKNFPHI |
| | AAATGCACAAGTGTAAATTCTGTGAATACGAGACAGCT | CVECGKGFRHPSE |
| | GAACAAGGGTTATTGAATCGCCACCTCTTGGCAGTCCA | LKKHMRIHTGEKP |
| | CAGCAAGAACTTTCCTCATATTTGTGTGGAGTGTGGTA | YQCQYCEYRSADS |
| | AGGGTTTTCGTCACCCGTCAGAGCTCAAAAAGCACAT | SNLKTHVKTKHSK |
| | GAGAATCCATACTGGGGAGAAGCCGTACCAATGCCAG | EMPFKCDICLLTFS |
| | TACTGCGAATATAGGTCTGCAGACTCTTCTAACTTGAA | DTKEVQQHALIHQ |
| | AACGCATGTCAAAACTAAGCATAGTAAAGAGATGCCAT | ESKTHQCLHCDHK |
| | TCAAGTGTGACATTTGTCTTCTGACTTTCTCGGATACC | SSNSSDLKRHIISV |
| | AAAGAGGTGCAGCAACATGCTCTTATCCACCAAGAAAG | HTKDYPHKCDMCD |
| | CAAAACACACCAGTGTTTGCATTGCGACCACAAGAGTT | KGFHRPSELKKHV |
| | CGAACTCAAGTGATTTGAAACGACACATAATTTCAGTT | AAHKGKKMHQCR |
| | CACACGAAAGACTACCCCCATAAGTGTGACATGTGTGA | HCDFKIADPFVLSR |
| | TAAAGGCTTTCACAGGCCTTCAGAACTCAAGAAACACG | HILSVHTKDLPFRC |
| | TGGCTGCCCACAAGGGCAAAAAAATGCACCAGTGTAG | KRCRKGFRQQSEL |
| | ACATTGTGACTTTAAGATTGCAGATCCATTTGTTCTAAG | KKHMKTHSGRKVY |
| | TCGCCATATTCTCTCAGTTCACACAAAGGATCTTCCATT | QCEYCEYSTTDAS |
| | TAGGTGCAAGAGATGTAGAAAGGGATTTAGGCAACAG | GFKRHVISIHTKDY |
| | AGTGAGCTTAAAAAGCATATGAAGACACACAGTGGCA | PHRCEYCKKGFRR |
| | GGAAAGTGTATCAGTGTGAGTACTGTGAGTATAGCACT | PSEKNQHIMRHHK |
| | ACAGATGCCTCAGGCTTTAAACGGCACGTTATTTCCAT | EVGLP |
| | TCACACGAAAGACTATCCTCACCGGTGTGAGTACTGCA | (SEQ ID NO: 398) |
| | AGAAAGGCTTCCGAAGACCTTCAGAAAAGAACCAGCA | |
| | CATAATGCGACATCATAAAGAAGTTGGCCTGCCCTAA | |
| | (SEQ ID NO: 365) | |
| ZNF217 | ATGCAATCGAAAGTGACAGGAAACATGCCAACTCAATC | MQSKVTGNMPTQ |
| | CCTCTTAATGTACATGGATGGGCCAGAAGTGATTGGCA | SLLMYMDGPEVIG |
| | GCTCTCTTGGCAGTCCGATGGAGATGGAGGATGCCTT | SSLGSPMEMEDAL |
| | GTCAATGAAAGGGACCGCTGTTGTTCCATTCCGAGCTA | SMKGTAVVPFRAT |
| | CACAAGAAAAAAATGTCATCCAAATCGAGGGGTATATG | QEKNVIQIEGYMPL |
| | CCCTTGGATTGCATGTTCTGCAGCCAGACCTTCACACA | DCMFCSQTFTHSE |
| | TTCAGAAGACCTTAATAAACATGTCTTAATGCAACACC | DLNKHVLMQHRPT |
| | GGCCTACCCTCTGTGAACCAGCAGTTCTTCGGGTTGA | LCEPAVLRVEAEYL |
| | AGCAGAGTATCTCAGTCCGCTTGATAAAAGTCAAGTGC | SPLDKSQVRTEPP |
| | GAACAGAACCTCCCAAGGAAAAGAATTGCAAGGAAAAT | KEKNCKENEFSCE |
| | GAATTTAGCTGTGAGGTATGTGGGCAGACATTTAGAGT | VCGQTFRVAFDVEI |
| | CGCTTTTGATGTTGAGATCCACATGAGAACACACAAAG | HMRTHKDSFTYGC |
| | ATTCTTTCACTTACGGGTGTAACATGTGCGGAAGAAGA | NMCGRRFKEPWFL |
| | TTCAAGGAGCCTTGGTTTCTTAAAAATCACATGCGGAC | KNHMRTHNGKSG |
| | ACATAATGGCAAATCGGGGGCCAGAAGCAAACTGCAG | ARSKLQQGLESSP |
| | CAAGGCTTGGAGAGTAGTCCAGCAACGATCAACGAGG | ATINEVVQVHAAES |
| | TCGTCCAGGTGCACGCGGCCGAGAGCATCTCCTCTCC | ISSPYKICMVCGFL |
| | TTACAAAATCTGCATGGTTTGTGGCTTCCTATTTCCAAA | FPNKESLIEHRKVH |
| | TAAAGAAAGTCTAATTGAGCACCGCAAGGTGCACACCA | TKKTAFGTSSAQT |
| | AAAAAACTGCTTTCGGTACCAGCAGCGCGCAGACAGA | DSPQGGMPSSRE |
| | CTCTCCACAAGGAGGAATGCCGTCCTCGAGGGAGGAC | DFLQLFNLRPKSH |
| | TTCCTGCAGTTGTTCAACTTGAGACCAAAATCTCACCC | PETGKKPVRCIPQL |
| | TGAAACGGGGAAGAAGCCTGTCAGATGCATCCCTCAG | DPFTTFQAWQLAT |
| | CTCGATCCGTTCACCACCTTCCAGGCTTGGCAGCTGG | KGKVAICQEVKES |
| | CTACCAAAGGAAAAGTTGCCATTTGCCAAGAAGTGAAG | GQEGSTDNDDSSS |
| | GAATCGGGGCAAGAAGGGAGCACCGACAACGACGATT | EKELGETNKGSCA |
| | CGAGTTCCGAGAAGGAGCTTGGAGAAACAAATAAGGG | GLSQEKEKCKHSH |
| | CAGTTGTGCAGGCCTCTCGCAAGAGAAAGAGAAGTGC | GEAPSVDADPKLP |
| | AAACACTCCCACGGCGAAGCGCCCTCCGTGGACGCG | SSKEKPTHCSECG |
| | GATCCCAAGTTACCCAGTAGCAAGGAGAAGCCCACTC | KAFRTYHQLVLHS |
| | ACTGCTCCGAGTGCGGCAAAGCTTTCAGAACCTACCA | RVHKKDRRAGAES |
| | CCAGCTGGTCTTGCACTCCAGGGTCCACAAGAAGGAC | PTMSVDGRQPGTC |
| | CGGAGGGCCGGCGCGGAGTCGCCCACCATGTCTGTG | SPDLAAPLDENGA |
| | GACGGGAGGCAGCCGGGGACGTGTTCTCCTGACCTC | VDRGEGGSEDGS |
| | GCCGCCCCTCTGGATGAAAATGGAGCCGTGGATCGAG | EDGLPEGIHLDKN |
| | GGGAAGGTGGTTCTGAAGACGGATCTGAGGATGGGCT | DDGGKIKHLTSSR |
| | TCCCGAAGGAATCCATCTGGATAAAAATGATGATGGAG | ECSYCGKFFRSNY |
| | GAAAAATAAAACATCTTACATCTTCAAGAGAGTGTAGTT | YLNIHLRTHTGEKP |
| | ATTGTGGAAAGTTTTTCCGTTCAAATTATTACCTCAATA | YKCEFCEYAAAQK |
| | TTCATCTCAGAACGCATACAGGTGAAAAACCATACAAA | TSLRYHLERHHKE |
| | TGTGAATTTTGTGAATATGCTGCAGCCCAGAAGACATC | KQTDVAAEVKNDG |
| | TCTGAGGTATCACTTGGAGAGACATCACAAGGAAAAAC | KNQDTEDALLTAD |
| | AAACCGATGTTGCTGCTGAAGTCAAGAACGATGGTAAA | SAQTKNLKRFFDG |
| | AATCAGGACACTGAAGATGCACTATTAACCGCTGACAG | AKDVTGSPPAKQL |
| | TGCGCAAACCAAAAATTTGAAAAGATTTTTTGATGGTG | KEMPSVFQNVLGS |
| | CCAAAGATGTTACAGGCAGTCCACCTGCAAAGCAGCT | AVLSPAHKDTQDF |
| | TAAGGAGATGCCTTCTGTTTTTCAGAATGTTCTGGGCA | HKNAADDSADKVN |
| | GCGCTGTCCTCTCACCAGCACACAAAGATACTCAGGA | KNPTPAYLDLLKKR |
| | TTTCCATAAAAATGCAGCTGATGACAGTGCTGATAAAG | SAVETQANNLICRT |
| | TGAATAAAAACCCTACCCCTGCTTACCTGGACCTGTTA | KADVTPPPDGSTT |
| | AAAAAGAGATCAGCAGTTGAAACTCAGGCAAATAACCT | HNLEVSPKEKQTE |
| | CATCTGTAGAACCAAGGCGGATGTTACTCCTCCTCCG | TAADCRYRPSVDC |
| | GATGGCAGTACCACCCATAACCTTGAAGTTAGCCCCAA | HEKPLNLSVGALH |
| | AGAGAAGCAAACGGAGACCGCAGCTGACTGCAGATAC | NCPAISLSKSLIPSI |
| | AGGCCAAGTGTGGATTGTCACGAAAAACCTTTAAATTT | TCPFCTFKTFYPEV |
| | ATCCGTGGGGGCTCTTCACAATTGCCCGGCAATTTCTT | LMMHQRLEHKYNP |
| | TGAGTAAAAGTTTGATTCCAAGTATCACCTGTCCATTTT | DVHKNCRNKSLLR |
| | GTACCTTCAAGACATTTTATCCAGAAGTTTTAATGATGC | SRRTGCPPALLGK |
| | ACCAGAGACTGGAGCATAAATACAATCCTGACGTTCAT | DVPPLSSFCKPKP |
| | AAAAACTGTCGAAACAAGTCCTTGCTTAGAAGTCGACG | KSAFPAQSKSLPS |
| | TACCGGATGCCCGCCAGCGTTGCTGGGAAAAGATGTG | AKGKQSPPGPGKA |
| | CCTCCCCTCTCTAGTTTCTGTAAACCCAAGCCCAAGTC | PLTSGIDSSTLAPS |
| | TGCTTTCCCGGCGCAGTCCAAATCCCTGCCATCTGCG | NLKSHRPQQNVGV |
| | AAGGGGAAGCAGAGCCCTCCTGGGCCAGGCAAGGCC | QGAATRQQQSEM |
| | CCTCTGACTTCAGGGATAGACTCTAGCACTTTAGCCCC | FPKTSVSPAPDKT |
| | AAGTAACCTGAAGTCCCACAGACCACAGCAGAATGTG | KRPETKLKPLPVAP |
| | GGGGTCCAAGGGGCCGCCACCAGGCAACAGCAATCT | SQPTLGSSNINGSI |
| | GAGATGTTTCCTAAAACCAGTGTTTCCCCTGCACCGGA | DYPAKNDSPWAPP |
| | TAAGACAAAAAGACCCGAGACAAAATTGAAACCTCTTC | GRDYFCNRSASNT |
| | CAGTAGCTCCTTCTCAGCCCACCCTCGGCAGCAGTAA | AAEFGEPLPKRLK |
| | CATCAATGGTTCCATCGACTACCCCGCCAAGAACGAC | SSWVALDVDQPGA |
| | AGCCCGTGGGCACCTCCGGGAAGAGACTATTTCTGTA | NYRRGYDLPKYHM |
| | ATCGGAGTGCCAGCAATACTGCAGCAGAATTTGGTGA | VRGITSLLPQDCVY |
| | GCCCCTTCCAAAAAGACTGAAGTCCAGCGTGGTTGCC | PSQALPPKPRFLS |
| | CTTGACGTTGACCAGCCCGGGGCCAATTACAGAAGAG | SSEVDSPNVLTVQ |
| | GCTATGACCTTCCCAAGTACCATATGGTCAGAGGCATC | KPYGGSGPLYTCV |
| | ACATCACTGTTACCGCAGGACTGTGTGTATCCGTCGCA | PAGSPASSSTLEG |
| | GGCGCTGCCTCCCAAACCAAGGTTCCTGAGCTCCAGC | KRPVSYQHLSNSM |
| | GAGGTCGATTCTCCAAATGTGCTGACTGTTCAGAAGCC | AQKRNYENFIGNA |
| | CTATGGTGGCTCCGGGCCACTTTACACTTGTGTGCCT | HYRPNDKKT |
| | GCTGGTAGTCCAGCATCCAGCTCGACGTTAGAAGGAA | (SEQ ID NO: 399) |
| | AAAGGCCTGTGTCATATCAACACTTATCTAACAGCATG | |
| | GCACAAAAGAGAAACTATGAGAATTTTATTGGGAATGC | |
| | ACATTATCGACCAAATGACAAAAAAACTTGA | |
| | (SEQ ID NO: 366) | |
| ZNF335 | ATGGAGGAGAACGAGGTGGAGAGCAGCAGCGACGCG | MEENEVESSSDAA |
| | GCCCCTGGGCCTGGCCGGCCCGAGGAGCCCTCTGAG | PGPGRPEEPSESG |
| | AGCGGCCTGGGTGTGGGCACCTCAGAAGCCGTGTCC | LGVGTSEAVSADS |
| | GCCGACAGCAGCGACGCCGCGGCCGCCCCGGGGCA | SDAAAAPGQAEAD |
| | GGCAGAGGCCGATGACTCTGGCGTGGGGCAAAGCTC | DSGVGQSSDRGS |
| | GGACCGCGGCAGCCGTTCTCAGGAGGAGGTATCTGA | RSQEEVSESSSSA |
| | GAGCAGCTCGAGCGCAGACCCCCTGCCTAATAGCTAC | DPLPNSYLPDSSS |
| | CTCCCTGATTCATCGTCTGTGTCTCATGGGCCAGTGG | VSHGPVAGVTGGP |
| | CAGGGGTGACAGGCGGTCCCCCAGCACTTGTGCACTC | PALVHSSALPDPN |
| | TAGTGCACTCCCAGACCCCAACATGCTGGTGTCCGAC | MLVSDCTASSSDL |
| | TGCACAGCTTCCTCCTCGGACCTGGGCTCGGCCATCG | GSAIDKIIESTIGPD |
| | ACAAGATCATCGAGTCCACCATCGGGCCCGACCTCAT | LIQNCITVTSAEDG |
| | CCAGAACTGCATCACTGTGACCAGTGCTGAGGATGGC | GAETTRYLILQGPD |
| | GGGGCCGAGACCACACGGTACCTGATCCTACAGGGC | DGAPMTSPMSSST |
| | CCAGATGATGGAGCCCCCATGACATCACCAATGTCCA | LAHSLAAIEALADG |
| | GTTCCACCTTGGCCCACAGCCTAGCAGCCATTGAGGC | PTSTSTCLEAQGG |
| | CCTGGCAGATGGCCCCACATCCACATCCACATGCCTG | PSSPVQLPPASGA |
| | GAGGCACAGGGTGGGCCCAGCTCCCCGGTGCAGCTG | EEPDLQSLEAMME |
| | CCCCCAGCCTCCGGTGCCGAAGAGCCGGACCTGCAG | VVVVQQFKCKMC |
| | AGCCTGGAGGCCATGATGGAGGTGGTGGTGGTGCAG | QYRSSTKATLLRH |
| | CAGTTCAAATGCAAGATGTGCCAGTACCGGAGCAGCA | MRERHFRPVAAAA |
| | CCAAGGCCACACTGCTGCGCCACATGCGGGAACGCC | AAAGKKGRLRKWS |
| | ACTTCCGTCCAGTAGCAGCAGCCGCAGCAGCAGCTGG | TSTKSQEEEGPEE |
| | TAAAAAAGGACGTCTACGGAAGTGGAGCACCTCCACC | EDDDDIVDAGAIDD |
| | AAGAGCCAAGAGGAAGAGGGACCAGAGGAGGAGGAC | LEEDSDYNPAEDE |
| | GATGATGACATTGTAGACGCTGGAGCCATTGATGACCT | PRGRQLRLQRPTP |
| | GGAGGAGGATAGCGACTATAATCCAGCTGAGGATGAG | STPRPRRRPGRPR |
| | CCCCGAGGCCGGCAGCTTCGGCTCCAGCGCCCCACC | KLPRLEISDLPDGV |
| | CCCAGTACCCCAAGGCCCCGAAGGAGACCTGGCCGG | EGEPLVSSQSGQS |
| | CCCCGGAAGCTGCCCCGCCTGGAGATCTCAGACCTCC | PPEPQDPEAPSSS |
| | CAGATGGTGTGGAAGGAGAGCCTCTAGTGAGTTCCCA | GPGHLVAMGKVSR |
| | GAGTGGACAGAGCCCTCCAGAGCCACAGGATCCCGA | TPVEAGVSQSDAE |
| | GGCTCCCAGCTCCTCAGGCCCAGGACACCTGGTGGC | NAAPSCPDEHDTL |
| | CATGGGCAAGGTGAGCAGGACCCCTGTGGAAGCTGG | PRRRGRPSRRFLG |
| | TGTGAGCCAGTCAGATGCAGAGAACGCAGCCCCCTCC | KKYRKYYYKSPKP |
| | TGCCCGGATGAGCATGACACTCTGCCCCGGCGCCGA | LLRPFLCRICGSRF |
| | GGTCGACCTTCCAGGCGCTTCCTAGGCAAGAAATACC | LSHEDLRFHVNSH |
| | GCAAGTACTATTACAAGTCGCCCAAACCACTTTTGAGG | EAGDPQLFKCLQC |
| | CCCTTCCTGTGCCGCATCTGTGGTTCTCGCTTTCTGTC | SYRSRRWSSLKEH |
| | CCACGAGGACCTGCGCTTCCACGTCAACTCCCATGAG | MFNHVGSKPYKCD |
| | GCTGGCGATCCCCAGCTCTTCAAGTGCCTGCAGTGCA | ECSYTSVYRKDVIR |
| | GCTATCGTTCCCGCCGCTGGTCCTCGCTCAAGGAGCA | HAAVHSRDRKKRP |
| | CATGTTCAACCACGTGGGCAGCAAGCCCTACAAGTGT | DPTPKLSSFPCPV |
| | GACGAGTGCAGCTACACCAGTGTCTACCGGAAGGACG | CGRVYPMQKRLTQ |
| | TCATTCGGCACGCCGCTGTGCACAGCCGGGACCGGA | HMKTHSTEKPHMC |
| | AGAAGAGGCCAGATCCGACTCCAAAGCTGAGCTCTTT | DKCGKSFKKRYTF |
| | CCCCTGCCCTGTGTGTGGCCGTGTGTACCCCATGCAG | KMHLLTHIQAVANR |
| | AAAAGACTCACGCAGCACATGAAGACGCACAGCACTG | RFKCEFCEFVCED |
| | AGAAGCCCCACATGTGTGACAAGTGTGGAAAGTCCTTT | KKALLNHQLSHVS |
| | AAGAAGCGCTACACCTTCAAAATGCACCTGCTCACGCA | DKPFKCSFCPYRT |
| | CATCCAGGCTGTTGCCAACCGCAGGTTCAAGTGTGAG | FREDFLLSHVAVK |
| | TTCTGTGAGTTTGTTTGTGAAGACAAGAAGGCACTGCT | HTGAKPFACEYCH |
| | GAACCACCAGTTGTCCCACGTCAGTGACAAGCCCTTC | FSTRHKKNLRLHV |
| | AAATGCAGCTTTTGTCCCTACCGCACCTTCCGAGAGGA | RCRHASSFEEWG |
| | CTTCTTGCTGTCCCATGTGGCTGTCAAGCACACAGGG | RRHPEEPPSRRRP |
| | GCCAAGCCCTTCGCCTGTGAGTACTGCCACTTCAGCA | FFSLQQIEELKQQH |
| | CACGGCACAAGAAGAACCTGCGCCTGCACGTACGGTG | SAAPGPPPSSPGP |
| | CCGACACGCAAGCAGCTTCGAGGAATGGGGGAGGCG | PEIPPEATTFQSSE |
| | CCACCCTGAGGAGCCCCCCTCCCGCCGTCGCCCCTTC | APSLLCSDTLGGA |
| | TTCTCTCTGCAGCAGATTGAGGAGCTGAAGCAGCAGC | TIIYQQGAEESTAM |
| | ACAGTGCGGCCCCTGGACCACCTCCCAGTTCCCCAGG | ATQTALDLLLNMSA |
| | ACCTCCTGAGATACCCCCAGAGGCGACAACTTTCCAG | QRELGGTALQVAV |
| | TCATCTGAGGCTCCCTCATTGCTCTGTTCTGACACCCT | VKSEDVEAGLASP |
| | GGGCGGCGCCACCATCATCTACCAGCAAGGAGCTGA | GGQPSPEGATPQV |
| | GGAGTCGACAGCGATGGCCACGCAGACAGCCTTGGAT | VTLHVAEPGGGAA |
| | CTTCTGCTGAACATGAGTGCTCAGCGGGAACTGGGGG | AESQLGPPDLPQIT |
| | GCACAGCCCTGCAGGTGGCTGTGGTGAAGTCGGAAG | LAPGPFGGTGYSVI |
| | ATGTGGAAGCAGGGTTAGCATCCCCTGGTGGGCAGCC | TAPPMEEGTSAPG |
| | CTCCCCTGAAGGTGCCACTCCACAGGTGGTCACCCTC | TPYSEEPAGEAAQ |
| | CACGTGGCAGAGCCAGGGGGGGGTGCAGCAGCCGAG | AVVVSDTLKEAGT |
| | AGCCAGCTAGGCCCTCCTGACCTACCGCAGATCACCC | HYIMATDGTQLHHI |
| | TGGCACCTGGTCCATTTGGTGGGACTGGCTACAGTGT | ELTADGSISFPSPD |
| | CATCACAGCACCCCCTATGGAGGAGGGAACATCAGCT | ALASGAKWPLLQC |
| | CCTGGCACACCTTACAGCGAGGAGCCCGCAGGAGAG | GGLPRDGPEPPSP |
| | GCAGCCCAGGCTGTGGTTGTGAGTGACACCCTAAAAG | AKTHCVGDSQSSA |
| | AAGCTGGCACCCACTACATCATGGCTACTGATGGTAC | SSPPATSKALGLAV |
| | CCAGTTGCACCACATTGAGCTCACCGCAGATGGCTCC | PPSPPSAATAASK |
| | ATCTCCTTCCCAAGTCCAGATGCTCTGGCCTCTGGTGC | KFSCKICAEAFPGR |
| | CAAATGGCCCCTGCTGCAGTGTGGGGGACTGCCCAGA | AEMESHKRAHAGP |
| | GACGGCCCTGAGCCCCCATCTCCAGCCAAGACCCACT | GAFKCPDCPFSAR |
| | GCGTAGGGGACTCCCAGAGCTCTGCCTCCTCACCTCC | QWPEVRAHMAQH |
| | TGCAACCAGCAAAGCCCTGGGCCTGGCAGTGCCCCC | SSLRPHQCSQCSF |
| | GTCACCGCCATCTGCAGCCACTGCTGCATCAAAGAAG | ASKNKKDLRRHML |
| | TTTTCCTGCAAGATCTGTGCCGAGGCCTTCCCTGGCC | THTKEKPFACHLC |
| | GAGCTGAGATGGAGAGTCACAAGCGGGCCCACGCTG | GQRFNRNGHLKFH |
| | GGCCTGGTGCCTTCAAGTGCCCCGACTGCCCCTTCAG | IQRLHSPDGRKSG |
| | TGCCCGCCAGTGGCCCGAGGTCCGGGCGCACATGGC | TPTARAPTQTPTQ |
| | ACAGCACTCAAGCCTACGGCCCCACCAGTGTAGCCAG | TIILNSDDETLATLH |
| | TGCAGCTTTGCCTCCAAGAACAAGAAGGACCTGCGTC | TALQSSHGVLGPE |
| | GGCACATGCTGACTCACACAAAGGAGAAGCCTTTTGC | RLQQALSQEHIIVA |
| | ATGCCACCTCTGCGGGCAGCGTTTCAACCGTAACGGG | QEQTVTNQEEAAY |
| | CACCTCAAGTTCCACATCCAGCGGCTGCACAGTCCTG | IQEITTADGQTVQH |
| | ATGGGAGGAAGTCAGGAACCCCTACAGCCCGGGCCC | LVTSDNQVQYIISQ |
| | CTACCCAGACCCCAACCCAGACCATCATCCTGAACAG | DGVQHLLPQEYVV |
| | TGATGACGAAACACTGGCCACCCTGCACACTGCACTC | VPEGHHIQVQEGQ |
| | CAGTCCAGTCACGGGGTCCTGGGCCCAGAGCGGCTA | ITHIQYEQGAPFLQ |
| | CAGCAGGCACTGAGCCAGGAACACATCATCGTTGCCC | ESQIQYVPVSPGQ |
| | AGGAACAGACAGTGACCAATCAGGAGGAAGCCGCCTA | QLVTQAQLEAAAH |
| | CATCCAAGAGATCACCACGGCAGATGGCCAGACCGTA | SAVTAVADAAMAQ |
| | CAGCACCTGGTGACCTCCGACAACCAGGTGCAGTATA | AQGLFGTDETVPE |
| | TCATCTCCCAGGATGGTGTCCAGCACCTGCTCCCCCA | HIQQLQHQGIEYDV |
| | GGAATATGTTGTGGTCCCTGAAGGCCATCACATCCAG | ITLADD |
| | GTACAGGAGGGCCAGATCACACACATCCAGTATGAAC | (SEQ ID NO: 400) |
| | AAGGAGCCCCGTTCCTTCAGGAGTCCCAGATCCAGTA | |
| | TGTGCCTGTGTCCCCAGGCCAGCAGCTTGTCACACAG | |
| | GCTCAACTTGAGGCTGCAGCACACTCAGCTGTCACAG | |
| | CAGTGGCTGATGCTGCCATGGCCCAAGCCCAGGGCCT | |
| | GTTTGGTACAGACGAGACAGTGCCCGAACACATTCAA | |
| | CAGCTGCAGCACCAGGGCATCGAGTACGACGTCATCA | |
| | CCCTGGCCGATGACTGA | |
| | (SEQ ID NO: 367) | |
| ZNF367 | ATGATCCGGGGCTTCGAGGCGCCCATGGCGGAGAAC | MIRGFEAPMAENP |
| | CCGCCGCCGCCGCCGCCGCCCGTCATCTTCTGCCAC | PPPPPPVIFCHDSP |
| | GACTCCCCGAAGCGGGTGCTGGTGTCGGTCATCAGGA | KRVLVSVIRTTPIKP |
| | CGACCCCGATCAAGCCAACGTGCGGCGGTGGAGGGG | TCGGGGEPEPPPP |
| | AGCCGGAGCCGCCGCCGCCGCTCATCCCCACCAGCC | LIPTSPGFSDFMVY |
| | CCGGCTTCAGCGACTTCATGGTGTACCCGTGGCGCTG | PWRWGENAHNVT |
| | GGGCGAGAACGCACACAACGTGACGCTCAGCCCTGG | LSPGAAGAAASAA |
| | GGCCGCGGGGGCCGCCGCCTCGGCCGCCCTGCCTG | LPAAAAAEHSGLR |
| | CAGCCGCAGCCGCCGAGCACTCGGGGCTTCGTGGCC | GRGAPPPAASASA |
| | GGGGCGCGCCCCCGCCCGCCGCCTCGGCCTCCGCC | AASGGEDEEEASS |
| | GCCGCCTCGGGAGGTGAGGACGAGGAGGAAGCGAGC | PDSGHLKDGIRRG |
| | AGCCCAGACAGCGGCCACCTCAAGGATGGAATCCGAC | RPRADTVRDLINE |
| | GTGGTAGGCCCAGAGCAGATACTGTCCGCGATTTAAT | GEHSSSRIRCNICN |
| | AAATGAAGGAGAGCATTCATCCAGCAGAATCCGTTGTA | RVFPREKSLQAHK |
| | ACATCTGTAATAGGGTGTTTCCACGGGAGAAATCGCTC | RTHTGERPYLCDY |
| | CAGGCTCACAAAAGGACTCATACAGGTGAGAGGCCCT | PDCGKAFVQSGQL |
| | ATCTGTGTGACTATCCAGACTGTGGAAAAGCCTTTGTT | KTHQRLHTGEKPF |
| | CAAAGTGGACAGCTCAAAACACATCAGCGTCTTCACAC | VCSENGCLSRFTH |
| | CGGAGAGAAACCTTTTGTTTGTTCAGAAAATGGCTGCC | ANRHCPKHPYARL |
| | TGAGCAGATTCACCCATGCAAACCGCCACTGTCCGAA | KREEPTDTLSKHQ |
| | GCACCCCTACGCCAGGCTGAAGAGAGAGGAGCCCAC | AADNKAAAEWLAR |
| | GGACACACTCAGCAAACATCAGGCTGCCGACAACAAG | YWEMREQRTPTLK |
| | GCCGCGGCCGAGTGGCTGGCGAGGTATTGGGAAATG | GKLVQKADQEQQ |
| | AGAGAGCAGCGCACCCCCACTTTGAAAGGCAAGCTGG | DPLEYLQSDEEDD |
| | TTCAGAAGGCTGATCAGGAGCAGCAGGACCCTCTGGA | EKRGAQRRLQEQ |
| | ATACCTTCAGTCTGATGAAGAGGACGACGAGAAGAGA | RERLHGALALIELA |
| | GGGGCCCAGCGCCGGCTGCAGGAGCAGCGGGAGCG | NLTGAPLRQ |
| | CCTGCATGGAGCCCTCGCGCTCATAGAGCTTGCCAAC | (SEQ ID NO: 401) |
| | CTGACTGGGGCGCCACTCCGACAGTAG | |
| | (SEQ ID NO: 368) | |
| ZNF626 | ATGGGACCATTGCAATTTAGAGATGTGGCCATAGAATT | MGPLQFRDVAIEF |
| | CTCTCTGGAGGAGTGGCATTGCCTGGACACTGCACAG | SLEEWHQLDTAQR |
| | CGGAATTTATATAGGAATGTGATGTTAGAGAACTACAG | NLYRNVMLENYSN |
| | TAACCTGGTCTTCCTTGGTATTACTGTTTCTAAGCCAG | LVFLGITVSKPDLIT |
| | ACCTGATCACCTGTCTGGAGCAAGGAAGAAAACCTTTG | CLEQGRKPLTMKR |
| | ACCATGAAGAGAAATGAGATGATAGCCAAACCCTCAGT | NEMIAKPSVMCSH |
| | AATGTGTTCTCATTTTGCCCAAGACCTTTGGCCAGAGC | FAQDLWPEQSMK |
| | AGAGCATGAAAGATTCTTTCCAAAAAGTGGTACTGAGA | DSFQKVVLRRYEK |
| | AGATATGAAAAATGTGAACATGACAATTTACAGTTAAAA | CEHDNLQLKKGCI |
| | AAAGGATGTATAAGTGTGGATGAGTGTAAGGTGCACAA | SVDECKVHKEGYN |
| | AGAAGGTTATAATGAACTTAACCAATGTTTGACAACTAC | ELNQCLTTTPRKIC |
| | CCCAAGAAAAATATGTCAATGTGATAAATATGTGAAAG | QCDKYVKVLHQFP |
| | TCCTTCATCAATTTCCAAATTCAAACGGACAAAAGAGA | NSNGQKRGHTGK |
| | GGACATACTGGGAAAAAACCTTTCAAATATATAGAATG | KPFKYIECGKAFKQ |
| | TGGCAAAGCTTTTAAGCAGTTCTCAACTCTTACTACAC | FSTLTTHKKIHTGG |
| | ATAAGAAAATTCATACTGGAGGGAAACCCTACAAATGT | KPYKCEECGKAFN |
| | GAAGAATGTGGCAAAGCCTTTAACCACTCTTGTAGCCT | HSCSLTRHKKIHTG |
| | TACTAGACATAAGAAAATTCATACTGGAGAGAAACCCT | EKPYKCEECGKAF |
| | ACAAATGTGAAGAATGTGGCAAAGCCTTTAAGCACTCC | KHSSTLTTHKRNH |
| | TCCACTCTTACTACACATAAGAGAAATCATACTGGAGA | TGEKPYKCDKCGK |
| | GAAACCCTACAAGTGTGATAAATGTGGCAAAGCCTTTA | AFMSSSTLSKHEII |
| | TGTCATCCTCAACCCTTAGTAAACATGAGATAATTCATA | HTEKKPYKCEECG |
| | CGGAAAAGAAACCCTACAAATGTGAAGAATGTGGCAAA | KAFNRSSTLTTHKII |
| | GCCTTCAACCGGTCCTCAACCCTTACTACACATAAGAT | HTGEKPYKCEECD |
| | AATTCATACTGGAGAAAAACCCTACAAATGTGAAGAAT | KAFKYSYTLTTHKR |
| | GTGACAAAGCCTTTAAGTACTCCTATACCCTTACTACA | IHTEDKPYKCEEC |
| | CATAAAAGAATTCATACTGAAGACAAACCCTACAAATG | GKAFKYSSTLTTHK |
| | TGAAGAATGTGGCAAAGCCTTTAAGTACTCCTCTACCC | RIHTGEKPYKCEE |
| | TTACTACACATAAGAGAATTCATACTGGAGAGAAACCC | CGKAFKRSSDLTT |
| | TACAAATGTGAAGAATGTGGCAAAGCCTTCAAGCGGTC | HKIIHTGEKPYKCE |
| | TTCAGACCTTACTACGCATAAGATAATTCATACTGGAG | ECGKAFKYSSNLT |
| | AGAAACCCTACAAATGTGAAGAATGTGGCAAAGCTTTT | THKKIHTGERPYKC |
| | AAGTACTCCTCTAACCTTACTACACATAAGAAAATTCAT | EECGKAFNQSSILT |
| | ACTGGAGAGAGACCCTACAAATGTGAAGAATGTGGCA | THRRIHTGEKFYKC |
| | AAGCCTTCAACCAGTCCTCAATCCTTACTACACATAGG | EECGKAFKCSSNL |
| | AGAATTCATACTGGAGAGAAATTCTACAAATGTGAAGA | TTHKKIHTGERPYK |
| | ATGTGGCAAAGCTTTTAAGTGCTCCTCTAACCTTACTA | CEECGKAFNQSSIL |
| | CACATAAGAAAATTCATACTGGAGAGAGACCCTACAAA | TTHERIILERNSTN |
| | TGTGAAGAATGTGGCAAAGCCTTCAACCAGTCCTCAAT | VKNVAKPSSGPHT |
| | CCTTACTACACATGAGAGAATCATACTGGAGAGAAATT | LLHIR |
| | CTACAAATGTGAAGAATGTGGCAAAGCCTTCAAGCGGT | (SEQ ID NO: 402) |
| | CCTCACACCTTACTACACATAAGATAA | |
| | (SEQ ID NO: 369) | |

T cells may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be modulated by about 5-95%, 10-90%, 15-85%, 20-80%, or 1.5-fold to 10-fold, relative to a control. T cells may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be reduced by about 5-95%, 10-90%, 15-85%, 20-80%, or 1.5-fold to 10-fold, relative to a control. T cells may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. T cells may be increased by about 5-95%, 10-90%, 15-85%, 20-80%, or 1.5-fold to 10-fold, relative to a control.

In some embodiments, the agent may be used in combination with a modulator of BATF3. The composition comprising the modulator of T cells may further include a modulator of BATF3. Further provided is a therapy including a first composition comprising the modulator of T cells and a second composition comprising a modulator of BATF3. The first composition may target a gene selected from ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory element thereof, as detailed above. The first and second compositions may be administered at the same time or sequentially. The second composition may be administered before or after or concomitantly with the first composition. The second composition may be administered at least about 1 minute, at least about 2 minutes, at least about 5 minutes, at least about 10 minutes, at least about 30 minutes, at least about 1 hour, at least about 2 hours, at least about 6 hours, at least about 12 hours, at least about 24 hours, at least about 36 hours, or at least about 48 hours before or after the first composition is administered. The modulator of BATF3 may reduce, inhibit, decrease, increase, activate, or enhance the expression or activity of the BATF3 gene or its gene protein product. In some embodiments, the modulator of BATF3 comprises an activator of the BATF3 gene or the BATF3 gene protein product. The activator of the BATF3 gene may comprise, for example, a polynucleotide encoding BATF3, or a BATF3 polypeptide, or a DNA targeting composition as detailed herein wherein the second polypeptide domain has, for example, transcription activation activity, or a combination thereof. BATF3 is described in, for example, WO 2023/164671, which is incorporated herein by reference in its entirety.

In some embodiments, the modulator of T cells is administered with or as a cancer therapy. The cancer therapy may include chemotherapy or immunotherapy. The cancer therapy may include adoptive T cell therapy (ACT) therapy. The cancer therapy may include a chimeric antigen receptor (CAR). A chimeric antigen receptor (CAR) may also be known as chimeric immunoreceptor, chimeric T cell receptor, or artificial T cell receptor. CARs are receptor proteins that have been engineered to give T cells the new ability to target a specific antigen. CARs are chimeric in that they may combine both antigen-binding and T cell activating functions into a single receptor. CARs may include an antigen binding domain specific for an antigen on a cancer cell. The premise of CAR-T immunotherapy is to modify T cells to recognize cancer cells in order to target and destroy them. T cells are harvested from a subject, the T cells are genetically altered to add a chimeric antigen receptor (CAR) that specifically recognizes cancer cells, and the resulting CAR-T cells may be administered to the subject to attack their tumors. CAR-T cell therapy and modification to T cells are described in, for example, WO2012/079000 and WO2012/129514 and WO2018/005712, each of which is incorporated herein by reference in its entirety. In some embodiments, the modulator of T cells is administered concurrently with a cancer therapy, or subsequent to a cancer therapy, or prior to a cancer therapy, or as a cancer therapy.

a. Inhibitory Polynucleotides

In some embodiments, the agent comprises an inhibitory polynucleotide. Inhibitors comprising polynucleotides may be referred to as inhibitory nucleic acids. Polynucleotides may include, for example, antisense oligonucleotides (ASOs) or polynucleotides, ribozymes, short hairpin RNA (shRNA), siRNA, single-stranded or double-stranded RNA interference (RNAi), modified bases/locked nucleic acids (LNAs), peptide nucleic acids (PNAs), and/or other oligomeric or oligonucleotides. See, for example, inhibitory nucleic acids disclosed in U.S. Patent Publication No. 2020/0216549, incorporated herein by reference. The polynucleotide may hybridize to at least a portion of a target nucleic acid, such as a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory portion thereof, or a transcribed portion thereof. Binding of the polynucleotide to the target nucleic acid may inhibit the function of the target nucleic acid.

In some embodiments, the polynucleotide is an antisense polynucleotide. Antisense polynucleotides may also be referred to as antisense oligonucleotides. Antisense polynucleotides are typically designed to block expression of a DNA or RNA target by binding to the target and halting expression at the level of transcription, translation, or splicing. Antisense polynucleotides are complementary nucleic acid sequences designed to hybridize under stringent conditions to an RNA. Polynucleotides may be chosen that are sufficiently complementary to the target in that they hybridize sufficiently well and with sufficient specificity to give the desired effect.

In some embodiments, the polynucleotide complementary to a target RNA is an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (for example, each strand comprises a nucleotide sequence that is complementary to a nucleotide sequence in the other strand, such as where the antisense strand and the sense strand form a duplex or double stranded structure); the antisense strand comprises a nucleotide sequence that is complementary to a nucleotide sequence in a target nucleic acid molecule or a portion thereof, and the sense strand comprises nucleotide sequence corresponding to the target nucleic acid sequence or a portion thereof. As another example, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin, or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region has a nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof, and the sense region has a nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region, and a loop region. Such an RNA molecule when expressed desirably forms a “hairpin” structure and may be referred to as an “shRNA.” The loop region may generally be between about 2 and about 10 nucleotides in length, or from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. See, for example, Brummelkamp et al. Science 2002, 296, 550-553; Lee et al. Nature Biotechnol. 2002, 20, 500-505; Miyagishi and Taira, Nature Biotechnol. 2002, 20, 497-500; Paddison et al. Genes & Dev. 2002, 16, 948-958; Paul, Nature Biotechnol, 2002, 20, 505-508; Sui, PNAS 2002, 99, 5515-5520; Yu et al. PNAS 2002, 99, 6047-6052.

The target RNA cleavage reaction guided by siRNAs may be highly sequence specific. In general, a siRNA containing a nucleotide sequence identical to a portion of the target nucleic acid may be preferred for inhibition. However, 100% sequence identity between the siRNA and the target gene may not be required. Sequence variations due to genetic mutation, strain polymorphism, or evolutionary divergence, for example, may be tolerated. For example, siRNA sequences with insertions, deletions, and single point mutations relative to the target sequence may be effective for inhibition. siRNA sequences with nucleotide analog substitutions or insertions may be effective for inhibition. siRNAs may retain specificity for their target, that is, they may not directly bind to, or directly significantly affect expression levels of, transcripts other than the intended target. In some embodiments, the agent comprises siRNA. In some embodiments, the agent comprises shRNA.

In some embodiments, the inhibitor is a ribozyme. Trans-cleaving enzymatic nucleic acid molecules such as ribozymes can be used and have shown promise as therapeutic agents for human disease (Usman & McSwiggen, Ann. Rep. Med. Chem. 1995, 30, 285-294; Christoffersen and Marr. J. Med. Chem. 1995, 38, 2023-2037). Enzymatic nucleic acid molecules can be designed to cleave specific RNA targets within the background of cellular RNA. Such a cleavage event can render the RNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA may destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, Proc. R. Soc. London, B 1979, 205, 435) have been used to evolve new nucleic acid catalysts capable of catalyzing a variety of reactions, such as cleavage and ligation of phosphodiester linkages and amide linkages (Joyce, Gene 1989, 82, 83-87; Beaudry et al. Science 1992, 257, 635-641; Joyce, Scientific American 1992, 267, 90-97; Breaker et al. TIBTECH 1994, 12, 268; Bartel et al. Science 1993, 261, 1411-1418; Szostak, TIBS 1993, 17, 89-93; Kumar et al. FASEB J. 1995, 9, 1183; Breaker, Curr. Op. Biotech. 1996, 1, 442). Ribozymes may be developed to optimize catalytic activity and contribute to any strategy that employs RNA-cleaving ribozymes for the purpose of regulating gene expression, such as, for example, the hammerhead ribozyme, modified hammerhead ribozymes, and other artificial “RNA ligase” ribozymes.

In some embodiments, the polynucleotide is modified. For example, the polynucleotide may be modified to include one or more modified bonds or bases. A number of modified bases may include phosphorothioate, methylphosphonate, peptide nucleic acids, or locked nucleic acid (LNA) molecules. A polynucleotide may be fully modified, while others may be chimeric and contain two or more chemically distinct regions, each made up of at least one nucleotide. These inhibitory nucleic acids may contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids may be formed as composite structures of two or more oligonucleotides, modified oligonucleotides, oligonucleosides, and/or oligonucleotide mimetics as described above. Such chimeric inhibitory nucleic acids may be referred to as hybrids or gapmers. In some embodiments, the polynucleotide is a gapmer, which contains a central stretch (gap) of DNA monomers sufficiently long to induce RNase H cleavage, flanked by blocks of LNA modified nucleotides (see, for example, Stanton et al. Nucleic Acid Ther. 2012, 22, 344-359; Nowotny et al. Cell, 2005, 121, 1005-1016; Kurreck, European Journal of Biochemistry 2003, 270, 1628-1644; Fluiter et al., Mol. Biosyst. 2009, 5, 838-843; incorporated herein by reference). In some embodiments, the polynucleotide is a mixmer, which includes alternating short stretches of LNA and DNA (see, for example, Naguibneva et al., Biomed Pharmacother. 2006, 60, 633-638; Orom et al. Gene 2006, 372, 137-141; incorporated herein by reference). Representative United States patents that disclose the preparation of such hybrid structures may include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is incorporated herein by reference.

In some embodiments, the modified polynucleotide comprises at least one nucleotide modified at the 2′ position of the sugar, such as a 2′-O-alkyl, 2′-O-alkyl-O-alkyl, or 2′-fluoro-modified nucleotide. In other embodiments, RNA modifications include 2′-fluoro, 2′-amino, and 2′ O-methyl modifications on the ribose of pyrimidines, abasic residues, or an inverted base at the 3′ end of the RNA. Such modifications are routinely incorporated into oligonucleotides, and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide. These modified polynucleotides may survive intact for a longer period of time than unmodified polynucleotides. Specific examples of modified polynucleotides may include those comprising modified backbones, for example, phosphorothioates, phosphotriesters, methyl phosphonates, short chain alkyl or cycloalkyl intersugar linkages, or short chain heteroatomic or heterocyclic intersugar linkages. Modified polynucleotides may also include phosphorothioate backbones and those with heteroatom backbones, particularly CH2-NH—O—CH2, CH, —N(CH3)-O-CH2 (known as a methylene(methylimino) or MMI backbone), CH2-O—N(CH3)-CH2, CH2-N(CH3)-N(CH3)-CH2, and O—N(CH3)-CH2-CH2 backbones, wherein the native phosphodiester backbone is represented as O—P—O—CH); amide backbones (see, for example, De Mesmaeker et al. Ace. Chem. Res. 1995, 28, 366-374); morpholino backbone structures (see, for example, Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone; see, for example, Nielsen et al., Science 1991, 254, 1497), all references incorporated herein by reference. Phosphorus-containing linkages may include, but are not limited to, phosphorothioates, chiral phosphorothioates, phosphorodithioates, phosphotriesters, aminoalkylphosphotriesters, methyl, and other alkyl phosphonates comprising 3′-alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5-3′ or 2′-5′ to 5′-2′ (see, for example, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050, incorporated herein by reference). Morpholino-based oligomeric compounds are described in Dwaine A. Braasch and David R. Corey, Biochemistry 2002, 41, 4503-4510); Genesis, volume 30, issue 3, 2001; Heasman, J., Dev. Biol. 2002, 243, 209-214; Nasevicius et al. Nat. Genet. 2000, 26, 216-220; Lacerra et al. Proc. Natl. Acad. Sci. 2000, 97, 9591-9596; and U.S. Pat. No. 5,034,506, all incorporated herein by reference. Cyclohexenyl nucleic acid oligonucleotide mimetics are described in Wang et al. J. Am. Chem. Soc. 2000, 122, 8595-8602, incorporated herein by reference.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These may comprise those having morpholino linkages, formed in part from the sugar portion of a nucleoside; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, for example, one of the following at the 2′ position: OH, SH, SCH3, F, OCN, OCH3 OCH3, OCH3 O(CH2)n CH3, O(CH2)n NH2 or O(CH2)n CH3 where n is from 1 to about 10; C1 to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A modification may include 2′-methoxyethoxy [2′-O—CH2CH2OCH3, also known as 2′-O-(2-methoxyethyl)](Martin et al, Helv. Chim. Acta, 1995, 78, 486). Other modifications may include 2′-methoxy (2′-O—CH3), 2′-propoxy (2′-OCH2CH2CH3) and 2′-fluoro (2′-F). Similar modifications may also be made at other positions on the oligonucleotide, such as the 3′ position of the sugar on the 3′ terminal nucleotide and the 5′ position of 5′ terminal nucleotide. Oligonucleotides may also have sugar mimetics such as cyclobutyls in place of the pentofuranosyl group.

Polynucleotides can include, additionally or alternatively, one or more nucleobase modifications or substitutions. As used herein, “unmodified” or “natural” nucleobases comprise the purine bases adenine (A) and guanine (G), and the pyrimidine bases thymine (T), cytosine (C), and uracil (U). Modified nucleobases may include nucleobases found only infrequently or transiently in natural nucleic acids, such as hypoxanthine, 6-methyladenine, 5-Me pyrimidines, 5-methylcytosine (also referred to as 5-methyl-2′ deoxycytosine and 5-Me-C), 5-hydroxymethylcytosine (HMC), glycosyl HMC, gentobiosyl HMC. Modified nucleobases may also include synthetic nucleobases, such as 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, 2,6-diaminopurine, xanthine, hypoxanthine, 6-methyl and other alkyl derivatives of adenine and guanine, 2-propyl and other alkyl derivatives of adenine and guanine, 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine (see, for example, Kornberg, DNA Replication, W. H. Freeman & Co., San Francisco, 1980, pp 75-77; Gebeyehu, G., et al. Nucl. Acids Res. 1987, 15, 4513). A “universal” base known in the art, such as inosine, can also be included. 5-Me-C substitutions may also be included and have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278). Some nucleobases may be useful for increasing the binding affinity of the polynucleotides. These may include 5-substituted pyrimidines, 6-azapyrimidines, and N-2, N-6, and 0-6 substituted purines, comprising 2-aminopropyladenine, 5-propynyluracil, and 5-propynylcytosine. 5-methylcytosine substitutions may be combined with 2′-O-methoxyethyl sugar modifications.

It is not necessary for all positions in a given oligonucleotide to be uniformly modified. More than one of the aforementioned modifications may be incorporated in a single oligonucleotide or even at within a single nucleoside within an oligonucleotide.

In some embodiments, both a sugar and an internucleoside linkage, that is, the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include U.S. Pat. Nos. 5,539,082; 5,714,331; and 5,719,262, each of which is herein incorporated by reference. Further teaching of PNA compounds can be found in Nielsen et al. Science 1991, 254, 1497-1500, incorporated herein by reference. Nucleobases are further described in U.S. Pat. No. 3,687,808; ‘The Concise Encyclopedia of Polymer Science And Engineering’, pages 858-859, Kroschwitz, J. I., ed. John Wiley & Sons, 1990; Englisch et al., Angewandle Chemie, International Edition’, 1991, 30, page 613; Sanghvi, Y. S., Chapter 15, Antisense Research and Applications', pages 289-302, Crooke, S. T.; and Lebleu, B. ea., CRC Press, 1993. Modified nucleobases are also described in U.S. Pat. Nos. 3,687,808; 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692; and 5,681,941, each of which is herein incorporated by reference.

In some embodiments, the polynucleotide is chemically linked to one or more moieties or conjugates that enhance the activity, cellular distribution, or cellular uptake of the oligonucleotide. Such moieties may include but are not limited to, lipid moieties such as a cholesterol moiety (Letsinger et al. Proc. Natl. Acad. Sci. USA 1989, 86, 6553-6556), cholic acid (Manoharan et al., Bioorg. Med. Chem. Let., 1994, 4, 1053-1060), a thioether, such as hexyl-S-tritylthiol (Manoharan et al. Ann. N. Y Acad. Sci. 1992, 660, 306-309; Manoharan et al. Bioorg. Med. Chem. Let. 1993, 3, 2765-2770), a thiocholesterol (Oberhauser et al., Nucl. Acids Res. 1992, 20, 533-538), an aliphatic chain, such as dodecandiol or undecyl residues (Kabanov et al. FEBS Lett. 1990, 259, 327-330; Svinarchuk et al. Biochimie. 1993, 75, 49-54), a phospholipid such as di-hexadecyl-rac-glycerol or triethylammonium 1,2-di-O-hexadecyl-rac-glycero-3-H-phosphonate (Manoharan et al. Tetrahedron Lett. 1995, 36, 3651-3654; Shea et al. Nucl. Acids Res. 1990, 18, 3777-3783), a polyamine or a polyethylene glycol chain (Mancharan et al. Nucleosides & Nucleotides, 1995, 14, 969-973), or adamantane acetic acid (Manoharan et al., Tetrahedron Lett. 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther. 1996, 277, 923-937), incorporated herein by reference. See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928; and 5,688,941, each of which is herein incorporated by reference.

These moieties or conjugates can include conjugate groups covalently bound to functional groups such as primary or secondary hydroxyl groups. Conjugate groups may include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups may include cholesterols, lipids, phospholipids, biotin, phenazine, folate, phenanthridine, anthraquinone, acridine, fluoresceins, rhodamines, coumarins, and dyes. Groups that enhance the pharmacodynamic properties may include groups that improve uptake, enhance resistance to degradation, and/or strengthen sequence-specific hybridization with the target nucleic acid. Groups that enhance the pharmacokinetic properties may include groups that improve uptake, distribution, metabolism or excretion of the inhibitors. Representative conjugate groups are also disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether such as hexyl-5-tritylthiol, a thiocholesterol, an aliphatic chain such as dodecandiol or undecyl residues, a phospholipid such as di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety (see, for example, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941, incorporated herein by reference.

In some embodiments, the polynucleotides comprise locked nucleic acid (LNA) molecules, such as those [alpha]-L-LNAs. LNAs comprise ribonucleic acid analogues wherein the ribose ring is “locked” by a methylene bridge between the 2′-oxygen and the 4′-carbon, such as oligonucleotides containing at least one LNA monomer, that is, one 2′-0,4′-C-methylene-.beta.-D-ribofuranosyl nucleotide. LNA bases may form standard Watson-Crick base pairs but the locked configuration increases the rate and stability of the basepairing reaction (Jensen et al., Oligonucleotides, 2004, 14, 130-146, incorporated herein by reference). LNAs may also have increased affinity to base pair with RNA as compared to DNA. These properties may render LNAs especially useful as probes for fluorescence in situ hybridization (FISH) and comparative genomic hybridization, as knockdown tools for miRNAs, and as antisense oligonucleotides to target mRNAs or other RNAs such as the RNAs as described herein.

LNA molecules can include molecules comprising 10-30 nucleotides, or 12-24 nucleotides, such as 12, 13, 14, 15, 16, 17, 18, 19, 20, 21, 22, 23, 24, 25, 26, 27, 28, 29, or 30 nucleotides in each strand. One of the strands may be substantially identical to a target region in the RNA. One of the strands may be at least 80%, at least 85%, at least 90%, at least 95%, or 100% identical to a target region in the RNA. One of the strands may have 3, 2, 1, or 0 mismatched nucleotide(s) relative to a target region in the RNA. The LNA molecules can be chemically synthesized using methods known in the art.

LNA molecules can be designed using any method known in the art; a number of algorithms are known and are commercially available (for example see exiqon.com; You et al., Nuc. Acids. Res. 2006, 34, e60; McTigue et al., Biochemistry 2004, 43, 5388-5405; and Levin et al., Nuc. Acids. Res. 2006, 34, e14; incorporated herein by reference). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, such as gaps of 5-10 nucleotides or more, can be left between the LNAs to reduce the number of oligonucleotides synthesized and tested. GC content may be, for example, between about 30-60%. General guidelines for designing LNAs are known in the art; for example, LNA sequences may bind very tightly to other LNA sequences, so it may be preferable to avoid significant complementarity within an LNA. Contiguous runs of more than four LNA residues may be avoided where possible (for example, it may not be possible with very short (such as about 9-10 nt) oligonucleotides). In some embodiments, the LNAs are xylo-LNAs. For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 1998, 54, 3607-3630; Obika et al. Tetrahedron Lett. 1998, 39, 5401-5404; Jepsen et al. Oligonucleotides 2004, 14, 130-146; Kauppinen et al. Drug Disc. Today 2005, 2, 287-290; and Ponting et al. Cell 2009, 136, 629-641, and references cited therein, all incorporated by reference.

In some embodiments, the inhibitor comprises an antisense oligonucleotide, siRNA, RNAi, shRNA, LNA, and/or PNA. In some embodiments, the inhibitor comprises siRNA. In some embodiments, the inhibitor includes a polynucleotide comprising one or more of a modified internucleoside linkage, a modified sugar moiety, and/or a modified nucleobase as detailed herein.

b. DNA Targeting Systems

In some embodiments, the agent comprises a DNA targeting composition or at least one component thereof. A “DNA Targeting System” as used herein is a system capable of specifically targeting a particular region of DNA and modulating gene expression by binding to that region. Non-limiting examples of these systems are CRISPR-Cas-based systems, meganucleases, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a Cas protein and guide RNA, or a meganuclease, or a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA targeting composition comprises a meganuclease. A meganuclease is an endodeoxyribonuclease characterized by a large recognition site, such as double-stranded DNA sequences of 12 to 40 base pairs. The recognition site may occur only once in any given genome. A meganuclease may be a homing endonuclease selected from an intron endonuclease or an intein endonuclease. Meganucleases may include, for example, the LAGLIDADG family of homing endonucleases.

In some embodiments, the DNA-binding portion comprises a Cas protein, such as a Cas9 protein. Some CRISPR-Cas-based systems can operate to activate or repress expression using the Cas protein alone, not linked to an activator or repressor. For example, a nuclease-null Cas9 can act as a repressor on its own, a nuclease-active Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

### i) DNA Binding Protein

The DNA Targeting System may include a DNA binding protein. The DNA binding protein may comprise, for example, a zinc finger protein or a transcription activator-like effector (TALE). The zinc finger protein or TALE may target a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory element thereof.

### (1) Zinc Finger Protein

A zinc finger protein is a protein that includes one or more zinc finger domains. Zinc finger domains are relatively small protein motifs that contain multiple finger-like protrusions that make tandem contacts with their target molecule such as a DNA target molecule. A zinc finger domain may bind one or more zinc ions or other metal ion such as iron, or in some cases a zinc finger domain forms salt bridges to stabilize the finger-like folds. The zinc binding portion of a zinc finger protein may include one or more cysteine residues and/or one or more histidine residues to coordinate the zinc or other metal ion. A zinc finger protein recognizes and binds to a particular DNA sequence via the zinc finger domain. In some embodiments, a zinc finger protein is fused to or includes a nuclease domain and may be referred to as a zinc finger nuclease (ZFN). The nuclease domain may include, for example, the endonuclease Fokl. ZFNs may recognize target sites that consist of two zinc-finger binding sites that flank a 5- to 7-base pair (bp) spacer sequence recognized by the endonuclease Fokl cleavage domain.

### (2) Transcription Activator-like Effector (TALE)

A TALE is another type of protein that recognizes and binds to a particular DNA sequence. The DNA-binding domain of a TALE includes an array of tandem 33-35 amino acid repeats, also known as RVD modules. Each RVD module specifically recognizes a single base pair of DNA. RVD modules may be arranged in any order to assemble an array that recognizes a defined DNA sequence. The binding specificity of a TALE DNA-binding domain is determined by the RVD array followed by a single truncated repeat of, for example, 20 amino acids. A TALE DNA-binding domain may have an array of 12 to 27 RVD modules, each RVD module recognizing a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains may then be combined with catalytic domains to create functional enzymes, including artificial transcription factors and/or nucleases. In some embodiments, a TALE is fused to or includes a nuclease domain and may be referred to as a TALE nuclease (TALEN). The nuclease domain may include, for example, the endonuclease Fokl. TALENs may recognize target sites that consist of two TALE DNA-binding sites that flank a 12-bp to 20-bp spacer sequence recognized by the Fokl cleavage domain.

### (3) DNA Binding Fusion Protein

Additionally or alternatively, a zinc finger protein or TALE can be fused to a polypeptide domain and referred to as a DNA binding fusion protein or fusion protein. The fusion protein may act as a synthetic transcription factor. The fusion protein comprises two heterologous polypeptide domains, including a first polypeptide domain comprising the zinc finger protein or the TALE or a Cas9 protein as further detailed below, and a second polypeptide domain having an activity selected from transcription activation activity, transcription repression activity, nuclease activity, transcription release factor activity, histone modification activity, nucleic acid association activity, methylase activity, and demethylase activity. A zinc finger protein or TALE can be fused to a polypeptide domain having epigenetic modifying activity to mediate targeted gene regulation. A fusion protein comprising a zinc finger protein or TALE, and a second polypeptide domain having transcription repression activity, may mediate targeted gene repression. A fusion protein comprising a zinc finger protein or TALE, and a second polypeptide domain having transcription activation activity, may mediate targeted gene activation. The second polypeptide domain is further detailed below (see “Cas Fusion Protein”).

### ii) CRISPR/Cas-Based Gene Editing System

Provided herein are CRISPR/Cas-based gene editing systems. The CRISPR/Cas-based gene editing system may be used to modulate T cells and/or enhance ACT. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, Cas13, and Cascade proteins. Cas12a may also be referred to as “Cpf1.” Cas12a causes a staggered cut in double stranded DNA, while Cas9 produces a blunt cut. In some embodiments, the Cas protein comprises Cas12a. Cas12a is described in, for example, WO 2018/017754, which is incorporated herein by reference. Cas13 is an RNA-guided RNA endonuclease. Cas13 cleaves single-stranded RNA, and it does not cleave DNA. In some embodiments, the Cas protein comprises Cas13. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

iii) Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 26. In certain embodiments, the Cas9 molecule is a Staphylococcus aureus Cas9 molecule (also referred herein as “SaCas9”). SaCas9 may comprise an amino acid sequence of SEQ ID NO: 27.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 20).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 28. A S. pyogenes Cas9 protein with D10A and H849A mutations may comprise an amino acid sequence of SEQ ID NO: 29. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 30. In certain embodiments, the mutant S. aureus Cas9 molecule comprises a N580A mutation. The nucleotide sequence encoding this mutant S. aureus Cas9 molecule is set forth in SEQ ID NO: 31.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 32. Exemplary codon optimized nucleic acid sequences encoding a Cas9 molecule of S. aureus, and optionally containing nuclear localization sequences (NLSs), are set forth in SEQ ID NOs: 33-39. Another exemplary codon optimized nucleic acid sequence encoding a Cas9 molecule of S. aureus comprises the nucleotides 1293-4451 of SEQ ID NO: 40.

### iv) Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. The second polypeptide domain may have any DNA editing activity. The second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. In some embodiments, the fusion protein comprises more than one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser)n, wherein n is an integer between 0 and 10 (SEQ ID NO: 21). In a GS linker, n can be adjusted to optimize the linker length and achieve appropriate separation of the functional domains. Other examples of linkers may include, for example, Gly-Gly-Gly-Gly-Gly (SEQ ID NO: 22), Gly-Gly-Ala-Gly-Gly (SEQ ID NO: 23), Gly/Ser rich linkers such as Gly-Gly-Gly-Gly-Ser-Ser-Ser (SEQ ID NO: 24), or Gly/Ala rich linkers such as Gly-Gly-Gly-Gly-Ala-Ala-Ala (SEQ ID NO: 25).

In some embodiments, the agent and/or Cas protein and/or the Cas fusion protein and/or gRNAs detailed herein may be used in compositions and methods for modulating expression of gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

### (1) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 41 or SEQ ID NO: 42. In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 43, encoded by the polynucleotide of SEQ ID NO: 44. VPH may comprise a polypeptide having the amino acid sequence of SEQ ID NO: 53, encoded by the polynucleotide of SEQ ID NO: 54. VPR may comprise a polypeptide having the amino acid sequence of SEQ ID NO: 55, encoded by the polynucleotide of SEQ ID NO: 56.

### (2) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. KRAB may comprise a polypeptide having the amino acid sequence of SEQ ID NO: 45, encoded by a polynucleotide comprising the sequence of SEQ ID NO: 46. For example, the fusion protein may be S. pyogenes dCas9-KRAB (protein sequence comprising SEQ ID NO: 47; polynucleotide sequence comprising SEQ ID NO: 48). The fusion protein may be S. aureus dCas9-KRAB (protein sequence comprising SEQ ID NO: 49; polynucleotide sequence comprising SEQ ID NO: 50).

### (3) Transcription Release Factor Activity

The second polypeptide domain can have transcription release factor activity.

The second polypeptide domain can have eukaryotic release factor 1 (ERF1) activity or eukaryotic release factor 3 (ERF3) activity.

### (4) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 41 or SEQ ID NO: 42.

### (5) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease. In some embodiments, the second polypeptide domain includes a meganuclease, as detailed above. In some embodiments, the polypeptide domain having nuclease activity comprises Fokl.

### (6) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

### (7) Base Editing Activity

The second polypeptide domain may have base editing activity. Base editing enables the direct, irreversible conversion of a specific DNA base into another base at a targeted genomic locus without requiring double-stranded DNA breaks (DSB). A base editing domain has sequence requirements for activity. In a 20 nucleotide protospacer, the target base may be within 4-8 nucleotides from the PAM-distal end. An exemplary splice acceptor is an “AG” immediately before the exon, and an exemplary splice donor is a “GT” immediately following the exon. Cas9 molecules from different species may use different PAMs, and thereby provide some flexibility in selecting the base to edit. Disruption of canonical splice sites can lead to exon skipping or activation of cryptic splice sites. Both adenine and cytosine base editors may be capable of disrupting an “AG” splice acceptor, converting it to either a “GG” or “AA”, respectively. In some embodiments, the base-editing domain includes an adenine base editor (ABE). Adenine base editors may include, for example, ecTadA, including wild-type and mutants thereof. The adenine base editor may be as described in Gaudelli et al. (Nature 2017, 551, 464-471), Koblan et al. (Nature Biotech. 2018, 36, 843-846), Richter et al. (Nature Biotech. 2020, 38, 883-891), and Gaudelli et al. (Nature Biotech. 2020, 38, 892-900), each of which is incorporated herein by reference. The ABE may comprise a polypeptide selected from SEQ ID NOs: 57-64 and/or be encoded by a polynucleotide comprising a sequence selected from SEQ ID NOs: 65-72, respectively. In some embodiments, the base-editing domain includes a cytidine deaminase domain. A cytidine deaminase domain can convert the DNA base cytosine to uracil. In some embodiments, the cytidine deaminase domain can include an apolipoprotein B mRNA-editing enzyme, catalytic polypeptide-like (APOBEC) family deaminase. In some embodiments, the cytidine deaminase domain can include an APOBEC 1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, or a combination thereof. Base editing domains are detailed in, for example, WO 2020/210776 and WO 2022/081612, each of which is incorporated herein by reference.

### (8) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

### (9) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; amino acid sequence comprising SEQ ID NO: 51; polynucleotide sequence comprising SEQ ID NO: 52). In some embodiments, the second polypeptide domain has histone demethylase activity. In some embodiments, the second polypeptide domain has DNA demethylase activity.

v) Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target the Cas9 protein or fusion protein to a gene or a regulatory element thereof. The gRNA may target the Cas protein or fusion protein to a non-open chromatin region, an open chromatin region, a transcribed region of the target gene, a region upstream of a transcription start site of the target gene, a regulatory element of the target gene, an intron of the target gene, or an exon of the target gene, or a combination thereof. In some embodiments, the gRNA targets the Cas9 protein or fusion protein to a promoter of a gene. In some embodiments, the target region is located between about 1 to about 1000 base pairs upstream of a transcription start site of a target gene. In some embodiments, the DNA targeting composition comprises two or more gRNAs, each gRNA binding to a different target region.

The gRNA may target a region of a gene that modulates T cells. The gRNA may target a region of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory element thereof. The gRNA may target a region of a gene selected from ZNF217 or ETS1, or a regulatory element thereof. The gRNA may target a region of a gene selected from RBSN, PRDM1, GATA3, or RUNX3, or a regulatory element thereof. The gRNA may target a region of a gene selected from ZNF217, ETS1, RBSN, PRDM1, GATA3, RUNX3, FOXP1, GFI1, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1, or a regulatory element thereof. The gRNA may target a region of a gene selected from FOXO1, MDB2, YY1, DNMT1, IRF1, CTCF, GTF2B, or HIC1, or a regulatory element thereof. In some embodiments, the gRNA targets a gene and is used in combination with a Cas9 fusion protein wherein the second polypeptide domain has transcription activation activity, to activate or enhance expression of the gene to increase T cells. In some embodiments, the gRNA targets a gene and is used in combination with a Cas9 fusion protein wherein the second polypeptide domain has transcription repression activity, to inhibit or reduce or decrease expression of the gene to increase T cells. The gRNA may comprise a polynucleotide selected from at least one of SEQ ID NOs: 205-336, or a complement thereof, or a variant thereof, or a truncation thereof. The gRNA may be encoded by a polynucleotide sequence comprising at least one of SEQ ID NOs: 73-204, or a complement thereof, or a variant thereof, or a truncation thereof. The gRNA may bind and target a polynucleotide sequence comprising at least one of SEQ ID NOs: 73-204, or a complement thereof, or a variant thereof, or a truncation thereof. A truncation may be 1, 2, 3, 4, 5, 6, 7, 8, or 9 nucleotides shorter than the sequence of any one of SEQ ID NOs: 73-336. Exemplary gRNA sequences for modulating T cells are shown in TABLE 2. Exemplary gRNA sequences are also detailed in McCutcheon et al., Nat. Genet. 2023; 55(12): 2211-2223, which is incorporated herein by reference in its entirety.

TABLE 2
Genes and gRNA sequences for modulating T cells.

| Target | | |
|---|---|---|
| Gene_ID | DNA sequence for gRNA | gRNA |
| ZNF217_1 | GGACACATAATGGCAAATCG | GGACACAUAAUGGCAAAUCG |
| | (SEQ ID NO: 73) | (SEQ ID NO: 205) |
| ZNF217_2 | CAAAATCTCACCCTGAAACG | CAAAAUCUCACCCUGAAACG |
| | (SEQ ID NO: 74) | (SEQ ID NO: 206) |
| ZNF217_3 | CCACGGCGAAGCGCCCTCCG | CCACGGCGAAGCGCCCUCCG |
| | (SEQ ID NO: 75) | (SEQ ID NO: 207) |
| ZNF217_4 | TGGGTGGTACTGCCATCCGG | UGGGUGGUACUGCCAUCCGG |
| | (SEQ ID NO: 76) | (SEQ ID NO: 208) |
| ETS1_1 | GGTCTCGGAGAATGACCGAG | GGUCUCGGAGAAUGACCGAG |
| | (SEQ ID NO: 77) | (SEQ ID NO: 209) |
| ETS1_2 | CTTACTAATGAAGTAATCCG | CUUACUAAUGAAGUAAUCCG |
| | (SEQ ID NO: 78) | (SEQ ID NO: 210) |
| ETS1_3 | TGCATGGGGAGGACCAGTCG | UGCAUGGGGAGGACCAGUCG |
| | (SEQ ID NO: 79) | (SEQ ID NO: 211) |
| ETS1_4 | GAGAAAGCAGTCTTTACCCA | GAGAAAGCAGUCUUUACCCA |
| | (SEQ ID NO: 80) | (SEQ ID NO: 212) |
| RBSN_1 | TGCACATAATAGACCCGCAG | UGCACAUAAUAGACCCGCAG |
| | (SEQ ID NO: 81) | (SEQ ID NO: 213) |
| RBSN_2 | TAGAATTGACCACTATGTTG | UAGAAUUGACCACUAUGUUG |
| | (SEQ ID NO: 82) | (SEQ ID NO: 214) |
| RBSN_3 | GGCTTCTCGAGCGGCCAACG | GGCUUCUCGAGCGGCCAACG |
| | (SEQ ID NO: 83) | (SEQ ID NO: 215) |
| RBSN_4 | GAGTGCGCACTTCATCCATG | GAGUGCGCACUUCAUCCAUG |
| | (SEQ ID NO: 84) | (SEQ ID NO: 216) |
| PRDM1_1 | GGGGAGCGAGTGATGTACGT | GGGGAGCGAGUGAUGUACGU |
| | (SEQ ID NO: 85) | (SEQ ID NO: 217) |
| PRDM1_2 | GGACGCGTTCAAGTAAGCGT | GGACGCGUUCAAGUAAGCGU |
| | (SEQ ID NO: 86) | (SEQ ID NO: 218) |
| PRDM1_3 | TTTGGACAGATCTATTCCAG | UUUGGACAGAUCUAUUCCAG |
| | (SEQ ID NO: 87) | (SEQ ID NO: 219) |
| PRDM1_4 | AGGATGCGGATATGACTCTG | AGGAUGCGGAUAUGACUCUG |
| | (SEQ ID NO: 88) | (SEQ ID NO: 220) |
| GATA3_1 | TCCAAGACGTCCATCCACCA | UCCAAGACGUCCAUCCACCA |
| | (SEQ ID NO: 89) | (SEQ ID NO: 221) |
| GATA3_2 | AGGTACCCTCCGACCCACCA | AGGUACCCUCCGACCCACCA |
| | (SEQ ID NO: 90) | (SEQ ID NO: 222) |
| GATA3_3 | GGAGCTGTACTCGGGCACGT | GGAGCUGUACUCGGGCACGU |
| | (SEQ ID NO: 91) | (SEQ ID NO: 223) |
| GATA3_4 | CAGGGAGTGTGTGAACTGTG | CAGGGAGUGUGUGAACUGUG |
| | (SEQ ID NO: 92) | (SEQ ID NO: 224) |
| RUNX3_1 | CCCCAGGATGCATTATCCCG | CCCCAGGAUGCAUUAUCCCG |
| | (SEQ ID NO: 93) | (SEQ ID NO: 225) |
| RUNX3_2 | CACTGCGGCCCACGAAGCGA | CACUGCGGCCCACGAAGCGA |
| | (SEQ ID NO: 94) | (SEQ ID NO: 226) |
| RUNX3_3 | CCGTGCCGTACCTTGGATTG | CCGUGCCGUACCUUGGAUUG |
| | (SEQ ID NO: 95) | (SEQ ID NO: 227) |
| RUNX3_4 | CCGTGCCGTACCTTGGATTG | CCGUGCCGUACCUUGGAUUG |
| | (SEQ ID NO: 96) | (SEQ ID NO: 228) |
| FOXP1_1 | AGAGGAGGAGACACATGTCG | AGAGGAGGAGACACAUGUCG |
| | (SEQ ID NO: 97) | (SEQ ID NO: 229) |
| FOXP1_2 | GTTCTGTAGACTTCACATGC | GUUCUGUAGACUUCACAUGC |
| | (SEQ ID NO: 98) | (SEQ ID NO: 230) |
| FOXP1_3 | CATACACCATGTCCATAGAG | CAUACACCAUGUCCAUAGAG |
| | (SEQ ID NO: 99) | (SEQ ID NO: 231) |
| FOXP1_4 | GCCTTCTGACAATTCAGCCC | GCCUUCUGACAAUUCAGCCC |
| | (SEQ ID NO: 100) | (SEQ ID NO: 232) |
| GFI1_1 | CCGGTACATTCTCTAAACGG | CCGGUACAUUCUCUAAACGG |
| | (SEQ ID NO: 101) | (SEQ ID NO: 233) |
| GFI1_2 | TGGGCACATTGACTTCTCCG | UGGGCACAUUGACUUCUCCG |
| | (SEQ ID NO: 102) | (SEQ ID NO: 234) |
| GFI1_3 | TTCAAACCGTACTCATGGAG | UUCAAACCGUACUCAUGGAG |
| | (SEQ ID NO: 103) | (SEQ ID NO: 235) |
| GFI1_4 | ACGTGCACCTCGAGCCCGTG | ACGUGCACCUCGAGCCCGUG |
| | (SEQ ID NO: 104) | (SEQ ID NO: 236) |
| AHR_1 | TTATAACAGACTACTGTCTG | UUAUAACAGACUACUGUCUG |
| | (SEQ ID NO: 105) | (SEQ ID NO: 237) |
| AHR_2 | CTTACTTCGGATATGGGACT | CUUACUUCGGAUAUGGGACU |
| | (SEQ ID NO: 106) | (SEQ ID NO: 238) |
| AHR_3 | ATCGCAAACAAAGCCAACTG | AUCGCAAACAAAGCCAACUG |
| | (SEQ ID NO: 107) | (SEQ ID NO: 239) |
| AHR_4 | TACCACATCCACTCTAAGCA | UACCACAUCCACUCUAAGCA |
| | (SEQ ID NO: 108) | (SEQ ID NO: 240) |
| ZNF626_1 | TAACCTGGTCTTCCTTGGTG | UAACCUGGUCUUCCUUGGUG |
| | (SEQ ID NO: 109) | (SEQ ID NO: 241) |
| ZNF626_2 | ACCATTGCAATTTAGAGATG | ACCAUUGCAAUUUAGAGAUG |
| | (SEQ ID NO: 110) | (SEQ ID NO: 242) |
| ZNF626_3 | GGCCATAGAATTCTCTCTGG | GGCCAUAGAAUUCUCUCUGG |
| | (SEQ ID NO: 111) | (SEQ ID NO: 243) |
| ZNF626_4 | CTGATCACCTGTCTGGAGCA | CUGAUCACCUGUCUGGAGCA |
| | (SEQ ID NO: 112) | (SEQ ID NO: 244) |
| FOXP3_1 | GAGGGTGCCACCATGACTAG | GAGGGUGCCACCAUGACUAG |
| | (SEQ ID NO: 113) | (SEQ ID NO: 245) |
| FOXP3_2 | CCCACCCACAGGGATCAACG | CCCACCCACAGGGAUCAACG |
| | (SEQ ID NO: 114) | (SEQ ID NO: 246) |
| FOXP3_3 | ACCCAGGCATCATCCGACAA | ACCCAGGCAUCAUCCGACAA |
| | (SEQ ID NO: 115) | (SEQ ID NO: 247) |
| FOXP3_4 | CCTACTTAGGCACTGCCAGG | CCUACUUAGGCACUGCCAGG |
| | (SEQ ID NO: 116) | (SEQ ID NO: 248) |
| ARNT_1 | AGATGTCTAACGATAAGGAG | AGAUGUCUAACGAUAAGGAG |
| | (SEQ ID NO: 117) | (SEQ ID NO: 249) |
| ARNT_2 | CAAGCTAACCATCTTACGCA | CAAGCUAACCAUCUUACGCA |
| | (SEQ ID NO: 118) | (SEQ ID NO: 250) |
| ARNT_3 | TGGATTGTGTTGGAGAGTGT | UGGAUUGUGUUGGAGAGUGU |
| | (SEQ ID NO: 119) | (SEQ ID NO: 251) |
| ARNT_4 | TGGGGAACCTCACTTCGTGG | UGGGGAACCUCACUUCGUGG |
| | (SEQ ID NO: 120) | (SEQ ID NO: 252) |
| MYC_1 | CTTCGGGGAGACAACGACGG | CUUCGGGGAGACAACGACGG |
| | (SEQ ID NO: 121) | (SEQ ID NO: 253) |
| MYC_2 | AGAGTGCATCGACCCCTCGG | AGAGUGCAUCGACCCCUCGG |
| | (SEQ ID NO: 122) | (SEQ ID NO: 254) |
| MYC_3 | CTGCGGGGAGGACTCCGTCG | CUGCGGGGAGGACUCCGUCG |
| | (SEQ ID NO: 123) | (SEQ ID NO: 255) |
| MYC_4 | GCTGCACCGAGTCGTAGTCG | GCUGCACCGAGUCGUAGUCG |
| | (SEQ ID NO: 124) | (SEQ ID NO: 256) |
| SON_1 | GGACATCATAGAGCGCTCGT | GGACAUCAUAGAGCGCUCGU |
| | (SEQ ID NO: 125) | (SEQ ID NO: 257) |
| SON_2 | GGATTCCAGCGCTGTCGTCG | GGAUUCCAGCGCUGUCGUCG |
| | (SEQ ID NO: 126) | (SEQ ID NO: 258) |
| SON_3 | GCCAGTTGTAACAATGTCAG | GCCAGUUGUAACAAUGUCAG |
| | (SEQ ID NO: 127) | (SEQ ID NO: 259) |
| SON_4 | GGTTGAAGGACTGACACCGC | GGUUGAAGGACUGACACCGC |
| | (SEQ ID NO: 128) | (SEQ ID NO: 260) |
| TBX21_1 | TAAACTTGGACCACAACAGG | UAAACUUGGACCACAACAGG |
| | (SEQ ID NO: 129) | (SEQ ID NO: 261) |
| TBX21_2 | CCTGGGGTCTCCCTACCCGG | CCUGGGGUCUCCCUACCCGG |
| | (SEQ ID NO: 130) | (SEQ ID NO: 262) |
| TBX21_3 | GCGGTACCAGAGCGGCAAGT | GCGGUACCAGAGCGGCAAGU |
| | (SEQ ID NO: 131) | (SEQ ID NO: 263) |
| TBX21_4 | CCGGGGCTGGTACTTATGGA | CCGGGGCUGGUACUUAUGGA |
| | (SEQ ID NO: 132) | (SEQ ID NO: 264) |
| DUX4_1 | GGCAGGCGGCCTGTGCAGCG | GGCAGGCGGCCUGUGCAGCG |
| | (SEQ ID NO: 133) | (SEQ ID NO: 265) |
| DUX4_2 | GGGGATCTCCCAACCTGCCC | GGGGAUCUCCCAACCUGCCC |
| | (SEQ ID NO: 134) | (SEQ ID NO: 266) |
| DUX4_3 | TCCAGGCATCGCCGCCCGGG | UCCAGGCAUCGCCGCCCGGG |
| | (SEQ ID NO: 135) | (SEQ ID NO: 267) |
| DUX4_4 | ACACCGGCGCGTGGGGAACG | ACACCGGCGCGUGGGGAACG |
| | (SEQ ID NO: 136) | (SEQ ID NO: 268) |
| YBX1_1 | AGACGCTATCCACGTCGTAG | AGACGCUAUCCACGUCGUAG |
| | (SEQ ID NO: 137) | (SEQ ID NO: 269) |
| YBX1_2 | GTCTTGCAGGAATGACACCA | GUCUUGCAGGAAUGACACCA |
| | (SEQ ID NO: 138) | (SEQ ID NO: 270) |
| YBX1_3 | GCAAATGTTACAGGTCCTGG | GCAAAUGUUACAGGUCCUGG |
| | (SEQ ID NO: 139) | (SEQ ID NO: 271) |
| YBX1_4 | GGCGGGGACAAGAAGGTCAT | GGCGGGGACAAGAAGGUCAU |
| | (SEQ ID NO: 140) | (SEQ ID NO: 272) |
| ZC3H8_1 | ATTCATCAACCAACATACAG | AUUCAUCAACCAACAUACAG |
| | (SEQ ID NO: 141) | (SEQ ID NO: 273) |
| ZC3H8_2 | AGTTTGACTCTTACCCTGTG | AGUUUGACUCUUACCCUGUG |
| | (SEQ ID NO: 142) | (SEQ ID NO: 274) |
| ZC3H8_3 | AAGGATCAAATGCTTTGCTG | AAGGAUCAAAUGCUUUGCUG |
| | (SEQ ID NO: 143) | (SEQ ID NO: 275) |
| ZC3H8_4 | TCACCTTTCGTCAGAGTCCG | UCACCUUUCGUCAGAGUCCG |
| | (SEQ ID NO: 144) | (SEQ ID NO: 276) |
| ZNF367_1 | CAACAGGATGGAATCCGACG | CAACAGGAUGGAAUCCGACG |
| | (SEQ ID NO: 145) | (SEQ ID NO: 277) |
| ZNF367_2 | TCGTGGCAGAAGATGACGGG | UCGUGGCAGAAGAUGACGGG |
| | (SEQ ID NO: 146) | (SEQ ID NO: 278) |
| ZNF367_3 | GCCGCACGTTGGCTTGATCG | GCCGCACGUUGGCUUGAUCG |
| | (SEQ ID NO: 147) | (SEQ ID NO: 279) |
| ZNF367_4 | GCCTGGAGCGATTTCTCCCG | GCCUGGAGCGAUUUCUCCCG |
| | (SEQ ID NO: 148) | (SEQ ID NO: 280) |
| ZNF335_1 | GAAGCCCCACATGTGTGACA | GAAGCCCCACAUGUGUGACA |
| | (SEQ ID NO: 149) | (SEQ ID NO: 281) |
| ZNF335_2 | ACACAGACGATGAATCAGGG | ACACAGACGAUGAAUCAGGG |
| | (SEQ ID NO: 150) | (SEQ ID NO: 282) |
| ZNF335_3 | ACACGGTACCTGATCCTACA | ACACGGUACCUGAUCCUACA |
| | (SEQ ID NO: 151) | (SEQ ID NO: 283) |
| ZNF335_4 | GATGATGACATTGTAGACGC | GAUGAUGACAUUGUAGACGC |
| | (SEQ ID NO: 152) | (SEQ ID NO: 284) |
| KCMF1_1 | GTACGTAGAATGTTTCACCC | GUACGUAGAAUGUUUCACCC |
| | (SEQ ID NO: 153) | (SEQ ID NO: 285) |
| KCMF1_2 | AATGCAGTGCATATTAACAA | AAUGCAGUGCAUAUUAACAA |
| | (SEQ ID NO: 154) | (SEQ ID NO: 286) |
| KCMF1_3 | AGGCGATCCTAATCATGTCA | AGGCGAUCCUAAUCAUGUCA |
| | (SEQ ID NO: 155) | (SEQ ID NO: 287) |
| KCMF1_4 | GTTGTAACTGAGAAGCGGAA | GUUGUAACUGAGAAGCGGAA |
| | (SEQ ID NO: 156) | (SEQ ID NO: 288) |
| TET2_1 | TGGAGAAAGACGTAACTTCG | UGGAGAAAGACGUAACUUCG |
| | (SEQ ID NO: 157) | (SEQ ID NO: 289) |
| TET2_2 | TCTGCCCTGAGGTATGCGAT | UCUGCCCUGAGGUAUGCGAU |
| | (SEQ ID NO: 158) | (SEQ ID NO: 290) |
| TET2_3 | GATTCCGCTTGGTGAAAACG | GAUUCCGCUUGGUGAAAACG |
| | (SEQ ID NO: 159) | (SEQ ID NO: 291) |
| TET2_4 | GTTCCAAAAACCCTCACACC | GUUCCAAAAACCCUCACACC |
| | (SEQ ID NO: 160) | (SEQ ID NO: 292) |
| ZBTB1_1 | GTGTGGACTTACAATAACCG | GUGUGGACUUACAAUAACCG |
| | (SEQ ID NO: 161) | (SEQ ID NO: 293) |
| ZBTB1_2 | GCACAGACGGACAAATACAG | GCACAGACGGACAAAUACAG |
| | (SEQ ID NO: 162) | (SEQ ID NO: 294) |
| ZBTB1_3 | AAATACACCACATAATAGAG | AAAUACACCACAUAAUAGAG |
| | (SEQ ID NO: 163) | (SEQ ID NO: 295) |
| ZBTB1_4 | GTGTGTCAAGACGCTTTGGG | GUGUGUCAAGACGCUUUGGG |
| | (SEQ ID NO: 164) | (SEQ ID NO: 296) |
| ZFX_1 | TCTATACATGTGTCTGACGT | UCUAUACAUGUGUCUGACGU |
| | (SEQ ID NO: 165) | (SEQ ID NO: 297) |
| ZFX_2 | GCTGGCAAAATAGAACACGA | GCUGGCAAAAUAGAACACGA |
| | (SEQ ID NO: 166) | (SEQ ID NO: 298) |
| ZFX_3 | CATTTGCTGCTCGTGCACGG | CAUUUGCUGCUCGUGCACGG |
| | (SEQ ID NO: 167) | (SEQ ID NO: 299) |
| ZFX_4 | TGTAATCCAAGATGTTATTG | UGUAAUCCAAGAUGUUAUUG |
| | (SEQ ID NO: 168) | (SEQ ID NO: 300) |
| RUNX1_1 | CTGATCGTAGGACCACGGTG | CUGAUCGUAGGACCACGGUG |
| | (SEQ ID NO: 169) | (SEQ ID NO: 301) |
| RUNX1_2 | CACTTCGACCGACAAACCTG | CACUUCGACCGACAAACCUG |
| | (SEQ ID NO: 170) | (SEQ ID NO: 302) |
| RUNX1_3 | TAGATGATCAGACCAAGCCC | UAGAUGAUCAGACCAAGCCC |
| | (SEQ ID NO: 171) | (SEQ ID NO: 303) |
| RUNX1_4 | GGCAGTGGAGTGGTTCAGGG | GGCAGUGGAGUGGUUCAGGG |
| | (SEQ ID NO: 172) | (SEQ ID NO: 304) |
| FOXO1_1 | TAGCATTTGAGCTAGTTCGA | UAGCAUUUGAGCUAGUUCGA |
| | (SEQ ID NO: 173) | (SEQ ID NO: 305) |
| FOXO1_2 | GGAGTTTAGCCAGTCCAACT | GGAGUUUAGCCAGUCCAACU |
| | (SEQ ID NO: 174) | (SEQ ID NO: 306) |
| FOXO1_3 | GGTGGCGCAAACGAGTAGCA | GGUGGCGCAAACGAGUAGCA |
| | (SEQ ID NO: 175) | (SEQ ID NO: 307) |
| FOXO1_4 | ACAGGTTGCCCCACGCGTTG | ACAGGUUGCCCCACGCGUUG |
| | (SEQ ID NO: 176) | (SEQ ID NO: 308) |
| MBD2_1 | AGCCGGTCCCTTTCCCGTCG | AGCCGGUCCCUUUCCCGUCG |
| | (SEQ ID NO: 177) | (SEQ ID NO: 309) |
| MBD2_2 | CCTCAGTTGGCAAGGTACCT | CCUCAGUUGGCAAGGUACCU |
| | (SEQ ID NO: 178) | (SEQ ID NO: 310) |
| MBD2_3 | CCTCTCAATCAAAATAAGGT | CCUCUCAAUCAAAAUAAGGU |
| | (SEQ ID NO: 179) | (SEQ ID NO: 311) |
| MBD2_4 | CGAAAATCTGGGCTAAGTGC | CGAAAAUCUGGGCUAAGUGC |
| | (SEQ ID NO: 180) | (SEQ ID NO: 312) |
| YY1_1 | TGAACAAACGCTGGTCACCG | UGAACAAACGCUGGUCACCG |
| | (SEQ ID NO: 181) | (SEQ ID NO: 313) |
| YY1_2 | AGATATTGACCATGAGACAG | AGAUAUUGACCAUGAGACAG |
| | (SEQ ID NO: 182) | (SEQ ID NO: 314) |
| YY1_3 | GGTCACCGACGACCCGACCC | GGUCACCGACGACCCGACCC |
| | (SEQ ID NO: 183) | (SEQ ID NO: 315) |
| YY1_4 | GGAGACCATCGAGACCACAG | GGAGACCAUCGAGACCACAG |
| | (SEQ ID NO: 184) | (SEQ ID NO: 316) |
| DNMT1_1 | GATTTCTGATGAAAAAGACG | GAUUUCUGAUGAAAAAGACG |
| | (SEQ ID NO: 185) | (SEQ ID NO: 317) |
| DNMT1_2 | GCTCTACTGGAGCGACGAGG | GCUCUACUGGAGCGACGAGG |
| | (SEQ ID NO: 186) | (SEQ ID NO: 318) |
| DNMT1_3 | GAGGCAAAAAGAAATCCCCA | GAGGCAAAAAGAAAUCCCCA |
| | (SEQ ID NO: 187) | (SEQ ID NO: 319) |
| DNMT1_4 | TCACCCAAAAAAATGCACCA | UCACCCAAAAAAAUGCACCA |
| | (SEQ ID NO: 188) | (SEQ ID NO: 320) |
| IRF1_1 | TTAATTCCAACCAAATCCCG | UUAAUUCCAACCAAAUCCCG |
| | (SEQ ID NO: 189) | (SEQ ID NO: 321) |
| IRF1_2 | TCTAGGCCGATACAAAGCAG | UCUAGGCCGAUACAAAGCAG |
| | (SEQ ID NO: 190) | (SEQ ID NO: 322) |
| IRF1_3 | GAACTCCCTGCCAGATATCG | GAACUCCCUGCCAGAUAUCG |
| | (SEQ ID NO: 191) | (SEQ ID NO: 323) |
| IRF1_4 | TCTCCCTCGACAGTCATGTG | UCUCCCUCGACAGUCAUGUG |
| | (SEQ ID NO: 192) | (SEQ ID NO: 324) |
| CTCF_1 | GAGCAAACTGCGTTATACAG | GAGCAAACUGCGUUAUACAG |
| | (SEQ ID NO: 193) | (SEQ ID NO: 325) |
| CTCF_2 | CGATCCAAATTTGAACGCCG | CGAUCCAAAUUUGAACGCCG |
| | (SEQ ID NO: 194) | (SEQ ID NO: 326) |
| CTCF_3 | TTTGTGCAGTTATGCCAGCA | UUUGUGCAGUUAUGCCAGCA |
| | (SEQ ID NO: 195) | (SEQ ID NO: 327) |
| CTCF_4 | TTACCCCAGAACCAGACGGA | UUACCCCAGAACCAGACGGA |
| | (SEQ ID NO: 196) | (SEQ ID NO: 328) |
| GTF2B_1 | AGTTGTAATCAAATCCACAC | AGUUGUAAUCAAAUCCACAC |
| | (SEQ ID NO: 197) | (SEQ ID NO: 329) |
| GTF2B_2 | GATATGATCTGTCCTGAATG | GAUAUGAUCUGUCCUGAAUG |
| | (SEQ ID NO: 198) | (SEQ ID NO: 330) |
| GTF2B_3 | ACAAAAGATCCATCTCGAGT | ACAAAAGAUCCAUCUCGAGU |
| | (SEQ ID NO: 199) | (SEQ ID NO: 331) |
| GTF2B_4 | TTTGTCTACCATGATTGGCA | UUUGUCUACCAUGAUUGGCA |
| | (SEQ ID NO: 200) | (SEQ ID NO: 332) |
| HIC1_1 | CCTTCCGACCCATTTCGCGG | CCUUCCGACCCAUUUCGCGG |
| | (SEQ ID NO: 201) | (SEQ ID NO: 333) |
| HIC1_2 | CTGTACGTGTGCATTCCGTG | CUGUACGUGUGCAUUCCGUG |
| | (SEQ ID NO: 202) | (SEQ ID NO: 334) |
| HIC1_3 | AAGAAACGCCTCAAGCGCCA | AAGAAACGCCUCAAGCGCCA |
| | (SEQ ID NO: 203) | (SEQ ID NO: 335) |
| HIC1_4 | CAGGTTGTCATGCACCACCA | CAGGUUGUCAUGCACCACCA |
| | (SEQ ID NO: 204) | (SEQ ID NO: 336) |

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

### vi) Repair Pathways

The CRISPR/Cas9-based gene editing system may be used to introduce site-specific double strand breaks at targeted genomic loci, such as a gene for modulating T cells as detailed herein. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### (1) Homology-Directed Repair (HDR)

Restoration of protein expression from a gene may involve homology-directed repair (HDR). A donor template may be administered to a cell. A donor sequence comprises a polynucleotide sequence to be inserted into a genome. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### (2) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 3. Genetic Constructs

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as polynucleotide encoding a reporter protein and/or a selectable marker, such as hygromycin (“Hygro”). The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise green fluorescent protein (“GFP”), YFP, RFP, CFP, DsRed, luciferase, and/or Thy1.

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 4. Additional Cancer Therapies

The compositions and methods detailed herein may further include at least one additional therapy or cancer therapy. As used herein, the term “standard of care treatment” or “additional therapy” or “additional treatment” are used interchangeably and refer to any other standard cancer treatments/additional cancer treatments that do not include the specific compositions detailed herein for modifying a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. Additional cancer therapies may comprise a small molecule, peptide, polypeptide, antibody, nucleotide, polynucleotide, lipid, or carbohydrate, or a combination thereof. Additional cancer therapies may be synthesized and/or extracted and/or purified by any suitable means known in the art. Additional cancer therapies may be commercially available. Additional cancer therapies may include, for example, chemotherapy, immunotherapy, radiation therapy, hormone therapy, targeted drug therapy, cryoablation, antibody drug conjugates, and surgery, or a combination thereof. Hormone therapy, for example, may block hormone synthesis such as blocking estrogen synthesis. An effective amount of the additional therapy may be administered.

Chemotherapy may include, for example, an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, and an inhibitor of a pathway that cross-talks with and activates ER transcriptional activity, or a combination thereof.

Alkylating agents may include, for example, cisplatin (PLATINOL®), oxaliplatin (ELOXATIN®), chlorambucil (LEUKERAN®), procarbazine (MATULANE®; NATULAN®), or carmustine (BiCNU®), or a combination thereof. Antimetabolites may include, for example, methotrexate (also known as amethopterin), 5-fluorouracil, cytarabine (also known as cytosine arabinoside or ara-C; CYTOSAR®), or gemcitabine (GEMZAR®), or a combination thereof. Antimicrotubule agents may include, for example, vinblastine (VELBAN®; VELBE®), or paclitaxel (TAXOL®), or a combination thereof. Topoisomerase inhibitors may include, for example, etoposide (VEPESID®), or doxorubicin (ADRIAMYCIN®; MYOCET®), or a combination thereof. Cytotoxic agents may include, for example, bleomycin (BLENOXANE®). Growth factor inhibitors may include, for example, human epidermal growth factor receptor 2 (HER2) inhibitors. HER2 inhibitors include, for example, trastuzumab (HERCEPTIN®), deruxtecan, sacitizumab, and/or ado-trastuzumab emtansine (KADCYLA®). HDAC inhibitors may include, for example, vorinostat (ZOLINZA®), romidepsin (ISTODAX®), chidamide (also known as tucidinostat; EPIDAZA®; HIYASTA™) panobinostat (FARYDAK®), belinostat (also known as BELEODAQ® or PXD101), valproic acid (DEPAKOTE®; DEPAKENE®; STAVZOR®)), mocetinostat (also known as MGCD0103), abexinostat (also known as PCI-24781), entinostat (also known as SNDX-275 or MS-275), pracinostat (also known as SB939), resminostat (also known as 4SC-201 or RAS2410), givinostat (also known as gavinostat or ITF2357), quisinostat (also known as JNJ-26481585), kevetrin, CUDC-101, AR-42, tefinostat (also known as CHR-2845), nanatinostat (also known as CHR-3996), domatinostat (also known as 4SC-202), ivaltinostat (also known as CG-200745), rocilinostat (also known as ACY-1215), or sulforaphane, or a combination thereof. Inhibitors of a pathway that cross-talks with and activates ER transcriptional activity may include, for example, a phosphoinositide 3-kinase (PI3K) inhibitor, a heat shock protein 90 (HSP90) inhibitor, or a mammalian target of rapamycin (mTOR) inhibitor. mTOR inhibitors include, for example, everolimus (AFINITOR®; VOTUBIA®; ZORTRESS®). In some embodiments, the HDAC inhibitor comprises vorinostat (ZOLINZA®) and/or romidepsin (ISTODAX®).

Immunotherapies may include, for example, a checkpoint inhibitor, or denosumab (PROLIA®; XGEVA®), or a combination thereof. “Checkpoint inhibitor” or “immune checkpoint inhibitor” may also be referred to as an immune checkpoint blockade (ICB) therapy. Checkpoint inhibitors may comprise an antibody. Checkpoint inhibitors may include, for example, an antibody to programmed cell death protein 1 (PD1) (anti-PD1), or an antibody to cytotoxic T-lymphocyte-associated protein 4 (CTLA4) (anti-CTLA4), or an antibody to programmed death-ligand 1 (PDL1) (anti-PDL1), or DMXAA (sting agonist; also known as ASA404, vadimezan, or dimethylxanthone acetic acid) or a combination thereof. “Anti-PD1” refers to an antibody that binds PD1, “anti-CTLA4” refers to an antibody that binds CTLA4, and “anti-PDL1” refers to an antibody that binds PDL1. In some embodiments, the PD-1 antibody comprises pembrolizumab (KEYTRUDA®) or nivolumab (OPDIVOo®). In some embodiments, the CTLA-4 antibody comprises ipilimumab (YERVOY®).

Antibody drug conjugates may include, for example, gemtuzumab ozogamicin (MYLOTARG™), brentuximab vedotin (ADCETRIS®), ado-trastuzumab emtansine (KADCYLA®), inotuzumab ozogamicin (BESPONSA®), polatuzumab vedotin (POLIVY®), enfortumab vedotin (PADCEV®), fam-trastuzumab deruxtecan (ENHERTU®), sacituzumab govitecan (TRODELVY®), loncastuximab tesirine (ZYNLONTA®), tisotumab vedotin (TIVDAK®), mirvetuximab soravtansinegynx (ELAHERE™), moxetumomab pasudotox (LUMOXITI™), belantamab mafodotin-blmf (BLENREP®), cetuximab saratolacan (AKALUX®), or disitamab vedotin (AIDIXI®), or a combination thereof.

### 5. Pharmaceutical Compositions

Further provided herein are pharmaceutical compositions comprising the above-described modulator of T cells or genetic constructs or gene editing systems. In some embodiments, the composition further includes at least one cancer therapy such as a chimeric antigen receptor (CAR). In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed modulator or T cells, a variety of effects may be elicited, such as, for example, T cells may be increased, T cell numbers may be increased, memory T cells may be increased, T cell exhaustion may be inhibited or prevented, T cell exhaustion may be reversed, cancer therapy may be enhanced or its effectiveness increased, or a combination thereof. Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. In some embodiments, the cell is a T cell. In some embodiments, the cell is a CD8+ T cell. In some embodiments, the cell is a CD4+ T cell.

### 7. Kits

Provided herein is a kit, which may be used to modulate, such as increase, T cells. The kit may be used in conjunction with ACT to enhance the ACT. The kit comprises genetic constructs or a composition comprising the same, as described above, and instructions for using said composition. The kit includes a modulator of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1. In some embodiments, the modulator is an inhibitor. In some embodiments, the modulator is an activator. In some embodiments, the kit comprises at least one polynucleotide sequence selected from SEQ ID NOs: 337-369, a complement thereof, a variant thereof, or fragment thereof. In some embodiments, the kit comprises at least one polypeptide sequence selected from SEQ ID NOs: 370-402, a variant thereof, or fragment thereof. In some embodiments, the kit comprises at least one gRNA comprising a polynucleotide sequence selected from SEQ ID NOs: 205-336, a complement thereof, a variant thereof, or fragment thereof, or gRNA targeting or encoded by a polynucleotide comprising a sequence selected from SEQ ID NOs: 73-204, a complement thereof, a variant thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for modulating T cells may include a modified AAV vector that includes a gRNA molecule(s) and a Cas9 protein or fusion protein, as described above, that specifically binds and cleaves a region of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1, or a regulatory element thereof.

### 8. Methods

a. Methods of Modulating T Cells

Provided herein are methods of modulating T cells. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof. In some embodiments, modulating T cells comprises increasing T cells, or increasing memory T cells, or preventing T cell exhaustions, or reversing T cell exhaustions, or a combination thereof.

b. Methods of Increasing T Cells

Provided herein are methods of increasing T cells. The methods may include administering to a cell or a subject a composition as detailed herein, or an isolated polynucleotide sequence as detailed herein, or a vector as detailed herein, or a cell as detailed herein, or a pharmaceutical composition as detailed herein, or a combination thereof.

c. Methods of Enhancing Adoptive T Cell Therapy (ACT)

Provided herein are methods of enhancing adoptive T cell therapy (ACT) in a subject. The methods may include administering to the subject a composition as detailed herein, or an isolated polynucleotide sequence as detailed herein, or a vector as detailed herein, or a cell as detailed herein, or a pharmaceutical composition as detailed herein, or a combination thereof.

d. Methods of Treating Cancer

Provided herein are methods of treating cancer in a subject. The methods may include administering to the subject a composition as detailed herein, or an isolated polynucleotide sequence as detailed herein, or a vector as detailed herein, or a cell as detailed herein, or a pharmaceutical composition as detailed herein, or a combination thereof.

### 9. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Plasmids. All plasmids used were cloned using Gibson assembly (NEB). The all-in-one HER2 CAR constructs used for in vivo tumor control studies were cloned by digesting an empty lentiviral vector for constitutive gene expression (Addgene) with MluI and amplifying the HER2-CAR⁷⁰ and 2A-GFP or 2A-BATF3 (gblock, IDT) fragments with appropriate overhangs for Gibson assembly. The following plasmids were deposited to Addgene: pLV hU6-gRNA hUbC-dSaCas9-KRAB-T2A-Thy1.1 (Addgene) and pLV hU6-gRNA hUbC-VP64-dSaCas9-VP64-T2A-Thy1.1 (Addgene).

Cell Lines. HEK293 Ts and SKBR3s were maintained in DMEM GlutaMAX supplemented with 10% fetal bovine serum (FBS), 1 mM sodium pyruvate, 1×MEM non-essential amino acids (NEAA), 10 mM HEPES, 100 U mL⁻¹ of penicillin, and 100 μg mL⁻¹ streptomycin. Jurkats lines were maintained in RPMI supplemented with 10% FBS, 100 U mL⁻¹ of penicillin, and 100 μg mL⁻¹ streptomycin. HCC1954s were maintained in DMEM/F12 supplemented with 10% FBS, 100 U mL⁻¹ of penicillin, and 100 μg mL⁻¹ streptomycin.

Isolation and Culture of Primary Human T Cells. Human CD8+ T cells were obtained from either pooled PBMC donors (ZenBio) using negative selection human CD8 isolation kits (StemCell Technologies) or directly from vials containing isolated CD8+ T cells from individual donors (StemCell Technologies). For all technology development experiments, T cells were cultured in Advanced RPMI (Thermo Fisher) supplemented with 10% FBS, 100 U mL⁻¹ of penicillin and 100 μg mL⁻¹ streptomycin. For all T cell reprogramming experiments, T cells were cultured in PRIME-XV T cell Expansion XSFM (FujiFilm) supplemented with 5% human platelet lysate (Compass Biomed), 100 U mL⁻¹ of penicillin and 100 μg mL⁻¹ streptomycin. All media was supplemented with 100 U mL⁻¹ human IL-2 (Peprotech). T cells were activated with a 3:1 ratio of CD3/CD28 dynabeads to T cells and split or expanded every 2 days to maintain T cells at a concentration of 1-2×10⁶ per mL unless otherwise indicated.

Lentivirus Generation and Transduction of Primary Human T Cells. For all technology development experiments, lentivirus was produced as previously described (Black et al., Cell Rep 33, 108460 (2020)). For all T cell reprogramming experiments, a recently optimized protocol for high-titer lentivirus was used (Schmidt et al., Science 375 (2022)). Briefly, 1.2×10⁶ or 7×10⁶ HEK293T cells were plated in a 6 well plate or 10 cm dish in the afternoon with 2 mL or 12 mL of complete opti-MEM (Opti-MEM™ I Reduced Serum Medium supplemented with 1× Glutamax, 5% FBS, 1 mM Sodium Pyruvate, and 1×MEM Non-Essential Amino Acids). The next morning, HEK293T cells were transfected with 0.5 μg pMD2.G, 1.5 μg psPAX2, and 0.5 μg transgene for 6 well transfections or 3.25 μg pMD2.G, 9.75 μg psPAX2, and 4.3 μg transgene for 10 cm dishes using Lipofectamine 3000. Media was exchanged 6 hours after transfection and lentiviral supernatant was collected and pooled at 24 hours and 48 hours after transfection. Lentiviral supernatant was centrifuged at 600×g for 10 min to remove cellular debris and concentrated to 50-100× the initial concentration using Lenti-X Concentrator (Takara Bio). T cells were transduced at 5-10% v/v of concentrated lentivirus at 24 hours post-activation. For dual transduction experiments, T cells were serially transduced at 24 hours and 48 hours post activation.

Design of CD2, B2M, and IL2RA gRNA Libraries. Saturation CD2 and B2M CRISPRi gRNA libraries were designed to tile a 1,050 bp window (−400 bp to 650 bp) around the TSS of each target gene using CRISPick (Sanson et al., Nat Commun 9, 5416 (2018)). The IL2RA CRISPRa gRNA library was designed to tile a 5 kb bp window (−4,000 bp to 1000 bp) around the TSS of IL2RA using ChopChop (Labun et al., Nucleic Acids Research 47, 171-174 (2019)). Any gRNA that aligned to another genomic site with fewer than four mismatches was removed from the library. Each gRNA library was designed to target dSaCas9's relaxed PAM variant: 5′-NNGRRN-3′. Non-targeting gRNAs were generated for each library to match the nucleotide composition of the targeting gRNAs. CD2, B2M, and II2RA gRNA libraries can be found in TABLE 1 of McCutcheon et al., Nat. Genet. 2023; 55(12): 2211-2223, which is incorporated herein by reference in its entirety.

gRNA Library Cloning. Oligonucleotide gRNA pools containing variable protospacer sequences and constant regions for PCR amplification were synthesized by Twist Bioscience. 2-4 ng of each oligonucleotide pool was input into a 7-cycle PCR with 2× Q5 mastermix and 10 μM of each amplification primer with the following cycling conditions: 98° C. for 10s, 65° C. for 30s, and 72° C. for 15s. The gRNA amplicon was gel extracted and then PCR purified. The purified gRNA amplicon was input into a 20 μL Gibson reaction at a 5:1 insert to backbone molar ratio with 200 ng of either all-in-one CRISPRi or CRISPRa backbones digested with Esp3I, dephosphorylated using QuickCIP, and 1×SPRI-selected. The Gibson reactions were ethanol precipitated overnight and transformed into Lucigen's Endura ElectroCompetent Cells. Cloned gRNA libraries were purified for lentivirus production by midi-prepping 100 mL of bacterial culture.

CD2 and B2M CRI5PRi Screens in Primary Human T Cells. CD8+ T cells from pooled PBMC donors were transduced with all-in-one lentivirus encoding for dSaCas9-KRAB-2A-GFP and either CD2 (n=2 replicates) or B2M (n=3 replicates) gRNA libraries. Cells were expanded for 9 days and then stained for the target gene (CD2 or B2M). Transduced GFP+ T cell in the lower and upper 10% tails of target gene expression were sorted for subsequent gRNA library construction and sequencing. All replicates were maintained and sorted at a minimum of 350× coverage.

Construction of CRI5PRa Jurkat Lines and IL2RA CRI5PRa Screens in Jurkats. Polyclonal dSaCas9^{VP64} and ^{VP64}dSaCas9^{VP64} Jurkat cell lines were generated by transducing 2×10⁶ Jurkats with 2% v/v of 50× lentivirus encoding for either dSaCas9^{VP64}-2A-PuroR or ^{VP64}dSaCas9^{VP64}-2A-PuroR. Cells were selected for five days (days 3-7 post-transduction) using 0.5 μg/mL of puromycin. After puromycin selection, 1×10⁶ dSaCas9^{VP64} and ^{VP64}dSaCasg^{VP64} Jurkat cells were plated and transduced in triplicate with the IL2RA gRNA library lentivirus at a multiplicity of infection (MOI=0.4). Cells were expanded for 10 days, selected for Thy1.1 using a CD90.1 Positive Selection Kit (StemCell Technologies), and then stained for Thy1.1 and IL2RA. Transduced Thy1.1+ Jurkats in the lower and upper 10% tails of IL2RA expression were sorted for subsequent gRNA library construction and sequencing. All replicates were maintained and sorted at a minimum of 500× coverage.

TF and Epi-Modifier CRI5PRi/a gRNA Library Construction. Genes were selected based on motif enrichment in differentially accessible chromatin across T cell subsets (Krishna et al., Science 370, 1328-1334 (2020); Philip et al., Nature 545, 452-456 (2017); Galletti et al., Nature Immunology (2020)) and a unified atlas of over 300 ATAC-seq and RNA-seq experiments from 12 studies of CD8 T cells in cancer and chronic infection (Pritykin et al., Mol Cell 81, 2477-2493 e2410 (2021)). The following transcriptional and epigenetic regulators: BACH2, TOX, TOX2, PRDM1, KLF2, BMI1, DNMT1, DNMT3A, DNMT3B, TET1, and TET2 were manually added to the gene list. The complete 121 member gene list can be found in TABLE 2 of McCutcheon et al., Nat. Genet. 2023; 55(12): 2211-2223, which is incorporated herein by reference in its entirety. The TSS for each gene was extracted using CRISPick and 1,000 bp windows were constructed around each TSS (−500 to +500 bp). After establishing an SaCas9 gRNA database with the strict PAM variant (NNGRRT) using guideScan (Perez et al., Nat Biotechnol (2017)), the genomic windows were input into the guidescan_guidequery function to generate the gRNA library. Any gRNA that aligned to another genomic site with fewer than four mismatches was removed from the library. The final gRNA library contained at least seven gRNAs targeting 120/121 target gene (there were no PBX2-targeting gRNAs) with an average of 16 gRNAs per gene. 120 non-targeting gRNAs were included in the library for a total of 2,099 gRNAs (TABLE 2 of McCutcheon et al., Nat. Genet. 2023; 55(12): 2211-2223, which is incorporated herein by reference in its entirety).

TF and Epi-Modifier CRI5PRi/a gRNA Screens. CD8+CCR7+ T cells were sorted and transduced with either CRISPRi (n=2 donors) or CRISPRa (n=3 donors) TF+epi-modifier gRNA libs. Cells were expanded for 10 days and then stained for Thy1.1 (a marker to identify transduced cells) and CCR7 (a marker associated with T cell state). Transduced Thy1.1+ T cells in the lower and upper 10% tails of CCR7 expression were sorted for subsequent gRNA library construction and sequencing. All replicates were maintained and sorted at a minimum of 300× coverage.

Genomic DNA Isolation, gRNA PCR, and Sequencing gRNA Libraries. Genomic DNA was isolated from sorted cells using Qiagen's DNeasy Blood and Tissue Kit. All genomic DNA was split across 100 μL PCR reactions with 05 2× Master Mix, up to 1 μg of genomic DNA per reaction, and forward and reverse primers. After initial amplicon denaturation at 98° C. for 30s, gRNA libraries were amplified through 25 PCR cycles at 98° C. for 10s, 60° C. for 30s, and 72° C. for 20s, followed by a final extension at 72° C. for 20s. PCRs were pooled together for each sample and purified using double-sided SPRI selection at 0.6× and 1.8× to remove gDNA and primer dimer. Libraries were run on a High Sensitivity D1000 tape (Agilent) to confirm the expected amplicon size and quantified using Qubit's dsDNA High Sensitivity assay. Libraries were individually diluted to 2 nM, pooled together at equal volumes, and sequenced using Illumina's MiSeq Reagent Kit v2 (50 cycles) according to manufacturer's recommendations. Read 1 was 22 cycles to sequence the 21 bp protospacers and index read 1 was 6 cycles to sequence the sample barcodes. Primers used in this study can be found in TABLE 5 of McCutcheon et al., Nat. Genet. 2023; 55(12): 2211-2223, which is incorporated herein by reference in its entirety.

Processing gRNA Sequencing and Enrichment Analysis for FAC5-based Screens. FASTQ files were aligned to custom indexes for each gRNA library (generated from the bowtie2-build function) using Bowtie 2 (Langmead and Salzberg, Nat Methods 9, 357-359 (2012)). Counts for each gRNA were extracted and used for further analysis. All enrichment analysis was done with R. Individual gRNA enrichment was determined using the DESeq2 (Love et al., Genome Biology (2014)) package to compare gRNA abundance between high and low conditions for each screen. gRNAs were selected as hits if they met a specific statistical significance threshold (defined in figured legends). DESeq2 results for each cell sorting based screen in this study can be found in TABLES 1-2 of McCutcheon et al., Nat. Genet. 2023; 55(12): 2211-2223, which is incorporated herein by reference in its entirety.

Individual gRNA Validation Using Flow Cytometry. For CD2 and B2M gRNA validations, CD8 T cells were transduced in triplicate with each individual gRNA and followed the same timeline as the CRISPRi screens. On day 9, cells were stained with either a CD2 or B2M antibody and measured using flow cytometry. For IL2RA gRNA validations, dSaCas9^{VP64} and ^{VP64}dSaCas9^{VP64} Jurkat lines were transduced with each gRNA hit and followed the same timeline as the CRISPRa screen. On day 9, cells were stained with a IL2RA antibody and measured using flow cytometry. The percentage of cells expressing the target gene or the mean fluorescence intensity (MFI) of the target gene were reported for flow cytometry data.

Flow Cytometry and Surface Marker Staining. An SH800 FACS Cell Sorter (Sony Biotechnology) was used for cell sorting and analysis unless otherwise indicated. For antibody staining of all surface markers except CCR7, cells were harvested, spun down at 300×g for 5 min, resuspended in flow buffer (1×PBS, 2 mM EDTA, 0.5% BSA) with the appropriate antibody dilutions and incubated for 30 min at 4° C. on a rocker. Antibody staining of CCR7 was carried out for 30 min at 37° C. Cells were then washed with 1 mL of flow buffer, spun down at 300×g for 5 min, and resuspended in flow buffer for cell sorting or analysis. Antibody details can be found in TABLE 5 of McCutcheon et al., Nat. Genet. 2023; 55(12): 2211-2223, which is incorporated herein by reference in its entirety. FMO controls were used to set appropriate gates for all flow panels.

Quantitative RT-qPCR. mRNA was isolated from transduced primary human CD8+ T cells or Jurkats using Norgen's Total RNA Purification Plus Kit. Reverse transcription was carried out by inputting an equal mass of mRNA for each sample into a 10 μL SuperScript Vilo cDNA Synthesis reaction. 2.0 μL of cDNA was used per PCR reaction with Perfecta SYBR Green Fastmix (Quanta BioSciences) using the CFX96 Real-Time PCR Detection System (Bio-Rad). All primers were designed to be highly specific using NCBI's primer blast tool and amplicon products were verified by melt curve analysis. All qRT-qPCR are presented as log 2 fold change in RNA normalized to GAPDH expression unless otherwise indicated. Primers used in this study can be found in TABLE 5 of McCutcheon et al., Nat. Genet. 2023; 55(12): 2211-2223, which is incorporated herein by reference in its entirety.

Characterization of TF Hits Using scRNA-seq. All 32 gRNA hits (as defined by a P_{adj}<0.05) from the CRISPRi/a screens and 8 non-targeting gRNAs were selected for scRNA-seq characterization. This 40-gRNA library (TABLE 3 of McCutcheon et al., Nat. Genet. 2023; 55(12): 2211-2223, which is incorporated herein by reference in its entirety) was cloned into the all-in-one CRISPRi and CRISPRa lentiviral plasmids. The experimental timeline for the scRNA-seq screens was identical to the cell sorting-based screens. CD8+CCR7+ T cells from three donors were transduced with CRISPRi and CRISPRa mini-TF gRNA libraries. T cells were expanded for 10 days and then stained and sorted for Thy1.1+ cells. Sorted cells were loaded into the Chromium X for a targeted recovery of 2×10⁴ cells per donor and treatment according to the Single Cell 5′-High-Throughput (HT) Reagent Kit v2 protocol (10× Genomics). SaCas9 gRNA sequences were captured by spiking in 2 μM of a custom primer into the reverse transcription master mix, as previously done for SpCas9 gRNA capture (Mimitou et al., Nat Methods 16, 409-412 (2019)). The custom primer was designed to bind to the constant region of SaCas9's gRNA scaffold. 5′-Gene Expression (GEX) and gRNA libraries were separated using double-sided SPRI selection in the initial cDNA clean up step. 5′-GEX libraries were constructed according to manufacturer's protocol. gRNA libraries were constructed using two sequential PCRs (PCR 1: 10 cycles, PCR 2: 25 cycles). The PCR 1 product was purified using double-sided SPRI selection at 0.6× and 2×. 20% of the purified PCR 1 product was input into PCR 2. The PCR2 product was purified using double-sided SPRI selection at 0.6× and 1×. All libraries were run on a High Sensitivity D1000 tape to measure the average amplicon size and quantified using Qubit's dsDNA High Sensitivity assay. Libraries were individually diluted to 20 nM, pooled together at desired ratios, and sequenced on an Illumina NovaSeq S4 Full Flow Cell (200 cycles) with the following read allocation: Read 1=26, 7 index=10, Read 2=90. All oligos used in this study can be found in TABLE 5 of McCutcheon et al., Nat. Genet. 2023; 55(12): 2211-2223, which is incorporated herein by reference in its entirety.

Processing and Analyzing scRNA-seq. CellRanger v6.0.1 was used to process, demultiplex, and generate UMI counts for each transcript and gRNA per cell barcode. UMI counts tables were extracted and used for subsequent analyses in R using the Seurat (Butler et al., Nat Biotechnol (2018)) v4.1.0 package. Low quality cells with <200 detected genes, >20% mitochondrial reads, or <5% ribosomal reads were discarded. DoubletFinder (McGinnis et al., Cell Systems (2019)) was used to identify and remove predicted doublets. All remaining high-quality cells across donors for each treatment (CRISPRi or CRISPRa) were aggregated for further analyses. gRNAs were assigned to cells if they met the threshold (gRNA UMI>4). Cells were then grouped based on gRNA identity. For differential gene expression analysis, we compared the transcriptomic profiles of cells sharing a gRNA to cells with only non-targeting gRNAs using Seurat's FindMarkers function to test for differentially expressed genes (DEGs) with the hurdle model implemented in MAST. All significant gRNA-to-gene links can be found in TABLE 3 of McCutcheon et al., Nat. Genet. 2023; 55(12): 2211-2223, which is incorporated herein by reference in its entirety. Upregulated DEGs were input into EnrichR's GO Biological Process 2021 database (Kuleshov et al., Nucleic Acids Research 44, 90-97 (2016)) for functional annotation.

RNA-sequencing with BATF3 Overexpression. CD8+ T cells were transduced with lentivirus encoding for BATF3-2A-GFP or GFP and expanded for 10 days. On day 10, 4×10⁵ GFP+ T cells were sorted for subsequent RNA isolation using Norgen's Total RNA Purification Plus Kit. RNA was submitted to Azenta (formerly Genewiz) for standard RNA-seq with polyA selection. Reads were first trimmed using Trimmomatic (Bolger et al., Bioinformatics 30 (2014)) v0.32 to remove adapters and then aligned to GRCh38 using STAR v2.4.1a aligner. Gene counts were obtained with featureCounts (Liao et al., Bioinformatics 30 (2013)) from the subread package (version 1.4.6-p4) using the comprehensive gene annotation in Gencode v22. Differential expression analysis was determined with DESeq2 (Love et al., Genome Biology (2014)) where gene counts are fitted into a negative binomial generalized linear model (GLM) and a Wald test determines significant DEGs (P_{adj}<0.01). All DEGs can be found in TABLE 4 of McCutcheon et al., Nat. Genet. 2023; 55(12): 2211-2223, which is incorporated herein by reference in its entirety. Upregulated and downregulated DEGs were input into EnrichR's GO Biological Processes 2021 database for functional annotation.

Single cell RNA-seq analysis of CD19 CAR T cell infusion product for responders and non-responders. scRNA-seq data of the infused CD19 CAR T cell products from patients treated with tisagenlecleucel (Haradhvala et al., Nature Medicine 28, 1848-1859 (2022)) were downloaded from GEO:GSE197268. Patient data in MarketMatrix format were classified as responders (R) and non-responders (NR) and processed with Seurat (Hao et al., Cell 184, 3573-3587 e3529 (2021)) 4.2.0. For each patient, cells with fewer than 20% mitochondrial UMI counts, more than 20 gene expression (GEX) UMI counts, and in the bottom 95th percentile of GEX UMI counts were selected. GEX UMI counts were log-normalized for further analysis. Individual patient data were merged (merge function in Seurat) into a combined Seurat object, preserving the group identity in the cellular barcodes. GEX UMI counts were linearly scaled and centered (ScaleData function with default parameters) before finding the most differentially expressed genes (Seurat FindVariableFeatures) using principal component analysis (PCA). Clustering was performed using the first 10 principal components to identify and select CD8+ T cells for subsequent analyses. MAST was used to identify differentially expressed genes between CD8+ T cells from responders and non-responders. All DEGs between responders and non-responders can be found in TABLE 4 of McCutcheon et al., Nat. Genet. 2023; 55(12): 2211-2223, which is incorporated herein by reference in its entirety.

ATAC-seq. 5×10⁴ transduced CD8+ T cells were sorted for Omni ATAC-seq as previously described (Corces et al., Nature Methods 14 (2017)). Libraries were sequenced on an Illumina NextSeq 2000 with paired-end 50 bp reads. Read quality was assessed with FastOC and adapters were trimmed with Trimmomatic (Bolger et al., Bioinformatics 30 (2014)). Trimmed reads were aligned to the Hg38 reference genome using Bowtie (v1.0.0; Langmead et al., Genome Biology (2009)) using parameters -v 2 --best --strata -m 1. Reads mapping to the ENCODE hg38 blacklisted regions were removed using bedtools2⁸³ intersect (v2.25.0). Duplicate reads were excluded using Picard MarkDuplicates (v1.130; broadinstitute.github.io/picard). Count per million normalized bigWig files were generated for visualization using deeptools bamCoverage (v3.0.1; Ramirez et al., Nucleic Acids Research 42 (2014)). Peak calling was performed using MACS2 narrowPeak (Zhang et al., Genome Biology (2008)) and filtered for P_{adj}≤0.001. Peak calls were merged across samples to make a union-peak set. A count matrix containing the number of reads in peaks for each sample was generated using featureCounts (subread v1.4.6; Liao et al., Bioinformatics 30 (2013)) and used for differential analysis in DESeq2 (v.1.36; Love et al., Genome Biology (2014)). ChIPSeeker (Yu et al., Bioinformatics 31, 2382-2383 (2015)) was used to annotate the genomic regions and retrieve the nearest gene around each peak.

In Vitro Tumor Killing Assay. CD8+ T cells were transduced with lentiviruses encoding for a HER2-CAR-mCherry at 24 hours post-activation and BATF3-2A-GFP or GFP at 48 hours post-activation. After 12 days of expansion, CAR+GFP+ T cells were sorted and counted for the co-culture assay. Four hours before starting the co-culture, 2×10⁵ HER2+SKBR3s were plated in a 24 well plate with cDMEM to allow the SKBR3s to adhere to the plate. After four hours, cDMEM was discarded and mCherry+GFP+ T cells in cPRIME media were added at the indicated effector to target (E:T) cell ratios. After 24 hours of co-culture, the cells were harvested by collecting the supernatant (containing T cells and dead tumor cells) and adherent cells (which were detached from the plate using trypsin). Cells were spun down at 600×g for 5 min and then stained with a fixable viability dye (FVD) and Annexin V to label dead and apoptotic cells according to manufacturer's protocol. Stained cells were analyzed using flow cytometry. The percentage of viable tumor cells was quantified using the following strict gating strategy. First, T cells were excluded based on cell size and GFP signal. Next, a gate was set around the double negative (FVD-, Annexin V-) fraction containing viable tumor cells and cellular debris. Visualizing these events on SSC vs. FSC, a gate was set to encompass events located in the bottom left quadrant. This gate was then inverted to exclude debris from the viability calculation and moved immediately beneath the T cell exclusion gate on the gating hierarchy. Tumor viability was reported using the percentage of tumor cells in the final double negative (FVD-, Annexin V-) gate.

CD3/CD2S and Tumor Repeat Stimulations. For repeated rounds of CD3/CD28 dynabead stimulation, CD3/CD28 beads were removed, cells were counted, replated at 1-2.5×10⁵ T cells, and restimulated with new CD3/CD28 beads at a 3:1 bead to cell ratio in a 24 well plate every 3 days. On day 12, cells were stained and analyzed for expression of exhaustion-associated markers using flow cytometry. For repeated rounds of tumor stimulation, 1×10⁵ HER2 CAR T cells were transferred to a new 24 well plate with 2×10⁵ SKBR3s for a 1:2 E:T ratio every 3 days. T cells were recovered without antigen stimulation for two days after the final round of tumor stimulation before ATAC-seq on day 14. For both modes of chronic stimulation, T cells were restimulated on days 3, 6, and 9.

Mice. All experiments involving animals were conducted with strict adherence to the guidelines for the care and use of laboratory animals of the National Institutes of Health (NIH). All experiments were approved by the Institutional Animal Care and Use Committee (IACUC) at Duke University (protocol number A130-22-07). 6-8-week-old female immunodeficient NOD/SCID gamma (NSG) mice were obtained from Jackson Laboratory and then housed and handled in pathogen-free conditions.

In Vivo Tumor Model. 2.5×10⁶ HCC1954 cells were implanted orthotopically into the mammary fat pad of NSG mice in 100 μL 50:50 (v:v) PBS:Matrigel. T cells were expanded for 9-11 days post-transduction before treatment. Transduction rates were measured on the day of treatment using flow cytometry. For all in vivo experiments, transduction rates exceeded 70% for both HER2-CAR-2A-GFP and HER2-CAR-2A-BATF3 constructs. T cells were resuspended at 50×10⁶ CAR+ cells mL⁻¹ in 1×PBS and serially diluted to the appropriate cell concentrations for 200 μL injections of either 10×10⁶, 2×10⁶, 5×10⁵, 2.5×10⁵, or 1×10⁵ HER2 CAR+ T cells. 20-21 days after tumor implantation, and immediately prior to CAR T cell injections, mice were randomized into groups and tumors measured. Tumor volumes were calculated based on caliper measurements using the formula volume: =½ (Length×Width²). CAR T cells were injected intravenously by tail vein injection. Tumors were measured every 4-6 days.

Flow cytometry analysis of input and tumor infiltrating CAR T cells. Mice bearing HCC1954 tumors were euthanized at days 3 and 19 post CAR T cell delivery under deep isoflurane anesthesia via exsanguination, from which blood was collected. Blood was processed via RBC lysis buffer (Sigma) treatment followed by washing in PBS. Tumors were resected, minced, and incubated in RPMI-1640 medium (Gibco) for 45 minutes in 100 μg/ml Liberase™ (Sigma-Aldrich) and 1 0 μg/ml DNAse I (Roche). Single cell suspensions for blood and tumor were filtered through a 70 mm cell strainer (Olympus Plastics), washed in PBS (Gibco), stained with Zombie NIR (1:250, Biolegend), washed in FACs buffer [2% FBS (Sigma)+PBS], and treated with 1:50 Mouse Tru-stain Fc block (Biolegend). Cells were then stained for cell surface markers followed by intracellular staining using the Transcription Factor Staining Buffer Set (Invitrogen) per manufacturer's instructions. Fluorophore conjugated antibodies against the following antigens were used for input and day 3 cells (All Biolegend unless otherwise noted): panel 1: myc-APC (Cell Signaling Technologies), CD3-BUV737 and CD8-BUV395 (BD Biosciences), TIGIT-BV605, LAG3-BV786, CD127-PERCPCy5.5, PD1-BV711, Tim3-PECy5, GranzymeB-PECy7, TCF1-BV421, Ki67-BV510, and IFN-γ; panel 2: myc-APC, CD3-BUV737, CD8-BUV395, CD39-PECF594, CD56-BV605, CD45RO-BV786, CD45RA-PEcy5, CD28-PECy7, CCR7-BV711, CD62L-BV510, CTLA4-BV421, Tbet-PERCPCy5.5, EOMEs-PE. For day 19 post CAR T cell delivery analyses anti-human CD45-FITC (Biolegend) staining was added to the above panels to increase sensitivity of CAR T cell detection, as we anticipated reduction in numbers, and the following additional panel was added against the following antigens: CD45-FITC, myc-APC, CD3-BUV737, CD8-BUV395, LAG3-BV786, TIM3-PECy5, CXCR3-BV711, CD4-BV510, TNF-βV605, ID2-PECy7, GATA3-BV421, IRF4-PERCPCy5.5, ID3-PE. All data were collected on a Fortessa X 20 (Duke Cancer Institute Flow Cytometry Core) and analyzed using Flow Jo V10.8.1. Blood/tumor from sham infused mice and fluorescence minus one controls were used to guide gating for CAR T cells and to confirm appropriate compensation, respectively.

TFome CRI5PRko gRNA library construction. The Brunello genome wide knockout (Doench et al., Nat Biotechnol 34, 184-191 (2016)) library was subset for 1,612 TFs (Lambert et al., Cell 172, 650-665 (2018)) and IL7R. 550 non-targeting gRNAs were included in the library for a total of 7,000 gRNAs (TABLE 6 of McCutcheon et al., Nat. Genet. 2023; 55(12): 2211-2223, which is incorporated herein by reference in its entirety). This gRNA library was cloned into SpCas9 gRNA lentiviral plasmids with either mCherry or BATF3.

TFome CRI5PRko screens and validations. 20×10⁶ CD8+ T cells from two donors were activated with CD3/CD28 dynabeads at a 1:1 ratio. At 24 hours post-activation, CD8+ T cells were split evenly and transduced in parallel with TFome CRISPRko gRNA libraries with mCherry or BATF3. At 48 hours post-activation, cells were electroporated with Cas9 protein. Briefly, the cells were collected, spun down at 90×g for 10 minutes, resuspended in 1 00 μL of Lonza P3 Primary Cell buffer with 3.2 μg Cas9 per 10⁶ cells, and electroporated with the pulse code EH115. After electroporation, warm media was immediately added to each cuvette and cells were recovered at 37° C. for 20 minutes before being transferred into a 6-well plate. On day 3 post transduction, cells were selected with 2 μg/mL of puromycin for 3 days. On day 9 post transduction, cells were stained for CD8, IL7R, and a viability dye. Viable CD8+ T cells in the lower and upper 10% tails of IL7R expression were sorted for subsequent gRNA library construction and sequencing. All replicates were maintained and sorted at a minimum of 75× coverage. Subsequent individual gRNA validations were scaled down to 3.5×10⁵ cells per electroporation in an 8-well cuvette strip, but otherwise followed the same protocol and timeline as the CRISPRko screens.

TFome CR15PRko screen analyses. gRNA enrichment was performed using DESeq2 as explained above. Gene level enrichment was performed using the MAGeCK (Li et al., Genome Biol 15, 554 (2014)) test module with—paired and—control sgma parameters, pairing samples by donors and non-targeting gRNAs as control, respectively.

Statistics. Statistical details for all experiments can be found in the figure legends. ns=not significant, * <0.05, ** <0.01, *** <0.001, **** <0.0001.

### Example 2

Development and Characterization of Compact and Efficient dSaCas9-Based Epigenome Editors for Targeted Gene Regulation in Primary Human T Cells

SaCas9 has been extensively used for genome editing in vivo as its compact size (3,159 bp) enables packaging into adeno-associated virus (AAV). However, SaCas9 has been used sparingly as an epigenome editor for targeted gene regulation and has not been used in the context of an epigenome editing screen. First, we evaluated dSaCas9 for targeted gene silencing in primary human T cells by conducting two high-throughput promoter tiling CRISPRi screens. Previous CRISPRi/a screens with dSpCas9 performed serial transductions with one lentivirus encoding the dCas9-effector and another lentivirus encoding for the gRNA-library. To minimize the number of transduction events, we constructed an all-in-one CRISPRi lentiviral plasmid encoding for dSaCas9 fused to the KRAB repressor domain and a gRNA cassette (FIG. 1A).

Next, we considered the protospacer adjacent motif (PAM) requirement for SaCas9. In the context of nuclease activity, SaCas9 is more active when targeting genomic regions upstream of the PAM (5′-NNGRRT-3′) compared to the more relaxed PAM (5′-NNGRRV-3′; where V=A, C, or G). However, several gRNA design tools do not require a thymine in the final position of the PAM. Moreover, the PAM preference for dSaCas9-based epigenetic effectors has not been rigorously characterized. To systematically evaluate this feature, we designed two independent gRNA libraries with the relaxed PAM variant (5′-NNGRRN-3′) and tiled -1,000 bp windows around the promoters of CD2 and B2M. We chose CD2 and B2M as gene targets because both are ubiquitously and high expressed genes encoding for surface markers and thus readily compatible with cell sorting-based screens. The CD2 and B2M gRNA libraries contained 141 and 217 targeting gRNAs, respectively, and 250 non-targeting gRNAs.

For each CRISPRi screen, we transduced primary human CD8+ T cells with the respective gRNA library and expanded the cells for 9-10 days before staining and sorting transduced cells in the lower and upper 10% tails of CD2 or B2M expression (FIG. 1B). We recovered 16 and 5 targeting gRNAs enriched in the CD2 low and B2M low populations, respectively (FIG. 1C and FIG. 7A). Many enriched gRNAs were within an optimal window relative to the transcriptional start site (TSS) for gene silencing (FIG. 1D and FIG. 7B). Although only a small fraction of the targeting gRNAs (11% of CD2 gRNAs and 2% of B2M gRNAs) were hits for each gene target, the gRNA hit rates (32% of CD2 gRNAs and 16% of B2M gRNAs) were significantly higher for gRNAs targeting the strict PAM (5′-NNGRRT-3′). This is consistent with previous PAM characterization of SaCas9 for nuclease activity and suggests that the thymine base in the final position of the PAM facilitates more efficient recognition and binding between dSaCas9 and the target DNA sequence (FIG. 1E and FIG. 7C).

Subsequent validation of CD2 and B2M gRNA screen hits revealed marked gene silencing and a wide range of activity across gRNAs, underscoring the unique capability of CRISPRi to tune gene expression levels (FIG. 1F, FIGS. 7D-E, and FIGS. 8A-B). For example, the percentage of CD2 silenced cells varied from 7% to 89% depending on the gRNA (FIG. 1F and FIG. 8A). The mean expression of CD2 in silenced cells was highly correlated with the percentage of silenced cells, indicating that the effect of a gRNA across a cell population is coupled to the magnitude of gRNA activity at a single cell level (FIG. 8C). The most potent CD2 and B2M gRNAs targeted genomic sites adjacent to 5′-NNGRRT-3′ PAMs (FIG. 1F and FIG. 7D). As previously observed, individual gRNA activity was strongly correlated with its fold-enrichment in the screen (FIG. 1G and FIG. 8D). Finally, we adapted this CRISPRi system for multiplex gene silencing by using a lentiviral plasmid with orthogonal mouse and human U6 promoters. We verified this system using the most potent CD2 and B2M gRNAs and only detected dual silenced cells when both CD2 and B2M gRNAs were delivered (FIGS. 8E-G).

Next, we developed efficient and compact dSaCas9-based activators using the small transactivation domain VP64. Using polyclonal Jurkat cell lines constitutively expressing dSaCas9 fused to either one copy of VP64 (dSaCas9^{VP64}) or two copies of VP64 (^{VP64}dSaCas9^{VP64}) we conducted parallel CRISPRa screens with a 400 gRNA library (306 IL2RA gRNAs and 94 non-targeting gRNAs) tiling a 5,000 bp window around the TSS of the transcriptionally silenced IL2RA gene (FIG. 9A). Interestingly, there were three more gRNA hits in the ^{VP64}dSaCas9^{VP64} CRISPRa screen along with a shared set of five gRNA hits (FIGS. 1H-I). All gRNA hits targeted sites within a prominent open chromatin peak within 350 bp of the TSS with the majority located upstream of the TSS (FIG. 9B). As with gene silencing, there was a marked preference for 5′-NNGRRT-3′ PAMs for gene activation with 75% of gRNA hits targeting this PAM variant (FIG. 9C). Together, the relative gRNA position and PAM sequence were major predictors of gRNA efficacy, similar to the CRISPRi screen, as 24% (6/25) of 5′-NNGRRT-3′ targeting IL2RA gRNAs within a -1,000 bp window around the TSS were hits. Individual validation of all eight gRNA hits in both cell lines showed a significant increase in IL2RA expression with ^{VP64}dSaCaS9^{VP64} consistently more potent than dSaCas9^{VP64} (FIGS. 1J-K and FIG. 9D). Moreover, the most potent ^{VP64}dSaCaS9^{VP64} gRNAs achieved equivalent levels of IL2RA gene activation as ^{VP64}dSpCaS9^{VP64} paired with the best IL2RA gRNA from a published CRISPRa screen tiling the IL2RA locus in Jurkats (FIG. 1K).

Given the robust activity of ^{VP64}dSaCas9^{VP64} in Jurkat cells, we constructed an all-in-one CRISPRa lentiviral vector encoding for ^{VP64}dSaCas9^{VP64} and gRNA cassette for assays in primary human T cells. We hypothesized that the smaller size of dSaCas9 would lead to higher titer lentivirus than S. pyogenes Cas9 (SpCas9), thus reducing the quantity of T cells and reagents required to perform CRISPR-based screens with equivalent coverage. We tested this by transducing CD8+ T cells from two donors with serial titrations of all-in-one ^{VP64}dSaCaS9^{VP64} and ^{VP64}dSpCas9^{VP64} lentiviruses. VP64dSaCas9^{VP64} produced nearly two-fold higher lentiviral titers than the equivalent VP64dSpCas9^{VP64} construct, thus requiring half the lentiviral volume to achieve the same transduction rate (FIGS. 10A-D). We then verified that ^{VP64}dSaCas9^{VP64} could potently activate endogenous gene expression of a transcriptionally silenced gene (EGFR) in primary human T cells (FIGS. 10E-F).

### Example 3

### CRISPR Interference and Activation Screens Identify Transcriptional and Epigenetic Regulators of Human CD8 T Cell State

Transcription factors (TFs) and epigenetic modifiers function to establish and maintain cell-type specific gene expression programs, mediate response to internal and external stimuli, and ultimately dictate cell fate and function. We therefore sought to interrogate this important class of genes using high-throughput CRISPRi and CRISPRa screens in primary human CD8+ T cells. We compiled a curated list of 110 TFs associated with T cell state and function based on motif enrichment in differentially accessible chromatin across T cell subsets and manually appended the following 11 transcriptional and epigenetic regulators: BACH2, TOX, TOX2, PROM1, KLF2, BMI1, ONMT1, ONMT3A, ONMT3B, TET1, and TET2 for a total of 121 candidate genes (TABLE 2 of McCutcheon et al., Nat. Genet. 2023; 55(12): 2211-2223, which is incorporated herein by reference in its entirety). Based on our characterization of dSaCas9-based epigenome editors, we generated a gRNA library containing all specific, 5′-NNGRRT-3′ PAM targeting gRNAs within a 1,000 bp window centered around the TSS of each gene. All genes were represented by at least 7 gRNAs with an average of 16 gRNAs per gene, except for PBX2 which did not have any gRNAs (FIGS. 11A-B). We added 120 non-targeting gRNAs as negative controls, bringing the final gRNA library to 2,099 gRNAs (TABLE 2 of McCutcheon et al., Nat. Genet. 2023; 55(12): 2211-2223, which is incorporated herein by reference in its entirety). We cloned the gRNA library into both all-in-one CRISPRi and CRISPRa lentiviral plasmids. Subsequent lentiviral titrations revealed a dose-dependent response to lentiviral volume with both CRISPRi and CRISPRa constructs eclipsing 90% transduction rates (FIG. 11C).

We selected CCR7 as the readout for our screens for several reasons. First, CCR7 is a well-characterized T cell marker and is highly expressed in specific T cell subsets such as naive, stem-cell memory, and central memory T cells. Second, we hypothesized it would enable us to capture more subtle changes in T cell state than other phenotypic readouts such as proliferation or cytokine production. To start with a homogenous T cell population for our screens, we sorted CD8⁺CCR7⁺ T cells from 2-3 donors and transduced each donor with CRISPRi and CRISPRa gRNA libraries at a low multiplicity of infection (MOI) to ensure that most cells only received a single gRNA (FIGS. 12A-B). We expanded the cells for 10 days post-transduction to allow enough time for both perturbation of the target gene and any downstream effects on gene regulatory networks, and then sorted transduced cells based on expression of CCR7 (FIG. 2A and FIGS. 12C-D).

The CRISPRi screen recovered many canonical regulators of memory T cells including FOXO1, MYB, and BACH2—all of which when silenced led to reduced expression of CCR7, indicative of T cell differentiation towards effector T cells (FIG. 2B and FIG. 13A). Interestingly, the most significant hit from the CRISPRi screen was ONMT1, which encodes for a DNA methyltransferase that maintains DNA methylation across cell divisions via recognition of hemi-methylated DNA. Genetic disruption of both TET2 and ONMT3A, which encode for proteins that regulate DNA methylation in opposite directions, can improve the therapeutic potential of T cells. There was a single non-targeting gRNA (1/120) hit in the CRISPRi screen. The same non-targeting gRNA emerged as a hit in multiple screens using CCR7 as the readout, suggesting a real off-target effect.

The CRISPRa screen identified several transcription factors that have been implicated in CD8+ T cell differentiation and function such as EOMES, BATF, and JUN (FIG. 2C). Importantly, gRNA enrichment was consistent across the three donors (FIG. 2D and FIG. 13B). Multiple gRNAs targeting basic leucine zipper ATF-like transcription factors BATF and BATF3 were enriched in reciprocal directions across CRISPRi and CRISPRa screens, highlighting the power of coupling loss- and gain-of-function perturbations, and this was not related to the number of library gRNAs targeting these genes (FIGS. 13C-D). We noticed that BATF and BATF3 gRNA hits in the CRISPRa screen generally co-localized to regions upstream of the promoter and near the summits of accessible chromatin (FIGS. 2E-F). This observation is consistent with our CRISPRa IL2RA gRNA tiling screens and recent recommendations for designing gRNAs for cis-regulatory elements.

### Example 4

### Single Cell RNA-Seq Characterization of Transcriptional and Epigenetic Regulators of T Cell State

We next characterized the transcriptomic effects of each candidate gene identified from our CRISPRi and CRISPRa screens using single cell RNA-seq (scRNA-seq). We adapted the ECCITE-seq protocol for SaCas9 by designing a reverse transcription primer complementary to the constant scaffold region of the SaCas9 gRNA. This enabled simultaneous capture of both non-polyadenylated gRNA transcripts and mRNA transcripts from individual cells. We cloned the union set of gRNA hits across CRISPRi/a screens (32 gRNAs) and 8 non-targeting gRNAs into both CRISPRi and CRISPRa plasmids. We then followed the same workflow as the sort-based screens, but instead of sorting the cells based on CCR7 expression, we profiled the transcriptomes and gRNA identity of -60,000 cells across three donors for each screen. After filtering for high-quality, gRNA-assigned cells, we aggregated the cells and compared the transcriptional profile of cells with the same gRNA to non-perturbed cells (cells with only non-targeting gRNAs). To assess the quality and statistical power of our scRNA-seq data, we compared the quantity and magnitude of effects between targeting and non-targeting gRNAs. Targeting gRNAs were associated with significantly more differentially expressed genes (DEGs) and these gRNA-to-gene links had larger effect sizes than non-targeting gRNAs (FIGS. 14A-D). We therefore proceeded to evaluate the transcriptomic effects of silencing or activating each candidate gene.

First, we focused on CCR7 expression across gRNAs to validate the results from our CRISPRi/a sort-based screens (FIGS. 3A-B). The scRNA-seq data revealed that half of the gRNA hits reproducibly affected CCR7 expression with a similar rank order as predicted by the sort-based screens. For example, both assays informed that targeted silencing of DNMT1 or FOXO1 drastically reduced CCR7 expression levels, which was further confirmed through individual gRNA validations (FIGS. 15A-C). We noticed that gRNA hits that failed to validate in the scRNA-seq characterization were represented by fewer cells than gRNAs that did successfully validate, reaffirming that higher gRNA coverage helps to resolve more subtle changes in gene expression (FIG. 15D). For example, the CRISPRa CREM-targeting gRNA that validated was represented by 635 cells, whereas the other CREM-targeting gRNA was represented only 142 cells and narrowly missed the significance threshold. Nevertheless, both gRNAs upregulated CREM and similarly affected gene expression programs (FIG. 15E). Several BATF-targeting gRNAs were also underrepresented. To evaluate CREM and BATF gRNAs, we individually assayed a pair of CRISPRa gRNA hits targeting each gene and validated that each gRNA regulated CCR7 expression as predicted by the screen (FIGS. 15A-C). We suspect that several gRNAs were underrepresented in the scRNA-seq experiment due to gRNA-intrinsic features that either interfered with reverse transcription, oligo capture by the beads, or subsequent amplification. Underrepresented gRNAs were not depleted in the initial gRNA plasmid pool nor did we observe any fitness defects in individual validations. The same gRNAs were underrepresented in both CRISPRi and CRISPRa assays, further pointing to gene-independent effects. In addition to the scRNA-seq data confirming predicted gRNA effects on CCR7 expression, the true negative rates were high for both CRISPRi (96%) and CRISPRa (82%), demonstrating the specificity of sort-based screens (FIGS. 3A-B).

We next measured on-target gene silencing or activation to confirm downstream-mediated effects, such as changes in CCR7 expression, were driven by each candidate gene. Of CRISPRi and CRISPRa gRNAs assigned to at least 5 cells, 56/61 gRNAs (92%) silenced or activated their gene target (FIG. 3C). Given that CCR7 was selected as a surrogate marker for a memory T cell phenotype, we expected some perturbations to regulate subset-defining gene expression programs. Indeed, scRNA-seq revealed that silencing the top predicted positive regulators of memory (ONMT1, FOXO1, MYB) led to decreased expression of CCR7 and other memory-associated genes (such as IL7R, SELL, C027, C028, TCF7) and increased expression of effector-associated genes (GZMA, GZMB, PRF1) (FIG. 3D). Conversely, silencing FL1 led to increased expression of several memory-associated genes. Finally, we examined all DEGs associated with each perturbation to gain an unbiased view of the transcriptomic effects of silencing and activating each gene. Endogenous regulation of several TFs and epigenetic-modifying proteins had widespread transcriptional effects with 6 gene perturbations (4 CRISPRi gene perturbations and 2 CRISPRa gene perturbations) altering expression of >1,000 genes (FIG. 3E). These widespread transcriptional changes were not attributed to impaired cell fitness as these gRNAs were well represented after 10 days of T cell expansion. Unsurprisingly, silencing ONMT1—a global epigenetic modifier—massively altered the transcriptome with 6,401 DEGs and affected general biological processes such as metabolism, endomembrane system organization, and mitotic spindle organization (FIG. 3H).

Interestingly, MYB repression with two unique gRNAs resulted in widespread and concordant gene expression changes with 8,976 and 7,899 DEGs (FIGS. 3E-F). Mouse models of acute and chronic infection have implicated MYB as an essential positive regulator of stem-like memory CD8+ T cells and a small and distinct CD62L+ precursor of exhausted T cell population. In both contexts, MYB-deficient CD8+ T cells lacked therapeutic potential due to either impaired recall response or the inability to respond to checkpoint blockage. An important and lingering question has been whether MYB plays a similar role in human CD8+ T cells. Our scRNA-seq data revealed that MYB does indeed regulate human CD8+ T cell stemness with MYB silencing driving CD8+ T cells towards terminal effector T cells. MYB silencing led to downregulation of memory-associated TFs (TCF7, KLF2), lymph homing molecules (CCR7, CO62L, S1PR1), and cell-cycle inhibitors (COKN1B). In addition, MYB-silenced cells had increased expression of effector-associated TFs (TBX21, PRMO1, ZNF683), effector molecules (GZMB, PRF1), inflammatory cytokines (IFNG, TNF), and positive cell-cycle regulators (E2F1, COC6, SKP2, COC25A and KIF14) (FIGS. 16A-B). The two MYB CRISPRi gRNAs were represented by the first and third most cells across both CRISPRi and CRISPRa screens, suggesting that MYB silencing promoted T cell proliferation (FIG. 3E).

Endogenous activation of several TFs including NR1O1, EOMES, and BATF3 had large effects on T cell state. Perturbation-driven single cell clustering revealed a distinct cluster with NR1D1 activation (FIG. 17A). NR101 encodes a nuclear receptor subfamily 1 transcription factor and negatively regulates expression of core clock proteins that govern cyclical gene expression patterns. Integrative analysis of bulk ATAC-seq data across 12 independent studies of CD8 T cell dysfunction in cancer and infection found that the NR1 D1 motif was enriched in open chromatin of exhausted T cells. The causal role of NR1 D1 in CD8 T cells, however, has not been studied. NR1 D1 activation resulted in 646 upregulated and 293 downregulated genes (FIG. 17B). In agreement with NR1D1 motif enrichment, a large set of effector and exhaustion-associated genes were markedly upregulated with NR1 D1 activation. To better understand the magnitude of exhaustion induction by NR1 D1, we calculated an exhaustion gene signature score using a defined set of 82 exhaustion-specific genes. NR101-perturbed cells had a significantly higher exhaustion gene signature score than non-perturbed cells (FIG. 17C). Many memory-associated surface markers (IL7R, CCR7, SELL, C05) and TFs (TCF7, LEF1) were downregulated, suggesting NR1D1 activation synthetically programs a transcriptional profile with features of T cell exhaustion.

Endogenous activation of EOMES, a regulator of effector T cells, drove markers associated with cytokine signaling and inflammatory response, but did not lead to an increase in exhaustion-related genes (FIG. 31). The top two BATF3 gRNA hits from our cell sorting CRISPRa screen had strong and concordant effects with 3,056 and 1,402 DEGs (FIG. 3E and FIG. 3G). Gene ontology analyses revealed that BATF3-induced genes were enriched for DNA and mRNA metabolic processing, ribosomal biogenesis, and cell-cycle pathways, suggesting that BATF3 improves T cell fitness (FIG. 31).

### Example 5

### BATF3 Overexpression Promotes Features of Memory T Cells and Counters Signatures of T Cell Exhaustion

BATF3 has been shown to promote survival and memory formation in mouse CD8+ T cells, however, the molecular and phenotypic effects of BATF3 in human CD8+ T cells has not been well defined. Moreover, it is not known whether manipulating BATF3 expression in CD8+ T cells can improve T cell-mediated control of infection or cancer. To better understand the kinetics of BATF3 expression in human CD8+ cells, we performed a time course experiment where we transduced CD8+ T cells with control lentiviral vector (GFP or CRISPRa+NT gRNA), CRISPRa+BATF3 gRNA, or BATF3 open reading frame (ORF) and measured BATF3 mRNA expression at five different time points (FIG. 18A). Consistent with other studies, BATF3 expression levels spiked after T cell activation and tapered back to baseline levels by day 10 post-transduction. Both endogenous activation and ectopic BATF3 expression increased BATF3 levels relative to the controls, however, ectopic expression led to significantly higher levels of BATF3. Given the higher expression and compact size of BATF3's ORF (only 381 bp), we decided to use ectopic BATF3 expression for all subsequent assays.

First, we found that BATF3 overexpression (OE) markedly increased expression of IL7R, a surface marker associated with T cell survival, long-term persistence, and positive clinical response to ACT (FIGS. 4A-B and FIG. 18B). Next, we performed RNA-seq across CD8+ T cells from five donors to gain an unbiased view of the transcriptomic changes induced by BATF3 OE. Compared to control cells, there were over 1,100 DEGs distributed almost equally between upregulated and downregulated genes (FIG. 4C). Gene ontology analyses revealed that BATF3 OE increased expression of genes involved in metabolic pathways such as glycolysis and gluconeogenesis, T cell proliferation (DNA replication), and translation (FIG. 4D and TABLE 4 of McCutcheon et al., Nat. Genet. 2023; 55(12): 2211-2223, which is incorporated herein by reference in its entirety). Metabolic fitness is strongly associated with the potency of T cell responses. For example, central memory CD8+ T cells require glycolysis to mount rapid-recall responses after secondary antigen exposure. TCF1 supports these bioenergetic demands by preprogramming the mobilization of glycolytic enzymes. Interestingly, BATF3 OE increased expression of the transcription factor ID3 (downstream of TCF1), which can activate glycolysis and partially rescue secondary response in the absence of TCF1.

In contrast, BATF3 OE dampened T cell effector programs with downregulation of activation markers (CD69, CD2), inflammatory cytokines and cytotoxic molecules (TNF, PRF1, GNLY, NKG7) (FIGS. 4E-F). Additionally, BATF3 OE reduced expression of several markers associated with regulatory T cells, which have recently emerged as a predictive cell type for clinical response to ACT. In a cohort of refractory B cell lymphoma patients treated with CD19 CAR T cell therapy, the infused T cell product of non-responders were enriched for Treg cells and FOXP3′ cells (across all CAR⁺ cells) compared to responders. Albeit less characterized than CD4+ Tregs, a subset of CD8⁺FOXP3⁺LAG3⁺ Tregs suppress T cell activity by secreting CC chemokine ligand 4 (CCL4). Interestingly, our RNA-seq data revealed that BATF3 OE reduced expression of FOXP3, LAG3, and CCL4 in CD8⁺ T cells (FIG. 4F and FIG. 18C). BATF3 has previously been shown to silence FOXP3 expression in CD4+ Tregs by directly binding to regulatory regions within the FOXP3 locus.

In addition to LAG3, BATF3 silenced several other canonical markers of T cell exhaustion including TIGIT, TIM3, and CISH (FIG. 4F and FIG. 18C). We speculated these effects might be amplified in the context of chronic antigen stimulation. To evaluate this, we acutely and chronically stimulated control and BATF3 OE T cells with CD3/CD28 antibody-coated beads and measured expression of exhaustion-associated surface markers (PD1, TIGIT, LAG3, and TIM3) (FIG. 19A). As previously observed, PD1 expression peaked after the initial stimulation and then tapered off over time, whereas TIGIT, LAG3, and TIM3 expression were maintained or increased after each subsequent round of stimulation (FIGS. 19B-C). Notably, BATF3 OE attenuated PD1 induction and restricted TIGIT, LAG3, and TIM3 expression to closely resemble that of acutely stimulated cells despite three additional rounds of TCR stimulation (FIG. 4G and FIGS. 19B-C). As terminally exhausted T cells often co-express multiple exhaustion-associated markers, we quantified the proportion of cells expressing each combination of TIGIT, LAG3, and TIM3. Only 13% of BATF3 OE T cells co-expressed all three markers compared to 65% and 59% of untreated and GFP T cells (FIG. 4H).

### Example 6

### BATF3 Overexpression Remodels the Epigenetic Landscape of CD8+ T Cells Under Acute and Chronic Stimulation

As an orthogonal method of inducing T cell exhaustion, we armed T cells with a human epidermal growth factor 2 (HER2) CAR with or without BATF3 OE and acutely and chronically stimulated the CAR T cells with human HER2+cancer cells. Using assay for transposase-accessible chromatin with sequencing (ATAC-seq), we profiled the epigenetic landscape of T cells in each group. As expected, chronic antigen stimulation induced widespread changes in chromatin accessibility with 23,322 differentially accessible regions between acutely and chronically stimulated control cells. Many of these regions were proximal to memory and effector/exhaustion-genes (FIG. 20A).

Next, we assessed chromatin remodeling in response to BATF3 OE under acute stimulation. There was extensive chromatin remodeling with 5,104 differentially accessible regions between the groups (FIG. 21A). Of these regions, roughly 60% were more accessible with BATF3 OE. Most of these changes were in intronic or intergenic regions consistent with cis-regulatory or enhancer elements (FIG. 21B). To better understand whether changes in chromatin accessibility corresponded to changes in gene expression, we jointly analyzed our ATAC-seq and RNA-seq data. We assigned each differential region to its closest gene to estimate genes that could be regulated in cis by these elements. We then quantified how many differential regions proximal to DEGs gained or lost accessibility. There was an enrichment of regions with increased or decreased accessibility proximal to upregulated and downregulated genes, respectively, indicative that BATF3-driven epigenetic changes affected transcription (FIG. 21C). Approximately 25% of the genes that changed expression were associated with a corresponding differentially accessible region (297 out of 1,160 genes). For example, BATF3 OE extensively remodeled the chromatin landscape at IL7R and TIGIT (FIGS. 21D-E). BATF3 OE increased accessibility at the IL7R promoter, intronic, 3′-UTR, and intergenic regions and decreased accessibility at distal intergenic, 5′-UTR, and exonic regions of TIGIT.

Finally, we compared the epigenetic landscapes of chronically stimulated T cells with or without BATF3 OE. There were 22,201 differentially accessible regions between control and BATF3 OE T cells with most regions in intronic and intergenic regions (FIG. 22A). Interestingly, we observed increased accessibility at regions near both memory (TCF7, MYB, IL7R, CCR7, SELL) and effector-associated genes (EOMES, TBX21) (FIGS. 22C-D). This may represent a hybrid T cell phenotype or the presence of heterogenous subpopulations of memory and effector T cells. Consistent with RNA-seq and FACS data, we observed reduced accessibility at exhaustion loci such as TIGIT, CTLA4, LAG3 with BATF3 OE.

### Example 7

BATF3 Overexpression Enhances Tumor Control and Programs a Transcriptional Signature Associated with Clinical Response

Given that BATF3 OE induced widespread changes in gene expression and chromatin accessibility, we hypothesized that BATF3 OE might improve CD8+ T cell function. To test the antitumor capacity of BATF3 OE T cells, we used an in vitro co-culture model with T cells engineered to express a HER2-CAR and human HER2+ cancer cells. We verified that cancer cell death was dependent on the presence of CAR+ T cells. (FIGS. 23A-B). At sub-curative doses of control CAR T cells, CAR T cells co-expressing BATF3 were more potent tumor killers than control CAR T cells across donors and multiple effector: target (E:T) ratios (FIG. 5A and FIG. 23B).

Next, we evaluated whether BATF3 OE could improve in vivo control of solid tumors, given the known role of T cell exhaustion in limiting ACT efficacy in the solid tumor setting. To simplify delivery of the CAR and BATF3 transgenes, we constructed all-in-one lentiviral vectors encoding a HER2 CAR coupled to either GFP or BATF3 via a 2A polypeptide skipping sequence. Using an orthotopic human breast cancer HER2+tumor model in immunodeficient NSG mice, we measured tumor volumes overtime as a function of control (GFP) CAR T cell doses (FIG. 23C). Tumor control was partial in the cohort of mice treated with 5×10⁵ CAR T cells and completely lost in the cohort treated with 105 CAR T cells. We proceeded to test whether BATF3 OE could improve the therapeutic potential of CAR T cells with several sub-curative doses. Strikingly, CAR T cells co-expressing BATF3 markedly enhanced tumor control at two sub-curative doses (2.5×10⁵ and 5×10⁵ CAR+ cells) compared to control CAR T cells (FIGS. 5B-C and FIG. 23F). Notably, the tumor growth of mice treated with the lower dose of 2.5×10⁵ control CAR T cells was completely unrestrained, mimicking that of untreated mice (FIG. 5C). In stark contrast, there was clear regression and delay of tumor growth with the matched dose of BATF3 OE CAR T cells.

To explore the mechanism driving superior tumor control with BATF3 OE, we repeated the in vivo experiment with T cells from two different donors and phenotypically characterized the CAR T cells before treatment and after collecting tumor infiltrating CAR T cells on day 3 and day 19 post-treatment (FIGS. 5D-K, FIG. 24, and FIG. 25). Across both sets of experiments, there were no differences in CAR transduction rates (>70% for all groups) or the total number of CAR+ T cells before intravenous injections between CAR constructs (FIGS. 23D-E). Again, we observed superior tumor control with BATF3 OE CAR T cells across both donors (FIGS. 24A-B). Although there were no statistically significant differences between the input CAR T cells given the small sample size, BATF3 OE cells tended to express lower levels of several exhaustion markers including LAG3, TIGIT, and TIM3 (FIGS. 24C-E).

More striking differences between the two groups emerged at the day 3 post-treatment timepoint. We detected equivalent proportions of CD8+ T cells within the tumor and circulating in peripheral blood, indicating that BATF3 OE was not improving tumor control by merely increasing T cell proliferation or tumor trafficking (FIG. 5D and FIG. 24F). Corroborating this, expression of the proliferative marker Ki-67 was equivalent between the groups (FIG. 5E). Rather, tumor infiltrating CAR T cells with BATF3 OE expressed higher levels of both TCF1 and IFNy (FIGS. 5F-G). BATF3 OE did not increase expression of TCF7 (which encodes for TCF1) under acute stimulation in vitro (FIG. 18C). However, there were seven differentially accessible sites near the TCF7 locus between control and BATF3 OE CAR T cells after chronic stimulation (FIGS. 22C, E). Notably, 5/7 sites were more accessible in BATF3 OE cells including all three intragenic regions, while four distal intergenic regions were split evenly between the two groups (FIG. 22E). These data suggest that BATF3 OE can partially counter heterochromatinization of the TCF7 locus during chronic antigen stimulation and retain higher levels of TCF1 expression.

As reflected in the tumor growth curves, we detected a higher proportion of tumor infiltrating CAR T cells in the BATF3 OE group at the final day 19 timepoint, likely due to smaller tumor sizes, as the absolute number of T cells were similar between the two groups (FIGS. 5H-I). We did not detect any CAR T cells in peripheral blood for either group. To gain further insight into transcriptional regulation, we stained the tumor infiltrating CAR T cells for the following TFs: TCF1, TBET, EOMES, GATA3, ID2, ID3, and IRF4. Interestingly, TCF1 was no longer differentially expressed, but ID3 (a downstream TF of TCF1) was upregulated in the BATF3 OE group (FIGS. 5J-K). Therefore, BATF3 OE T cells may have gradually transitioned from transcriptional programs driven by TCF1 to ID3.

Given the enhanced tumor control conferred by BATF3 OE in CD8+ T cells, we were curious whether BATF3 OE programmed a transcriptional signature associated with clinical response to ACT. Suggestive of this, in a recent clinical trial, non-responders to CD19-targeting CAR T cell therapy had a significantly higher proportion of CD8+ T cells in a cytotoxic or exhausted phenotype than responders. This prompted us to systematically identify DEGs between the infused CD8+CD19 CAR T cell product of responders and non-responders (FIG. 26A). There were 147 DEGs between CD8+ T cells of responders and non-responders in this dataset. We then subset our bulk RNA-seq data with BATF3 OE to query the expression of these genes. Of the 147 DEGs, 144 genes were detected in our RNA-seq data. Strikingly, BATF3 OE silenced 35% (23/65) of genes associated with nonresponse and activated 20% (16/79) of genes associated with response (FIG. 5L). Seven of the ten genes most strongly associated with clinical outcome were regulated in a favorable direction. Conversely, only 4.9% (7/144) of genes were regulated in a direction opposing positive clinical response, providing further evidence that BATF3 OE drives a transcriptional program associated with positive clinical outcomes.

### Example 8

### CRISPR Knockout Screens Reveal Co-Factors of BATF3 and Novel Targets for Cancer Immunotherapy

BATF3 is a member of the AP-1 TF family, which regulates diverse biological processes across many cell types through complex and highly specific transcriptional control. This transcriptional specificity is enabled by combinatorial interactions between AP-1 TFs, which form cell-type specific homo- or hetero-dimers to regulate distinct gene expression programs. Several AP-1 complexes such as BATF-JUN heterodimers can interact with interferon-regulatory factors (IRF) at AP-1-IRF consensus elements, providing further flexibility in gene regulation. BATF3 is a compact AP-1 TF with only a basic DNA binding domain and a leucine zipper motif. Unlike other AP-1 TFs, BATF3 lacks additional protein domains such as a transactivation domain for gene activation. We therefore speculated that BATF3 was interacting with other TFs to impact gene expression and chromatin accessibility. Additionally, we reasoned that other TFs might compete with or inhibit BATF3 and that deleting these factors would further amplify BATF3's effects.

To identify cooperative TFs, downstream factors, and barriers to T cell reprogramming, we conducted parallel CRISPR knockout (CRISPRko) screens with gRNA libraries targeting all human transcription factors genes (TFome), with or without BATF3 OE. We selected IL7R expression as the readout for these screens for two reasons. First, IL7R is expressed in 20-50% of CD8+ T cells at baseline, making it feasible to recover gene hits in both directions, unlike ubiquitously silenced and highly expressed genes. Second, BATF3 OE profoundly increases IL7R expression (FIGS. 4A-B), thus providing a proxy for BATF3 activity. We expected that IL7R induction by BATF3 would be attenuated if cooperative or downstream TFs were deleted. We designed the TFome gRNA library by subsetting a genome wide knockout library (4 gRNAs per gene) for 1,612 TFs. We included four IL7R-targeting gRNAs as positive controls and 550 non-targeting gRNAs as negative controls. We cloned the 7,000 gRNA library into two lentiviral plasmids encoding for either mCherry or BATF3 and transduced CD8+ T cells from two donors in parallel with each library. The following day, we electroporated Cas9 protein to facilitate gene editing and then expanded the edited cells. After nine days of expansion, we sorted the cells into the lower and upper 10% tails of IL7R expression and sequenced the gRNA libraries from each population (FIG. 6A).

As expected, multiple IL7R gRNAs were the most enriched gRNAs in the IL7R low population across both screens (FIG. 6B). Notably, BATF3 gRNAs only emerged in the screen with BATF3 OE as BATF3 is lowly expressed at baseline (FIG. 6B). BATF3 gRNAs indiscriminately target endogenous and exogenous BATF3, indicating that knocking out exogenous BATF3 nullified its effects. Many DNMT1 and FOXO1 gRNAs were strongly enriched in the IL7R low population, corroborating findings from our CRISPRi cell-sorting screens and subsequent scRNA-seq characterization of DNMT1 and FOXO1 gene silencing. Additionally, we recovered multiple gRNA hits for many genes with all four gRNAs emerging for several genes (e.g FOXO1, FOXP1, and RUNX3) (FIG. 27A). The baseline expression of target gene hits was significantly higher than that of non-hit genes, as knockout screens can only capture the effects of expressed genes (FIG. 27B).

We then compared gRNA and gene-level enrichment between the CRISPRko screens with or without BATF3 OE (FIGS. 6C-D). This enabled us to classify genes that regulate IL7R expression in a BATF3-independent or BATF3-dependent manner. For example, FOXO1 and DNMT1 were among the strongest gene hits in the IL7R low population across both screens, indicating BATF3-independent effects. Because BATF3 OE increased the dynamic range of IL7R expression, we were also able to capture unique genes enriched in IL7R low population in the CRISPRko screen with BATF3-OE. These genes represent potential co-factors or downstream actuators of BATF3-mediated effects. Given the known interaction between AP-1 and IRF TFs, we were particularly interested in members of these families that were exclusively enriched in the IL7R low population with BATF3 OE. BATF3, JUNB, and IRF4 were the top genes meeting these criteria, suggesting that BATF3 interacts with JUNB and IRF4 to mediate transcriptional control in CD8+ T cells (FIG. 6C and FIG. 27C-D).

Both screens also revealed candidate gene targets for further improving ACT as ablating these TFs led to higher levels of IL7R expression (FIG. 6C). The most enriched genes in the TF-KO only screen included ZNF217, RUNX3, FOXP1, GATA3, GF11, AHR, ETS1, ZNF626, and FOXP3. Because BATF3 OE induces IL7R expression, it was more challenging to detect genes enriched in the IL7R high population in the screen with BATF3 OE. In addition, we speculated that some TFs whose effects were lost with BATF3 OE might be downstream targets of BATF3. Indeed, our RNA-seq analysis (FIG. 4, FIG. 18) revealed that several of the top TFs including FOXP1, ETS1, and FOXP3 were all downregulated by BATF3 OE. This indicates that further reducing the expression levels of these TFs did not affect IL7R expression.

Interestingly, there were three overlapping hits in the IL7R high population between screens: ZNF217, GATA3, and AHR, suggesting that knocking out these genes increased IL7R expression individually and in combination with BATF3 OE. ZNF217 was the top hit across both screens and has not previously been characterized in the context of T cell biology. GATA3 has been shown to promote CD8+ T cell dysfunction with features reminiscent of a regulatory T cell phenotype and targeted deletion of GATA3 improves tumor control. Moreover, both GATA3 and AHR can activate FOXP3 expression in regulatory T cells, providing further evidence of a link between T cell dysfunction and T cell regulatory activity.

We individually validated the effects of knocking out IL7R, BATF3, JUNB, IRF4, ZNF217, and GATA3 with and without BATF3 OE (FIGS. 6E-G). Consistent with previous findings, BATF3 increased IL7R expression by >40% in control CD8+ T cells (~33% to 77% IL7R+) with a non-targeting gRNA (FIG. 6E). Ablating BATF3 led to partial restoration back to control IL7R levels, presumably due to incomplete nuclease activity across ectopic lentiviral copies of BATF3 in all cells. The effects of BATF3 OE were profoundly negated with either JUNB and IRF4 knockouts with both reducing IL7R expression similarly (~30% decrease for JUNB and -28% decrease for IRF4) (FIGS. 6E-F). Conversely, genetic disruption of GATA3 and ZNF217 increased baseline IL7R levels by -10% and -18%, respectively (FIG. 6E). Combined with BATF3 OE, the fraction of IL7R+ cells did not increase with GATA3 deletion, however the relative fluorescent intensity increased by 36%.

Finally, the combined effect of BATF3 OE and ZNF217 knockout led to a significant proportion of IL7R+ T cells (>84%) (FIG. 6G and FIG. 27E).

### Example 9

### Discussion

In this study, we developed and characterized compact and efficient dSaCas9-based epigenome editors to systematically map transcriptional and epigenetic regulators of primary human CD8+ T cell state through complementary loss-of-function and gain-of-function CRISPRi/a screens. Although we assayed the effects of 120 genes in our CRISPRi/a screens, this technology could readily be scaled to profile all catalogued human genes for their coordination of complex T cell phenotypes. Nevertheless, our CRISPRi/a screens recovered many known and novel regulators of CD8+ T cell state with a striking convergence on BATF3. A prominent effect of BATF3 overexpression was activation of IL7R, which encodes for the IL-7 receptor. A primary reason for lymphodepleting regimens before CAR T cell infusion in clinical protocols is to maximize the availability of homeostatic cytokines (IL-2, IL-7, and IL-15) by eliminating competing immune cells. Increased IL7R expression on engineered T cells therefore might increase their sensitivity to IL-7 signaling and enable lower doses of conditioning lymphodepletion agents, which increase the risk of infection and have other associated toxicities.

BATF3 overexpression markedly enhanced the ability of CD8+ T cells to control tumor growth in vitro and in vivo. The compact size of BATF3 could seamlessly integrate into current manufacturing processes of FDA-approved ACTs, which all use lentivirus to deliver the CAR or TCR to donor T cells. Before translating promising gene modules such as BATF3 overexpression into the clinic, it will be important to carefully assess the safety of engineered T cells. Although the progeny of a single TET2^{null} CAR T cell clone cured an advanced refractory chronic lymphocytic leukemia (CLL) patient, a recent study highlighted that biallelic deletion of TET2 in combination with sustained expression of BATF3 can lead to antigen-independent clonal T cell expansion. BATF3 OE alone does not induce adverse effects in T cells, but the BATF-IRF axis can be oncogenic in the context of other genetic and epigenetic aberrations such as mutations, deletions, translocations, and duplications. We did not detect increased levels of MYC or Ki-67 expression in our RNA-seq data nor did we detect elevated numbers of CD8+ CAR+ T cells with BATF3 OE after 9 days of in vitro expansion and nearly three weeks of in vivo surveillance in tumor-bearing mice. Nevertheless, future work could focus on alternative delivery strategies such as transient delivery of mRNA or self-amplifying mRNA encoding for the transgene, tuning transgene expression through regulatory elements or genetic circuits, or suicide switches to control the activity of T cells in vivo.

To our knowledge, this work is the first example that combines TF overexpression with a TFome knockout screen to dissect co-factors and downstream factors and highlights the power of this approach. These screens provided insight into the mechanism by which BATF3 programs transcriptional changes. Specifically, our data combined with existing data from other cell types supports a model where BATF3 heterodimerizes with JUNB and interacts with IRF4 to drive transcriptional programs in CD8+ T cells. The dynamic and combinatorial interactions between AP-1 TFs have repeatedly been shown to control biological processes that dictate T cell state and function and are promising therapeutic candidates for ACT. This investigation also identified novel factors, such as ZNF217, which have not previously been associated with controlling T cell state or AP-1 gene regulation, which will be worthy targets of additional study. Overall, this work expands the toolkit of epigenome editors and our understanding of regulators of CD8+ T cell state and function. This catalogue of genes could serve as a basis for engineering the next generation of cancer immunotherapies.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A composition comprising a modulator of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1.

Clause 2. The composition of clause 1, wherein the gene is ZNF217 or ETS1.

Clause 3. The composition of clause 1, wherein the gene is RBSN, PRDM1, GATA3, or RUNX3.

Clause 4. The composition of clause 1, wherein the modulator is an inhibitor and the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

Clause 5. The composition of clause 1, wherein the modulator is an activator and the gene is FOXO1, MDB2, YY1, DNMT1, IRF1, CTCF, GTF2B, or HIC1.

Clause 6. The composition of any one of clauses 1-5, further comprising a modulator of BATF3.

Clause 7. The composition of any one of clauses 1-6, wherein the composition modulates T cells, and wherein modulating T cells comprises increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof.

Clause 8. The composition of clause 7, wherein the composition modulates gene expression within the T cell.

Clause 9. The composition of clause 7 or 8, wherein the composition increases expression of IL7RA in the T cell, or decreases expression of CCR7 in the T cell, or a combination thereof.

Clause 10. The composition of any one of clauses 1-9, wherein the modulator comprises a polypeptide, or a polynucleotide, or a small molecule, or siRNA, or shRNA, or a combination thereof.

Clause 11. The composition of clause 10, wherein the modulator comprises siRNA or shRNA, or a polynucleotide selected from SEQ ID NOs: 337-369 or a fragment thereof, or a polypeptide selected from SEQ ID NOs: 370-402 or a fragment thereof.

Clause 12. The composition of any one of clauses 1-11, wherein the modulator comprises a DNA targeting composition, the DNA targeting composition comprising: (a) a Cas9 protein and at least one guide RNA (gRNA) that targets the Cas9 protein to the gene or a regulatory element thereof; or (b) a meganuclease, or (c) a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof.

Clause 13. The composition of clause 12, wherein the second polypeptide domain comprises a meganuclease.

Clause 14. A therapy comprising: a first composition comprising the composition of any one of clauses 1-13; and a second composition comprising a modulator of BATF3.

Clause 15. A DNA targeting composition comprising: a Cas9 protein or a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity; and at least one guide RNA (gRNA) that targets the Cas9 protein to a target gene or a regulatory element thereof when the DNA targeting composition comprises a Cas9 protein, wherein the target gene is selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1.

Clause 16. The composition of clause 15, wherein the gene is ZNF217 or ETS1.

Clause 17. The composition of clause 15, wherein the gene is RBSN, PRDM1, GATA3, or RUNX3.

Clause 18. The composition of clause 15, wherein the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1 and wherein the DNA targeting composition is an inhibitor of the gene.

Clause 19. The composition of clause 15, wherein the gene is FOXO1, MDB2, YY1, DNMT1, IRF1, CTCF, GTF2B, or HIC1 and wherein the DNA targeting composition is an activator of the gene.

Clause 20. The composition of any one of clauses 15-19, wherein the gRNA is encoded by a polynucleotide comprising a sequence selected from SEQ ID NOs: 73-204, or comprises a sequence selected from SEQ ID NOs: 205-336.

Clause 21. The composition of any one of clauses 15-20, wherein the Cas protein comprises a Streptococcus pyogenes Cas9 protein, or a Staphylococcus aureus Cas9 protein, or any fragment thereof.

Clause 22. The composition of any one of clauses 15-21, wherein the Cas9 protein comprises the amino acid sequence of one of SEQ ID NOs: 26-29, or any fragment thereof, and/or wherein the Cas9 protein is encoded by a polynucleotide comprising a sequence selected from SEQ ID NOs: 30-39 and/or wherein the Cas9 protein comprises an amino acid sequence having at least 90% or greater identity to a sequence selected from SEQ ID NOs: 26-29, or any fragment thereof, and/or wherein the Cas9 protein is encoded by a polynucleotide comprising a sequence having at least 90% or greater identity to a sequence selected from SEQ ID NOs: 30-39, or any fragment thereof, and/or wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof, and/or wherein the Cas9 protein is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

Clause 23. The composition of any one of clauses 15-22, wherein the fusion protein comprises more than one second polypeptide domain.

Clause 24. The composition of any one of clauses 15-23, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

Clause 25. The composition of any one of clauses 15-24, wherein the second polypeptide domain has transcription repression activity.

Clause 26. The composition of clause 21, wherein the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

Clause 27. The composition of clause 25 or 26, wherein the second polypeptide domain comprises KRAB or Fokl.

Clause 28. The composition of clause 27, wherein KRAB comprises the amino acid sequence of SEQ ID NO: 45, or any fragment thereof, and/or wherein KRAB is encoded by a polynucleotide comprising the sequence of SEQ ID NO: 46, and/or wherein KRAB comprises an amino acid sequence having at least 90% or greater identity to SEQ ID NO: 45, or any fragment thereof, and/or wherein KRAB is encoded by a polynucleotide comprising a sequence having at least 90% or greater identity to SEQ ID NO: 46, or any fragment thereof, and/or wherein KRAB comprises an amino acid sequence having one, two, three, four, five or more changes selected from amino acid substitutions, insertions, or deletions, relative to SEQ ID NO: 45, or any fragment thereof, and/or wherein KRAB is encoded by a polynucleotide comprising a sequence having one, two, three, four, five or more changes selected from nucleotide substitutions, insertions, or deletions, relative to SEQ ID NO: 46, or any fragment thereof.

Clause 29. The composition of any one of clauses 25-28, wherein the fusion protein comprises the amino acid sequence of SEQ ID NO: 47 or 49, or any fragment thereof, and/or wherein the fusion protein is encoded by a polynucleotide comprising the sequence of SEQ ID NO: 48 or 50, and/or wherein the fusion protein comprises an amino acid sequence having at least 90% or greater identity to SEQ ID NO: 47 or 49, or any fragment thereof, and/or wherein the fusion protein is encoded by a polynucleotide comprising a sequence having at least 90% or greater identity to SEQ ID NO: 48 or 50, or any fragment thereof, and/or wherein the fusion protein comprises an amino acid sequence having one, two, three, four, five or more changes selected from amino acid substitutions, insertions, or deletions, relative to SEQ ID NO: 47 or 49, or any fragment thereof, and/or wherein the fusion protein is encoded by a polynucleotide having one, two, three, four, five or more changes selected from nucleotide substitutions, insertions, or deletions, relative to SEQ ID NO: 48 or 50.

Clause 30. The composition of any one of clauses 15-24, wherein the second polypeptide domain has transcription activation activity.

Clause 31. The composition of clause 30, wherein the gene is FOXO1, MDB2, YY1, DNMT1, IRF1, CTCF, GTF2B, or HIC1.

Clause 32. The composition of any one of clauses 29-31, wherein the second polypeptide domain comprises a polypeptide selected from VP16, VP64, p65, TET1, VPR, VPH, Rta, and p300, or a fragment thereof.

Clause 33. The composition of clause 32, wherein the second polypeptide domain comprises VP64, p300, VPH, or VPR, or a fragment thereof.

Clause 34. The composition of clause 32 or 33, wherein the second polypeptide domain comprises the amino acid sequence of SEQ ID NO: 41, 42, 53, or 55, or any fragment thereof, and/or wherein the second polypeptide domain is encoded by a polynucleotide comprising the sequence of SEQ ID NO: 54 or 56, and/or wherein the second polypeptide domain comprises an amino acid sequence having at least 90% or greater identity to SEQ ID NO: 41, 42, 53, or 55, or any fragment thereof, and/or wherein the second polypeptide domain is encoded by a polynucleotide comprising a sequence having at least 90% or greater identity to SEQ ID NO: 54 or 56, or any fragment thereof, and/or wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof, and/or wherein the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof.

Clause 35. The composition of any one of clauses 30-35, wherein the fusion protein comprises the amino acid sequence of SEQ ID NO: 43, or any fragment thereof, and/or wherein the fusion protein is encoded by a polynucleotide comprising the sequence of SEQ ID NO: 44, and/or wherein the fusion protein comprises an amino acid sequence having at least 90% or greater identity to SEQ ID NO: 43, or any fragment thereof, and/or wherein the fusion protein is encoded by a polynucleotide comprising a sequence having at least 90% or greater identity to SEQ ID NO: 44, or any fragment thereof, and/or wherein the fusion protein comprises an amino acid sequence having one, two, three, four, five or more changes selected from amino acid substitutions, insertions, or deletions, relative to SEQ ID NO: 43, or any fragment thereof, and/or wherein the fusion protein is encoded by a polynucleotide comprising a sequence having one, two, three, four, five or more changes selected from nucleotide substitutions, insertions, or deletions, relative to SEQ ID NO: 44.

Clause 36. A composition for increasing T cells, the composition comprising an inhibitor of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

Clause 37. The composition of clause 36, wherein the inhibitor comprises a shRNA or siRNA targeting the gene or a fragment thereof.

Clause 38. The composition of clause 36, wherein the inhibitor comprises the DNA targeting composition of any one of clauses 15-29, and wherein the second polypeptide domain has transcription repression activity.

Clause 39. A composition for increasing T cells, the composition comprising an activator of a gene selected from FOXO1, MDB2, YY1, DNMT1, IRF1, CTCF, GTF2B, or HIC1.

Clause 40. The composition of clause 39, wherein the activator comprises a polynucleotide encoding the gene, or a polypeptide encoded by the gene, or a combination thereof.

Clause 41. The composition of clause 39 or 40, wherein the activator comprises the DNA targeting composition of any one of clauses 15-24 and 30-35, and wherein the second polypeptide domain has transcription activation activity.

Clause 42. The composition of any one of clauses 1-41, wherein the composition further comprises an activator of the BATF3 gene.

Clause 43. The composition of clause 42, wherein the activator of the BATF3 gene comprises a polynucleotide encoding BATF3, or a BATF3 polypeptide, or the DNA targeting composition of any one of clauses 15-24 and 30-35 wherein the second polypeptide domain has transcription activation activity, or a combination thereof.

Clause 44. The composition of any one of clauses 1-43, further comprising at least one cancer therapy.

Clause 45. An isolated polynucleotide sequence encoding the composition of any one of clauses 1-44.

Clause 46. A vector comprising the isolated polynucleotide sequence of clause 45.

Clause 47. A cell comprising the composition of any one of clauses 1-44, or the isolated polynucleotide sequence of clause 45, or the vector of clause 46, or a combination thereof.

Clause 48. The cell of clause 47, wherein the cell is a CD8+ T cell.

Clause 49. A pharmaceutical composition comprising: the composition of any one of clauses 1-44, or the isolated polynucleotide sequence of clause 45, or the vector of clause 46, or a combination thereof.

Clause 50. A method of modulating T cells, the method comprising administering to a cell or a subject the composition of any one of clauses 1-44, or the isolated polynucleotide sequence of clause 45, or the vector of clause 46, or the cell of clause 47 or 48, or the pharmaceutical composition of clause 49, or a combination thereof.

Clause 51. The method of clause 50, wherein modulating T cells comprises increasing T cells, or increasing memory T cells, or preventing T cell exhaustions, or reversing T cell exhaustions, or a combination thereof.

Clause 52. The method of clause 51, wherein the composition or isolated polynucleotide sequence or vector is administered to a T cell, and wherein the T cell thereby increases expression of IL7RA, or decreases expression of CCR7, or a combination thereof.

Clause 53. A method of increasing T cells, the method comprising administering to a cell or a subject the composition of any one of clauses 1-44, or the isolated polynucleotide sequence of clause 45, or the vector of clause 46, or the cell of clause 47 or 48, or the pharmaceutical composition of clause 49, or a combination thereof.

Clause 54. A method of enhancing adoptive T cell therapy (ACT) in a subject, the method comprising administering to the subject the composition of any one of clauses 1-44, or the isolated polynucleotide sequence of clause 45, or the vector of clause 46, or the cell of clause 47 or 48, or the pharmaceutical composition of clause 49, or a combination thereof.

Clause 55. A method of treating cancer in a subject, the method comprising administering to the subject the composition of any one of clauses 1-44, or the isolated polynucleotide sequence of clause 45, or the vector of clause 46, or the cell of clause 47 or 48, or the pharmaceutical composition of clause 49, or a combination thereof.

SEQUENCES

SEQ ID NO: 1
NRG (R = A or G; N can be any nucleotide residue, e.g., any of A, G, C, or T)
SEQ ID NO: 2
NGG (N can be any nucleotide residue, e.g., any of A, G, C, or T)
SEQ ID NO: 3
NAG (N can be any nucleotide residue, e.g., any of A, G, C, or T)
SEQ ID NO: 4
NGGNG (N can be any nucleotide residue, e.g., any of A, G, C, or T)
SEQ ID NO: 5
NNAGAAW (W = A or T; N can be any nucleotide residue, e.g., any of A, G, C, or T)
SEQ ID NO: 6
NAAR (R = A or G; N can be any nucleotide residue, e.g., any of A, G, C, or T)
SEQ ID NO: 7
NNGRR (R = A or G; N can be any nucleotide residue, e.g., any of A, G, C, or T)
SEQ ID NO: 8
NNGRRN (R = A or G; N can be any nucleotide residue, e.g., any of A, G, C, or T)
SEQ ID NO: 9
NNGRRT (R = A or G; N can be any nucleotide residue, e.g., any of A, G, C, or T)
SEQ ID NO: 10
NNGRRV (R = A or G; N can be any nucleotide residue, e.g., any of A, G, C, or T; V = A or
C or G)
SEQ ID NO: 11
NNNNGATT (N can be any nucleotide residue, e.g., any of A, G, C, or T)
SEQ ID NO: 12
NNNNGNNN (N can be any nucleotide residue, e.g., any of A, G, C, or T)
SEQ ID NO: 13
NGA (N can be any nucleotide residue, e.g., any of A, G, C, or T)
SEQ ID NO: 14
NNNRRT (R = A or G; N can be any nucleotide residue, e.g., any of A, G, C, or T)
SEQ ID NO: 15
ATTCCT
SEQ ID NO: 16
NGAN (N can be any nucleotide residue, e.g., any of A, G, C, or T)
SEQ ID NO: 17
NGNG (N can be any nucleotide residue, e.g., any of A, G, C, or T)
SEQ ID NO: 18
DNA sequence of the gRNA constant region
gtttaagagctatgctggaaacagcatagcaagtttaaataaggctagtccgttatcaacttgaaaaa
gtggcaccgagtcggtgc
SEQ ID NO: 19
RNA sequence of the gRNA constant region
guuuaagagcuaugcuggaaacagcauagcaaguuuaaauaaggcuaguccguuaucaacuugaaaaa
guggcaccgagucggugc
SEQ ID NO: 20
SV40 NLS (Pro-Lys-Lys-Lys-Arg-Lys-Val)
SEQ ID NO: 21
GS linker (Gly-Gly-Gly-Gly-Ser)ₙ, wherein n is an integer between 0 and 10
SEQ ID NO: 22
Gly-Gly-Gly-Gly-Gly
SEQ ID NO: 23
Gly-Gly-Ala-Gly-Gly
SEQ ID NO: 24
Gly-Gly-Gly-Gly-Ser-Ser-Ser
SEQ ID NO: 25
Gly-Gly-Gly-Gly-Ala-Ala-Ala
SEQ ID NO: 26
Streptococcus pyogenes Cas9
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 27
Staphylococcus aureus Cas9
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 28
Streptococcus pyogenes Cas9 (with D10A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 29
Streptococcus pyogenes Cas9 (with D10A, H849A)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 30
Polynucleotide sequence of D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 31
Polynucleotide sequence of N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 32
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
SEQ ID NO: 33
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 34
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 35
codon optimized nucleic acid sequence encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 36
codon optimized nucleic acid sequence encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 37
codon optimized nucleic acid sequence S. aureus Cas9 gaaaaagcgc
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 38
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 39
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 40
Vector (pDO242) encoding codon optimized nucleic acid sequence encoding S. aureus
Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 41
Human p300 (with L553M mutation) protein
MAENVVEPGPPSAKRPKLSSPALSASASDGTDFGSLFDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 42
Human p300 Core Effector protein (aa 1048-1664 of SEQ ID NO: 41)
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 43
VP64-dCas9-VP64 protein
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
SEQ ID NO: 44
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 45
Polypeptide sequence of KRAB protein
RTLVTFKDVFVDFTREEWKLLDTAQQILYRNVMLENYKNLVSLGYQLTKPDVILRLEKGEEP
WLV
SEQ ID NO: 46
Polynucleotide sequence for KRAB
cggacactggtgaccttcaaggatgtgtttgtggacttcaccagggaggagtggaagctgct
ggacactgctcagcagatcctgtacagaaatgtgatgctggagaactataagaacctggttt
ccttgggttatcagcttactaagccagatgtgatcctccggttggagaagggagaagagccc
tggctggtg
SEQ ID NO: 47
Polypeptide sequence of Streptococcus pyogenes dCas9-KRAB protein
MDYKDHDGDYKDHDIDYKDDDDKMAPKKKRKVGRGMDKKYSIGLAIGTNSVGWAVITDEYKVPSKKFK
VLGNTDRHSIKKNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAHLFDD
KVMKQLKRRRYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFI
KRQLVETRQITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
SEQ ID NO: 48
Polynucleotide sequence encoding Streptococcus pyogenes dCas9-KRAB
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
SEQ ID NO: 49
Polypeptide sequence of Staphylococcus aureus dCas9-KRAB protein
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVFKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSFNNKVLVKQEEASKKGNRTPFQYLSSSD
SKISYETFKKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
SEQ ID NO: 50
Polynucleotide sequence of Staphylococcus aureus dCas9-KRAB protein
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
SEQ ID NO: 51
Polypeptide sequence of Tet1CD
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASFSFGCSWSMYFNGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSFLTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
SEQ ID NO: 52
Polynucleotide sequence of Tet1CD
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 53
Protein sequence for VPH
DALDDFDLDMLGSDALDDFDLDMLGSDALDDFDLDMLGSDALDDFDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDPAPTPLGTSGLPNGLSGDEDFSSIADMDFSALLSQISSSGQGGGGSGFSVDTSALLDLFSPSVT
VPDMSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
SEQ ID NO: 54
DNA sequence for VPH
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
SEQ ID NO: 55
Protein sequence for VPR
DALDDFDLDMLGSDALDDFDLDMLGSDALDDFDLDMLGSDALDDFDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDFSSIADMDFSALLSQISSGSGSGSRDSREGMF
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTFLNDECLLHAMHISTGLSIFDTSLF
SEQ ID NO: 56
DNA sequence for VPR
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt
SEQ ID NO: 57
ABE ecTadA wild-type, protein
SEVEFSHEYWMRHALTLAKRAWDEREVPVGAVLVHNNRVIGEGWNRPIGRHDPTAHAEIMALRQGGLV
MQNYRLIDATLYVTLEPCVMCAGAMIHSRIGRVVFGARDAKTGAAGSLMDVLHHPGMNHRVEITEGIL
ADECAALLSDFFRMRRQEIKAQKKAQSSTD
SEQ ID NO: 58
ABE ecTadA*7.9, protein
SEVEFSHEYWMRHALTLAKRALDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHYPGMNHRVEITEGIL
ADECNALLCYFFRMPRQVFNAQKKAQSSTD
SEQ ID NO: 59
ABE ecTadA*7.10, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHYPGMNHRVEITEGIL
ADECAALLCYFFRMPRQVFNAQKKAQSSTD
SEQ ID NO: 60
ABE ecTadA*8e, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNSKRGAAGSLMNVLNYPGMNHRVEITEGIL
ADECAALLCDFYRMPRQVFNAQKKAQSSIN
SEQ ID NO: 61
ABE ecTadA*8.8, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 62
ABE ecTadA*8.13, protein
SEVEFSHEYWMRHALTLAKRARDEREVPVGAVLVLNNRVIGEGWNRAIGLHDPTAHAEIMALRQGGLV
MQNYRLYDATLYVTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 63
ABE ecTadA*8.17, protein
SEVEFSHEYWMRHALTLAKRARDEREVPVGAVLVLNNRVIGEGWNRAIGLHDPTAHAEIMALRQGGLV
MQNYRLIDATLYSTFEPCVMCAGAMIHSRIGRVVFGVRNAKTGAAGSLMDVLHYPGMNHRVEITEGIL
ADECAALLCYFFRMPRRVFNAQKKAQSSTD
SEQ ID NO: 64
ABE ecTadA*8.20, protein
ecTadA*8.20
SEVEFSHEYWMRHALTLAKRARDEREVPVGAVLVLNNRVIGEGWNRAIGLHDPTAHAEIMALRQGGLV
MQNYRLYDATLYSTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 65
ABE ecTadA wild-type, DNA
tctgaagtcgagtttagccacgagtattggatgaggcacgcactgaccctggcaaagcgagcatggga
tgaaagagaagtccccgtgggcgccgtgctggtgcacaacaatagagtgatcggagagggatggaaca
ggccaatcggccgccacgaccctaccgcacacgcagagatcatggcactgaggcagggaggcctggtc
atgcagaattaccgcctgatcgatgccaccctgtatgtgacactggagccatgcgtgatgtgcgcagg
agcaatgatccacagcaggatcggaagagtggtgttcggagcacgggacgccaagaccggcgcagcag
gctccctgatggatgtgctgcaccaccccggcatgaaccaccgggtggagatcacagagggaatcctg
gcagacgagtgcgccgccctgctgagcgatttctttagaatgcggagacaggagatcaaggcccagaa
gaaggcacagagctccaccgac
SEQ ID NO: 66
ABE ecTadA*7.9, DNA
tccgaagtcgagttttcccatgagtactggatgagacacgcattgactctcgcaaagagggctctcga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtaacgcgctgttgtgttacttttttcgcatgcccaggcaggtctttaacgcccagaa
aaaagcacaatcctctactgac
SEQ ID NO: 67
ABE ecTadA*7.10, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtgcggcgctgttgtgttacttttttcgcatgcccaggcaggtctttaacgcccagaa
aaaagcacaatcctctactgac
SEQ ID NO: 68
ABE ecTadA*8e, DNA
tctgaggtggagttttcccacgagtactggatgagacatgccctgaccctggccaagagggcacggga
tgagagggaggtgcctgtgggagccgtgctggtgctgaacaatagagtgatcggcgagggctggaaca
gagccatcggcctgcacgacccaacagcccatgccgaaattatggccctgagacagggcggcctggtc
atgcagaactacagactgattgacgccaccctgtacgtgacattcgagccttgcgtgatgtgcgccgg
cgccatgatccactctaggatcggccgcgtggtgtttggcgtgaggaactcaaaaagaggcgccgcag
gctccctgatgaacgtgctgaactaccccggcatgaatcaccgcgtcgaaattaccgagggaatcctg
gcagatgaatgtgccgccctgctgtgcgatttctatcggatgcctagacaggtgttcaatgctcagaa
gaaggcccagagctccatcaac
SEQ ID NO: 69
ABE ecTadA*8.8, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgactctggtggttcttctggtggttctagcggcagcgagactcccggga
cctcagagtccgccacacccgaaagttctggtggttcttctggtggttct
SEQ ID NO: 70
ABE ecTadA*8.13, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgactttatgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgac
SEQ ID NO: 71
ABE ecTadA*8.17, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtactcgacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtgcggcgctgttgtgttacttttttcgcatgcccaggcgtgtctttaacgcccagaa
aaaagcacaatcctctactgac
SEQ ID NO: 72
ABE ecTadA*8.20, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgactttatgatgcgacgctgtactcgacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgac

1. A composition comprising a modulator of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1.

2. The composition of claim 1, wherein the gene is ZNF217 or ETS1.

3. The composition of claim 1, wherein the gene is RBSN, PRDM1, GATA3, or RUNX3.

4. The composition of claim 1, wherein the modulator is an inhibitor and the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

5. The composition of claim 1, wherein the modulator is an activator and the gene is FOXO1, MDB2, YY1, DNMT1, IRF1, CTCF, GTF2B, or HIC1.

6. The composition of any one of claims 1-5, further comprising a modulator of BATF3.

7. The composition of any one of claims 1-6, wherein the composition modulates T cells, and wherein modulating T cells comprises increasing T cells, or increasing memory T cells, or increasing the lifetime of a T cell, or preventing T cell exhaustions, or reversing T cell exhaustions, or reducing T cell exhaustion, or enhancing the therapeutic potential of T cells, or a combination thereof.

8. The composition of claim 7, wherein the composition modulates gene expression within the T cell.

9. The composition of claim 7 or 8, wherein the composition increases expression of IL7RA in the T cell, or decreases expression of CCR7 in the T cell, or a combination thereof.

10. The composition of any one of claims 1-9, wherein the modulator comprises a polypeptide, or a polynucleotide, or a small molecule, or siRNA, or shRNA, or a combination thereof.

11. The composition of claim 10, wherein the modulator comprises siRNA or shRNA, or a polynucleotide selected from SEQ ID NOs: 337-369 or a fragment thereof, or a polypeptide selected from SEQ ID NOs: 370-402 or a fragment thereof.

12. The composition of any one of claims 1-11, wherein the modulator comprises a DNA targeting composition, the DNA targeting composition comprising:
(a) a Cas9 protein and at least one guide RNA (gRNA) that targets the Cas9 protein to the gene or a regulatory element thereof; or
(b) a meganuclease, or
(c) a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity, wherein when the first polypeptide domain comprises a Cas9 protein the DNA targeting composition further comprises at least one guide RNA (gRNA) that targets the Cas9 protein to the gene or a regulatory element thereof.

13. The composition of claim 12, wherein the second polypeptide domain comprises a meganuclease.

14. A therapy comprising:
a first composition comprising the composition of any one of claims 1-13; and
a second composition comprising a modulator of BATF3.

15. A DNA targeting composition comprising:
a Cas9 protein or a fusion protein, wherein the fusion protein comprises two heterologous polypeptide domains, wherein the first polypeptide domain comprises a zinc finger protein or a TALE or a Cas12 protein or a Cas13 protein or a Cas9 protein, and wherein the second polypeptide domain has an activity selected from transcription activation activity, transcription repression activity, nuclease activity, base editing activity, prime editing activity, transcription release factor activity, histone modification activity, nucleic acid association activity, methylase activity, and demethylase activity; and
at least one guide RNA (gRNA) that targets the Cas9 protein to a target gene or a regulatory element thereof when the DNA targeting composition comprises a Cas9 protein, wherein the target gene is selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, RUNX1, FOXO1, MBD2, YY1, DNMT1, IRF1, CTCF, GTF2B, and HIC1.

16. The composition of claim 15, wherein the gene is ZNF217 or ETS1.

17. The composition of claim 15, wherein the gene is RBSN, PRDM1, GATA3, or RUNX3.

18. The composition of claim 15, wherein the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1 and wherein the DNA targeting composition is an inhibitor of the gene.

19. The composition of claim 15, wherein the gene is FOXO1, MDB2, YY1, DNMT1, IRF1, CTCF, GTF2B, or HIC1 and wherein the DNA targeting composition is an activator of the gene.

20. The composition of any one of claims 15-19, wherein the gRNA is encoded by a polynucleotide comprising a sequence selected from SEQ ID NOs: 73-204, or comprises a sequence selected from SEQ ID NOs: 205-336.

21. The composition of any one of claims 15-20, wherein the Cas protein comprises a Streptococcus pyogenes Cas9 protein, or a Staphylococcus aureus Cas9 protein, or any fragment thereof.

22. The composition of any one of claims 15-21, wherein the Cas9 protein comprises the amino acid sequence of one of SEQ ID NOs: 26-29, or any fragment thereof,
and/or wherein the Cas9 protein is encoded by a polynucleotide comprising a sequence selected from SEQ ID NOs: 30-39
and/or wherein the Cas9 protein comprises an amino acid sequence having at least 90% or greater identity to a sequence selected from SEQ ID NOs: 26-29, or any fragment thereof,
and/or wherein the Cas9 protein is encoded by a polynucleotide comprising a sequence having at least 90% or greater identity to a sequence selected from SEQ ID NOs: 30-39, or any fragment thereof,
and/or wherein the Cas9 protein comprises an amino acid sequence having one, two, three, four, five or more changes selected from amino acid substitutions, insertions, or deletions, relative to a sequence selected from SEQ ID NOs: 26-29, or any fragment thereof,
and/or wherein the Cas9 protein is encoded by a polynucleotide comprising a sequence having one, two, three, four, five or more changes selected from nucleotide substitutions, insertions, or deletions, relative to a sequence selected from SEQ ID NOs: 30-39, or any fragment thereof.

23. The composition of any one of claims 15-22, wherein the fusion protein comprises more than one second polypeptide domain.

24. The composition of any one of claims 15-23, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su(var)3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

25. The composition of any one of claims 15-24, wherein the second polypeptide domain has transcription repression activity.

26. The composition of claim 21, wherein the gene is ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

27. The composition of claim 25 or 26, wherein the second polypeptide domain comprises KRAB or Fokl.

28. The composition of claim 27, wherein KRAB comprises the amino acid sequence of SEQ ID NO: 45, or any fragment thereof,
and/or wherein KRAB is encoded by a polynucleotide comprising the sequence of SEQ ID NO: 46,
and/or wherein KRAB comprises an amino acid sequence having at least 90% or greater identity to SEQ ID NO: 45, or any fragment thereof,
and/or wherein KRAB is encoded by a polynucleotide comprising a sequence having at least 90% or greater identity to SEQ ID NO: 46, or any fragment thereof,
and/or wherein KRAB comprises an amino acid sequence having one, two, three, four, five or more changes selected from amino acid substitutions, insertions, or deletions, relative to SEQ ID NO: 45, or any fragment thereof,
and/or wherein KRAB is encoded by a polynucleotide comprising a sequence having one, two, three, four, five or more changes selected from nucleotide substitutions, insertions, or deletions, relative to SEQ ID NO: 46, or any fragment thereof.

29. The composition of any one of claims 25-28, wherein the fusion protein comprises the amino acid sequence of SEQ ID NO: 47 or 49, or any fragment thereof,
and/or wherein the fusion protein is encoded by a polynucleotide comprising the sequence of SEQ ID NO: 48 or 50,
and/or wherein the fusion protein comprises an amino acid sequence having at least 90% or greater identity to SEQ ID NO: 47 or 49, or any fragment thereof,
and/or wherein the fusion protein is encoded by a polynucleotide comprising a sequence having at least 90% or greater identity to SEQ ID NO: 48 or 50, or any fragment thereof,
and/or wherein the fusion protein comprises an amino acid sequence having one, two, three, four, five or more changes selected from amino acid substitutions, insertions, or deletions, relative to SEQ ID NO: 47 or 49, or any fragment thereof,
and/or wherein the fusion protein is encoded by a polynucleotide having one, two, three, four, five or more changes selected from nucleotide substitutions, insertions, or deletions, relative to SEQ ID NO: 48 or 50.

30. The composition of any one of claims 15-24, wherein the second polypeptide domain has transcription activation activity.

31. The composition of claim 30, wherein the gene is FOXO1, MDB2, YY1, DNMT1, IRF1, CTCF, GTF2B, or HIC1.

32. The composition of any one of claims 29-31, wherein the second polypeptide domain comprises a polypeptide selected from VP16, VP64, p65, TET1, VPR, VPH, Rta, and p300, or a fragment thereof.

33. The composition of claim 32, wherein the second polypeptide domain comprises VP64, p300, VPH, or VPR, or a fragment thereof.

34. The composition of claim 32 or 33, wherein the second polypeptide domain comprises the amino acid sequence of SEQ ID NO: 41, 42, 53, or 55, or any fragment thereof,
and/or wherein the second polypeptide domain is encoded by a polynucleotide comprising the sequence of SEQ ID NO: 54 or 56,
and/or wherein the second polypeptide domain comprises an amino acid sequence having at least 90% or greater identity to SEQ ID NO: 41, 42, 53, or 55, or any fragment thereof,
and/or wherein the second polypeptide domain is encoded by a polynucleotide comprising a sequence having at least 90% or greater identity to SEQ ID NO: 54 or 56, or any fragment thereof,
and/or wherein the second polypeptide domain comprises an amino acid sequence having one, two, three, four, five or more changes selected from amino acid substitutions, insertions, or deletions, relative to SEQ ID NO: 41, 42, 53, or 55, or any fragment thereof,
and/or wherein the second polypeptide domain is encoded by a polynucleotide comprising a sequence having one, two, three, four, five or more changes selected from nucleotide substitutions, insertions, or deletions, relative to SEQ ID NO: 54 or 56, or any fragment thereof.

35. The composition of any one of claims 30-35, wherein the fusion protein comprises the amino acid sequence of SEQ ID NO: 43, or any fragment thereof,
and/or wherein the fusion protein is encoded by a polynucleotide comprising the sequence of SEQ ID NO: 44,
and/or wherein the fusion protein comprises an amino acid sequence having at least 90% or greater identity to SEQ ID NO: 43, or any fragment thereof,
and/or wherein the fusion protein is encoded by a polynucleotide comprising a sequence having at least 90% or greater identity to SEQ ID NO: 44, or any fragment thereof,
and/or wherein the fusion protein comprises an amino acid sequence having one, two, three, four, five or more changes selected from amino acid substitutions, insertions, or deletions, relative to SEQ ID NO: 43, or any fragment thereof,
and/or wherein the fusion protein is encoded by a polynucleotide comprising a sequence having one, two, three, four, five or more changes selected from nucleotide substitutions, insertions, or deletions, relative to SEQ ID NO: 44.

36. A composition for increasing T cells, the composition comprising an inhibitor of a gene selected from ZNF217, ETS1, RBSN, PRDM1, GATA3, RUNX3, FOXP1, GF11, AHR, ZNF626, FOXP3, ARNT, MYC, SON, TBX21, DUX4, YBX1, ZC3H8, ZNF367, ZNF335, KCMF1, TET2, ZBTB1, ZFX, or RUNX1.

37. The composition of claim 36, wherein the inhibitor comprises a shRNA or siRNA targeting the gene or a fragment thereof.

38. The composition of claim 36, wherein the inhibitor comprises the DNA targeting composition of any one of claims 15-29, and wherein the second polypeptide domain has transcription repression activity.

39. A composition for increasing T cells, the composition comprising an activator of a gene selected from FOXO1, MDB2, YY1, DNMT1, IRF1, CTCF, GTF2B, or HIC1.

40. The composition of claim 39, wherein the activator comprises a polynucleotide encoding the gene, or a polypeptide encoded by the gene, or a combination thereof.

41. The composition of claim 39 or 40, wherein the activator comprises the DNA targeting composition of any one of claims 15-24 and 30-35, and wherein the second polypeptide domain has transcription activation activity.

42. The composition of any one of claims 1-41, wherein the composition further comprises an activator of the BATF3 gene.

43. The composition of claim 42, wherein the activator of the BATF3 gene comprises a polynucleotide encoding BATF3, or a BATF3 polypeptide, or the DNA targeting composition of any one of claims 15-24 and 30-35 wherein the second polypeptide domain has transcription activation activity, or a combination thereof.

44. The composition of any one of claims 1-43, further comprising at least one cancer therapy.

45. An isolated polynucleotide sequence encoding the composition of any one of claims 1-44.

46. A vector comprising the isolated polynucleotide sequence of claim 45.

47. A cell comprising the composition of any one of claims 1-44, or the isolated polynucleotide sequence of claim 45, or the vector of claim 46, or a combination thereof.

48. The cell of claim 47, wherein the cell is a CD8+ T cell.

49. A pharmaceutical composition comprising: the composition of any one of claims 1-44, or the isolated polynucleotide sequence of claim 45, or the vector of claim 46, or a combination thereof.

50. A method of modulating T cells, the method comprising administering to a cell or a subject the composition of any one of claims 1-44, or the isolated polynucleotide sequence of claim 45, or the vector of claim 46, or the cell of claim 47 or 48, or the pharmaceutical composition of claim 49, or a combination thereof.

51. The method of claim 50, wherein modulating T cells comprises increasing T cells, or increasing memory T cells, or preventing T cell exhaustions, or reversing T cell exhaustions, or a combination thereof.

52. The method of claim 51, wherein the composition or isolated polynucleotide sequence or vector is administered to a T cell, and wherein the T cell thereby increases expression of IL7RA, or decreases expression of CCR7, or a combination thereof.

53. A method of increasing T cells, the method comprising administering to a cell or a subject the composition of any one of claims 1-44, or the isolated polynucleotide sequence of claim 45, or the vector of claim 46, or the cell of claim 47 or 48, or the pharmaceutical composition of claim 49, or a combination thereof.

54. A method of enhancing adoptive T cell therapy (ACT) in a subject, the method comprising administering to the subject the composition of any one of claims 1-44, or the isolated polynucleotide sequence of claim 45, or the vector of claim 46, or the cell of claim 47 or 48, or the pharmaceutical composition of claim 49, or a combination thereof.

55. A method of treating cancer in a subject, the method comprising administering to the subject the composition of any one of claims 1-44, or the isolated polynucleotide sequence of claim 45, or the vector of claim 46, or the cell of claim 47 or 48, or the pharmaceutical composition of claim 49, or a combination thereof.


SEQUENCE LISTING (402 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 7 aa, synthetic construct
PKKKRKV

SEQ ID NO: 21 - protein, 5 aa, synthetic construct
  REPEAT 1..5: note = 0 to 10 times
GGGGS

SEQ ID NO: 22 - protein, 5 aa, synthetic construct
GGGGG

SEQ ID NO: 23 - protein, 5 aa, synthetic construct
GGAGG

SEQ ID NO: 24 - protein, 7 aa, synthetic construct
GGGGSSS

SEQ ID NO: 25 - protein, 7 aa, synthetic construct
GGGGAAA

SEQ ID NO: 26 - protein, 1368 aa, Streptococcus pyogenes [sequence omitted]

SEQ ID NO: 27 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 31 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 33 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 34 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 36 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 37 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 38 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 39 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 40 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 42 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 43 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 46 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 47 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 48 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 49 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 50 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 51 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 53 - protein, 378 aa, synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 54 - DNA, 1134 bp, synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 55 - protein, 522 aa, synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 1566 bp, synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 57 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 58 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 59 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 60 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 61 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 62 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 63 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 64 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 65 - DNA, 498 bp, synthetic construct
  source 1..498: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgac

SEQ ID NO: 66 - DNA, 498 bp, synthetic construct
  source 1..498: mol_type = other DNA
tccgaagtcgagttttcccatgagtactggatgagacacgcattgactctcgcaaagagggctctcgatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtaacgcgctgttgtgttacttttttcgcatgcccaggcaggtctttaacgcccagaaaaaagcacaatcctctactgac

SEQ ID NO: 67 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgac

SEQ ID NO: 68 - DNA, 498 bp, synthetic construct
  source 1..498: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 69 - DNA, 594 bp, synthetic construct
  source 1..594: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttct

SEQ ID NO: 70 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 71 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcgtgtctttaacgcccagaaaaaagcacaatcctctactgac

SEQ ID NO: 72 - DNA, 498 bp, synthetic construct
  source 1..498: mol_type = other DNA
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 73 - DNA, 20 bp, synthetic construct
  source 1..20: mol_type = other DNA
ggacacataatggcaaatcg

SEQ ID NO: 74 - DNA, 20 bp, synthetic construct
  source 1..20: mol_type = other DNA
caaaatctcaccctgaaacg

SEQ ID NO: 75 - DNA, 20 bp, synthetic construct
  source 1..20: mol_type = other DNA
ccacggcgaagcgccctccg

SEQ ID NO: 76 - DNA, 20 bp, synthetic construct
  source 1..20: mol_type = other DNA
tgggtggtactgccatccgg

SEQ ID NO: 77 - DNA, 20 bp, synthetic construct
  source 1..20: mol_type = other DNA
ggtctcggagaatgaccgag

SEQ ID NO: 78 - DNA, 20 bp, synthetic construct
  source 1..20: mol_type = other DNA
cttactaatgaagtaatccg

SEQ ID NO: 79 - DNA, 20 bp, synthetic construct
  source 1..20: mol_type = other DNA
tgcatggggaggaccagtcg

SEQ ID NO: 80 - DNA, 20 bp, synthetic construct
  source 1..20: mol_type = other DNA
gagaaagcagtctttaccca

SEQ ID NO: 81 - DNA, 20 bp, synthetic construct
  source 1..20: mol_type = other DNA
tgcacataatagacccgcag

SEQ ID NO: 82 - DNA, 20 bp, synthetic construct
  source 1..20: mol_type = other DNA
tagaattgaccactatgttg

SEQ ID NO: 83 - DNA, 20 bp, synthetic construct
  source 1..20: mol_type = other DNA
ggcttctcgagcggccaacg

SEQ ID NO: 84 - DNA, 20 bp, synthetic construct
  source 1..20: mol_type = other DNA
gagtgcgcacttcatccatg

SEQ ID NO: 85 - DNA, 20 bp, synthetic construct
  source 1..20: mol_type = other DNA
ggggagcgagtgatgtacgt

SEQ ID NO: 86 - DNA, 20 bp, synthetic construct
  source 1..20: mol_type = other DNA
ggacgcgttcaagtaagcgt

SEQ ID NO: 87 - DNA, 20 bp, synthetic construct
  source 1..20: mol_type = other DNA
tttggacagatctattccag

SEQ ID NO: 88 - DNA, 20 bp, synthetic construct
  source 1..20: mol_type = other DNA
aggatgcggatatgactctg

SEQ ID NO: 89 - DNA, 20 bp, synthetic construct
  source 1..20: mol_type = other DNA
tccaagacgtccatccacca

SEQ ID NO: 90 - DNA, 20 bp, synthetic construct
  source 1..20: mol_type = other DNA
aggtaccctccgacccacca

SEQ ID NO: 91 - DNA, 20 bp, synthetic construct
  source 1..20: mol_type = other DNA
ggagctgtactcgggcacgt

SEQ ID NO: 92 - DNA, 20 bp, synthetic construct
  source 1..20: mol_type = other DNA
cagggagtgtgtgaactgtg

SEQ ID NO: 93 - DNA, 20 bp, synthetic construct
  source 1..20: mol_type = other DNA
ccccaggatgcattatcccg

SEQ ID NO: 94 - DNA, 20 bp, synthetic construct
  source 1..20: mol_type = other DNA
cactgcggcccacgaagcga

SEQ ID NO: 95 - DNA, 20 bp, synthetic construct
  source 1..20: mol_type = other DNA
ccgtgccgtaccttggattg

SEQ ID NO: 96 - DNA, 20 bp, synthetic construct
  source 1..20: mol_type = other DNA
ccgtgccgtaccttggattg

SEQ ID NO: 97 - DNA, 20 bp, synthetic construct
  source 1..20: mol_type = other DNA
agaggaggagacacatgtcg

SEQ ID NO: 98 - DNA, 20 bp, synthetic construct
  source 1..20: mol_type = other DNA
gttctgtagacttcacatgc

SEQ ID NO: 99 - DNA, 20 bp, synthetic construct
  source 1..20: mol_type = other DNA
catacaccatgtccatagag

SEQ ID NO: 100 - DNA, 20 bp, synthetic construct
  source 1..20: mol_type = other DNA
gccttctgacaattcagccc

SEQ ID NO: 101 - DNA, 20 bp, synthetic construct
  source 1..20: mol_type = other DNA
ccggtacattctctaaacgg

SEQ ID NO: 102 - DNA, 20 bp, synthetic construct
  source 1..20: mol_type = other DNA
tgggcacattgacttctccg

SEQ ID NO: 103 - DNA, 20 bp, synthetic construct
  source 1..20: mol_type = other DNA
ttcaaaccgtactcatggag

SEQ ID NO: 104 - DNA, 20 bp, synthetic construct
  source 1..20: mol_type = other DNA
acgtgcacctcgagcccgtg

SEQ ID NO: 105 - DNA, 20 bp, synthetic construct
  source 1..20: mol_type = other DNA
ttataacagactactgtctg

SEQ ID NO: 106 - DNA, 20 bp, synthetic construct
  source 1..20: mol_type = other DNA
cttacttcggatatgggact

SEQ ID NO: 107 - DNA, 20 bp, synthetic construct
  source 1..20: mol_type = other DNA
atcgcaaacaaagccaactg

SEQ ID NO: 108 - DNA, 20 bp, synthetic construct
  source 1..20: mol_type = other DNA
taccacatccactctaagca

SEQ ID NO: 109 - DNA, 20 bp, synthetic construct
  source 1..20: mol_type = other DNA
taacctggtcttccttggtg

SEQ ID NO: 110 - DNA, 20 bp, synthetic construct
  source 1..20: mol_type = other DNA
accattgcaatttagagatg

SEQ ID NO: 111 - DNA, 20 bp, synthetic construct
  source 1..20: mol_type = other DNA
ggccatagaattctctctgg

SEQ ID NO: 112 - DNA, 20 bp, synthetic construct
  source 1..20: mol_type = other DNA
ctgatcacctgtctggagca

SEQ ID NO: 113 - DNA, 20 bp, synthetic construct
  source 1..20: mol_type = other DNA
gagggtgccaccatgactag

SEQ ID NO: 114 - DNA, 20 bp, synthetic construct
  source 1..20: mol_type = other DNA
cccacccacagggatcaacg

SEQ ID NO: 115 - DNA, 20 bp, synthetic construct
  source 1..20: mol_type = other DNA
acccaggcatcatccgacaa

SEQ ID NO: 116 - DNA, 20 bp, synthetic construct
  source 1..20: mol_type = other DNA
cctacttaggcactgccagg

SEQ ID NO: 117 - DNA, 20 bp, synthetic construct
  source 1..20: mol_type = other DNA
agatgtctaacgataaggag

SEQ ID NO: 118 - DNA, 20 bp, synthetic construct
  source 1..20: mol_type = other DNA
caagctaaccatcttacgca

SEQ ID NO: 119 - DNA, 20 bp, synthetic construct
  source 1..20: mol_type = other DNA
tggattgtgttggagagtgt

SEQ ID NO: 120 - DNA, 20 bp, synthetic construct
  source 1..20: mol_type = other DNA
tggggaacctcacttcgtgg

SEQ ID NO: 121 - DNA, 20 bp, synthetic construct
  source 1..20: mol_type = other DNA
cttcggggagacaacgacgg

SEQ ID NO: 122 - DNA, 20 bp, synthetic construct
  source 1..20: mol_type = other DNA
agagtgcatcgacccctcgg

SEQ ID NO: 123 - DNA, 20 bp, synthetic construct
  source 1..20: mol_type = other DNA
ctgcggggaggactccgtcg

SEQ ID NO: 124 - DNA, 20 bp, synthetic construct
  source 1..20: mol_type = other DNA
gctgcaccgagtcgtagtcg

SEQ ID NO: 125 - DNA, 20 bp, synthetic construct
  source 1..20: mol_type = other DNA
ggacatcatagagcgctcgt

SEQ ID NO: 126 - DNA, 20 bp, synthetic construct
  source 1..20: mol_type = other DNA
ggattccagcgctgtcgtcg

SEQ ID NO: 127 - DNA, 20 bp, synthetic construct
  source 1..20: mol_type = other DNA
gccagttgtaacaatgtcag

SEQ ID NO: 128 - DNA, 20 bp, synthetic construct
  source 1..20: mol_type = other DNA
ggttgaaggactgacaccgc

SEQ ID NO: 129 - DNA, 20 bp, synthetic construct
  source 1..20: mol_type = other DNA
taaacttggaccacaacagg

SEQ ID NO: 130 - DNA, 20 bp, synthetic construct
  source 1..20: mol_type = other DNA
cctggggtctccctacccgg

SEQ ID NO: 131 - DNA, 20 bp, synthetic construct
  source 1..20: mol_type = other DNA
gcggtaccagagcggcaagt

SEQ ID NO: 132 - DNA, 20 bp, synthetic construct
  source 1..20: mol_type = other DNA
ccggggctggtacttatgga

SEQ ID NO: 133 - DNA, 20 bp, synthetic construct
  source 1..20: mol_type = other DNA
ggcaggcggcctgtgcagcg

SEQ ID NO: 134 - DNA, 20 bp, synthetic construct
  source 1..20: mol_type = other DNA
ggggatctcccaacctgccc

SEQ ID NO: 135 - DNA, 20 bp, synthetic construct
  source 1..20: mol_type = other DNA
tccaggcatcgccgcccggg

SEQ ID NO: 136 - DNA, 20 bp, synthetic construct
  source 1..20: mol_type = other DNA
acaccggcgcgtggggaacg

SEQ ID NO: 137 - DNA, 20 bp, synthetic construct
  source 1..20: mol_type = other DNA
agacgctatccacgtcgtag

SEQ ID NO: 138 - DNA, 20 bp, synthetic construct
  source 1..20: mol_type = other DNA
gtcttgcaggaatgacacca

SEQ ID NO: 139 - DNA, 20 bp, synthetic construct
  source 1..20: mol_type = other DNA
gcaaatgttacaggtcctgg

SEQ ID NO: 140 - DNA, 20 bp, synthetic construct
  source 1..20: mol_type = other DNA
ggcggggacaagaaggtcat

SEQ ID NO: 141 - DNA, 20 bp, synthetic construct
  source 1..20: mol_type = other DNA
attcatcaaccaacatacag

SEQ ID NO: 142 - DNA, 20 bp, synthetic construct
  source 1..20: mol_type = other DNA
agtttgactcttaccctgtg

SEQ ID NO: 143 - DNA, 20 bp, synthetic construct
  source 1..20: mol_type = other DNA
aaggatcaaatgctttgctg

SEQ ID NO: 144 - DNA, 20 bp, synthetic construct
  source 1..20: mol_type = other DNA
tcacctttcgtcagagtccg

SEQ ID NO: 145 - DNA, 20 bp, synthetic construct
  source 1..20: mol_type = other DNA
caacaggatggaatccgacg

SEQ ID NO: 146 - DNA, 20 bp, synthetic construct
  source 1..20: mol_type = other DNA
tcgtggcagaagatgacggg

SEQ ID NO: 147 - DNA, 20 bp, synthetic construct
  source 1..20: mol_type = other DNA
gccgcacgttggcttgatcg

SEQ ID NO: 148 - DNA, 20 bp, synthetic construct
  source 1..20: mol_type = other DNA
gcctggagcgatttctcccg

SEQ ID NO: 149 - DNA, 20 bp, synthetic construct
  source 1..20: mol_type = other DNA
gaagccccacatgtgtgaca

SEQ ID NO: 150 - DNA, 20 bp, synthetic construct
  source 1..20: mol_type = other DNA
acacagacgatgaatcaggg

SEQ ID NO: 151 - DNA, 20 bp, synthetic construct
  source 1..20: mol_type = other DNA
acacggtacctgatcctaca

SEQ ID NO: 152 - DNA, 20 bp, synthetic construct
  source 1..20: mol_type = other DNA
gatgatgacattgtagacgc

SEQ ID NO: 153 - DNA, 20 bp, synthetic construct
  source 1..20: mol_type = other DNA
gtacgtagaatgtttcaccc

SEQ ID NO: 154 - DNA, 20 bp, synthetic construct
  source 1..20: mol_type = other DNA
aatgcagtgcatattaacaa

SEQ ID NO: 155 - DNA, 20 bp, synthetic construct
  source 1..20: mol_type = other DNA
aggcgatcctaatcatgtca

SEQ ID NO: 156 - DNA, 20 bp, synthetic construct
  source 1..20: mol_type = other DNA
gttgtaactgagaagcggaa

SEQ ID NO: 157 - DNA, 20 bp, synthetic construct
  source 1..20: mol_type = other DNA
tggagaaagacgtaacttcg

SEQ ID NO: 158 - DNA, 20 bp, synthetic construct
  source 1..20: mol_type = other DNA
tctgccctgaggtatgcgat

SEQ ID NO: 159 - DNA, 20 bp, synthetic construct
  source 1..20: mol_type = other DNA
gattccgcttggtgaaaacg

SEQ ID NO: 160 - DNA, 20 bp, synthetic construct
  source 1..20: mol_type = other DNA
gttccaaaaaccctcacacc

SEQ ID NO: 161 - DNA, 20 bp, synthetic construct
  source 1..20: mol_type = other DNA
gtgtggacttacaataaccg

SEQ ID NO: 162 - DNA, 20 bp, synthetic construct
  source 1..20: mol_type = other DNA
gcacagacggacaaatacag

SEQ ID NO: 163 - DNA, 20 bp, synthetic construct
  source 1..20: mol_type = other DNA
aaatacaccacataatagag

SEQ ID NO: 164 - DNA, 20 bp, synthetic construct
  source 1..20: mol_type = other DNA
gtgtgtcaagacgctttggg

SEQ ID NO: 165 - DNA, 20 bp, synthetic construct
  source 1..20: mol_type = other DNA
tctatacatgtgtctgacgt

SEQ ID NO: 166 - DNA, 20 bp, synthetic construct
  source 1..20: mol_type = other DNA
gctggcaaaatagaacacga

SEQ ID NO: 167 - DNA, 20 bp, synthetic construct
  source 1..20: mol_type = other DNA
catttgctgctcgtgcacgg

SEQ ID NO: 168 - DNA, 20 bp, synthetic construct
  source 1..20: mol_type = other DNA
tgtaatccaagatgttattg

SEQ ID NO: 169 - DNA, 20 bp, synthetic construct
  source 1..20: mol_type = other DNA
ctgatcgtaggaccacggtg

SEQ ID NO: 170 - DNA, 20 bp, synthetic construct
  source 1..20: mol_type = other DNA
cacttcgaccgacaaacctg

SEQ ID NO: 171 - DNA, 20 bp, synthetic construct
  source 1..20: mol_type = other DNA
tagatgatcagaccaagccc

SEQ ID NO: 172 - DNA, 20 bp, synthetic construct
  source 1..20: mol_type = other DNA
ggcagtggagtggttcaggg

SEQ ID NO: 173 - DNA, 20 bp, synthetic construct
  source 1..20: mol_type = other DNA
tagcatttgagctagttcga

SEQ ID NO: 174 - DNA, 20 bp, synthetic construct
  source 1..20: mol_type = other DNA
ggagtttagccagtccaact

SEQ ID NO: 175 - DNA, 20 bp, synthetic construct
  source 1..20: mol_type = other DNA
ggtggcgcaaacgagtagca

SEQ ID NO: 176 - DNA, 20 bp, synthetic construct
  source 1..20: mol_type = other DNA
acaggttgccccacgcgttg

SEQ ID NO: 177 - DNA, 20 bp, synthetic construct
  source 1..20: mol_type = other DNA
agccggtccctttcccgtcg

SEQ ID NO: 178 - DNA, 20 bp, synthetic construct
  source 1..20: mol_type = other DNA
cctcagttggcaaggtacct

SEQ ID NO: 179 - DNA, 20 bp, synthetic construct
  source 1..20: mol_type = other DNA
cctctcaatcaaaataaggt

SEQ ID NO: 180 - DNA, 20 bp, synthetic construct
  source 1..20: mol_type = other DNA
cgaaaatctgggctaagtgc

SEQ ID NO: 181 - DNA, 20 bp, synthetic construct
  source 1..20: mol_type = other DNA
tgaacaaacgctggtcaccg

SEQ ID NO: 182 - DNA, 20 bp, synthetic construct
  source 1..20: mol_type = other DNA
agatattgaccatgagacag

SEQ ID NO: 183 - DNA, 20 bp, synthetic construct
  source 1..20: mol_type = other DNA
ggtcaccgacgacccgaccc

SEQ ID NO: 184 - DNA, 20 bp, synthetic construct
  source 1..20: mol_type = other DNA
ggagaccatcgagaccacag

SEQ ID NO: 185 - DNA, 20 bp, synthetic construct
  source 1..20: mol_type = other DNA
gatttctgatgaaaaagacg

SEQ ID NO: 186 - DNA, 20 bp, synthetic construct
  source 1..20: mol_type = other DNA
gctctactggagcgacgagg

SEQ ID NO: 187 - DNA, 20 bp, synthetic construct
  source 1..20: mol_type = other DNA
gaggcaaaaagaaatcccca

SEQ ID NO: 188 - DNA, 20 bp, synthetic construct
  source 1..20: mol_type = other DNA
tcacccaaaaaaatgcacca

SEQ ID NO: 189 - DNA, 20 bp, synthetic construct
  source 1..20: mol_type = other DNA
ttaattccaaccaaatcccg

SEQ ID NO: 190 - DNA, 20 bp, synthetic construct
  source 1..20: mol_type = other DNA
tctaggccgatacaaagcag

SEQ ID NO: 191 - DNA, 20 bp, synthetic construct
  source 1..20: mol_type = other DNA
gaactccctgccagatatcg

SEQ ID NO: 192 - DNA, 20 bp, synthetic construct
  source 1..20: mol_type = other DNA
tctccctcgacagtcatgtg

SEQ ID NO: 193 - DNA, 20 bp, synthetic construct
  source 1..20: mol_type = other DNA
gagcaaactgcgttatacag

SEQ ID NO: 194 - DNA, 20 bp, synthetic construct
  source 1..20: mol_type = other DNA
cgatccaaatttgaacgccg

SEQ ID NO: 195 - DNA, 20 bp, synthetic construct
  source 1..20: mol_type = other DNA
tttgtgcagttatgccagca

SEQ ID NO: 196 - DNA, 20 bp, synthetic construct
  source 1..20: mol_type = other DNA
ttaccccagaaccagacgga

SEQ ID NO: 197 - DNA, 20 bp, synthetic construct
  source 1..20: mol_type = other DNA
agttgtaatcaaatccacac

SEQ ID NO: 198 - DNA, 20 bp, synthetic construct
  source 1..20: mol_type = other DNA
gatatgatctgtcctgaatg

SEQ ID NO: 199 - DNA, 20 bp, synthetic construct
  source 1..20: mol_type = other DNA
acaaaagatccatctcgagt

SEQ ID NO: 200 - DNA, 20 bp, synthetic construct
  source 1..20: mol_type = other DNA
tttgtctaccatgattggca

SEQ ID NO: 201 - DNA, 20 bp, synthetic construct
  source 1..20: mol_type = other DNA
ccttccgacccatttcgcgg

SEQ ID NO: 202 - DNA, 20 bp, synthetic construct
  source 1..20: mol_type = other DNA
ctgtacgtgtgcattccgtg

SEQ ID NO: 203 - DNA, 20 bp, synthetic construct
  source 1..20: mol_type = other DNA
aagaaacgcctcaagcgcca

SEQ ID NO: 204 - DNA, 20 bp, synthetic construct
  source 1..20: mol_type = other DNA
caggttgtcatgcaccacca

SEQ ID NO: 205 - RNA, 20 nt, synthetic construct
  source 1..20: mol_type = other RNA
ggacacataatggcaaatcg

SEQ ID NO: 206 - RNA, 20 nt, synthetic construct
  source 1..20: mol_type = other RNA
caaaatctcaccctgaaacg

SEQ ID NO: 207 - RNA, 20 nt, synthetic construct
  source 1..20: mol_type = other RNA
ccacggcgaagcgccctccg

SEQ ID NO: 208 - RNA, 20 nt, synthetic construct
  source 1..20: mol_type = other RNA
tgggtggtactgccatccgg

SEQ ID NO: 209 - RNA, 20 nt, synthetic construct
  source 1..20: mol_type = other RNA
ggtctcggagaatgaccgag

SEQ ID NO: 210 - RNA, 20 nt, synthetic construct
  source 1..20: mol_type = other RNA
cttactaatgaagtaatccg

SEQ ID NO: 211 - RNA, 20 nt, synthetic construct
  source 1..20: mol_type = other RNA
tgcatggggaggaccagtcg

SEQ ID NO: 212 - RNA, 20 nt, synthetic construct
  source 1..20: mol_type = other RNA
gagaaagcagtctttaccca

SEQ ID NO: 213 - RNA, 20 nt, synthetic construct
  source 1..20: mol_type = other RNA
tgcacataatagacccgcag

SEQ ID NO: 214 - RNA, 20 nt, synthetic construct
  source 1..20: mol_type = other RNA
tagaattgaccactatgttg

SEQ ID NO: 215 - RNA, 20 nt, synthetic construct
  source 1..20: mol_type = other RNA
ggcttctcgagcggccaacg

SEQ ID NO: 216 - RNA, 20 nt, synthetic construct
  source 1..20: mol_type = other RNA
gagtgcgcacttcatccatg

SEQ ID NO: 217 - RNA, 20 nt, synthetic construct
  source 1..20: mol_type = other RNA
ggggagcgagtgatgtacgt

SEQ ID NO: 218 - RNA, 20 nt, synthetic construct
  source 1..20: mol_type = other RNA
ggacgcgttcaagtaagcgt

SEQ ID NO: 219 - RNA, 20 nt, synthetic construct
  source 1..20: mol_type = other RNA
tttggacagatctattccag

SEQ ID NO: 220 - RNA, 20 nt, synthetic construct
  source 1..20: mol_type = other RNA
aggatgcggatatgactctg

SEQ ID NO: 221 - RNA, 20 nt, synthetic construct
  source 1..20: mol_type = other RNA
tccaagacgtccatccacca

SEQ ID NO: 222 - RNA, 20 nt, synthetic construct
  source 1..20: mol_type = other RNA
aggtaccctccgacccacca

SEQ ID NO: 223 - RNA, 20 nt, synthetic construct
  source 1..20: mol_type = other RNA
ggagctgtactcgggcacgt

SEQ ID NO: 224 - RNA, 20 nt, synthetic construct
  source 1..20: mol_type = other RNA
cagggagtgtgtgaactgtg

SEQ ID NO: 225 - RNA, 20 nt, synthetic construct
  source 1..20: mol_type = other RNA
ccccaggatgcattatcccg

SEQ ID NO: 226 - RNA, 20 nt, synthetic construct
  source 1..20: mol_type = other RNA
cactgcggcccacgaagcga

SEQ ID NO: 227 - RNA, 20 nt, synthetic construct
  source 1..20: mol_type = other RNA
ccgtgccgtaccttggattg

SEQ ID NO: 228 - RNA, 20 nt, synthetic construct
  source 1..20: mol_type = other RNA
ccgtgccgtaccttggattg

SEQ ID NO: 229 - RNA, 20 nt, synthetic construct
  source 1..20: mol_type = other RNA
agaggaggagacacatgtcg

SEQ ID NO: 230 - RNA, 20 nt, synthetic construct
  source 1..20: mol_type = other RNA
gttctgtagacttcacatgc

SEQ ID NO: 231 - RNA, 20 nt, synthetic construct
  source 1..20: mol_type = other RNA
catacaccatgtccatagag

SEQ ID NO: 232 - RNA, 20 nt, synthetic construct
  source 1..20: mol_type = other RNA
gccttctgacaattcagccc

SEQ ID NO: 233 - RNA, 20 nt, synthetic construct
  source 1..20: mol_type = other RNA
ccggtacattctctaaacgg

SEQ ID NO: 234 - RNA, 20 nt, synthetic construct
  source 1..20: mol_type = other RNA
tgggcacattgacttctccg

SEQ ID NO: 235 - RNA, 20 nt, synthetic construct
  source 1..20: mol_type = other RNA
ttcaaaccgtactcatggag

SEQ ID NO: 236 - RNA, 20 nt, synthetic construct
  source 1..20: mol_type = other RNA
acgtgcacctcgagcccgtg

SEQ ID NO: 237 - RNA, 20 nt, synthetic construct
  source 1..20: mol_type = other RNA
ttataacagactactgtctg

SEQ ID NO: 238 - RNA, 20 nt, synthetic construct
  source 1..20: mol_type = other RNA
cttacttcggatatgggact

SEQ ID NO: 239 - RNA, 20 nt, synthetic construct
  source 1..20: mol_type = other RNA
atcgcaaacaaagccaactg

SEQ ID NO: 240 - RNA, 20 nt, synthetic construct
  source 1..20: mol_type = other RNA
taccacatccactctaagca

SEQ ID NO: 241 - RNA, 20 nt, synthetic construct
  source 1..20: mol_type = other RNA
taacctggtcttccttggtg

SEQ ID NO: 242 - RNA, 20 nt, synthetic construct
  source 1..20: mol_type = other RNA
accattgcaatttagagatg

SEQ ID NO: 243 - RNA, 20 nt, synthetic construct
  source 1..20: mol_type = other RNA
ggccatagaattctctctgg

SEQ ID NO: 244 - RNA, 20 nt, synthetic construct
  source 1..20: mol_type = other RNA
ctgatcacctgtctggagca

SEQ ID NO: 245 - RNA, 20 nt, synthetic construct
  source 1..20: mol_type = other RNA
gagggtgccaccatgactag

SEQ ID NO: 246 - RNA, 20 nt, synthetic construct
  source 1..20: mol_type = other RNA
cccacccacagggatcaacg

SEQ ID NO: 247 - RNA, 20 nt, synthetic construct
  source 1..20: mol_type = other RNA
acccaggcatcatccgacaa

SEQ ID NO: 248 - RNA, 20 nt, synthetic construct
  source 1..20: mol_type = other RNA
cctacttaggcactgccagg

SEQ ID NO: 249 - RNA, 20 nt, synthetic construct
  source 1..20: mol_type = other RNA
agatgtctaacgataaggag

SEQ ID NO: 250 - RNA, 20 nt, synthetic construct
  source 1..20: mol_type = other RNA
caagctaaccatcttacgca

SEQ ID NO: 251 - RNA, 20 nt, synthetic construct
  source 1..20: mol_type = other RNA
tggattgtgttggagagtgt

SEQ ID NO: 252 - RNA, 20 nt, synthetic construct
  source 1..20: mol_type = other RNA
tggggaacctcacttcgtgg

SEQ ID NO: 253 - RNA, 20 nt, synthetic construct
  source 1..20: mol_type = other RNA
cttcggggagacaacgacgg

SEQ ID NO: 254 - RNA, 20 nt, synthetic construct
  source 1..20: mol_type = other RNA
agagtgcatcgacccctcgg

SEQ ID NO: 255 - RNA, 20 nt, synthetic construct
  source 1..20: mol_type = other RNA
ctgcggggaggactccgtcg

SEQ ID NO: 256 - RNA, 20 nt, synthetic construct
  source 1..20: mol_type = other RNA
gctgcaccgagtcgtagtcg

SEQ ID NO: 257 - RNA, 20 nt, synthetic construct
  source 1..20: mol_type = other RNA
ggacatcatagagcgctcgt

SEQ ID NO: 258 - RNA, 20 nt, synthetic construct
  source 1..20: mol_type = other RNA
ggattccagcgctgtcgtcg

SEQ ID NO: 259 - RNA, 20 nt, synthetic construct
  source 1..20: mol_type = other RNA
gccagttgtaacaatgtcag

SEQ ID NO: 260 - RNA, 20 nt, synthetic construct
  source 1..20: mol_type = other RNA
ggttgaaggactgacaccgc

SEQ ID NO: 261 - RNA, 20 nt, synthetic construct
  source 1..20: mol_type = other RNA
taaacttggaccacaacagg

SEQ ID NO: 262 - RNA, 20 nt, synthetic construct
  source 1..20: mol_type = other RNA
cctggggtctccctacccgg

SEQ ID NO: 263 - RNA, 20 nt, synthetic construct
  source 1..20: mol_type = other RNA
gcggtaccagagcggcaagt

SEQ ID NO: 264 - RNA, 20 nt, synthetic construct
  source 1..20: mol_type = other RNA
ccggggctggtacttatgga

SEQ ID NO: 265 - RNA, 20 nt, synthetic construct
  source 1..20: mol_type = other RNA
ggcaggcggcctgtgcagcg

SEQ ID NO: 266 - RNA, 20 nt, synthetic construct
  source 1..20: mol_type = other RNA
ggggatctcccaacctgccc

SEQ ID NO: 267 - RNA, 20 nt, synthetic construct
  source 1..20: mol_type = other RNA
tccaggcatcgccgcccggg

SEQ ID NO: 268 - RNA, 20 nt, synthetic construct
  source 1..20: mol_type = other RNA
acaccggcgcgtggggaacg

SEQ ID NO: 269 - RNA, 20 nt, synthetic construct
  source 1..20: mol_type = other RNA
agacgctatccacgtcgtag

SEQ ID NO: 270 - RNA, 20 nt, synthetic construct
  source 1..20: mol_type = other RNA
gtcttgcaggaatgacacca

SEQ ID NO: 271 - RNA, 20 nt, synthetic construct
  source 1..20: mol_type = other RNA
gcaaatgttacaggtcctgg

SEQ ID NO: 272 - RNA, 20 nt, synthetic construct
  source 1..20: mol_type = other RNA
ggcggggacaagaaggtcat

SEQ ID NO: 273 - RNA, 20 nt, synthetic construct
  source 1..20: mol_type = other RNA
attcatcaaccaacatacag

SEQ ID NO: 274 - RNA, 20 nt, synthetic construct
  source 1..20: mol_type = other RNA
agtttgactcttaccctgtg

SEQ ID NO: 275 - RNA, 20 nt, synthetic construct
  source 1..20: mol_type = other RNA
aaggatcaaatgctttgctg

SEQ ID NO: 276 - RNA, 20 nt, synthetic construct
  source 1..20: mol_type = other RNA
tcacctttcgtcagagtccg

SEQ ID NO: 277 - RNA, 20 nt, synthetic construct
  source 1..20: mol_type = other RNA
caacaggatggaatccgacg

SEQ ID NO: 278 - RNA, 20 nt, synthetic construct
  source 1..20: mol_type = other RNA
tcgtggcagaagatgacggg

SEQ ID NO: 279 - RNA, 20 nt, synthetic construct
  source 1..20: mol_type = other RNA
gccgcacgttggcttgatcg

SEQ ID NO: 280 - RNA, 20 nt, synthetic construct
  source 1..20: mol_type = other RNA
gcctggagcgatttctcccg

SEQ ID NO: 281 - RNA, 20 nt, synthetic construct
  source 1..20: mol_type = other RNA
gaagccccacatgtgtgaca

SEQ ID NO: 282 - RNA, 20 nt, synthetic construct
  source 1..20: mol_type = other RNA
acacagacgatgaatcaggg

SEQ ID NO: 283 - RNA, 20 nt, synthetic construct
  source 1..20: mol_type = other RNA
acacggtacctgatcctaca

SEQ ID NO: 284 - RNA, 20 nt, synthetic construct
  source 1..20: mol_type = other RNA
gatgatgacattgtagacgc

SEQ ID NO: 285 - RNA, 20 nt, synthetic construct
  source 1..20: mol_type = other RNA
gtacgtagaatgtttcaccc

SEQ ID NO: 286 - RNA, 20 nt, synthetic construct
  source 1..20: mol_type = other RNA
aatgcagtgcatattaacaa

SEQ ID NO: 287 - RNA, 20 nt, synthetic construct
  source 1..20: mol_type = other RNA
aggcgatcctaatcatgtca

SEQ ID NO: 288 - RNA, 20 nt, synthetic construct
  source 1..20: mol_type = other RNA
gttgtaactgagaagcggaa

SEQ ID NO: 289 - RNA, 20 nt, synthetic construct
  source 1..20: mol_type = other RNA
tggagaaagacgtaacttcg

SEQ ID NO: 290 - RNA, 20 nt, synthetic construct
  source 1..20: mol_type = other RNA
tctgccctgaggtatgcgat

SEQ ID NO: 291 - RNA, 20 nt, synthetic construct
  source 1..20: mol_type = other RNA
gattccgcttggtgaaaacg

SEQ ID NO: 292 - RNA, 20 nt, synthetic construct
  source 1..20: mol_type = other RNA
gttccaaaaaccctcacacc

SEQ ID NO: 293 - RNA, 20 nt, synthetic construct
  source 1..20: mol_type = other RNA
gtgtggacttacaataaccg

SEQ ID NO: 294 - RNA, 20 nt, synthetic construct
  source 1..20: mol_type = other RNA
gcacagacggacaaatacag

SEQ ID NO: 295 - RNA, 20 nt, synthetic construct
  source 1..20: mol_type = other RNA
aaatacaccacataatagag

SEQ ID NO: 296 - RNA, 20 nt, synthetic construct
  source 1..20: mol_type = other RNA
gtgtgtcaagacgctttggg

SEQ ID NO: 297 - RNA, 20 nt, synthetic construct
  source 1..20: mol_type = other RNA
tctatacatgtgtctgacgt

SEQ ID NO: 298 - RNA, 20 nt, synthetic construct
  source 1..20: mol_type = other RNA
gctggcaaaatagaacacga

SEQ ID NO: 299 - RNA, 20 nt, synthetic construct
  source 1..20: mol_type = other RNA
catttgctgctcgtgcacgg

SEQ ID NO: 300 - RNA, 20 nt, synthetic construct
  source 1..20: mol_type = other RNA
tgtaatccaagatgttattg

SEQ ID NO: 301 - RNA, 20 nt, synthetic construct
  source 1..20: mol_type = other RNA
ctgatcgtaggaccacggtg

SEQ ID NO: 302 - RNA, 20 nt, synthetic construct
  source 1..20: mol_type = other RNA
cacttcgaccgacaaacctg

SEQ ID NO: 303 - RNA, 20 nt, synthetic construct
  source 1..20: mol_type = other RNA
tagatgatcagaccaagccc

SEQ ID NO: 304 - RNA, 20 nt, synthetic construct
  source 1..20: mol_type = other RNA
ggcagtggagtggttcaggg

SEQ ID NO: 305 - RNA, 20 nt, synthetic construct
  source 1..20: mol_type = other RNA
tagcatttgagctagttcga

SEQ ID NO: 306 - RNA, 20 nt, synthetic construct
  source 1..20: mol_type = other RNA
ggagtttagccagtccaact

SEQ ID NO: 307 - RNA, 20 nt, synthetic construct
  source 1..20: mol_type = other RNA
ggtggcgcaaacgagtagca

SEQ ID NO: 308 - RNA, 20 nt, synthetic construct
  source 1..20: mol_type = other RNA
acaggttgccccacgcgttg

SEQ ID NO: 309 - RNA, 20 nt, synthetic construct
  source 1..20: mol_type = other RNA
agccggtccctttcccgtcg

SEQ ID NO: 310 - RNA, 20 nt, synthetic construct
  source 1..20: mol_type = other RNA
cctcagttggcaaggtacct

SEQ ID NO: 311 - RNA, 20 nt, synthetic construct
  source 1..20: mol_type = other RNA
cctctcaatcaaaataaggt

SEQ ID NO: 312 - RNA, 20 nt, synthetic construct
  source 1..20: mol_type = other RNA
cgaaaatctgggctaagtgc

SEQ ID NO: 313 - RNA, 20 nt, synthetic construct
  source 1..20: mol_type = other RNA
tgaacaaacgctggtcaccg

SEQ ID NO: 314 - RNA, 20 nt, synthetic construct
  source 1..20: mol_type = other RNA
agatattgaccatgagacag

SEQ ID NO: 315 - RNA, 20 nt, synthetic construct
  source 1..20: mol_type = other RNA
ggtcaccgacgacccgaccc

SEQ ID NO: 316 - RNA, 20 nt, synthetic construct
  source 1..20: mol_type = other RNA
ggagaccatcgagaccacag

SEQ ID NO: 317 - RNA, 20 nt, synthetic construct
  source 1..20: mol_type = other RNA
gatttctgatgaaaaagacg

SEQ ID NO: 318 - RNA, 20 nt, synthetic construct
  source 1..20: mol_type = other RNA
gctctactggagcgacgagg

SEQ ID NO: 319 - RNA, 20 nt, synthetic construct
  source 1..20: mol_type = other RNA
gaggcaaaaagaaatcccca

SEQ ID NO: 320 - RNA, 20 nt, synthetic construct
  source 1..20: mol_type = other RNA
tcacccaaaaaaatgcacca

SEQ ID NO: 321 - RNA, 20 nt, synthetic construct
  source 1..20: mol_type = other RNA
ttaattccaaccaaatcccg

SEQ ID NO: 322 - RNA, 20 nt, synthetic construct
  source 1..20: mol_type = other RNA
tctaggccgatacaaagcag

SEQ ID NO: 323 - RNA, 20 nt, synthetic construct
  source 1..20: mol_type = other RNA
gaactccctgccagatatcg

SEQ ID NO: 324 - RNA, 20 nt, synthetic construct
  source 1..20: mol_type = other RNA
tctccctcgacagtcatgtg

SEQ ID NO: 325 - RNA, 20 nt, synthetic construct
  source 1..20: mol_type = other RNA
gagcaaactgcgttatacag

SEQ ID NO: 326 - RNA, 20 nt, synthetic construct
  source 1..20: mol_type = other RNA
cgatccaaatttgaacgccg

SEQ ID NO: 327 - RNA, 20 nt, synthetic construct
  source 1..20: mol_type = other RNA
tttgtgcagttatgccagca

SEQ ID NO: 328 - RNA, 20 nt, synthetic construct
  source 1..20: mol_type = other RNA
ttaccccagaaccagacgga

SEQ ID NO: 329 - RNA, 20 nt, synthetic construct
  source 1..20: mol_type = other RNA
agttgtaatcaaatccacac

SEQ ID NO: 330 - RNA, 20 nt, synthetic construct
  source 1..20: mol_type = other RNA
gatatgatctgtcctgaatg

SEQ ID NO: 331 - RNA, 20 nt, synthetic construct
  source 1..20: mol_type = other RNA
acaaaagatccatctcgagt

SEQ ID NO: 332 - RNA, 20 nt, synthetic construct
  source 1..20: mol_type = other RNA
tttgtctaccatgattggca

SEQ ID NO: 333 - RNA, 20 nt, synthetic construct
  source 1..20: mol_type = other RNA
ccttccgacccatttcgcgg

SEQ ID NO: 334 - RNA, 20 nt, synthetic construct
  source 1..20: mol_type = other RNA
ctgtacgtgtgcattccgtg

SEQ ID NO: 335 - RNA, 20 nt, synthetic construct
  source 1..20: mol_type = other RNA
aagaaacgcctcaagcgcca

SEQ ID NO: 336 - RNA, 20 nt, synthetic construct
  source 1..20: mol_type = other RNA
caggttgtcatgcaccacca

SEQ ID NO: 337 - DNA, 2547 bp, synthetic construct
  source 1..2547: mol_type = other DNA
atgaacagcagcagcgccaacatcacctacgccagtcgcaagcggcggaagccggtgcagaaaacagtaaagccaatcccagctgaaggaatcaagtcaaatccttccaagcggcatagagaccgacttaatacagagttggaccgtttggctagcctgctgcctttcccacaagatgttattaataagttggacaaactttcagttcttaggctcagcgtcagttacctgagagccaagagcttctttgatgttgcattaaaatcctcccctactgaaagaaacggaggccaggataactgtagagcagcaaatttcagagaaggcctgaacttacaagaaggagaattcttattacaggctctgaatggctttgtattagttgtcactacagatgctttggtcttttatgcttcttctactatacaagattatctagggtttcagcagtctgatgtcatacatcagagtgtatatgaacttatccataccgaagaccgagctgaatttcagcgtcagctacactgggcattaaatccttctcagtgtacagagtctggacaaggaattgaagaagccactggtctcccccagacagtagtctgttataacccagaccagattcctccagaaaactctcctttaatggagaggtgcttcatatgtcgtctaaggtgtctgctggataattcatctggttttctggcaatgaatttccaagggaagttaaagtatcttcatggacagaaaaagaaagggaaagatggatcaatacttccacctcagttggctttgtttgcgatagctactccacttcagccaccatccatacttgaaatccggaccaaaaattttatctttagaaccaaacacaaactagacttcacacctattggttgtgatgccaaaggaagaattgttttaggatatactgaagcagagctgtgcacgagaggctcaggttatcagtttattcatgcagctgatatgctttattgtgccgagtcccatatccgaatgattaagactggagaaagtggcatgatagttttccggcttcttacaaaaaacaaccgatggacttgggtccagtctaatgcacgcctgctttataaaaatggaagaccagattatatcattgtaactcagagaccactaacagatgaggaaggaacagagcatttacgaaaacgaaatacgaagttgccttttatgtttaccactggagaagctgtgttgtatgaggcaaccaacccttttcctgccataatggatcccttaccactaaggactaaaaatggcactagtggaaaagactctgctaccacatccactctaagcaaggactctctcaatcctagttccctcctggctgccatgatgcaacaagatgagtctatttatctctatcctgcttcaagtacttcaagtactgcaccttttgaaaacaactttttcaacgaatctatgaatgaatgcagaaattggcaagataatactgcaccgatgggaaatgatactatcctgaaacatgagcaaattgaccagcctcaggatgtgaactcatttgctggaggtcacccagggctctttcaagatagtaaaaacagtgacttgtacagcataatgaaaaacctaggcattgattttgaagacatcagacacatgcagaatgaaaaatttttcagaaatgatttttctggtgaggttgacttcagagacattgacttaacggatgaaatcctgacgtatgtccaagattctttaagtaagtctcccttcataccttcagattatcaacagcaacagtccttggctctgaactcaagctgtatggtacaggaacacctacatctagaacagcaacagcaacatcaccaaaagcaagtagtagtggagccacagcaacagctgtgtcagaagatgaagcacatgcaagttaatggcatgtttgaaaattggaactctaaccaattcgtgcctttcaattgtccacagcaagacccacaacaatataatgtctttacagacttacatgggatcagtcaagagttcccctacaaatctgaaatggattctatgccttatacacagaactttatttcctgtaatcagcctgtattaccacaacattccaaatgtacagagctggactaccctatggggagttttgaaccatccccataccccactacttctagtttagaagattttgtcacttgtttacaacttcctgaaaaccaaaagcatggattaaatccacagtcagccataataactcctcagacatgttatgctggggccgtgtcgatgtatcagtgccagccagaacctcagcacacccacgtgggtcagatgcagtacaatccagtactgccaggccaacaggcatttttaaacaagtttcagaatggagttttaaatgaaacatatccagctgaattaaataacataaataacactcagactaccacacatcttcagccacttcatcatccgtcagaagccagaccttttcctgatttgacatccagtggattcctgtaa

SEQ ID NO: 338 - DNA, 2370 bp, synthetic construct
  source 1..2370: mol_type = other DNA
atggcggcgactactgccaaccccgaaatgacatcagatgtaccatcactgggtccagccattgcctctggaaactctggacctggaattcaaggtggaggagccattgtccagagggctattaagcggcgaccagggctggattttgatgatgatggagaagggaacagtaaatttttgaggtgtgatgatgatcagatgtctaacgataaggagcggtttgccaggtcggatgatgagcagagctctgcggataaagagagacttgccagggaaaatcacagtgaaattgaacggcggcgacggaacaagatgacagcctacatcacagaactgtcagatatggtacccacctgtagtgccctggctcgaaaaccagacaagctaaccatcttacgcatggcagtttctcacatgaagtccttgcggggaactggcaacacatccactgatggctcctataagccgtctttcctcactgatcaggaactgaaacatttgatcttggaggcagcagatggctttctgtttattgtctcatgtgagacaggcagggtggtgtatgtgtctgactccgtgactcctgttttgaaccagccacagtctgaatggtttggcagcacactctatgatcaggtgcacccagatgatgtggataaacttcgtgagcagctttccacttcagaaaatgccctgacagggcgtatcctggatctaaagactggaacagtgaaaaaggaaggtcagcagtcttccatgagaatgtgtatgggctcaaggagatcgtttatttgccgaatgaggtgtggcagtagctctgtggacccagtttctgtgaataggctgagctttgtgaggaacagatgcaggaatggacttggctctgtaaaggatggggaacctcacttcgtggtggtccactgcacaggctacatcaaggcctggcccccagcaggtgtttccctcccagatgatgacccagaggctggccagggaagcaagttttgcctagtggccattggcagattgcaggtaactagttctcccaactgtacagacatgagtaatgtttgtcaaccaacagagttcatctcccgacacaacattgagggtatcttcacttttgtggatcaccgctgtgtggctactgttggctaccagccacaggaactcttaggaaagaatattgtagaattctgtcatcctgaagaccagcagcttctaagagacagcttccaacaggtagtgaaattaaaaggccaagtgctgtctgtcatgttccggttccggtctaagaaccaagaatggctctggatgagaaccagctcctttactttccagaacccttactcagatgaaattgagtacatcatctgtaccaacaccaatgtgaagaactctagccaagaaccacggcctacactctccaacacaatccagaggccacaactaggtcccacagctaatttacccctggagatgggctcaggacagctggcacccaggcagcagcaacagcaaacagaattggacatggtaccaggaagagatggactggccagctacaatcattcccaggtggttcagcctgtgacaaccacaggaccagaacacagcaagccccttgagaagtcagatggtttatttgcccaggatagagatccaagattttcagaaatctatcacaacatcaatgcggatcagagtaaaggcatctcctccagcactgtccctgccacccaacagctattctcccagggcaacacattccctcctaccccccggccggcagagaatttcaggaatagtggcctagcccctcctgtaaccattgtccagccatcagcttctgcaggacagatgttggcccagatttcccgccactccaaccccacccaaggagcaaccccaacttggacccctactacccgctcaggcttttctgcccagcaggtggctacccaggctactgctaagactcgtacttcccagtttggtgtgggcagctttcagactccatcctccttcagctccatgtccctccctggtgccccaactgcatcgcctggtgctgctgcctaccctagtctcaccaatcgtggatctaactttgctcctgagactggacagactgcaggacaattccagacacggacagcagagggtgtgggtgtctggccacagtggcagggccagcagcctcatcatcgttcaagttctagtgagcaacatgttcaacaaccgccagcacagcaacctggccagcctgaggtcttccaggagatgctgtccatgctgggagatcagagcaacagctacaacaatgaagaattccctgatctaactatgtttccccccttttcagaatag

SEQ ID NO: 339 - DNA, 2184 bp, synthetic construct
  source 1..2184: mol_type = other DNA
atggaaggtgatgcagtcgaagccattgtggaggagtccgaaacttttattaaaggaaaggagagaaagacttaccagagacgccgggaagggggccaggaagaagatgcctgccacttaccccagaaccagacggatgggggtgaggtggtccaggatgtcaacagcagtgtacagatggtgatgatggaacagctggaccccacccttcttcagatgaagactgaagtaatggagggcacagtggctccagaagcagaggctgctgtggacgatacccagattataactttacaggttgtaaatatggaggaacagcccataaacataggagaacttcagcttgttcaagtacctgttcctgtgactgtacctgttgctaccacttcagtagaagaacttcagggggcttatgaaaatgaagtgtctaaagagggccttgcggaaagtgaacccatgatatgccacaccctacctttgcctgaagggtttcaggtggttaaagtgggggccaatggagaggtggagacactagaacaaggggaacttccaccccaggaagatcctagttggcaaaaagacccagactatcagccaccagccaaaaaaacaaagaaaaccaaaaagagcaaactgcgttatacagaggagggcaaagatgtagatgtgtctgtctacgattttgaggaagaacagcaggagggtctgctatcagaggttaatgcagagaaagtggttggtaatatgaagcctccaaagccaacaaaaattaaaaagaaaggtgtaaagaagacattccagtgtgagctttgcagttacacgtgtccacggcgttcaaatttggatcgtcacatgaaaagccacactgatgagagaccacacaagtgccatctctgtggcagggcattcagaacagtcaccctcctgaggaatcaccttaacacacacacaggtactcgtcctcacaagtgcccagactgcgacatggcctttgtgaccagtggagaattggttcggcatcgtcgttacaaacacacccacgagaagccattcaagtgttccatgtgcgattacgccagtgtagaagtcagcaaattaaaacgtcacattcgctctcatactggagagcgtccgtttcagtgcagtttgtgcagttatgccagcagggacacatacaagctgaaaaggcacatgagaacccattcaggggaaaagccttatgaatgttatatttgtcatgctcggtttacccaaagtggtaccatgaagatgcacattttacagaagcacacagaaaatgtggccaaatttcactgtccccactgtgacacagtcatagcccgaaaaagtgatttgggtgtccacttgcgaaagcagcattcctatattgagcaaggcaagaaatgccgttactgtgatgctgtgtttcatgagcgctatgccctcatccagcatcagaagtcacacaagaatgagaagcgctttaagtgtgaccagtgtgattacgcttgtagacaggagaggcacatgatcatgcacaagcgcacccacaccggggagaagccttacgcctgcagccactgcgataagaccttccgccagaagcagcttctcgacatgcacttcaagcgctatcacgaccccaacttcgtccctgcggcttttgtctgttctaagtgtgggaaaacatttacacgtcggaataccatggcaagacatgctgataattgtgctggcccagatggcgtagagggggaaaatggaggagaaacgaagaagagtaaacgtggaagaaaaagaaagatgcgctctaagaaagaagattcctctgacagtgaaaatgctgaaccagatctggacgacaatgaggatgaggaggagcctgccgtagaaattgaacctgagccagagcctcagcctgtgaccccagccccaccacccgccaagaagcggagaggacgaccccctggcagaaccaaccagcccaaacagaaccagccaacagctatcattcaggttgaagaccagaatacaggtgcaattgagaacattatagttgaagtaaaaaaagagccagatgctgagcccgcagagggagaggaagaggaggcccagccagctgccacagatgcccccaacggagacctcacgcccgagatgatcctcagcatgatggaccggtga

SEQ ID NO: 340 - DNA, 4851 bp, synthetic construct
  source 1..4851: mol_type = other DNA
atgccggcgcgtaccgccccagcccgggtgcccacactggccgtcccggccatctcgctgcccgacgatgtccgcaggcggctcaaagatttggaaagagacagcttaacagaaaaggaatgtgtgaaggagaaattgaatctcttgcacgaatttctgcaaacagaaataaagaatcagttatgtgacttggaaaccaaattacgtaaagaagaattatccgaggagggctacctggctaaagtcaaatcccttttaaataaagatttgtccttggagaacggtgctcatgcttacaaccgggaagtgaatggacgtctagaaaacgggaaccaagcaagaagtgaagcccgtagagtgggaatggcagatgccaacagcccccccaaacccctttccaaacctcgcacgcccaggaggagcaagtccgatggagaggctaagcctgaaccttcacctagccccaggattacaaggaaaagcaccaggcaaaccaccatcacatctcattttgcaaagggccctgccaaacggaaacctcaggaagagtctgaaagagccaaatcggatgagtccatcaaggaagaagacaaagaccaggatgagaagagacgtagagttacatccagagaacgagttgctagaccgcttcctgcagaagaacctgaaagagcaaaatcaggaacgcgcactgaaaaggaagaagaaagagatgaaaaagaagaaaagagactccgaagtcaaaccaaagaaccaacacccaaacagaaactgaaggaggagccggacagagaagccagggcaggcgtgcaggctgacgaggacgaagatggagacgagaaagatgagaagaagcacagaagtcaacccaaagatctagctgccaaacggaggcccgaagaaaaagaacctgaaaaagtaaatccacagatttctgatgaaaaagacgaggatgaaaaggaggagaagagacgcaaaacgacccccaaagaaccaacggagaaaaaaatggctcgcgccaaaacagtcatgaactccaagacccaccctcccaagtgcattcagtgcgggcagtacctggacgaccctgacctcaaatatgggcagcacccaccagacgcggtggatgagccacagatgctgacaaatgagaagctgtccatctttgatgccaacgagtctggctttgagagttatgaggcgcttccccagcacaaactgacctgcttcagtgtgtactgtaagcacggtcacctgtgtcccatcgacaccggcctcatcgagaagaatatcgaactcttcttttctggttcagcaaaaccaatctatgatgatgacccatctcttgaaggtggtgttaatggcaaaaatcttggccccataaatgaatggtggatcactggctttgatggaggtgaaaaggccctcatcggcttcagcacctcatttgccgaatacattctgatggatcccagtcccgagtatgcgcccatatttgggctgatgcaggagaagatctacatcagcaagattgtggtggagttcctgcagagcaattccgactcgacctatgaggacctgatcaacaagatcgagaccacggttcctccttctggcctcaacttgaaccgcttcacagaggactccctcctgcgacacgcgcagtttgtggtggagcaggtggagagttatgacgaggccggggacagtgatgagcagcccatcttcctgacaccctgcatgcgggacctgatcaagctggctggggtcacgctgggacagaggcgagcccaggcgaggcggcagaccatcaggcattctaccagggagaaggacaggggacccacgaaagccaccaccaccaagctggtctaccagatcttcgatactttcttcgcagagcaaattgaaaaggatgacagagaagacaaggagaacgcctttaagcgccggcgatgtggcgtctgtgaggtgtgtcagcagcctgagtgtgggaaatgtaaagcctgcaaggacatggttaaatttggtggcagtggacggagcaagcaggcttgccaagagcggaggtgtcccaatatggccatgaaggaggcagatgacgatgaggaagtcgatgataacatcccagagatgccgtcacccaaaaaaatgcaccaggggaagaagaagaaacagaacaagaatcgcatctcttgggtcggagaagccgtcaagactgatgggaagaagagttactataagaaggtgtgcattgatgcggaaaccctggaagtgggggactgtgtctctgttattccagatgattcctcaaaaccgctgtatctagcaagggtcacggcgctgtgggaggacagcagcaacgggcagatgtttcacgcccactggttctgcgctgggacagacacagtcctcggggccacgtcggaccctctggagctgttcttggtggatgaatgtgaggacatgcagctttcatatatccacagcaaagtgaaagtcatctacaaagccccctccgaaaactgggccatggagggaggcatggatcccgagtccctgctggagggggacgacgggaagacctacttctaccagctgtggtatgatcaagactacgcgagattcgagtcccctccaaaaacccagccaacagaggacaacaagttcaaattctgtgtgagctgtgcccgtctggctgagatgaggcaaaaagaaatccccagggtcctggagcagctcgaggacctggatagccgggtcctctactactcagccaccaagaacggcatcctgtaccgagttggtgatggtgtgtacctgccccctgaggccttcacgttcaacatcaagctgtccagtcccgtgaaacgcccacggaaggagcccgtggatgaggacctgtacccagagcactaccggaaatactccgactacatcaaaggcagcaacctggatgcccctgagccctaccgaattggccggatcaaagagatcttctgtcccaagaagagcaacggcaggcccaatgagactgacatcaaaatccgggtcaacaagttctacaggcctgagaacacccacaagtccactccagcgagctaccacgcagacatcaacctgctctactggagcgacgaggaggccgtggtggacttcaaggctgtgcagggccgctgcaccgtggagtatggggaggacctgcccgagtgcgtccaggtgtactccatgggcggccccaaccgcttctacttcctcgaggcctataatgcaaagagcaaaagctttgaagatcctcccaaccatgcccgtagccctggaaacaaagggaagggcaagggaaaagggaagggcaagcccaagtcccaagcctgtgagccgagcgagccagagatagagatcaagctgcccaagctgcggaccctggatgtgttttctggctgcggggggttgtcggagggattccaccaagcaggcatctctgacacgctgtgggccatcgagatgtgggaccctgcggcccaggcgttccggctgaacaaccccggctccacagtgttcacagaggactgcaacatcctgctgaagctggtcatggctggggagaccaccaactcccgcggccagcggctgccccagaagggagacgtggagatgctgtgcggcgggccgccctgccagggcttcagcggcatgaaccgcttcaattcgcgcacctactccaagttcaaaaactctctggtggtttccttcctcagctactgcgactactaccggccccggttcttcctcctggagaatgtcaggaactttgtctccttcaagcgctccatggtcctgaagctcaccctccgctgcctggtccgcatgggctatcagtgcaccttcggcgtgctgcaggccggtcagtacggcgtggcccagactaggaggcgggccatcatcctggccgcggcccctggagagaagctccctctgttcccggagccactgcacgtgtttgctccccgggcctgccagctgagcgtggtggtggatgacaagaagtttgtgagcaacataaccaggttgagctcgggtcctttccggaccatcacggtgcgagacacgatgtccgacctgccggaggtgcggaatggagcctcggcactggagatctcctacaacggggagcctcagtcctggttccagaggcagctccggggcgcacagtaccagcccatcctcagggaccacatctgtaaggacatgagtgcattggtggctgcccgcatgcggcacatccccttggccccagggtcagactggcgcgatctgcccaacatcgaggtgcggctctcagacggcaccatggccaggaagctgcggtatacccaccatgacaggaagaacggccgcagcagctctggggccctccgtggggtctgctcctgcgtggaagccggcaaagcctgcgaccccgcagccaggcagttcaacaccctcatcccctggtgcctgccccacaccgggaaccggcacaaccactgggctggcctctatggaaggctcgagtgggacggcttcttcagcacaaccgtcaccaaccccgagcccatgggcaagcagggccgcgtgctccacccagagcagcaccgtgtggtgagcgtgcgggagtgtgcccgctcccagggcttccctgacacctaccggctcttcggcaacatcctggacaagcaccggcaggtgggcaatgccgtgccaccgcccctggccaaagccattggcttggagatcaagctttgtatgttggccaaagcccgagagagtgcctcagctaaaataaaggaggaggaagctgctaaggactag

SEQ ID NO: 341 - DNA, 1275 bp, synthetic construct
  source 1..1275: mol_type = other DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttag

SEQ ID NO: 342 - DNA, 1326 bp, synthetic construct
  source 1..1326: mol_type = other DNA
atgaaggcggccgtcgatctcaagccgactctcaccatcatcaagacggaaaaagtcgatctggagcttttcccctccccggatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtga

SEQ ID NO: 343 - DNA, 1968 bp, synthetic construct
  source 1..1968: mol_type = other DNA
atggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctga

SEQ ID NO: 344 - DNA, 2034 bp, synthetic construct
  source 1..2034: mol_type = other DNA
atgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtga

SEQ ID NO: 345 - DNA, 1296 bp, synthetic construct
  source 1..1296: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 346 - DNA, 1332 bp, synthetic construct
  source 1..1332: mol_type = other DNA
atggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttag

SEQ ID NO: 347 - DNA, 1269 bp, synthetic construct
  source 1..1269: mol_type = other DNA
atgccgcgctcatttctcgtcaaaagcaagaaggctcacagctaccaccagccgcgctccccaggaccagactattccctccgtttagagaatgtaccggcgcctagccgagcagacagcacttcaaatgcaggcggggcgaaggcggagccccgggaccgtttgtcccccgaatcgcagctgaccgaagccccagacagagcctccgcatccccagacagctgcgaaggcagcgtctgcgaacggagctcggagtttgaggacttctggaggcccccgtcaccctccgcgtctccagcctcggagaagtcaatgtgcccatcgctggacgaagcccagcccttccccctgcctttcaaaccgtactcatggagcggcctggcgggttctgacctgcggcacctggtgcagagctaccgaccgtgtggggccctggagcgtggcgctggcctgggcctcttctgcgaacccgccccggagcctggccacccggccgcgctgtacggcccgaagcgggctgccggcggcgcgggggccggggcgccagggagctgcagcgcaggggccggtgccaccgctggccctggcctagggctctacggcgacttcgggtctgcggcagccgggctgtatgagaggcccacggcagcggcgggcttgctgtaccccgagcgtggccacgggctgcacgcagacaagggcgctggcgtcaaggtggagtcggagctgctgtgcacccgcctgctgctgggcggcggctcctacaagtgcatcaagtgcagcaaggtgttctccacgccgcacgggctcgaggtgcacgtgcgcaggtcccacagcggtaccagaccctttgcctgcgagatgtgcggcaagaccttcgggcacgcggtgagcctggagcagcacaaagccgtgcactcgcaggaacggagctttgactgtaagatctgtgggaagagcttcaagaggtcatccacactgtccacacacctgcttatccactcagacactcggccctacccctgtcagtactgtggcaagaggttccaccagaagtcagacatgaagaaacacactttcatccacactggtgagaagcctcacaagtgccaggtgtgcggcaaggcattcagccagagctccaacctcatcacccacagccgcaaacacacaggcttcaagcccttcggctgcgacctctgtgggaagggtttccagaggaaggtggacctccgaaggcaccgggagacgcagcatgggctcaaatga

SEQ ID NO: 348 - DNA, 951 bp, synthetic construct
  source 1..951: mol_type = other DNA
atggcgtctaccagccgtttggatgctcttccaagagtcacatgtccaaaccatccagatgcgattttagtggaggactacagagccggtgatatgatctgtcctgaatgtggcttggttgtaggtgaccgggttattgatgtgggatctgaatggcgaactttcagcaatgacaaagcaacaaaagatccatctcgagttggagattctcagaatcctcttctgagtgatggagatttgtctaccatgattggcaagggcacaggagctgcaagttttgacgaatttggcaattctaagtaccagaatcggagaacaatgagcagttctgatcgggcaatgatgaatgcattcaaagaaatcactaccatggcagacagaatcaatctacctcgaaatatagttgatcgaacaaataatttattcaagcaagtatatgaacagaagagcctgaagggaagagctaatgatgctatagcttctgcttgtctctatattgcctgtagacaagaaggggttcctaggacatttaaagaaatatgtgccgtatcacgaatttctaagaaagaaattggtcggtgttttaaacttattttgaaagcgctagaaaccagtgtggatttgattacaactggggacttcatgtccaggttctgttccaacctttgtcttcctaaacaagtacagatggcagctacacatatagcccgtaaagctgtggaactggacttggttcctgggaggagccccatctctgtggcagcggcagctatttacatggcctcacaggcatcagctgaaaagaggacccaaaaagaaattggagatattgctggtgttgctgatgttacaatcagacagtcctatagactgatctatcctcgagccccagatctgtttcctacagacttcaaatttgacaccccagtggacaaactaccacagctataa

SEQ ID NO: 349 - DNA, 2145 bp, synthetic construct
  source 1..2145: mol_type = other DNA
atgctggacacgatggaggcgcccggccactccaggcagctgctgctgcagctcaacaaccagcgcaccaagggcttcttgtgcgacgtgatcatcgtggtgcagaacgccctcttccgcgcgcacaagaacgtgctggcggccagcagcgcctacctcaagtccctggtggtgcatgacaacctgctcaacctggaccatgacatggtgagcccggccgtgttccgcctggtgctggacttcatctacaccggccgcctggctgacggcgcagaggcggctgcggccgcggccgtggccccgggggctgagccgagcctgggcgccgtgctggccgccgccagctacctgcagatccccgacctcgtggcgctgtgcaagaaacgcctcaagcgccacggcaagtactgccacctgcggggcggcggcggcggcggcggcggctacgcgccctatggtcggccgggccggggcctgcgggccgccacgccggtcatccaggcctgctacccgtccccagtcgggcctccgccgccgcctgccgcggagccgccctcgggcccagaggccgcggtcaacacgcactgcgccgagctgtacgcgtcgggacccggcccggccgccgcactctgtgcctcggagcgccgctgctcccctctttgtggcctggacctgtccaagaagagcccgccgggctccgcggcgccagagcggccgctggctgagcgcgagctgcccccgcgcccggacagccctcccagcgccggccccgccgcctacaaggagccgcctctcgccctgccgtcgctgccgccgctgcccttccagaagctggaggaggccgcaccgccttccgacccatttcgcggcggcagcggcagcccgggacccgagccccccggccgccccgacgggcctagtctcctctatcgctggatgaagcacgagccgggcctgggtagctatggcgacgagctgggccgggagcgcggctcccccagcgagcgctgcgaagagcgtggtggggacgcggccgtctcgcccggggggcccccgctcggcctggcgccgccgccgcgctaccctggcagcctggacgggcccggcgcgggcggcgacggcgacgactacaagagcagcagcgaggagaccggtagcagcgaggaccccagcccgcctggcggccacctcgagggctacccatgcccgcacctggcctatggcgagcccgagagcttcggtgacaacctgtacgtgtgcattccgtgcggcaagggcttccccagctctgagcagctgaacgcgcacgtggaggctcacgtggaggaggaggaagcgctgtacggcagggccgaggcggccgaagtggccgctggggccgccggcctagggcccccttttggaggcggcggggacaaggtcgccggggctccgggtggcctgggagagctgctgcggccctaccgctgcgcgtcgtgcgacaagagctacaaggacccggccacgctgcggcagcacgagaagacgcactggctgacccggccctacccatgcaccatctgcgggaagaagttcacgcagcgtgggaccatgacgcgccacatgcgcagccacctgggcctcaagcccttcgcgtgcgacgcgtgcggcatgcggttcacgcgccagtaccgcctcacggagcacatgcgcatccactcgggcgagaagccctacgagtgccaggtgtgcggcggcaagttcgcacagcaacgcaacctcatcagccacatgaagatgcacgccgtggggggcgcggccggcgcggccggggcgctggcgggcttgggggggctccccggcgtccccggccccgacggcaagggcaagctcgacttccccgagggcgtctttgctgtggctcgcctcacggccgagcagctgagcctgaagcagcaggacaaggcggccgcggccgagctgctggcgcagaccacgcacttcctgcacgaccccaaggtggcgctggagagcctctacccgctggccaagttcacggccgagctgggcctcagccccgacaaggcggccgaggtgctgagccagggcgctcacctggcggccgggcccgacggccggaccatcgaccgtttctctcccacctag

SEQ ID NO: 350 - DNA, 978 bp, synthetic construct
  source 1..978: mol_type = other DNA
atgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtag

SEQ ID NO: 351 - DNA, 1146 bp, synthetic construct
  source 1..1146: mol_type = other DNA
atgtcccgacatgaaggtgtcagctgtgatgcatgtttaaaaggaaattttcgaggtcgcagatataagtgtttaatttgctacgattacgatctttgtgcatcttgttatgaaagtggtgcaacaacaacaaggcatacaactgaccacccaatgcagtgcatattaacaagggtagattttgatttatactatggtggggaagctttctctgtagagcagccacagtcttttacttgtccctattgtggaaaaatgggctatacggagacatctcttcaagaacatgttacttctgaacatgcagaaacatcaacagaagtgatttgtccaatatgtgcagcgttacctggaggcgatcctaatcatgtcacggatgactttgcagctcatcttacacttgaacacagagcccctagagatttagatgaatcgagtggtgttcgacatgtacgtagaatgtttcaccctggccggggattaggaggtcctcgtgctcgtagatcaaacatgcactttactagcagttctactggtggactttcttcttctcagagttcatattctccaagcaatagggaagccatggatcctatagctgagcttttatctcagttatcaggagtgagacgttctgcaggaggacagcttaattcctctggcccttccgcttctcagttacaacaactgcagatgcagctgcagctagaacggcagcatgcccaggcagcacggcaacaactggagaccgcacgcaacgcaacccggcgtactaacacaagcagtgtcaccactacaatcacacaatccacagcaacaaccaacatagctaatacagaaagcagtcagcagactctacagaattcccagtttcttttaacaaggttgaatgatcctaaaatgtctgaaacggagcgccagtccatggaaagcgagcgtgcagaccgcagcctgtttgtccaagagctccttctgtccactttagtgcgtgaagagagctcatcctcagatgaggatgatcggggggagatggcagattttggtgctatgggctgtgtagatattatgcctttagatgttgctttagaaaacctaaatttaaaagagagtaataaaggaaatgagcctccaccacctcctctttga

SEQ ID NO: 352 - DNA, 1236 bp, synthetic construct
  source 1..1236: mol_type = other DNA
atgcgcgcgcacccggggggaggccgctgctgcccggagcaggaggagggggagagtgcggcgggcggcagcggcgctggcggcgactccgccatagagcaggggggccagggcagcgcgctcgccccgtccccggtgagcggcgtgcgcagggaaggcgctcggggcggcggccgtggccgggggcggtggaagcaggcgggccggggcggcggcgtctgtggccgtggccggggccggggccgtggccggggacggggacggggccggggccggggccgcggccgtcccccgagtggcggcagcggccttggcggcgacggcggcggctgcggcggcggcggcagcggtggcggcggcgccccccggcgggagccggtccctttcccgtcggggagcgcggggccggggcccaggggaccccgggccacggagagcgggaagaggatggattgcccggccctcccccccggatggaagaaggaggaagtgatccgaaaatctgggctaagtgctggcaagagcgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatcaaaataagggtaaaccagacttgaatacaacattgccaattagacaaacagcatcaattttcaaacaaccggtaaccaaagtcacaaatcatcctagtaataaagtgaaatcagacccacaacgaatgaatgaacagccacgtcagcttttctgggagaagaggctacaaggacttagtgcatcagatgtaacagaacaaattataaaaaccatggaactacccaaaggtcttcaaggagttggtccaggtagcaatgatgagacccttttatctgctgttgccagtgctttgcacacaagctctgcgccaatcacagggcaagtctccgctgctgtggaaaagaaccctgctgtttggcttaacacatctcaacccctctgcaaagcttttattgtcacagatgaagacatcaggaaacaggaagagcgagtacagcaagtacgcaagaaattggaagaagcactgatggcagacatcttgtcgcgagctgctgatacagaagagatggatattgaaatggacagtggagatgaagcctaa

SEQ ID NO: 353 - DNA, 1362 bp, synthetic construct
  source 1..1362: mol_type = other DNA
ctggatttttttcgggtagtggaaaaccagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 354 - DNA, 2370 bp, synthetic construct
  source 1..2370: mol_type = other DNA
atgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaa

SEQ ID NO: 355 - DNA, 2355 bp, synthetic construct
  source 1..2355: mol_type = other DNA
atggcttctctggacgacccaggggaagtgagggagggcttcctctgccctctgtgcctgaaggatctgcagtctttctatcagcttcactcacattacgaggaagaacactcaggggaagaccgtgatgtcaaagggcaaattaaaagtcttgtccagaaggctaaaaaagcaaaggacaggttgttgaaacgagaaggggatgatcgagcagagtcagggacccaaggatatgagtctttcagctatggaggggttgatccttacatgtgggaaccccaggagcttggtgctgtgagaagccatctttccgacttcaaaaaacaccgagctgctagaattgaccactatgttgtggaagtcaataaactaataatcaggttagagaagctcactgcatttgacagaacaaatactgagtctgcaaagattcgagcaatagaaaagtctgtggtgccttgggtcaacgaccaggatgtccctttctgtccagactgtgggaataagttcagcatccggaaccgccgccaccactgccgcctctgcgggtctattatgtgcaagaagtgtatggagctcatcagccttcccttggcaaacaagctcaccagtgccagcaaggagtccctgagcacccacaccagccccagccagtcacccaacagtgtccatggctcccgccgaggcagcatcagcagcatgagcagtgtcagctcggtcctggatgagaaggacgatgaccggatccgctgctgtacacactgcaaggacacgctgctcaagagagagcagcagattgatgagaaggagcacacacctgacatcgtgaagctctacgagaaattacgactttgcatggagaaagttgaccagaaagctccagaatacatcaggatggcagcatcattaaatgctggggagacaacctacagtctggaacatgccagtgaccttcgagtggaagtgcagaaagtgtatgagctgatagacgctttaagtaagaagatcttaaccttgggcttgaaccaggaccctccaccacatccaagcaatttgcggctgcagagaatgatcagatactcagctacactttttgtgcaggaaaagttgcttggtttgatgtcactgccaaccaaagaacagtttgaggaactgaaaaagaaaaggaaggaggaaatggagaggaagagggccgtggagagacaagctgccctggagtcccagcgaaggcttgaggaaaggcagagtggcctggcttctcgagcggccaacggggaggtggcatctctccgcaggggccctgcccccttgagaaaggctgagggctggctcccactgtcaggaggtcaggggcagagtgaggactcagacccgctcctccagcagatccacaacatcacatcattcatcaggcaggccaaggccgcgggccgcatggatgaagtgcgcactctgcaggagaacctgcggcagctgcaggacgagtatgaccagcagcagacagagaaggccatcgagctgtcccggaggcaggctgaggaggaggacctgcagcgggaacagctgcagatgttgcgtgaacgggagttggaacgagaaagggagcagtttcgggtggcatccctgcacacacggactcggtccctggacttcagagaaatcggcccttttcagctggagcccagcagagagcctcgcacccaccttgcttatgctttggatctaggctcttccccagttccaagcagcacagctcccaagaccccttcacttagctcaactcaacccaccagagtgtggtctgggcccccagccgttggccaggagcgcttaccccagagcagcatgccacagcaacatgaggggccctccttaaacccctttgatgaggaagacctctccagccccatggaagaggccactactggtcctcctgctgcaggggtttccttagacccttcagcccgcatcctgaaagagtacaatcctttcgaggaagaggacgaggaggaggaagcagtggcagggaatccattcattcagccagacagcccagctcctaaccccttcagtgaggaagacgaacatccccagcagaggctctcaagccctctggttcctggtaacccctttgaggaacccacctgtatcaacccctttgagatggacagtgacagtgggccagaggctgaggagcccatagaggaagagctcctcctgcagcagatcgataacatcaaggcatacatctttgatgccaagcagtgcggccgcctggatgaggtagaggtgctgacagagaatctgcgggagctgaagcacaccctggccaagcagaaggggggcactgactga

SEQ ID NO: 356 - DNA, 1362 bp, synthetic construct
  source 1..1362: mol_type = other DNA
atgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactga

SEQ ID NO: 357 - DNA, 1290 bp, synthetic construct
  source 1..1290: mol_type = other DNA
atggcatcgaacagcatcttcgactccttcccgacctactcgccgaccttcatccgcgacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctactga

SEQ ID NO: 358 - DNA, 7281 bp, synthetic construct
  source 1..7281: mol_type = other DNA
atggcgaccaacatcgagcagatttttaggtctttcgtggtcagtaaattccgggaaattcaacaggagctttccagtggaaggaatgaaggccagctgaatggtgaaacaaatacacccattgaaggaaaccaggcgggtgatgcagctgcctctgccaggagtctaccaaatgaagaaatagtgcagaagatagaggaagtactttctggggtcttagatacagaactacgatataagccagacttgaaagagggctccagaaaaagtagatgcgtatctgtacaaacagatcctactgatgaaattcccactaaaaagtcaaagaagcataaaaagcacaaaaacaaaaagaagaaaaagaagaaagaaaaggaaaaaaaatataaaagacagccagaagaatctgagtcaaagacgaaatctcatgatgatgggaacatagatttagaatctgattcctttttaaagtttgattctgaaccttcagctgtggcgctggagcttcctacaagagcatttggcccatctgagaccaatgaatcccctgcagttgtgctagaacctcctgtagtatcaatggaggtatcagagccacacatcttagaaactctgaagccagctacaaaaactgcagaactgtcagttgtatctacatcagtaatctcagagcagtcagagcagtctgtggcagtaatgccagaaccatccatgacaaagattctggattcctttgcagcagcaccagtgcctactacaacactggtgttgaagtcatctgagccagttgtaacaatgtcagtggagtatcagatgaagtctgtgctgaaatctgtggagagcacatctccagagccatcaaagatcatgttggtagagcccccagtagcaaaagtgttagagccttcagaaacccttgtggtatcatcagagacacctactgaggtgtaccctgagccaagcacatcaacaacaatggattttccagagtcatctgcaattgaagcgctaagattgccagagcagcctgtagacgtaccatcggagattgcagattcatccatgacaagaccgcaggagttgccggagctgcctaagaccacagcgttggagctgcaggagtcgtcggtggcctcagcgatggagttgccggggccacctgcgacctccatgccggagttgcaggggccccctgtgactccagtgctggagttacctgggccctctgctaccccggtgccagagttgccagggcccctttctaccccagtgcctgagttgccagggccccctgcgacagcagtgcctgagttgccagggccctctgtgacaccagtgccacagttgtcgcaggaattgccagggcttccagcaccatccatggggttggagccaccacaggaggtaccagagccacctgtgatggcacaggagttgccagggctgcctttggtgacagcagcagtagagttgccagagcagcctgcggtaacagtagcaatggagttgaccgaacaacctgtgacgacgacagagttggagcagcctgtggggatgacaacggtggaacatcctgggcatcctgaggtgacaacggcaacagggttgctggggcagcctgaggcaacgatggtgctggagttgccaggacagccagtggcaacgacagcgctggagttgccggggcagccttcggtgactggggtgccagagttgccagggctgccttcggcaactagggcactggagttgtcggggcagcctgtggcaactggggcactagagttgcctgggccgctcatggcagctggggcactggagttctcggggcagtctggggcagctggagcactggagcttttggggcagcctctggcaacaggggtgctggagttgccagggcagcctggggcgccagagttgcctgggcagcctgtggcaactgtggcgctggagatctctgttcagtctgtggtgacaacatcggagctgtcaacgatgaccgtgtcgcagtccctggaggtgccctcgacgacagcgctggaatcctataatacggtagcacaggagctgcctactacattagtgggggagacttctgtaacagtaggagtggatcccttgatggccccagaatcccatatattagcttctaacaccatggagacccatatattagcatccaacaccatggactcccaaatgctagcgtccaacaccatggactcccagatgctagcatccaacaccatggactcccagatgttagcgtctagcaccatggactcccagatgttagcaactagctccatggactcccagatgttagcaactagctccatggactcccagatgttagcaactagcactatggactcccagatgttagcaaccagttccatggactcccagatgttagcaaccagctccatggactcccagatgttagcaaccagctccatggactcccagatgttagcaaccagctccatggactcccagatgttagcaaccagcaccatggattctcagatgttagcaaccagcaccatggactcccagatgttagcaactagctcaatggattcccagatgttagcatctggcactatggactctcaaatgttagcttctggcaccatggatgctcagatgttagcgtctggtaccatggatgcccagatgttagcgtctagtacccaagattctgctatgttgggttcaaaatctcctgatccctataggttagctcaggatccttacaggttagctcaggatccctataggttgggccatgacccctatagattaggtcatgatgcttacaggttaggacaagacccttatagattaggccatgatccctacagactaactcctgatccctataggatgtcacctagaccctacaggatagcacccaggtcctatagaatagcacccaggccatataggttagcacctagacccctgatgttagcatctagacgttctatgatgatgtcctatgctgcagaacgttccatgatgtcatcttacgaacgctctatgatgtcttatgagcggtctatgatgtcccctatggctgaacgctctatgatgtcagcctacgagcgctctatgatgtcagcctacgagcgctctatgatgtcccctatggctgagcgctctatgatgtcagcttatgaacgctccatgatgtcagcttatgaacgctccatgatgtccccaatggctgatcgatctatgatgtccatgggtgctgaccggtctatgatgtcgtcatactctgctgctgaccggtctatgatgtcatcgtactctgcagctgaccgatctatgatgtcatcttatactgctgatcgttcaatgatgtctatggctgctgattcttacaccgattcttacactgacacatatacagaggcatatatggtgccacctttgcctcctgaagagcccccaacaatgccaccgttgccacctgaggagccaccaatgacaccaccattgcctcctgaggaaccaccagagggtccagcattgcccactgagcagtcagcattaacagctgaaaatacttggcctacagaggtgccatcatcaccatctgaagagtctgtatcgcagcctgagcctcctgtgagtcaaagtgagatttcggagccttcagcagtgcctactgattattcagtgtcagcatcagatccctcagttttagtatcagaggctgctgtgactgttccagaaccaccaccagagccagaatcttcaattacgttaacacctgtagagtctgcagtagtagcagaagaacatgaagttgttccagagagaccagtgacttgtatggtatctgaaactcccgccatgtcagctgaaccaactgtgttagcatcagagcctcctgttatgtcagagacagcagaaacatttgattccatgagagcctcaggacatgttgcctcagaagtatctacatccttgttggttccagcagtaactactccagtgctggcagagagcattctggagccgccagccatggctgccccagagtcttcagctatggctgtcctggagtcttcggctgtgaccgtcctggagtcttcgactgtgactgtcctggagtcttcgactgtaactgtcctggagccttcggttgtgactgtcccggagcctcctgttgtggctgagccagactatgttaccattcctgtgccagttgtttctgcgctggagccttctgtgcctgttctggaaccagcggtgtcagtccttcaaccttctatgattgtttcagaaccatctgtttctgtccaggaatcgactgtgacagtttcagagcctgctgtcacagtctcagagcagactcaagtaataccaactgaggtggctatagagtccacaccaatgatactggaatctagtatcatgtcatcacatgttatgaaaggaattaatctatcctctggtgatcaaaatcttgctccagagattggcatgcaggagattgcattgcattcaggtgaagaaccacatgctgaggaacacctgaaaggtgacttttacgaaagtgaacatggtataaatatagaccttaatataaataatcatttaattgctaaagagatggaacataatacagtgtgtgctgctggtactagtcctgttggggaaattggtgaagagaaaattttgcccaccagtgagactaaacagcgcacagtattggatacctaccctggtgttagtgaagctgatgcaggagaaactctatcttctactggtccttttgctctggaacctgatgcaacaggaactagtaagggtattgaatttaccacagcatctactctcagtttagttaataaatatgatgttgatttatctttaactactcaagatactgaacatgacatggtaatttccaccagtcctagtggtggtagtgaagctgacattgaagggcctttgcctgctaaagatattcatcttgatttaccatctaataataaccttgttagtaaggatacagaagaaccattacctgtaaaagagagtgaccagacattagcagctctgctcagccctaaagaaagtagtggaggagaaaaagaagtacctccccctcctaaagagacactgcctgattcaggattttctgccaatattgaggatattaatgaagcagatttagtgagaccgttacttcctaaggacatggaacgtcttacaagccttagagctggcattgaaggacctttacttgcaagtgatgttggacgtgacagatctgctgccagcccggttgtaagtagtatgccagaaagagcttcagagtcttcttcagaggaaaaagatgattatgaaatttttgtaaaagttaaggacactcacgaaaaaagcaagaaaaataagaaccgtgataagggggagaaagagaagaaaagagactcttcattaagatctcgaagtaagcgttccaaatcttctgaacacaaatcacgcaagcgtaccagtgaatctcgttctagggcaagaaagagatcatctaagtccaagtctcatcgctctcagacacgttcacggtcacgttcaagacgcaggaggagaagcagcagatcaagatcaaagtctagaggaagaagatctgtatcaaaagagaagcgcaaaagatctccaaagcacagatccaagtctagggaaagaaaaagaaaaagatcaagctccagggataaccgaaagacagttagagctcgaagtcgaaccccaagtcgtcggagtcggagtcatactccaagtcgtcgacgaaggtctagatctgtgggtagaagaaggagctttagcatttccccaagccgccgcagccgcacccccagccgccgcagccgcacccccagccgccgcagccgcacccccagccgccgcagccgcacccccagccgccggagccgcacccctagccgtcggagccgcaccccaagccgccggagaagatcaaggtctgtggtaagaagacgaagcttcagtatctcaccagtcagattaaggcgatcaagaacacccttaagaagaaggtttagcagatctcccatccgtcgtaaaagatccaggtcttctgaacgaggcagatcacccaaacgtctgacagatttggataaggctcaattacttgaaatagccaaagctaatgcagctgccatgtgtgctaaggctggtgtccctttaccaccaaacctaaagcctgcacctccacctactatagaagagaaagttgctaaaaagtcaggaggagctactatagaagaactaactgagaaatgtaaacagatcgcacagagtaaagaagatgatgatgtaatagtgaataaacctcatgtttcggatgaagaggaagaagaacctcctttttatcatcatccctttaaactcagtgaacccaaacctatttttttcaatctgaatattgctgcagcaaaaccaactccaccaaaaagccaggtaacattaacaaaagaattccctgtatcatctggatctcaacatcggaaaaaagaagcggatagtgtttatggagaatgggttcctgtggagaaaaatggtgaagaaaacaaagatgatgataatgttttcagcagcaatttgccctcagagcctgtggacatctctacagcaatgagtgaacgggcacttgctcagaaaagactcagtgagaatgcatttgatcttgaagccatgagcatgttaaatagagctcaggaaaggattgatgcctgggctcagctgaactctattcctggccagttcacaggaagtacaggagtacaggttttgacacaagaacagttggccaatactggtgcccaagcctggattaaaaaggatcagttcttaagagcagccccggtaactggaggaatgggagccgttttgatgagaaaaatgggctggagagaaggagaaggattaggaaaaaacaaagaaggcaataaggaacccatcctagttgattttaagacagaccgaaaaggtcttgttgcagtaggagaaagagcacaaaagaggtctgggaacttctctgctgcaatgaaagatctgtcaggcaaacatcctgtgtctgctttgatggagatctgtaataaaagaaggtggcaaccacctgaatttctattggtccatgatagtggccctgatcatcgcaaacattttctctttagggtattgagaaatggagcccttaccagacccaattgtatgtttttcttgaataggtattga

SEQ ID NO: 359 - DNA, 1608 bp, synthetic construct
  source 1..1608: mol_type = other DNA
atgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactga

SEQ ID NO: 360 - DNA, 6072 bp, synthetic construct
  source 1..6072: mol_type = other DNA
atgatccaggaagagcaaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtagagcaaattattgaaaaagatgaaggtcctttttatacccatctaggagcaggtcctaatgtggcagctattagagaaatcatggaagaaaggtttggacagaagggtaaagctattaggattgaaagagtcatctatactggtaaagaaggcaaaagttctcagggatgtcctattgctaagtgggtggttcgcagaagcagcagtgaagagaagctactgtgtttggtgcgggagcgagctggccacacctgtgaggctgcagtgattgtgattctcatcctggtgtgggaaggaatcccgctgtctctggctgacaaactctactcggagcttaccgagacgctgaggaaatacggcacgctcaccaatcgccggtgtgccttgaatgaagagagaacttgcgcctgtcaggggctggatccagaaacctgtggtgcctccttctcttttggttgttcatggagcatgtactacaatggatgtaagtttgccagaagcaagatcccaaggaagtttaagctgcttggggatgacccaaaagaggaagagaaactggagtctcatttgcaaaacctgtccactcttatggcaccaacatataagaaacttgcacctgatgcatataataatcagattgaatatgaacacagagcaccagagtgccgtctgggtctgaaggaaggccgtccattctcaggggtcactgcatgtttggacttctgtgctcatgcccacagagacttgcacaacatgcagaatggcagcacattggtatgcactctcactagagaagacaatcgagaatttggaggaaaacctgaggatgagcagcttcacgttctgcctttatacaaagtctctgacgtggatgagtttgggagtgtggaagctcaggaggagaaaaaacggagtggtgccattcaggtactgagttcttttcggcgaaaagtcaggatgttagcagagccagtcaagacttgccgacaaaggaaactagaagccaagaaagctgcagctgaaaagctttcctccctggagaacagctcaaataaaaatgaaaaggaaaagtcagccccatcacgtacaaaacaaactgaaaacgcaagccaggctaaacagttggcagaacttttgcgactttcaggaccagtcatgcagcagtcccagcagccccagcctctacagaagcagccaccacagccccagcagcagcagagaccccagcagcagcagccacatcaccctcagacagagtctgtcaactcttattctgcttctggatccaccaatccatacatgagacggcccaatccagttagtccttatccaaactcttcacacacttcagatatctatggaagcaccagccctatgaacttctattccacctcatctcaagctgcaggttcatatttgaattcttctaatcccatgaacccttaccctgggcttttgaatcagaatacccaatatccatcatatcaatgcaatggaaacctatcagtggacaactgctccccatatctgggttcctattctccccagtctcagccgatggatctgtataggtatccaagccaagaccctctgtctaagctcagtctaccacccatccatacactttaccagccaaggtttggaaatagccagagttttacatctaaatacttaggttatggaaaccaaaatatgcagggagatggtttcagcagttgtaccattagaccaaatgtacatcatgtagggaaattgcctccttatcccactcatgagatggatggccacttcatgggagccacctctagattaccacccaatctgagcaatccaaacatggactataaaaatggtgaacatcattcaccttctcacataatccataactacagtgcagctccgggcatgttcaacagctctcttcatgccctgcatctccaaaacaaggagaatgacatgctttcccacacagctaatgggttatcaaagatgcttccagctcttaaccatgatagaactgcttgtgtccaaggaggcttacacaaattaagtgatgctaatggtcaggaaaagcagccattggcactagtccagggtgtggcttctggtgcagaggacaacgatgaggtctggtcagacagcgagcagagctttctggatcctgacattgggggagtggccgtggctccaactcatgggtcaattctcattgagtgtgcaaagcgtgagctgcatgccacaacccctttaaagaatcccaataggaatcaccccaccaggatctccctcgtcttttaccagcataagagcatgaatgagccaaaacatggcttggctctttgggaagccaaaatggctgaaaaagcccgtgagaaagaggaagagtgtgaaaagtatggcccagactatgtgcctcagaaatcccatggcaaaaaagtgaaacgggagcctgctgagccacatgaaacttcagagcccacttacctgcgtttcatcaagtctcttgccgaaaggaccatgtccgtgaccacagactccacagtaactacatctccatatgccttcactcgggtcacagggccttacaacagatatatatga

SEQ ID NO: 361 - DNA, 975 bp, synthetic construct
  source 1..975: mol_type = other DNA
atgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaa

SEQ ID NO: 362 - DNA, 1245 bp, synthetic construct
  source 1..1245: mol_type = other DNA
atggcctcgggcgacaccctctacatcgccacggacggctcggagatgccggccgagatcgtggagctgcacgagatcgaggtggagaccatcccggtggagaccatcgagaccacagtggtgggcgaggaggaggaggaggacgacgacgacgaggacggcggcggtggcgaccacggcggcgggggcggccacgggcacgccggccaccaccaccaccaccatcaccaccaccaccacccgcccatgatcgctctgcagccgctggtcaccgacgacccgacccaggtgcaccaccaccaggaggtgatcctggtgcagacgcgcgaggaggtggtgggcggcgacgactcggacgggctgcgcgccgaggacggcttcgaggatcagattctcatcccggtgcccgcgccggccggcggcgacgacgactacattgaacaaacgctggtcaccgtggcggcggccggcaagagcggcggcggcggctcgtcgtcgtcgggaggcggccgcgtcaagaagggcggcggcaagaagagcggcaagaagagttacctcagcggcggggccggcgcggcgggcggcggcggcgccgacccgggcaacaagaagtgggagcagaagcaggtgcagatcaagaccctggagggcgagttctcggtcaccatgtggtcctcagatgaaaaaaaagatattgaccatgagacagtggttgaagaacagatcattggagagaactcacctcctgattattcagaatatatgacaggaaagaaacttcctcctggaggaatacctggcattgacctctcagatcccaaacaactggcagaatttgctagaatgaagccaagaaaaattaaagaagatgatgctccaagaacaatagcttgccctcataaaggctgcacaaagatgttcagggataactcggccatgagaaaacatctgcacacccacggtcccagagtccacgtctgtgcagaatgtggcaaagcttttgttgagagttcaaaactaaaacgacaccaactggttcatactggagagaagccctttcagtgcacgttcgaaggctgtgggaaacgcttttcactggacttcaatttgcgcacacatgtgcgaatccataccggagacaggccctatgtgtgccccttcgatggttgtaataagaagtttgctcagtcaactaacctgaaatctcacatcttaacacatgctaaggccaaaaacaaccagtga

SEQ ID NO: 363 - DNA, 2142 bp, synthetic construct
  source 1..2142: mol_type = other DNA
atggcaaagcccagccacagcagctatgtccttcagcagctaaacaaccaaagagaatggggttttctctgtgactgctgtattgcaattgatgacatttactttcaagcacacaaggcagttctagctgcctgtagctcctattttagaatgtttttcatgaaccatcagcatagtactgcacaactgaatctcagcaacatgaaaattagtgcagaatgttttgatctcattttgcagtttatgtatttaggaaaaattatgacagctccctccagttttgagcagtttaaagtggcaatgaactacctacagctatacaatgttcctgactgtttagaagacatccaggatgcagattgttctagttcaaaatgttcctcttctgcttccagcaaacagaacagcaaaatgatatttggggtaagaatgtatgaagatactgtggctcgaaatggcaatgaagccaacaggtggtgtgcagagccaagttcaacggtaaatacaccacataatagagaggctgatgaagagtctttacaattaggtaattttcctgagccactatttgatgtatgtaaaaaaagttccgtgtccaaattatctactccaaaagaacgtgtgtcaagacgctttgggcggagttttacctgtgatagctgtggatttggctttagctgtgaaaaattattagatgagcatgtgctaacctgtactaacagacatttataccaaaacacaagatcttaccatagaatagtagatattagagatggaaaagacagtaacatcaaagctgaatttggtgaaaaagattcttccaaaacattttctgcacagacggacaaatacagaggagacacaagccaggctgctgatgattcagcttcaaccactggaagcagaaaaagtagcacagtggagtctgaaatagcaagcgaagagaaaagcagagctgctgagaggaaaaggattattattaagatggagccagaagatattcctacagatgaactgaaagactttaacattattaaagttactgataaagactgtaatgaatccactgacaatgatgaattagaagatgaacctgaagagccattttatagatactatgttgaagaagatgtcagcataaaaaaaagtggtaggaaaactctaaaacctcgaatgtcagtaagtgctgatgaaagaggtggtttagagaatatgaggccccctaacaacagcagtccagtacaagaggatgctgaaaatgcatcttgtgagctgtgtggacttacaataaccgaggaggacctgtcatctcattacttagccaaacacattgaaaatatctgtgcatgtggtaaatgtggacaaatacttgtaaagggtaggcagcttcaggaacatgctcaacgatgtggcgagccccaagatctgaccatgaatgggttaggaaatactgaggagaaaatggacttggaagagaatcctgatgagcagtccgaaataagagatatgtttgttgaaatgctggatgattttagggacaatcattaccagataaacagtatccaaaaaaagcagttatttaaacattctgcctgcccttttcgatgtcctaattgtggccagcgttttgaaactgaaaatctagtggttgaacatatgtctagctgcttagatcaagatatgtttaagagtgccatcatggaagaaaatgaaagagatcacagacgaaagcatttttgtaatctgtgtggaaaaggattttatcagcggtgtcacttaagagaacactatactgttcatactaaggaaaaacagtttgtttgtcaaacatgtggaaagcagtttttaagagagcgtcagttgcgactgcacaatgatatgcacaaaggcatggccaggtatgtctgttccatttgtgatcaaggaaacttcagaaaacatgaccatgtacggcatatgatttctcatttatctgctggtgagactatatgccaggtctgctttcagatattcccaaataatgaacagttggagcagcacatggatgttcatctgtatacatgtggaatatgtggagcaaaatttaatttgaggaaagatatgagatcacattataatgccaagcatttgaaaagaacctga

SEQ ID NO: 364 - DNA, 876 bp, synthetic construct
  source 1..876: mol_type = other DNA
atggattttgagaatcttttctcaaaaccccccaacccggccctcggcaaaacggccacggactctgacgaaagaatcgatgatgaaatagatacagaagttgaagaaacacaagaagagaaaattaaactggagtgcgagcaaattcccaaaaaatttagacactctgcaatatcaccaaaaagttcgctgcatagaaaatcaagaagtaaggactatgatgtatatagtgataatgatatctgcagtcaggaatcagaagataattttgccaaagagcttcaacagtacatacaagccagagaaatggcaaatgctgctcaacctgaagaatctacaaagaaagaaggagtaaaagataccccacaggctgctaaacaaaaaaataaaaatcttaaagctggtcacaagaatggcaaacagaagaaaatgaagcgaaaatggcctggccctggaaacaaaggatcaaatgctttgctgaggaacagcggctcacaggaagaggatggtaaacctaaagagaagcagcagcatttgagtcaggcattcatcaaccaacatacagtggaacgcaagggaaaacaaatttgtaaatattttcttgaaaggaaatgtattaagggagaccagtgtaaatttgatcatgatgcagagatagaaaagaaaaaggaaatgtgtaagttttatgtacaaggatattgtaccagaggtgaaaactgtctgtatttgcataatgaatatccttgtaagttttaccatacaggaacaaaatgttatcagggagaatactgcaagttttctcatgctccactgactcctgaaacacaagaattgttggctaaagttttggatactgaaaagaagtcatgtaaataa

SEQ ID NO: 365 - DNA, 2418 bp, synthetic construct
  source 1..2418: mol_type = other DNA
atggatgaagatgggcttgaattacaacaagagccaaactcattttttgatgcaacaggagctgatggtacacacatggatggtgatcaaattgttgtggaagtacaagaaactgtttttgtttcagatgttgtggattcagacataactgtgcataactttgttcctgatgacccagattcagttgtaatccaagatgttattgaggacgttgttatagaagatgttcagtgcccagatatcatggaagaagcagatgtgtctgaaacggtcatcattcctgagcaagtgctggactcagatgtaactgaagaagtttctttagcacattgcacagtcccagatgatgttttagcttctgacattacttcagcctcaatgtctatgccagaacacgtcttgacgggtgattctatacatgtgtctgacgttggacatgttggacatgttggacatgttgaacatgtggttcatgatagtgtagtggaagcagaaattgtcactgatcctctgactaccgacgtagtttcagaagaagtattggtagcagactgtgcctctgaagcagtcatagatgccaatgggatccctgtggaccagcaggatgatgacaaaggcaactgtgaggactaccttatgatttccttggatgatgctggcaaaatagaacacgatggttcttctggaatgaccatggacacagagtcggaaattgatccttgtaaagtggatggcacttgccctgaggtcatcaaggtgtacatttttaaagctgaccctggagaagatgacttaggtggaactgtagacattgtggagagtgagcctgagaatgatcatggagttgaactgcttgatcagaacagcagtattcgtgttcccagggaaaagatggtttatatgactgtcaatgactctcagccagaagatgaagatttaaatgttgctgaaatcgctgacgaagtttatatggaagtgatcgtaggagaggaggatgctgcagcagcagcggcagccgccgccgtgcacgagcagcaaatggatgacaatgaaatcaaaaccttcatgccgattgcatgggcagcagcttatggtaataattctgatggaattgaaaaccggaatggcactgcaagtgccctcttgcacatagatgagtctgctggcctcggcagactggctaaacaaaaaccaaagaaaaggagaagacctgattccaggcagtaccaaacagcaataattattggccctgatggacatcctttgactgtctatccttgcatgatttgtgggaagaagtttaagtcgagaggttttttgaaaaggcacatgaaaaaccatcccgaacaccttgccaagaagaaataccgctgtactgactgtgattacactaccaacaagaagataagtttacacaaccacctggagagccacaagctgaccagcaaggcagagaaggccattgaatgcgatgagtgtgggaagcatttctctcatgcaggggctttgtttactcacaaaatggtgcataaggaaaaaggagccaacaaaatgcacaagtgtaaattctgtgaatacgagacagctgaacaagggttattgaatcgccacctcttggcagtccacagcaagaactttcctcatatttgtgtggagtgtggtaagggttttcgtcacccgtcagagctcaaaaagcacatgagaatccatactggggagaagccgtaccaatgccagtactgcgaatataggtctgcagactcttctaacttgaaaacgcatgtcaaaactaagcatagtaaagagatgccattcaagtgtgacatttgtcttctgactttctcggataccaaagaggtgcagcaacatgctcttatccaccaagaaagcaaaacacaccagtgtttgcattgcgaccacaagagttcgaactcaagtgatttgaaacgacacataatttcagttcacacgaaagactacccccataagtgtgacatgtgtgataaaggctttcacaggccttcagaactcaagaaacacgtggctgcccacaagggcaaaaaaatgcaccagtgtagacattgtgactttaagattgcagatccatttgttctaagtcgccatattctctcagttcacacaaaggatcttccatttaggtgcaagagatgtagaaagggatttaggcaacagagtgagcttaaaaagcatatgaagacacacagtggcaggaaagtgtatcagtgtgagtactgtgagtatagcactacagatgcctcaggctttaaacggcacgttatttccattcacacgaaagactatcctcaccggtgtgagtactgcaagaaaggcttccgaagaccttcagaaaagaaccagcacataatgcgacatcataaagaagttggcctgccctaa

SEQ ID NO: 366 - DNA, 3147 bp, synthetic construct
  source 1..3147: mol_type = other DNA
atgcaatcgaaagtgacaggaaacatgccaactcaatccctcttaatgtacatggatgggccagaagtgattggcagctctcttggcagtccgatggagatggaggatgccttgtcaatgaaagggaccgctgttgttccattccgagctacacaagaaaaaaatgtcatccaaatcgaggggtatatgcccttggattgcatgttctgcagccagaccttcacacattcagaagaccttaataaacatgtcttaatgcaacaccggcctaccctctgtgaaccagcagttcttcgggttgaagcagagtatctcagtccgcttgataaaagtcaagtgcgaacagaacctcccaaggaaaagaattgcaaggaaaatgaatttagctgtgaggtatgtgggcagacatttagagtcgcttttgatgttgagatccacatgagaacacacaaagattctttcacttacgggtgtaacatgtgcggaagaagattcaaggagccttggtttcttaaaaatcacatgcggacacataatggcaaatcgggggccagaagcaaactgcagcaaggcttggagagtagtccagcaacgatcaacgaggtcgtccaggtgcacgcggccgagagcatctcctctccttacaaaatctgcatggtttgtggcttcctatttccaaataaagaaagtctaattgagcaccgcaaggtgcacaccaaaaaaactgctttcggtaccagcagcgcgcagacagactctccacaaggaggaatgccgtcctcgagggaggacttcctgcagttgttcaacttgagaccaaaatctcaccctgaaacggggaagaagcctgtcagatgcatccctcagctcgatccgttcaccaccttccaggcttggcagctggctaccaaaggaaaagttgccatttgccaagaagtgaaggaatcggggcaagaagggagcaccgacaacgacgattcgagttccgagaaggagcttggagaaacaaataagggcagttgtgcaggcctctcgcaagagaaagagaagtgcaaacactcccacggcgaagcgccctccgtggacgcggatcccaagttacccagtagcaaggagaagcccactcactgctccgagtgcggcaaagctttcagaacctaccaccagctggtcttgcactccagggtccacaagaaggaccggagggccggcgcggagtcgcccaccatgtctgtggacgggaggcagccggggacgtgttctcctgacctcgccgcccctctggatgaaaatggagccgtggatcgaggggaaggtggttctgaagacggatctgaggatgggcttcccgaaggaatccatctggataaaaatgatgatggaggaaaaataaaacatcttacatcttcaagagagtgtagttattgtggaaagtttttccgttcaaattattacctcaatattcatctcagaacgcatacaggtgaaaaaccatacaaatgtgaattttgtgaatatgctgcagcccagaagacatctctgaggtatcacttggagagacatcacaaggaaaaacaaaccgatgttgctgctgaagtcaagaacgatggtaaaaatcaggacactgaagatgcactattaaccgctgacagtgcgcaaaccaaaaatttgaaaagattttttgatggtgccaaagatgttacaggcagtccacctgcaaagcagcttaaggagatgccttctgtttttcagaatgttctgggcagcgctgtcctctcaccagcacacaaagatactcaggatttccataaaaatgcagctgatgacagtgctgataaagtgaataaaaaccctacccctgcttacctggacctgttaaaaaagagatcagcagttgaaactcaggcaaataacctcatctgtagaaccaaggcggatgttactcctcctccggatggcagtaccacccataaccttgaagttagccccaaagagaagcaaacggagaccgcagctgactgcagatacaggccaagtgtggattgtcacgaaaaacctttaaatttatccgtgggggctcttcacaattgcccggcaatttctttgagtaaaagtttgattccaagtatcacctgtccattttgtaccttcaagacattttatccagaagttttaatgatgcaccagagactggagcataaatacaatcctgacgttcataaaaactgtcgaaacaagtccttgcttagaagtcgacgtaccggatgcccgccagcgttgctgggaaaagatgtgcctcccctctctagtttctgtaaacccaagcccaagtctgctttcccggcgcagtccaaatccctgccatctgcgaaggggaagcagagccctcctgggccaggcaaggcccctctgacttcagggatagactctagcactttagccccaagtaacctgaagtcccacagaccacagcagaatgtgggggtccaaggggccgccaccaggcaacagcaatctgagatgtttcctaaaaccagtgtttcccctgcaccggataagacaaaaagacccgagacaaaattgaaacctcttccagtagctccttctcagcccaccctcggcagcagtaacatcaatggttccatcgactaccccgccaagaacgacagcccgtgggcacctccgggaagagactatttctgtaatcggagtgccagcaatactgcagcagaatttggtgagccccttccaaaaagactgaagtccagcgtggttgcccttgacgttgaccagcccggggccaattacagaagaggctatgaccttcccaagtaccatatggtcagaggcatcacatcactgttaccgcaggactgtgtgtatccgtcgcaggcgctgcctcccaaaccaaggttcctgagctccagcgaggtcgattctccaaatgtgctgactgttcagaagccctatggtggctccgggccactttacacttgtgtgcctgctggtagtccagcatccagctcgacgttagaaggaaaaaggcctgtgtcatatcaacacttatctaacagcatggcacaaaagagaaactatgagaattttattgggaatgcacattatcgaccaaatgacaaaaaaacttga

SEQ ID NO: 367 - DNA, 4029 bp, synthetic construct
  source 1..4029: mol_type = other DNA
atggaggagaacgaggtggagagcagcagcgacgcggcccctgggcctggccggcccgaggagccctctgagagcggcctgggtgtgggcacctcagaagccgtgtccgccgacagcagcgacgccgcggccgccccggggcaggcagaggccgatgactctggcgtggggcaaagctcggaccgcggcagccgttctcaggaggaggtatctgagagcagctcgagcgcagaccccctgcctaatagctacctccctgattcatcgtctgtgtctcatgggccagtggcaggggtgacaggcggtcccccagcacttgtgcactctagtgcactcccagaccccaacatgctggtgtccgactgcacagcttcctcctcggacctgggctcggccatcgacaagatcatcgagtccaccatcgggcccgacctcatccagaactgcatcactgtgaccagtgctgaggatggcggggccgagaccacacggtacctgatcctacagggcccagatgatggagcccccatgacatcaccaatgtccagttccaccttggcccacagcctagcagccattgaggccctggcagatggccccacatccacatccacatgcctggaggcacagggtgggcccagctccccggtgcagctgcccccagcctccggtgccgaagagccggacctgcagagcctggaggccatgatggaggtggtggtggtgcagcagttcaaatgcaagatgtgccagtaccggagcagcaccaaggccacactgctgcgccacatgcgggaacgccacttccgtccagtagcagcagccgcagcagcagctggtaaaaaaggacgtctacggaagtggagcacctccaccaagagccaagaggaagagggaccagaggaggaggacgatgatgacattgtagacgctggagccattgatgacctggaggaggatagcgactataatccagctgaggatgagccccgaggccggcagcttcggctccagcgccccacccccagtaccccaaggccccgaaggagacctggccggccccggaagctgccccgcctggagatctcagacctcccagatggtgtggaaggagagcctctagtgagttcccagagtggacagagccctccagagccacaggatcccgaggctcccagctcctcaggcccaggacacctggtggccatgggcaaggtgagcaggacccctgtggaagctggtgtgagccagtcagatgcagagaacgcagccccctcctgcccggatgagcatgacactctgccccggcgccgaggtcgaccttccaggcgcttcctaggcaagaaataccgcaagtactattacaagtcgcccaaaccacttttgaggcccttcctgtgccgcatctgtggttctcgctttctgtcccacgaggacctgcgcttccacgtcaactcccatgaggctggcgatccccagctcttcaagtgcctgcagtgcagctatcgttcccgccgctggtcctcgctcaaggagcacatgttcaaccacgtgggcagcaagccctacaagtgtgacgagtgcagctacaccagtgtctaccggaaggacgtcattcggcacgccgctgtgcacagccgggaccggaagaagaggccagatccgactccaaagctgagctctttcccctgccctgtgtgtggccgtgtgtaccccatgcagaaaagactcacgcagcacatgaagacgcacagcactgagaagccccacatgtgtgacaagtgtggaaagtcctttaagaagcgctacaccttcaaaatgcacctgctcacgcacatccaggctgttgccaaccgcaggttcaagtgtgagttctgtgagtttgtttgtgaagacaagaaggcactgctgaaccaccagttgtcccacgtcagtgacaagcccttcaaatgcagcttttgtccctaccgcaccttccgagaggacttcttgctgtcccatgtggctgtcaagcacacaggggccaagcccttcgcctgtgagtactgccacttcagcacacggcacaagaagaacctgcgcctgcacgtacggtgccgacacgcaagcagcttcgaggaatgggggaggcgccaccctgaggagcccccctcccgccgtcgccccttcttctctctgcagcagattgaggagctgaagcagcagcacagtgcggcccctggaccacctcccagttccccaggacctcctgagatacccccagaggcgacaactttccagtcatctgaggctccctcattgctctgttctgacaccctgggcggcgccaccatcatctaccagcaaggagctgaggagtcgacagcgatggccacgcagacagccttggatcttctgctgaacatgagtgctcagcgggaactggggggcacagccctgcaggtggctgtggtgaagtcggaagatgtggaagcagggttagcatcccctggtgggcagccctcccctgaaggtgccactccacaggtggtcaccctccacgtggcagagccagggggcggtgcagcagccgagagccagctaggccctcctgacctaccgcagatcaccctggcacctggtccatttggtgggactggctacagtgtcatcacagcaccccctatggaggagggaacatcagctcctggcacaccttacagcgaggagcccgcaggagaggcagcccaggctgtggttgtgagtgacaccctaaaagaagctggcacccactacatcatggctactgatggtacccagttgcaccacattgagctcaccgcagatggctccatctccttcccaagtccagatgctctggcctctggtgccaaatggcccctgctgcagtgtgggggactgcccagagacggccctgagcccccatctccagccaagacccactgcgtaggggactcccagagctctgcctcctcacctcctgcaaccagcaaagccctgggcctggcagtgcccccgtcaccgccatctgcagccactgctgcatcaaagaagttttcctgcaagatctgtgccgaggccttccctggccgagctgagatggagagtcacaagcgggcccacgctgggcctggtgccttcaagtgccccgactgccccttcagtgcccgccagtggcccgaggtccgggcgcacatggcacagcactcaagcctacggccccaccagtgtagccagtgcagctttgcctccaagaacaagaaggacctgcgtcggcacatgctgactcacacaaaggagaagccttttgcatgccacctctgcgggcagcgtttcaaccgtaacgggcacctcaagttccacatccagcggctgcacagtcctgatgggaggaagtcaggaacccctacagcccgggcccctacccagaccccaacccagaccatcatcctgaacagtgatgacgaaacactggccaccctgcacactgcactccagtccagtcacggggtcctgggcccagagcggctacagcaggcactgagccaggaacacatcatcgttgcccaggaacagacagtgaccaatcaggaggaagccgcctacatccaagagatcaccacggcagatggccagaccgtacagcacctggtgacctccgacaaccaggtgcagtatatcatctcccaggatggtgtccagcacctgctcccccaggaatatgttgtggtccctgaaggccatcacatccaggtacaggagggccagatcacacacatccagtatgaacaaggagccccgttccttcaggagtcccagatccagtatgtgcctgtgtccccaggccagcagcttgtcacacaggctcaacttgaggctgcagcacactcagctgtcacagcagtggctgatgctgccatggcccaagcccagggcctgtttggtacagacgagacagtgcccgaacacattcaacagctgcagcaccagggcatcgagtacgacgtcatcaccctggccgatgactga

SEQ ID NO: 368 - DNA, 1053 bp, synthetic construct
  source 1..1053: mol_type = other DNA
atgatccggggcttcgaggcgcccatggcggagaacccgccgccgccgccgccgcccgtcatcttctgccacgactccccgaagcgggtgctggtgtcggtcatcaggacgaccccgatcaagccaacgtgcggcggtggaggggagccggagccgccgccgccgctcatccccaccagccccggcttcagcgacttcatggtgtacccgtggcgctggggcgagaacgcacacaacgtgacgctcagccctggggccgcgggggccgccgcctcggccgccctgcctgcagccgcagccgccgagcactcggggcttcgtggccggggcgcgcccccgcccgccgcctcggcctccgccgccgcctcgggaggtgaggacgaggaggaagcgagcagcccagacagcggccacctcaaggatggaatccgacgtggtaggcccagagcagatactgtccgcgatttaataaatgaaggagagcattcatccagcagaatccgttgtaacatctgtaatagggtgtttccacgggagaaatcgctccaggctcacaaaaggactcatacaggtgagaggccctatctgtgtgactatccagactgtggaaaagcctttgttcaaagtggacagctcaaaacacatcagcgtcttcacaccggagagaaaccttttgtttgttcagaaaatggctgcctgagcagattcacccatgcaaaccgccactgtccgaagcacccctacgccaggctgaagagagaggagcccacggacacactcagcaaacatcaggctgccgacaacaaggccgcggccgagtggctggcgaggtattgggaaatgagagagcagcgcacccccactttgaaaggcaagctggttcagaaggctgatcaggagcagcaggaccctctggaataccttcagtctgatgaagaggacgacgagaagagaggggcccagcgccggctgcaggagcagcgggagcgcctgcatggagccctcgcgctcatagagcttgccaacctgactggggcgccactccgacagtag

SEQ ID NO: 369 - DNA, 1587 bp, synthetic construct
  source 1..1587: mol_type = other DNA
atgggaccattgcaatttagagatgtggccatagaattctctctggaggagtggcattgcctggacactgcacagcggaatttatataggaatgtgatgttagagaactacagtaacctggtcttccttggtattactgtttctaagccagacctgatcacctgtctggagcaaggaagaaaacctttgaccatgaagagaaatgagatgatagccaaaccctcagtaatgtgttctcattttgcccaagacctttggccagagcagagcatgaaagattctttccaaaaagtggtactgagaagatatgaaaaatgtgaacatgacaatttacagttaaaaaaaggatgtataagtgtggatgagtgtaaggtgcacaaagaaggttataatgaacttaaccaatgtttgacaactaccccaagaaaaatatgtcaatgtgataaatatgtgaaagtccttcatcaatttccaaattcaaacggacaaaagagaggacatactgggaaaaaacctttcaaatatatagaatgtggcaaagcttttaagcagttctcaactcttactacacataagaaaattcatactggagggaaaccctacaaatgtgaagaatgtggcaaagcctttaaccactcttgtagccttactagacataagaaaattcatactggagagaaaccctacaaatgtgaagaatgtggcaaagcctttaagcactcctccactcttactacacataagagaaatcatactggagagaaaccctacaagtgtgataaatgtggcaaagcctttatgtcatcctcaacccttagtaaacatgagataattcatacggaaaagaaaccctacaaatgtgaagaatgtggcaaagccttcaaccggtcctcaacccttactacacataagataattcatactggagaaaaaccctacaaatgtgaagaatgtgacaaagcctttaagtactcctatacccttactacacataaaagaattcatactgaagacaaaccctacaaatgtgaagaatgtggcaaagcctttaagtactcctctacccttactacacataagagaattcatactggagagaaaccctacaaatgtgaagaatgtggcaaagccttcaagcggtcttcagaccttactacgcataagataattcatactggagagaaaccctacaaatgtgaagaatgtggcaaagcttttaagtactcctctaaccttactacacataagaaaattcatactggagagagaccctacaaatgtgaagaatgtggcaaagccttcaaccagtcctcaatccttactacacataggagaattcatactggagagaaattctacaaatgtgaagaatgtggcaaagcttttaagtgctcctctaaccttactacacataagaaaattcatactggagagagaccctacaaatgtgaagaatgtggcaaagccttcaaccagtcctcaatccttactacacatgagagaatcatactggagagaaattctacaaatgtgaagaatgtggcaaagccttcaagcggtcctcacaccttactacacataagataa

SEQ ID NO: 370 - protein, 848 aa, synthetic construct
MNSSSANITYASRKRRKPVQKTVKPIPAEGIKSNPSKRHRDRLNTELDRLASLLPFPQDVINKLDKLSVLRLSVSYLRAKSFFDVALKSSPTERNGGQDNCRAANFREGLNLQEGEFLLQALNGFVLVVTTDALVFYASSTIQDYLGFQQSDVIHQSVYELIHTEDRAEFQRQLHWALNPSQCTESGQGIEEATGLPQTVVCYNPDQIPPENSPLMERCFICRLRCLLDNSSGFLAMNFQGKLKYLHGQKKKGKDGSILPPQLALFAIATPLQPPSILEIRTKNFIFRTKHKLDFTPIGCDAKGRIVLGYTEAELCTRGSGYQFIHAADMLYCAESHIRMIKTGESGMIVFRLLTKNNRWTWVQSNARLLYKNGRPDYIIVTQRPLTDEEGTEHLRKRNTKLPFMFTTGEAVLYEATNPFPAIMDPLPLRTKNGTSGKDSATTSTLSKDSLNPSSLLAAMMQQDESIYLYPASSTSSTAPFENNFFNESMNECRNWQDNTAPMGNDTILKHEQIDQPQDVNSFAGGHPGLFQDSKNSDLYSIMKNLGIDFEDIRHMQNEKFFRNDFSGEVDFRDIDLTDEILTYVQDSLSKSPFIPSDYQQQQSLALNSSCMVQEHLHLEQQQQHHQKQVVVEPQQQLCQKMKHMQVNGMFENWNSNQFVPFNCPQQDPQQYNVFTDLHGISQEFPYKSEMDSMPYTQNFISCNQPVLPQHSKCTELDYPMGSFEPSPYPTTSSLEDFVTCLQLPENQKHGLNPQSAIITPQTCYAGAVSMYQCQPEPQHTHVGQMQYNPVLPGQQAFLNKFQNGVLNETYPAELNNINNTQTTTHLQPLHHPSEARPFPDLTSSGFL

SEQ ID NO: 371 - protein, 789 aa, synthetic construct
MAATTANPEMTSDVPSLGPAIASGNSGPGIQGGGAIVQRAIKRRPGLDFDDDGEGNSKFLRCDDDQMSNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPEAGQGSKFCLVAIGRLQVTSSPNCTDMSNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEPRPTLSNTIQRPQLGPTANLPLEMGSGQLAPRQQQQQTELDMVPGRDGLASYNHSQVVQPVTTTGPEHSKPLEKSDGLFAQDRDPRFSEIYHNINADQSKGISSSTVPATQQLFSQGNTFPPTPRPAENFRNSGLAPPVTIVQPSASAGQMLAQISRHSNPTQGATPTWTPTTRSGFSAQQVATQATAKTRTSQFGVGSFQTPSSFSSMSLPGAPTASPGAAAYPSLTNRGSNFAPETGQTAGQFQTRTAEGVGVWPQWQGQQPHHRSSSSEQHVQQPPAQQPGQPEVFQEMLSMLGDQSNSYNNEEFPDLTMFPPFSE

SEQ ID NO: 372 - protein, 727 aa, synthetic construct
MEGDAVEAIVEESETFIKGKERKTYQRRREGGQEEDACHLPQNQTDGGEVVQDVNSSVQMVMMEQLDPTLLQMKTEVMEGTVAPEAEAAVDDTQIITLQVVNMEEQPINIGELQLVQVPVPVTVPVATTSVEELQGAYENEVSKEGLAESEPMICHTLPLPEGFQVVKVGANGEVETLEQGELPPQEDPSWQKDPDYQPPAKKTKKTKKSKLRYTEEGKDVDVSVYDFEEEQQEGLLSEVNAEKVVGNMKPPKPTKIKKKGVKKTFQCELCSYTCPRRSNLDRHMKSHTDERPHKCHLCGRAFRTVTLLRNHLNTHTGTRPHKCPDCDMAFVTSGELVRHRRYKHTHEKPFKCSMCDYASVEVSKLKRHIRSHTGERPFQCSLCSYASRDTYKLKRHMRTHSGEKPYECYICHARFTQSGTMKMHILQKHTENVAKFHCPHCDTVIARKSDLGVHLRKQHSYIEQGKKCRYCDAVFHERYALIQHQKSHKNEKRFKCDQCDYACRQERHMIMHKRTHTGEKPYACSHCDKTFRQKQLLDMHFKRYHDPNFVPAAFVCSKCGKTFTRRNTMARHADNCAGPDGVEGENGGETKKSKRGRKRKMRSKKEDSSDSENAEPDLDDNEDEEEPAVEIEPEPEPQPVTPAPPPAKKRRGRPPGRTNQPKQNQPTAIIQVEDQNTGAIENIIVEVKKEPDAEPAEGEEEEAQPAATDAPNGDLTPEMILSMMDR

SEQ ID NO: 373 - protein, 1616 aa, synthetic construct
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 374 - protein, 424 aa, synthetic construct
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 375 - protein, 441 aa, synthetic construct
MKAAVDLKPTLTIIKTEKVDLELFPSPDMECADVPLLTPSSKEMMSQALKATFSGFTKEQQRLGIPKDPRQWTETHVRDWVMWAVNEFSLKGVDFQKFCMNGAALCALGKDCFLELAPDFVGDILWEHLEILQKEDVKPYQVNGVNPAYPESRYTSDYFISYGIEHAQCVPPSEFSEPSFITESYQTLHPISSEELLSLKYENDYPSVILRDPLQTDTLQNDYFAIKQEVVTPDNMCMGRTSRGKLGGQDSFESIESYDSCDRLTQSWSSQSSFNSLQRVPSYDSFDSEDYPAALPNHKPKGTFKDYVRDRADLNKDKPVIPAAALAGYTGSGPIQLWQFLLELLTDKSCQSFISWTGDGWEFKLSDPDEVARRWGKRKNKPKMNYEKLSRGLRYYYDKNIIHKTAGKRYVYRFVCDLQSLLGYTPEELHAMLDVKPDADE

SEQ ID NO: 376 - protein, 655 aa, synthetic construct
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 377 - protein, 677 aa, synthetic construct
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 378 - protein, 431 aa, synthetic construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 379 - protein, 443 aa, synthetic construct
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 380 - protein, 422 aa, synthetic construct
MPRSFLVKSKKAHSYHQPRSPGPDYSLRLENVPAPSRADSTSNAGGAKAEPRDRLSPESQLTEAPDRASASPDSCEGSVCERSSEFEDFWRPPSPSASPASEKSMCPSLDEAQPFPLPFKPYSWSGLAGSDLRHLVQSYRPCGALERGAGLGLFCEPAPEPGHPAALYGPKRAAGGAGAGAPGSCSAGAGATAGPGLGLYGDFGSAAAGLYERPTAAAGLLYPERGHGLHADKGAGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK

SEQ ID NO: 381 - protein, 316 aa, synthetic construct
MASTSRLDALPRVTCPNHPDAILVEDYRAGDMICPECGLVVGDRVIDVGSEWRTFSNDKATKDPSRVGDSQNPLLSDGDLSTMIGKGTGAASFDEFGNSKYQNRRTMSSSDRAMMNAFKEITTMADRINLPRNIVDRTNNLFKQVYEQKSLKGRANDAIASACLYIACRQEGVPRTFKEICAVSRISKKEIGRCFKLILKALETSVDLITTGDFMSRFCSNLCLPKQVQMAATHIARKAVELDLVPGRSPISVAAAAIYMASQASAEKRTQKEIGDIAGVADVTIRQSYRLIYPRAPDLFPTDFKFDTPVDKLPQL

SEQ ID NO: 382 - protein, 714 aa, synthetic construct
MLDTMEAPGHSRQLLLQLNNQRTKGFLCDVIIVVQNALFRAHKNVLAASSAYLKSLVVHDNLLNLDHDMVSPAVFRLVLDFIYTGRLADGAEAAAAAAVAPGAEPSLGAVLAAASYLQIPDLVALCKKRLKRHGKYCHLRGGGGGGGGYAPYGRPGRGLRAATPVIQACYPSPVGPPPPPAAEPPSGPEAAVNTHCAELYASGPGPAAALCASERRCSPLCGLDLSKKSPPGSAAPERPLAERELPPRPDSPPSAGPAAYKEPPLALPSLPPLPFQKLEEAAPPSDPFRGGSGSPGPEPPGRPDGPSLLYRWMKHEPGLGSYGDELGRERGSPSERCEERGGDAAVSPGGPPLGLAPPPRYPGSLDGPGAGGDGDDYKSSSEETGSSEDPSPPGGHLEGYPCPHLAYGEPESFGDNLYVCIPCGKGFPSSEQLNAHVEAHVEEEEALYGRAEAAEVAAGAAGLGPPFGGGGDKVAGAPGGLGELLRPYRCASCDKSYKDPATLRQHEKTHWLTRPYPCTICGKKFTQRGTMTRHMRSHLGLKPFACDACGMRFTRQYRLTEHMRIHSGEKPYECQVCGGKFAQQRNLISHMKMHAVGGAAGAAGALAGLGGLPGVPGPDGKGKLDFPEGVFAVARLTAEQLSLKQQDKAAAAELLAQTTHFLHDPKVALESLYPLAKFTAELGLSPDKAAEVLSQGAHLAAGPDGRTIDRFSPT

SEQ ID NO: 383 - protein, 325 aa, synthetic construct
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 384 - protein, 381 aa, synthetic construct
MSRHEGVSCDACLKGNFRGRRYKCLICYDYDLCASCYESGATTTRHTTDHPMQCILTRVDFDLYYGGEAFSVEQPQSFTCPYCGKMGYTETSLQEHVTSEHAETSTEVICPICAALPGGDPNHVTDDFAAHLTLEHRAPRDLDESSGVRHVRRMFHPGRGLGGPRARRSNMHFTSSSTGGLSSSQSSYSPSNREAMDPIAELLSQLSGVRRSAGGQLNSSGPSASQLQQLQMQLQLERQHAQAARQQLETARNATRRTNTSSVTTTITQSTATTNIANTESSQQTLQNSQFLLTRLNDPKMSETERQSMESERADRSLFVQELLLSTLVREESSSSDEDDRGEMADFGAMGCVDIMPLDVALENLNLKESNKGNEPPPPPL

SEQ ID NO: 385 - protein, 411 aa, synthetic construct
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 386 - protein, 453 aa, synthetic construct
MDFFRVVENQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 387 - protein, 789 aa, synthetic construct
MKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 388 - protein, 784 aa, synthetic construct
MASLDDPGEVREGFLCPLCLKDLQSFYQLHSHYEEEHSGEDRDVKGQIKSLVQKAKKAKDRLLKREGDDRAESGTQGYESFSYGGVDPYMWEPQELGAVRSHLSDFKKHRAARIDHYVVEVNKLIIRLEKLTAFDRTNTESAKIRAIEKSVVPWVNDQDVPFCPDCGNKFSIRNRRHHCRLCGSIMCKKCMELISLPLANKLTSASKESLSTHTSPSQSPNSVHGSRRGSISSMSSVSSVLDEKDDDRIRCCTHCKDTLLKREQQIDEKEHTPDIVKLYEKLRLCMEKVDQKAPEYIRMAASLNAGETTYSLEHASDLRVEVQKVYELIDALSKKILTLGLNQDPPPHPSNLRLQRMIRYSATLFVQEKLLGLMSLPTKEQFEELKKKRKEEMERKRAVERQAALESQRRLEERQSGLASRAANGEVASLRRGPAPLRKAEGWLPLSGGQGQSEDSDPLLQQIHNITSFIRQAKAAGRMDEVRTLQENLRQLQDEYDQQQTEKAIELSRRQAEEEDLQREQLQMLRERELEREREQFRVASLHTRTRSLDFREIGPFQLEPSREPRTHLAYALDLGSSPVPSSTAPKTPSLSSTQPTRVWSGPPAVGQERLPQSSMPQQHEGPSLNPFDEEDLSSPMEEATTGPPAAGVSLDPSARILKEYNPFEEEDEEEEAVAGNPFIQPDSPAPNPFSEEDEHPQQRLSSPLVPGNPFEEPTCINPFEMDSDSGPEAEEPIEEELLLQQIDNIKAYIFDAKQCGRLDEVEVLTENLRELKHTLAKQKGGTD

SEQ ID NO: 389 - protein, 453 aa, synthetic construct
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 390 - protein, 429 aa, synthetic construct
MASNSIFDSFPTYSPTFIRDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 391 - protein, 2426 aa, synthetic construct
MATNIEQIFRSFVVSKFREIQQELSSGRNEGQLNGETNTPIEGNQAGDAAASARSLPNEEIVQKIEEVLSGVLDTELRYKPDLKEGSRKSRCVSVQTDPTDEIPTKKSKKHKKHKNKKKKKKKEKEKKYKRQPEESESKTKSHDDGNIDLESDSFLKFDSEPSAVALELPTRAFGPSETNESPAVVLEPPVVSMEVSEPHILETLKPATKTAELSVVSTSVISEQSEQSVAVMPEPSMTKILDSFAAAPVPTTTLVLKSSEPVVTMSVEYQMKSVLKSVESTSPEPSKIMLVEPPVAKVLEPSETLVVSSETPTEVYPEPSTSTTMDFPESSAIEALRLPEQPVDVPSEIADSSMTRPQELPELPKTTALELQESSVASAMELPGPPATSMPELQGPPVTPVLELPGPSATPVPELPGPLSTPVPELPGPPATAVPELPGPSVTPVPQLSQELPGLPAPSMGLEPPQEVPEPPVMAQELPGLPLVTAAVELPEQPAVTVAMELTEQPVTTTELEQPVGMTTVEHPGHPEVTTATGLLGQPEATMVLELPGQPVATTALELPGQPSVTGVPELPGLPSATRALELSGQPVATGALELPGPLMAAGALEFSGQSGAAGALELLGQPLATGVLELPGQPGAPELPGQPVATVALEISVQSVVTTSELSTMTVSQSLEVPSTTALESYNTVAQELPTTLVGETSVTVGVDPLMAPESHILASNTMETHILASNTMDSQMLASNTMDSQMLASNTMDSQMLASSTMDSQMLATSSMDSQMLATSSMDSQMLATSTMDSQMLATSSMDSQMLATSSMDSQMLATSSMDSQMLATSSMDSQMLATSTMDSQMLATSTMDSQMLATSSMDSQMLASGTMDSQMLASGTMDAQMLASGTMDAQMLASSTQDSAMLGSKSPDPYRLAQDPYRLAQDPYRLGHDPYRLGHDAYRLGQDPYRLGHDPYRLTPDPYRMSPRPYRIAPRSYRIAPRPYRLAPRPLMLASRRSMMMSYAAERSMMSSYERSMMSYERSMMSPMAERSMMSAYERSMMSAYERSMMSPMAERSMMSAYERSMMSAYERSMMSPMADRSMMSMGADRSMMSSYSAADRSMMSSYSAADRSMMSSYTADRSMMSMAADSYTDSYTDTYTEAYMVPPLPPEEPPTMPPLPPEEPPMTPPLPPEEPPEGPALPTEQSALTAENTWPTEVPSSPSEESVSQPEPPVSQSEISEPSAVPTDYSVSASDPSVLVSEAAVTVPEPPPEPESSITLTPVESAVVAEEHEVVPERPVTCMVSETPAMSAEPTVLASEPPVMSETAETFDSMRASGHVASEVSTSLLVPAVTTPVLAESILEPPAMAAPESSAMAVLESSAVTVLESSTVTVLESSTVTVLEPSVVTVPEPPVVAEPDYVTIPVPVVSALEPSVPVLEPAVSVLQPSMIVSEPSVSVQESTVTVSEPAVTVSEQTQVIPTEVAIESTPMILESSIMSSHVMKGINLSSGDQNLAPEIGMQEIALHSGEEPHAEEHLKGDFYESEHGINIDLNINNHLIAKEMEHNTVCAAGTSPVGEIGEEKILPTSETKQRTVLDTYPGVSEADAGETLSSTGPFALEPDATGTSKGIEFTTASTLSLVNKYDVDLSLTTQDTEHDMVISTSPSGGSEADIEGPLPAKDIHLDLPSNNNLVSKDTEEPLPVKESDQTLAALLSPKESSGGEKEVPPPPKETLPDSGFSANIEDINEADLVRPLLPKDMERLTSLRAGIEGPLLASDVGRDRSAASPVVSSMPERASESSSEEKDDYEIFVKVKDTHEKSKKNKNRDKGEKEKKRDSSLRSRSKRSKSSEHKSRKRTSESRSRARKRSSKSKSHRSQTRSRSRSRRRRRSSRSRSKSRGRRSVSKEKRKRSPKHRSKSRERKRKRSSSRDNRKTVRARSRTPSRRSRSHTPSRRRRSRSVGRRRSFSISPSRRSRTPSRRSRTPSRRSRTPSRRSRTPSRRSRTPSRRSRTPSRRRRSRSVVRRRSFSISPVRLRRSRTPLRRRFSRSPIRRKRSRSSERGRSPKRLTDLDKAQLLEIAKANAAAMCAKAGVPLPPNLKPAPPPTIEEKVAKKSGGATIEELTEKCKQIAQSKEDDDVIVNKPHVSDEEEEEPPFYHHPFKLSEPKPIFFNLNIAAAKPTPPKSQVTLTKEFPVSSGSQHRKKEADSVYGEWVPVEKNGEENKDDDNVFSSNLPSEPVDISTAMSERALAQKRLSENAFDLEAMSMLNRAQERIDAWAQLNSIPGQFTGSTGVQVLTQEQLANTGAQAWIKKDQFLRAAPVTGGMGAVLMRKMGWREGEGLGKNKEGNKEPILVDFKTDRKGLVAVGERAQKRSGNFSAAMKDLSGKHPVSALMEICNKRRWQPPEFLLVHDSGPDHRKHFLFRVLRNGALTRPNCMFFLNRY

SEQ ID NO: 392 - protein, 535 aa, synthetic construct
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 393 - protein, 2023 aa, synthetic construct
MIQEEQIQLEGSLVDGPEASLMEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 394 - protein, 324 aa, synthetic construct
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 395 - protein, 414 aa, synthetic construct
MASGDTLYIATDGSEMPAEIVELHEIEVETIPVETIETTVVGEEEEEDDDDEDGGGGDHGGGGGHGHAGHHHHHHHHHHHPPMIALQPLVTDDPTQVHHHQEVILVQTREEVVGGDDSDGLRAEDGFEDQILIPVPAPAGGDDDYIEQTLVTVAAAGKSGGGGSSSSGGGRVKKGGGKKSGKKSYLSGGAGAAGGGGADPGNKKWEQKQVQIKTLEGEFSVTMWSSDEKKDIDHETVVEEQIIGENSPPDYSEYMTGKKLPPGGIPGIDLSDPKQLAEFARMKPRKIKEDDAPRTIACPHKGCTKMFRDNSAMRKHLHTHGPRVHVCAECGKAFVESSKLKRHQLVHTGEKPFQCTFEGCGKRFSLDFNLRTHVRIHTGDRPYVCPFDGCNKKFAQSTNLKSHILTHAKAKNNQ

SEQ ID NO: 396 - protein, 713 aa, synthetic construct
MAKPSHSSYVLQQLNNQREWGFLCDCCIAIDDIYFQAHKAVLAACSSYFRMFFMNHQHSTAQLNLSNMKISAECFDLILQFMYLGKIMTAPSSFEQFKVAMNYLQLYNVPDCLEDIQDADCSSSKCSSSASSKQNSKMIFGVRMYEDTVARNGNEANRWCAEPSSTVNTPHNREADEESLQLGNFPEPLFDVCKKSSVSKLSTPKERVSRRFGRSFTCDSCGFGFSCEKLLDEHVLTCTNRHLYQNTRSYHRIVDIRDGKDSNIKAEFGEKDSSKTFSAQTDKYRGDTSQAADDSASTTGSRKSSTVESEIASEEKSRAAERKRIIIKMEPEDIPTDELKDFNIIKVTDKDCNESTDNDELEDEPEEPFYRYYVEEDVSIKKSGRKTLKPRMSVSADERGGLENMRPPNNSSPVQEDAENASCELCGLTITEEDLSSHYLAKHIENICACGKCGQILVKGRQLQEHAQRCGEPQDLTMNGLGNTEEKMDLEENPDEQSEIRDMFVEMLDDFRDNHYQINSIQKKQLFKHSACPFRCPNCGQRFETENLVVEHMSSCLDQDMFKSAIMEENERDHRRKHFCNLCGKGFYQRCHLREHYTVHTKEKQFVCQTCGKQFLRERQLRLHNDMHKGMARYVCSICDQGNFRKHDHVRHMISHLSAGETICQVCFQIFPNNEQLEQHMDVHLYTCGICGAKFNLRKDMRSHYNAKHLKRT

SEQ ID NO: 397 - protein, 291 aa, synthetic construct [sequence omitted]

SEQ ID NO: 398 - protein, 805 aa, synthetic construct
MDEDGLELQQEPNSFFDATGADGTHMDGDQIVVEVQETVFVSDVVDSDITVHNFVPDDPDSVVIQDVIEDVVIEDVQCPDIMEEADVSETVIIPEQVLDSDVTEEVSLAHCTVPDDVLASDITSASMSMPEHVLTGDSIHVSDVGHVGHVGHVEHVVHDSVVEAEIVTDPLTTDVVSEEVLVADCASEAVIDANGIPVDQQDDDKGNCEDYLMISLDDAGKIEHDGSSGMTMDTESEIDPCKVDGTCPEVIKVYIFKADPGEDDLGGTVDIVESEPENDHGVELLDQNSSIRVPREKMVYMTVNDSQPEDEDLNVAEIADEVYMEVIVGEEDAAAAAAAAAVHEQQMDDNEIKTFMPIAWAAAYGNNSDGIENRNGTASALLHIDESAGLGRLAKQKPKKRRRPDSRQYQTAIIIGPDGHPLTVYPCMICGKKFKSRGFLKRHMKNHPEHLAKKKYRCTDCDYTTNKKISLHNHLESHKLTSKAEKAIECDECGKHFSHAGALFTHKMVHKEKGANKMHKCKFCEYETAEQGLLNRHLLAVHSKNFPHICVECGKGFRHPSELKKHMRIHTGEKPYQCQYCEYRSADSSNLKTHVKTKHSKEMPFKCDICLLTFSDTKEVQQHALIHQESKTHQCLHCDHKSSNSSDLKRHIISVHTKDYPHKCDMCDKGFHRPSELKKHVAAHKGKKMHQCRHCDFKIADPFVLSRHILSVHTKDLPFRCKRCRKGFRQQSELKKHMKTHSGRKVYQCEYCEYSTTDASGFKRHVISIHTKDYPHRCEYCKKGFRRPSEKNQHIMRHHKEVGLP

SEQ ID NO: 399 - protein, 1048 aa, synthetic construct
MQSKVTGNMPTQSLLMYMDGPEVIGSSLGSPMEMEDALSMKGTAVVPFRATQEKNVIQIEGYMPLDCMFCSQTFTHSEDLNKHVLMQHRPTLCEPAVLRVEAEYLSPLDKSQVRTEPPKEKNCKENEFSCEVCGQTFRVAFDVEIHMRTHKDSFTYGCNMCGRRFKEPWFLKNHMRTHNGKSGARSKLQQGLESSPATINEVVQVHAAESISSPYKICMVCGFLFPNKESLIEHRKVHTKKTAFGTSSAQTDSPQGGMPSSREDFLQLFNLRPKSHPETGKKPVRCIPQLDPFTTFQAWQLATKGKVAICQEVKESGQEGSTDNDDSSSEKELGETNKGSCAGLSQEKEKCKHSHGEAPSVDADPKLPSSKEKPTHCSECGKAFRTYHQLVLHSRVHKKDRRAGAESPTMSVDGRQPGTCSPDLAAPLDENGAVDRGEGGSEDGSEDGLPEGIHLDKNDDGGKIKHLTSSRECSYCGKFFRSNYYLNIHLRTHTGEKPYKCEFCEYAAAQKTSLRYHLERHHKEKQTDVAAEVKNDGKNQDTEDALLTADSAQTKNLKRFFDGAKDVTGSPPAKQLKEMPSVFQNVLGSAVLSPAHKDTQDFHKNAADDSADKVNKNPTPAYLDLLKKRSAVETQANNLICRTKADVTPPPDGSTTHNLEVSPKEKQTETAADCRYRPSVDCHEKPLNLSVGALHNCPAISLSKSLIPSITCPFCTFKTFYPEVLMMHQRLEHKYNPDVHKNCRNKSLLRSRRTGCPPALLGKDVPPLSSFCKPKPKSAFPAQSKSLPSAKGKQSPPGPGKAPLTSGIDSSTLAPSNLKSHRPQQNVGVQGAATRQQQSEMFPKTSVSPAPDKTKRPETKLKPLPVAPSQPTLGSSNINGSIDYPAKNDSPWAPPGRDYFCNRSASNTAAEFGEPLPKRLKSSVVALDVDQPGANYRRGYDLPKYHMVRGITSLLPQDCVYPSQALPPKPRFLSSSEVDSPNVLTVQKPYGGSGPLYTCVPAGSPASSSTLEGKRPVSYQHLSNSMAQKRNYENFIGNAHYRPNDKKT

SEQ ID NO: 400 - protein, 1342 aa, synthetic construct
MEENEVESSSDAAPGPGRPEEPSESGLGVGTSEAVSADSSDAAAAPGQAEADDSGVGQSSDRGSRSQEEVSESSSSADPLPNSYLPDSSSVSHGPVAGVTGGPPALVHSSALPDPNMLVSDCTASSSDLGSAIDKIIESTIGPDLIQNCITVTSAEDGGAETTRYLILQGPDDGAPMTSPMSSSTLAHSLAAIEALADGPTSTSTCLEAQGGPSSPVQLPPASGAEEPDLQSLEAMMEVVVVQQFKCKMCQYRSSTKATLLRHMRERHFRPVAAAAAAAGKKGRLRKWSTSTKSQEEEGPEEEDDDDIVDAGAIDDLEEDSDYNPAEDEPRGRQLRLQRPTPSTPRPRRRPGRPRKLPRLEISDLPDGVEGEPLVSSQSGQSPPEPQDPEAPSSSGPGHLVAMGKVSRTPVEAGVSQSDAENAAPSCPDEHDTLPRRRGRPSRRFLGKKYRKYYYKSPKPLLRPFLCRICGSRFLSHEDLRFHVNSHEAGDPQLFKCLQCSYRSRRWSSLKEHMFNHVGSKPYKCDECSYTSVYRKDVIRHAAVHSRDRKKRPDPTPKLSSFPCPVCGRVYPMQKRLTQHMKTHSTEKPHMCDKCGKSFKKRYTFKMHLLTHIQAVANRRFKCEFCEFVCEDKKALLNHQLSHVSDKPFKCSFCPYRTFREDFLLSHVAVKHTGAKPFACEYCHFSTRHKKNLRLHVRCRHASSFEEWGRRHPEEPPSRRRPFFSLQQIEELKQQHSAAPGPPPSSPGPPEIPPEATTFQSSEAPSLLCSDTLGGATIIYQQGAEESTAMATQTALDLLLNMSAQRELGGTALQVAVVKSEDVEAGLASPGGQPSPEGATPQVVTLHVAEPGGGAAAESQLGPPDLPQITLAPGPFGGTGYSVITAPPMEEGTSAPGTPYSEEPAGEAAQAVVVSDTLKEAGTHYIMATDGTQLHHIELTADGSISFPSPDALASGAKWPLLQCGGLPRDGPEPPSPAKTHCVGDSQSSASSPPATSKALGLAVPPSPPSAATAASKKFSCKICAEAFPGRAEMESHKRAHAGPGAFKCPDCPFSARQWPEVRAHMAQHSSLRPHQCSQCSFASKNKKDLRRHMLTHTKEKPFACHLCGQRFNRNGHLKFHIQRLHSPDGRKSGTPTARAPTQTPTQTIILNSDDETLATLHTALQSSHGVLGPERLQQALSQEHIIVAQEQTVTNQEEAAYIQEITTADGQTVQHLVTSDNQVQYIISQDGVQHLLPQEYVVVPEGHHIQVQEGQITHIQYEQGAPFLQESQIQYVPVSPGQQLVTQAQLEAAAHSAVTAVADAAMAQAQGLFGTDETVPEHIQQLQHQGIEYDVITLADD

SEQ ID NO: 401 - protein, 350 aa, synthetic construct
MIRGFEAPMAENPPPPPPPVIFCHDSPKRVLVSVIRTTPIKPTCGGGGEPEPPPPLIPTSPGFSDFMVYPWRWGENAHNVTLSPGAAGAAASAALPAAAAAEHSGLRGRGAPPPAASASAAASGGEDEEEASSPDSGHLKDGIRRGRPRADTVRDLINEGEHSSSRIRCNICNRVFPREKSLQAHKRTHTGERPYLCDYPDCGKAFVQSGQLKTHQRLHTGEKPFVCSENGCLSRFTHANRHCPKHPYARLKREEPTDTLSKHQAADNKAAAEWLARYWEMREQRTPTLKGKLVQKADQEQQDPLEYLQSDEEDDEKRGAQRRLQEQRERLHGALALIELANLTGAPLRQ

SEQ ID NO: 402 - protein, 528 aa, synthetic construct
MGPLQFRDVAIEFSLEEWHCLDTAQRNLYRNVMLENYSNLVFLGITVSKPDLITCLEQGRKPLTMKRNEMIAKPSVMCSHFAQDLWPEQSMKDSFQKVVLRRYEKCEHDNLQLKKGCISVDECKVHKEGYNELNQCLTTTPRKICQCDKYVKVLHQFPNSNGQKRGHTGKKPFKYIECGKAFKQFSTLTTHKKIHTGGKPYKCEECGKAFNHSCSLTRHKKIHTGEKPYKCEECGKAFKHSSTLTTHKRNHTGEKPYKCDKCGKAFMSSSTLSKHEIIHTEKKPYKCEECGKAFNRSSTLTTHKIIHTGEKPYKCEECDKAFKYSYTLTTHKRIHTEDKPYKCEECGKAFKYSSTLTTHKRIHTGEKPYKCEECGKAFKRSSDLTTHKIIHTGEKPYKCEECGKAFKYSSNLTTHKKIHTGERPYKCEECGKAFNQSSILTTHRRIHTGEKFYKCEECGKAFKCSSNLTTHKKIHTGERPYKCEECGKAFNQSSILTTHERIILERNSTNVKNVAKPSSGPHTLLHIR